(19) United States
(12) Patent Application Publication
(10) Pub. No.: US 2023/0050791 A1
(43) Pub. Date: Feb. 16, 2023

(54) Medication Delivery System and Method

(71) Applicant: Sadleir Laboratories Pty Ltd, Swanbourne, WA (US)

(72) Inventors: Paul Harold Martin Sadleir, Swanbourne (AU); John Willoughby Sadleir, Swanbourne (AU)

(21) Appl. No.: 17/837,273

(22) Filed: Oct. 24, 2022

Related U.S. Application Data
(63) Continuation-in-part of application No. PCT/AU2020/051363, filed on Dec. 11, 2020.

(30) Foreign Application Priority Data
Dec. 11, 2019 (AU) 2019904695
Oct. 6, 2020 (AU) 2020903614

(51) Int. Cl. A61M 5/14

(52) U.S. Cl. CPC A61M 5/14; A61M 2005/14268

(57) ABSTRACT

According to one example of the present disclosure an infusion device is configured to control a medication delivery apparatus to intravenously deliver a pharmaceutical preparation to a patient. The infusion device comprises a processor and a memory storing instructions executable by the processor to cause the medication delivery apparatus to deliver the pharmaceutical preparation to the patient according to a predetermined dose profile. The predetermined dose profile is designed to deliver a therapeutic dose of the pharmaceutical preparation to the patient over a predetermined infusion time in a manner which facilitates safe detection of an adverse reaction of the patient to the pharmaceutical preparation, or desensitization the patient to the pharmaceutical preparation, during a first stage of administering the therapeutic dose.

### TECHNICAL FIELD

The present disclosure relates to systems and methods for administering pharmaceutical preparations to patients.

The disclosure has been devised particularly, although not necessarily solely, in relation to administering pharmaceutical preparations to patients in particular test doses for, for example, detecting an adverse reaction during the administration of the pharmaceutical preparation, desensitising the patient to the pharmaceutical preparation s or challenging a patient with the pharmaceutical preparation/s to determine if the pharmaceutical preparation/s are responsible for any adverse reaction in the patient.

### BACKGROUND ART

The following discussion of the background art is intended to facilitate an understanding of the present disclosure only. The discussion is not an acknowledgement or admission that any of the material referred to is or was part of the common general knowledge as at the priority date of the application.

Administering a pharmaceutical preparation/s (such as intravenous drugs) to patients has its risks. This is particularly true in patients that may have a drug hypersensitivity reaction to a particular intravenous drug during administration of the particular intravenous drug to these particular patients.

Unfortunately, drug hypersensitivity reactions to particular intravenous drugs are typically unpredictable; and in particular, it is unpredictable the specific dose of the drug that may induce a drug hypersensitivity reaction in a particular patient.

In order to reduce the risk of any patient suffering a life-threatening reaction to a drug, one method of administering a particular intravenous drug is to give the patient a specific dose (referred to as a test dose) that would cause a submaximal adverse response. Upon detection of any submaximal or minor adverse reaction in the particular patient, the process of administering the intravenous drug may be immediately aborted to impede that any more of the pharmaceutical preparation (drug) be administered to the patient and preventing a more serious adverse reaction from developing, or ultimate death of the patient.

However, the practice of administering a test dose is not routine nor recommended. This is particularly true because:

the test dose that will typically elicit a submaximal reaction is typically of the order of 0.01% or 0.1% of the total therapeutic dose to be given to the patient, the preparation of which is time-consuming and difficult; and

test doses that will elicit a detectable submaximal reaction vary between patients, and may be 0.01%, 1%, 10% or 100% of the therapeutic dose.

These two reasons among others make it difficult or even impossible for a clinician to choose the appropriate test dose with which to conduct a trial to confirm whether an adverse reaction will occur during administration of the total therapeutic dose. In particular: administering a test dose that is relatively small may not elicit or result in detection of an adverse reaction in the patient. In contrast, a relatively large dose (above a specific threshold particular to each patient) may cause an adverse reaction that may result in a life-threatening reaction in the patient. This reaction may lead to death of the patient. Thus, administering the test dose may lead to a life-threatening condition that the provision of the test dose had the intention to prevent.

The process for confirming that a particular drug is responsible for a particular adverse reaction in a particular patient by administering a test dose of the particular drug in one or more incremental steps is called Drug Challenge.

Another process where a relative low dose (a test dose) of a drug may be administered to a patient prior to administering the full dose is called Drug Desensitisation. Drug desensitization is the process of administering a test dose below the threshold that will produce an adverse reaction to a patient who is hypersensitive or allergic to a particular drug to induce a state of tolerance and allow administration of the therapeutic dose while avoiding any adverse reaction or inducing only minor non-life threatening reactions.

Typically, drug desensitisation comprises initially administering a dose (the test dose) that is lower than the actual dose that will elicit an adverse reaction in a patient. Subsequently, depending on whether the patient's reaction is favourable to the drug, larger doses are administered to the patient. Administration, typically, occurs at intervals of usually days or weeks; but, on occasions, it may take hours if express desensitisation is required in, for example, emergencies. The process of drug desensitisation is continued until it is certain that the actual dose can be safely administered to the patient without adverse reaction. In particular, for intravenous drugs, administration of the drug occurs as a constant infusion rate of the lower dose for a particular interval, and then the drug is administered as a constant infusion at a higher rate or higher concentration for an interval, and so on, until the therapeutic dose is tolerated.

Unfortunately, due the difficulties in determining what specific percentage of the total therapeutic dose to be administered to the patient is an appropriate test dose for that particular patient, the current practice is to administer intravenous drugs via constant infusion (either brief (‘push’) or over a fixed time period). This has its risks, as mentioned above. Administering the total therapeutic dose of a drug without confirming whether the patients is hypersensitive or allergic to that particular may result in administering a lethal drug dose to a particular patient, or cause a serious negative reaction.

Furthermore, currently any test dose that may be administered to a patient is necessarily done prior to, and separate from, infusion of the therapeutic dose that a particular patient requires. Preparation of separate test doses requires preparation of a multitude of pharmaceutical preparations for each test dose and also for the therapeutic dose. This process is cumbersome and therefore, typically, test doses are not provided to patients. Instead the therapeutic dose is provided to the patient without having tested the reaction of the patient to the drug. This increases the risk that particular patients (that have a drug hypersensitivity reaction to a particular drug) may suffer life threatening conditions while being administered this particular drug. This is particularly true because the current methods for administering the full therapeutic dose (a constant infusion or ‘push’) provide relatively large doses at the start of the infusion process compared to that typically required to cause a serious adverse reaction. This does not allow enough time for the clinician to detect that the patient being infused the pharmaceutical preparation is having a negative (i.e., adverse) reaction to the drug.

### SUMMARY OF DISCLOSURE

According to one aspect of the present disclosure there is provided an infusion device configured to control a medication delivery apparatus to intravenously deliver a pharmaceutical preparation to a patient, the infusion device comprising a processor and a memory storing instructions executable by the processor to cause the medication delivery apparatus to deliver the pharmaceutical preparation to the patient according to a predetermined dose profile, wherein the predetermined dose profile is designed to deliver a therapeutic dose of the pharmaceutical preparation to the patient over a predetermined infusion time in a manner which facilitates safe detection of an adverse reaction of the patient to the pharmaceutical preparation, or desensitization the patient to the pharmaceutical preparation, during a first stage of administering the therapeutic dose.

In some examples, the predetermined dose profile is such that the dose rate varies over the predetermined infusion time. In some examples, the cumulative dose delivered to the patient increases exponentially, or increases at a rate that increases over time, for at least a portion of the predetermined infusion time.

In some examples, the dose profile is such that there is a first time period between the cumulative dose reaching 0.01% and 0.1% and a second time period between the cumulative dose reaching 0.1% and 1% of the therapeutic dose; and the first period of time and the second period of time are selected from the group comprising: at least 6 minutes, at least 5 minutes, at least 4 minutes, at least 3 minutes, between 2 minutes and 10 minutes, and at least the latent period of adverse reaction.

The processor may control the medication delivery apparatus to deliver the pharmaceutical preparation according to the predetermined profile by controlling an actuator of the infusion device. For example the actuator may controlled to drive a plunger, or a pump, of the medication delivery apparatus such that the pharmaceutical preparation is delivered according to the predetermined dose profile. For instance, the processor may divide the predetermined infusion time into a number of infusion steps and determine a target flow rate or a target output volume for each infusion step such that the predetermined dose profile is realized when the actuator is controlled according to the target flow rate or target output volume for each infusion step. The target flow rates or target output volumes for the infusion steps for a predetermined dose profile may be determined by referring to a lookup table stored in the memory. The lookup table may be populated according to the techniques described herein, for instance calculating the target flow rate or target output volume for each infusion step based on modelling of the predetermined dose profile. In other examples, target flow rate or target output volume for each infusion step may be calculated by the processor in real time.

### BRIEF DESCRIPTION OF THE DRAWINGS

Further features of the present disclosure are more fully described in the following description of several non-limiting embodiments thereof. This description is included solely for the purposes of exemplifying the present disclosure. It should not be understood as a restriction on the broad summary, disclosure or description of the disclosure as set out above. The description will be made with reference to the accompanying drawings in which:

FIG. 1A is a perspective view of a particular arrangement of a medication delivery apparatus for delivery of a pharmaceutical preparation in accordance with a first embodiment of the disclosure;

FIG. 1B is a block diagram of a particular arrangement of a medication delivery apparatus for delivery of a pharmaceutical preparation, according to some embodiments;

FIG. 2 is a perspective view of a particular arrangement of an apparatus for delivery of a pharmaceutical preparation (a medication delivery apparatus), according to some embodiments;

FIG. 3 is a perspective view of a particular arrangement of a medication delivery apparatus comprising a dilution chamber, the medication delivery apparatus being connected to an infusion device, according to some embodiments;

FIG. 4 is a top view of the dilution chamber of the medication delivery apparatus shown in FIG. 3, according to some embodiments;

FIG. 5 is a close up view of a portion of the dilution chamber shown in FIG. 4, showing a particular arrangement of a catheter that is inserted in the dilution chamber, according to some embodiments;

FIG. 6 is a top view of the dilution chamber shown in FIG. 3 showing the catheter shown in FIG. 5 extracted from the dilution chamber, according to some embodiments;

FIG. 7A is a top view of the catheter shown in FIG. 5 extracted from the dilution chamber, according to some embodiments;

FIG. 7B is a top view of a lower portion of the manifold, according to some embodiments;

FIG. 8A is a top view of the catheter, according to some embodiments;

FIG. 8B is a top view of the distal end of a catheter, according to some embodiments;

FIG. 8C is a top view of a distal end of the catheter, where the catheter is attached to the manifold, according to some embodiments;

FIG. 8D is a top view of the distal end of the catheter, where the catheter is attached to the manifold, according to some embodiments;

FIG. 9A is schematic figure of an alternative arrangement of a catheter, according to some embodiments;

FIG. 9B is perspective view the catheter shown in FIG. 9A, according to some embodiments;

FIG. 10 is a perspective view of a distal end of an alternative arrangement of a catheter, according to some embodiments;

FIG. 11A is a top view of an alternative arrangement of a catheter, the catheter being attached to the lower portion of a manifold, according to some embodiments;

FIG. 11B is top view of the catheter shown in FIG. 11A, according to some embodiments;

FIG. 11C is a top view of the catheter shown in FIG. 11A with a manifold attached to a connecting body, according to some embodiments;

FIGS. 11D and 11E are top views of the catheter shown in FIGS. 11A and 11B attached to the dilution chamber, according to some embodiments;

FIGS. 12A and 12B depicts a flowchart illustrating a method of delivering a therapeutic dose of a drug, according to some embodiments, which may be referred to as a Tansy method;

FIG. 12C depicts a flowchart illustrating the Tansy method including the process of programming the infusion pump, according to some embodiments;

FIG. 13A depicts a flowchart illustrating a method of delivering the therapeutic dose of a drug, according to some embodiments, which may be referred to as a Sadleir method;

FIGS. 13B and 13C depicts a flowchart illustrating the Sadleir method, comprising a Sadleir function configured to enable calculation of the infusion rates and volumes delivered at various points in time during the Sadleir method, according to some embodiments;

FIGS. 13D and 13E depicts a flowchart illustrating a method of approximating the infusion rates and volumes calculated in FIGS. 13B and 13C, using an infusion pump, according to some embodiments;

FIG. 13F illustrates utilisation of the flowchart of FIGS. 13B and 13C, for an example where the Sadleir method was used for each interval n in the first 0.04 minutes of a 30 minute infusion of 50 mL of pharmaceutical preparation, each interval n in the first 0.04 minutes of the infusion. Illustrated are the value of: the target dose to be delivered (the modified Tansy function dose), the flow rates (infusion rate) as dictated by the Sadleir function, the concentration in dilution chamber and the % dose delivered in each interval, n;

FIGS. 14A (logarithmic y axis scale) and 14B (linear y axis scale) illustrate rates of drug administration, comparing a constant infusion method and the Tansy method for an infusion duration of 30 minutes;

FIGS. 15A (logarithmic y axis scale) and 15B (linear y axis scale) illustrate the difference in cumulative dose administered at each stage of a 30 minute infusion by the infusion method in accordance with the first embodiment of the disclosure (referred to as the Tansy Method) versus a constant infusion method;

FIG. 16 tabulates the infusion times and cumulative percentage of total dose delivered to the patient for a 30 minute infusion of 50 mL of pharmaceutical preparation with the constant infusion method, Tansy method and Sadleir method (using the same initial pharmaceutical preparation concentration and a 10 ml dilution chamber, τ=1200/min);

FIGS. 17A (calculating the Sadleir function using 60 integration intervals per minute, i.e. τ=60) and 17C (calculating the Sadleir function using 1200 integration intervals per minute, i.e. τ=1200) illustrate variations of flow rates for different instances of the second embodiment of the disclosure that differ with respect to each as a consequence of selection of different initiating interval rates (30 minute infusion duration, 10 ml dilution chamber, 50 ml pharmaceutical preparation volume);

FIGS. 17B and 17D illustrate the differences in minimum flow rates for the Sadleir function as a consequence of the different initiating interval rates in FIGS. 17A and 17C, respectively;

FIG. 17E shows a graph plotting the value of minimum flow rate for each of the instances (shown in FIG. 17D) of the Sadleir method that differ with respect to each other in the initiating interval rate;

FIG. 18 illustrates the volume of administered drug during the first minute of Sadleir method according to different degrees of precision of calculation (number of integration intervals per minute, or tau), including or not including the volume of the initiating interval;

FIGS. 19A (linear y axis scale) and 19B (logarithmic y axis scale) illustrate the rates of infusion of the pharmaceutical preparation fluid from the dilution chamber into the patient when using the Tansy method for 50 ml infusions over various example durations of infusion (20 minutes, 25 minutes, 30 minutes, 45 minutes, 60 minutes, 120 minutes and 180 minutes);

FIG. 20A illustrates and compares the rates of infusion of the pharmaceutical preparation from the dilution chamber when using the Sadleir (10 mL dilution chamber, Vd) or Tansy methods for a 50 mL infusion over the first 4 minutes of various example durations of infusion (20 minutes, 25 minutes, 30 minutes, 45 minutes, 60 minutes, 120 minutes and 180 minutes);

FIG. 20B illustrates the rates of infusion of the pharmaceutical preparation from the dilution chamber when using the Sadleir method (10 mL Vd in this example) for a 50 mL infusion over various example durations of infusion (20 minutes, 25 minutes, 30 minutes, 45 minutes, 60 minutes, 120 minutes and 180 minutes);

FIG. 20C illustrates the rates of infusion of the pharmaceutical preparation fluid from the pharmaceutical container when using the Sadleir method for 50 mL infusion and using a 10 mL dilution chamber, for the first 10 minutes of various example durations of infusion (20 minutes, 25 minutes, 30 minutes, 45 minutes, 60 minutes, 120 minutes and 180 minutes);

FIG. 21A (linear y axis scale) and FIG. 21B (logarithmic y axis scale) illustrate the rate of pharmaceutical preparation dose administered to a patient (in percentage of the total dose given during each 1/1500 minute period of the infusion when using the Sadleir method for a 50 ml infusion volume, 10 ml dilution chamber, τ=1200) for various total infusion durations (20 minutes, 25 minutes, 30 minutes, 45 minutes, 60 minutes, 120 minutes, 180 minutes);

FIG. 21C illustrates the cumulative dose administered to the patient (in percentage of the total dose using the Sadleir method for a 50 ml infusion volume, 10 ml dilution chamber, τ=1200) over the course of the infusion for various total infusion durations (20 minutes, 25 minutes, 30 minutes, 45 minutes, 60 minutes, 120 minutes, 180 minutes);

FIG. 22A is a table of the calculated values of instantaneous rate, cumulative volume delivered and cumulative dose delivered for the Tansy method and Sadleir method at 45-second intervals over a 30-minute infusion of 50 mL pharmaceutical preparation; in this particular example the Sadleir function values were calculated using an integration interval of duration 50 milliseconds (τ=1200/min) and a dilution chamber of 10 mL volume;

FIGS. 22B and 22C illustrate the difference in pharmaceutical preparation fluid injection or infusion rates (ml/min) when using the first (Tansy) or second (Sadleir with 10 ml dilution chamber) embodiments of the disclosure for a 50 ml infusion over 30 minutes wherein FIG. 22B illustrates the first 15 minutes of the 30 minute infusion);

FIGS. 22D and 22E illustrate the difference in cumulative volume infused from the pharmaceutical preparation fluid syringe or container over the course of a 30 minute infusion when using the first (Tansy) or second (Sadleir with 10 ml dilution chamber) embodiments of the disclosure for a 50 ml infusion, wherein FIG. 22D illustrate the first 15 minutes of a 30 minute infusion);

FIG. 23A is a table of the instantaneous rates of the Tansy function at various points in time of a 60 minute infusion of 1000 mL of pharmaceutical preparation, and the values for approximating this function with an infusion device using forty constant-rate steps or forty ramp-rate steps methods;

FIGS. 23B (linear y axis scale) and 23C (logarithmic y axis scale) illustrate the flow rate of the two approximations of the Tansy function using 40 infusion steps over a 30-minute infusion of 1000 ml;

FIG. 24A is a table of values of two examples of approximating the Sadleir function for a 50 mL infusion of pharmaceutical preparation over 30 minutes, using a 10 mL dilution chamber and τ of 1200/min.

FIGS. 24B and 24C illustrate the infusion rate over the duration of an infusion period resulting from approximations of the Sadleir method using either a ramp-rate step or constant-rate step program (40 infusion steps of 45 seconds each as for FIG. 24A) over a 30 minute infusion, wherein FIG. 24C illustrates the (first 15 minutes of a 30 minute infusion).

FIGS. 24D and 24E (first 5 minutes of a 30 minute infusion) illustrate the dose of drug administered when using the approximations in FIGS. 24B and 24C with the second embodiment of the disclosure (Sadleir method), wherein FIG. 24E illustrates the first 15 minutes of a 30 minute infusion;

FIGS. 25A and 25B illustrate the flow rate of the Sadleir function compared to three different approximations of the Sadleir method constant infusion rate steps, with infusion steps of duration 45 seconds over a 30-minute infusion (40 steps, τ=1200), rate in ml/min, wherein the FIG. 25B illustrates the first 4 minutes of a 30 minute infusion;

FIGS. 25C (linear y axis scale) and 25D (logarithmic y axis scale) illustrate the cumulative dose administered to the patient as a percentage of total drug dose over the first 3 minutes of a 30 minute infusion comparing the Sadleir function to five examples of approximations of the Sadleir infusion rate using an infusion step duration of 45 seconds (40 infusion steps in total);

FIGS. 26A to 26D illustrate the experimental results of two particular realisations of the second embodiment of the disclosure using a 10 mL dilution chamber with a balloon-tipped catheter as shown in FIG. 8c, with three 30 g (0.25 mm) perforations delivering 50 ml of pharmaceutical preparation over 30 minutes using 40 ramp-rate infusion steps;

FIG. 26A illustrates the experimental results of two particular realizations of the second embodiment of the invention (dashed and dotted lines), comparing dilution chamber concentration over time to that of the theoretical result assuming perfect mixing (dash-dotted line);

FIG. 26B illustrates the percentage dose delivered per 1/1200 min period over the duration of the 30 minute infusion with a logarithmic y scale;

FIG. 26C illustrates cumulative percentage dose (percentage of pharmaceutical preparation concentration) delivered over time for a 30 minute infusion, using a 10 ml dilution chamber with a balloon-tipped catheter shown in FIG. 8c with three 30 g (0.25 mm) perforations (solid line) versus that predicted by the Sadleir function (dashed line);

FIG. 26D illustrates the cumulative percentage dose delivered over a 30 minute infusion for the particular realisation of the second embodiment of the disclosure as for FIG. 26C, except that cumulative percentage dose (percentage of pharmaceutical preparation concentration) is plotted on a logarithmic y scale. The separation of orders of magnitude of cumulative percentage dose is also presented;

FIGS. 27A to 27C is software code, written in Python 3, to calculate the values that can be sent to the infusion device to realise the Tansy method (first embodiment of the disclosure);

FIGS. 28A to 28E is software code, written in Python 3, to calculate the values that can be sent to the infusion device to realise the Sadleir method (second embodiment of the disclosure);

FIGS. 29A and 29B depicts a flowchart illustrating a modified Sadleir function which is applied to calculate the infusion flow rates for the ‘Increased Volume Sadleir method’;

FIGS. 29C and 29D illustrate the cumulative volume infused using the alternative embodiment (‘Increased Volume Sadleir method’) of the second embodiment of the disclosure with various dilution chamber volumes (10 mL, 20 mL and 30 mL), wherein FIG. 29D illustrate the first 15 minutes of a 30 minute infusion;

FIGS. 29E and 29F illustrate the infusion rates using the alternative embodiment (‘Increased Volume Sadleir method’) of the second embodiment of the disclosure with various dilution chamber volumes (10 mL, 20 mL and 30 mL), wherein FIG. 29F illustrate the first 10 minutes of a 30 minute infusion;

FIG. 29G illustrates the similar dosing of active ingredient over the infusion period when using the ‘Increased Volume Sadleir method’ with various dilution chamber volumes, compared to the equivalent Tansy method;

FIG. 30 shows a side view of a medication delivery apparatus, according to some embodiments;

FIG. 31 illustrates the process of filling the medication delivery apparatus, according to some embodiments;

FIG. 32 shows a side perspective view of the medication delivery apparatus shown in FIG. 30 filled with active agent and diluent, according to some embodiments;

FIG. 33 is a perspective view of the medication delivery apparatus shown in FIG. 32 during mounting on an infusion driver in the form of a syringe driver, according to some embodiments;

FIG. 34A illustrates a process for mixing the active agent and the diluent within the dilution chamber, according to some embodiments;

FIG. 34B illustrates a method of operation of the medication delivery apparatus, according to some embodiments;

FIGS. 34C and 34D is a block diagram for calculating a method of delivering a therapeutic dose of a drug, according to some embodiments, which may be referred to as a Diocles infusion protocol or a Diocles method. The Diodes method is used during operation of the medication delivery apparatus depicted in FIGS. 30 to 41 while being mounted on an infusion device in the form of a syringe driver;

FIGS. 34E and 34F is a flowchart illustrating a method of approximating the infusion rates and volumes calculated in FIG. 34C, using an infusion pump, according to some embodiments;

FIG. 35 shows a front perspective view of a medication delivery apparatus, according to some embodiments;

FIG. 36 shows a perspective view of a longitudinal cross-section of the medication delivery apparatus shown in FIG. 35, according to some embodiments;

FIG. 37 shows a view of the medication delivery apparatus shown in FIG. 36, according to some embodiments, depicting a proximal side of a separating plunger having a first arrangement of valve means;

FIG. 38 shows a perspective view of the separating plunger with a stirring means extracted from the medication delivery apparatus, according to some embodiments;

FIG. 39 shows a view of the medication delivery apparatus of FIG. 36, according to some embodiments, depicting a proximal side of the separating plunger having a second arrangement of valve means;

FIG. 40 shows a front perspective view of a medication delivery apparatus, according to some embodiments, having a separating plunger having a third arrangement of valve means;

FIG. 41 shows a view of the dilution chamber shown in FIG. 40, according to some embodiments, depicting the proximal side of the separating plunger having a fourth arrangement of valve means;

FIG. 42 shows a side view of the medication delivery apparatus shown in FIG. 35 filled with active agent and diluent, according to some embodiments;

FIG. 43A shows a side view of the medication delivery apparatus shown in FIG. 35, filled with active agent and diluent, being fed with the active agent remotely from the syringe driver, according to some embodiments;

FIG. 43B illustrates the method of operation of the medication delivery apparatus depicted in FIG. 43A, according to some embodiments;

FIG. 43C is a block diagram illustrating a method of delivering a therapeutic dose of a drug, according to some embodiments. The method may be for calculating a Sadleir infusion protocol used during operation of dilution chamber depicted in FIG. 43A;

FIG. 43D is a flowchart illustrating a method of approximating the infusion rates and volumes calculated in FIG. 43C, using an infusion pump, according to some embodiments;

FIG. 44 shows a perspective view of an arrangement of a medication delivery apparatus during mounting on a. infusion device in the form of a syringe driver, according to some embodiments;

FIGS. 45 and 46 are, respectively, a distal perspective view and a side view of the medication delivery apparatus shown in FIG. 44, during assembly thereof, according to some embodiments;

FIGS. 47A and 47B illustrate a process of operating the medication delivery apparatus shown in FIG. 44, according to some embodiments;

FIGS. 47C to 47F is a block diagram for calculating the infusion protocol used during operation of dilution chamber depicted in FIGS. 44 to 47A, according to some embodiments;

FIG. 48 illustrates particular arrangements of the Pulse-Width Modulation (PWM) digital dilution for controlling the infusion process;

FIGS. 49A to 49H illustrate results of an example infusion performed according to the Diocles method;

FIG. 50 illustrates a dilution chamber drug concentration profile of an example infusion performed according to the Diocles method over a sub-section of the infusion;

FIG. 51 illustrates a comparison of an infusion performed in accordance with the Diocles method and an infusion performed in accordance with the Tansy method;

FIGS. 52A to 52F illustrate a test comparison of a 30 minute infusion using 60 30 second steps of the Diocles method, with each step being a constant infusion (darker grey) (‘constant’) and a 30 minute infusion using 60 bursts at a higher infusion rate (lighter grey) (‘burst’). FIG. 52A indicates the flow rate versus time of fluid leaving the medication delivery apparatus with the two programs, with either the volume of each step being given at a constant rate over the step (‘constant’, darker gray), or at a rate of 15 mL/min for the portion of the step that would result in the same volume being given for each step (‘burst’, lighter gray). FIG. 52B indicates the concentration of drug entering the patient (percentage of total dose initially in drug chamber, per mL) with respect to time, with ‘constant’ program (darker gray), and ‘burst’ program (lighter gray). FIG. 52C indicates the rate of drug delivery (percentage of total dose per minute) administered to the patient over time with the ‘constant’ (darker gray) and ‘burst’ (lighter gray) program. FIG. 52D indicates the cumulative percentage dose (percentage of total dose) administered to the patient over time, with a logarithmic y scale for the ‘constant’ (darker gray) and ‘burst’ (lighter gray) program. FIG. 52E demonstrates the ratio of cumulative dose administered to the patient at a point 5-minutes after the time indicated on the x-axis, to the cumulative dose administered to the patient at the time indicated on the x-axis with the ‘constant’ (darker gray) and ‘burst’ (lighter gray) programs. FIG. 52F indicates the delay in minutes between the time indicated on the x-axis, and the time when the cumulative dose administered is 10-times that of the time indicated on the x-axis, for ‘constant’ (darker grey) and ‘burst’ (lighter grey) programs;

FIGS. 52G to 52L illustrate a test comparison of a 25 minute infusion using 50 30 second steps with a double burst at 15 mL/min separated by 1 second (darker colour) compared to a single burst at 15 mL/min with the volume of the second burst spread throughout the interval (i.e. no closure of the valve and no cracking (lighter colour); and

FIGS. 53A to 53D illustrate constant step, burst, burst-constant and burst-burst infusion delivery programs of the Diocles method and resultant pharmaceutical preparation delivery results. FIG. 53A indicates the fluid injection rate for four alternative modifications of the Diocles method. The ‘constant’ program delivers the volume to be delivered in each step of the Diocles method at a constant rate over the whole duration of the step (531). The ‘burst’ program delivers the volume to be delivered in each step of the Diocles method at a rate of 15 mL/min for a period that is shorter than the step (533). The ‘burst-burst’ method delivers the volume to be delivered in each step of the Diocles method in two periods of infusion at 15 mL/min for each step, with each period separated by 1 second (535). ‘The burst-constant’ method delivers half of the volume to be delivered during each step at 15 mL/min, with the remaining half of the volume delivered over the remaining duration of the step at a constant rate (535). FIG. 53B indicates the concentration of drug delivered to the patient (percentage of total dose initially in drug chamber per mL) over time for each of the four programs. FIG. 53C indicates the cumulative percentage dose administered to the patient over time on a logarithmic y scale for each of the four programs. FIG. 53D indicates the ratio of cumulative dose administered to the patient 5 minutes after the point in time indicated on the x-axis, to the cumulative dose administered at the point in time on the x-axis;

FIGS. 53E and 53F illustrates a constant step, single burst, burst-constant and double burst infusion step programs, according to some embodiments; and

FIGS. 54A to 54E show software code, written in Python 3, to calculate the values that can be sent to the infusion device to realise the Diocles method, according to some embodiments;

FIGS. 55A to 55D are graphs showing cumulative dose delivered and instantaneous dose rate against time for various Tansy functions.

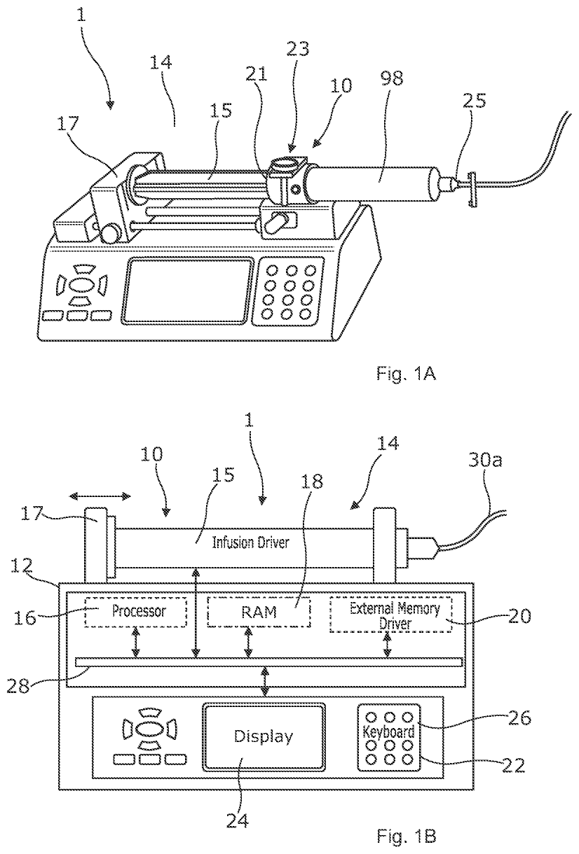

It should be noted that the FIGS. 1 to 11E and FIGS. 30 to 34A, 35 to 43 and 44 to 47A are schematic only and the location and disposition of the components can vary according to the particular arrangements of the embodiments of the present disclosure as well as of the particular applications of the present disclosure.

### DESCRIPTION OF EMBODIMENTS

Part 1—General Principles

A pharmaceutical preparation may be delivered to a patient by a medication delivery apparatus such as, but not limited to, a syringe or other intravenous pharmaceutical delivery device. The medication delivery device may be controlled by an infusion device, such as an infusion driver which may be programmed to control the medication delivery device to deliver the pharmaceutical preparation to the patient in accordance with a predetermined dose profile. Examples of infusion device and medication delivery apparatus are described, inter-alia, in Parts 2, 5 and 10 of this application.

Part 1 of this application describes how the medication delivery apparatus may be controlled to deliver the pharmaceutical preparation to the patient according to a predetermined dose profile, wherein the predetermined dose profile is designed to deliver a therapeutic dose of the pharmaceutical preparation to the patient over a predetermined infusion time in a manner which facilitates safe detection of an adverse reaction of the patient to the pharmaceutical preparation, or desensitization the patient to the pharmaceutical preparation. Part 3 of this application describes examples of the dose profile and ways in which the dose profile may be designed, while Parts 3A-3C, 4 and Parts 6-9 describe various examples of implementing a system in which the medication delivery apparatus may be accurately controlled to deliver such dose profiles.

The methods and system in accordance with the present embodiments of the disclosure allow administration in a single infusion process of a therapeutic dose of a particular drug in conjunction with test doses. These methods and systems are particularly useful because they do not require given patients a multitude of test doses prior the infusion of the therapeutic dose. Instead, the test doses are effectively given during infusion of the full therapeutic dose due to the test doses being part of the therapeutic dose. Provision of test doses without the use of the embodiments of the present embodiments of the disclosure requires (1) preparation of a multitude of pharmaceutical preparations (including the test doses) having different concentrations and (2) infusing the multitude of pharmaceutical preparations for each test dose to the patient for each of the pharmaceutical preparations. This process of infusing a multitude of pharmaceutical preparations containing test doses (prior infusion of the therapeutic dose) can be a cumbersome and time consuming task and can be unsuitable in situations where infusion of the therapeutic dose must be done immediately to, for example, preserve the life of a patient. In contrast, the present disclosure proposes that one or more test doses are delivered as a first part of the therapeutic dose. For example, this may be done by delivering the therapeutic dose in accordance with a predetermined dose profile, in which a first part of the therapeutic dose is delivered very slowly to the patient.

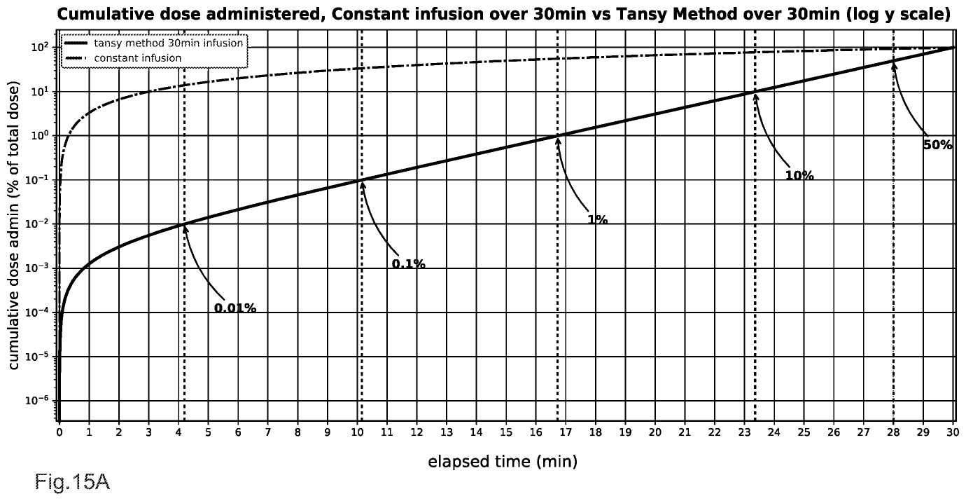

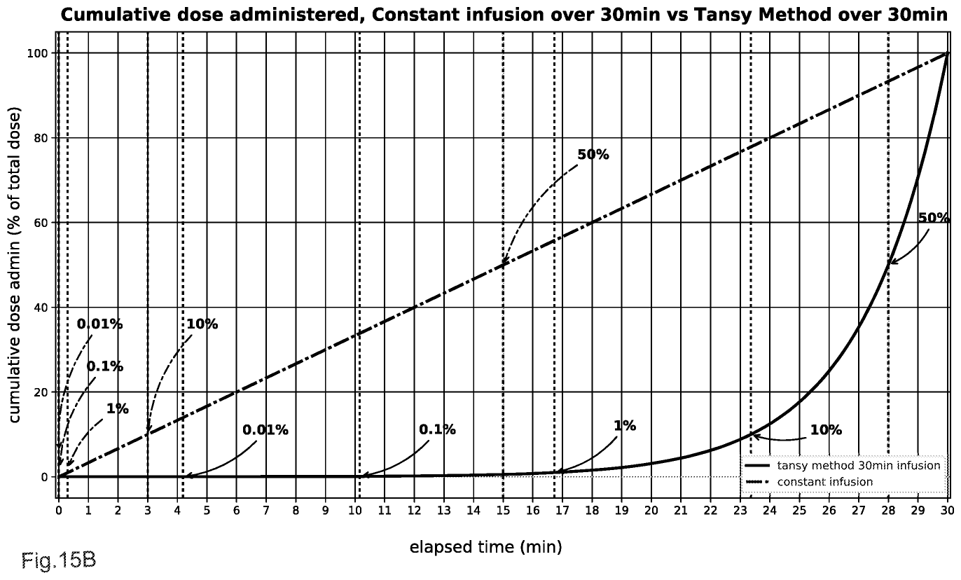

These methods and systems in accordance with the present disclosure are particularly useful because they increase the likelihood that an adverse reaction will be recognized before a specific dose (a particular amount of drug), that will induce a more severe negative reaction in the patient, has been administered (see FIGS. 15A and 15B). FIGS. 15A and 15B illustrate two example dose profiles. The dose profile in dashed and dotted lines is a constant rate infusion in which the dose rate is constant throughout the infusion. The dose profile in a solid line is a dose profile in which the dose rate at which a drug is delivered to the patient increases over time. It can be seen that for the constant dose rate profile, shown in dashed and dotted line, the first 1% of the therapeutic dose is delivered in the first few seconds of the infusion, specifically the first 1% of the infusion (the first 18 seconds for a 30 minute infusion). In contrast, for the example variable dose rate profile, shown in the solid line, successive orders of magnitude of the cumulative percentage of the therapeutic dose delivered to the patient (e.g. (e.g. 0.01%, 0.1%, 1% and 10% of the therapeutic dose), are relatively broadly spread out. Thus, these methods and systems are adapted to safely provide the therapeutic dose to a patient when one or more specific doses, that will cause a submaximal reaction in the patient, are not known.

The present embodiments of the disclosure provide methods and system for the provision of test doses of a drug to a particular patient who may suffer a hypersensitivity reaction (hypersensitivity, or allergy or other adverse reaction) with a short latency.

It will be understood that the term “active agent” as used in the description, may correspond to, or also be referred to as an “active ingredient” or a “drug”. That is, throughout this disclosure, the terms “active ingredient”, “active agent” and “drug” have been used to describe the active agent that is to be administered to a patient. In some embodiments, a pharmaceutical preparation can be delivered to a patient. The pharmaceutical preparation may comprise the active agent. The pharmaceutical preparation may also comprise one or more other constituents. For example, the pharmaceutical preparation may comprise a solvent. That is, in some embodiments, the pharmaceutical preparation may comprise the active agent and a solvent. The pharmaceutical preparation may comprise the active agent at a particular concentration. This may be referred to as an active agent concentration.

The pharmaceutical preparation may be a solution. It will be understood that in some embodiments, the term “drug” as used in the description may correspond to the active agent of the “pharmaceutical preparation”.

The therapeutic dose of the pharmaceutical preparation refers to the dose of active ingredient which is to be delivered to the patient over a predetermined infusion time and may for example be specified by the physician. The therapeutic dose may for example be specified in mg of active ingredient. The predetermined infusion time is a predetermined period of time over which the therapeutic dose is to be delivered. The term “predetermined dose profile” refers to a predetermined manner in which a therapeutic dose of the drug is delivered to the patient over a predetermined infusion time. The dose profile may include a dose rate and/or a cumulative dose that are to be achieved at various points in time over the infusion time. The dose rate, sometimes also referred to as the drug administration rate, refers to the rate at which an active ingredient of the pharmaceutical preparation is to be delivered to the patient. The cumulative dose at a particular point in time refers to the amount of active ingredient delivered to the patient from the beginning of the infusion until that particular point in time. The cumulative dose may, for example, be measured as a percentage of the therapeutic dose which has been delivered.

The methods and system in accordance with the first embodiment of the disclosure uses a particular function (Tansy function) for delivering (infusing) sequentially to a patient a wide range of test doses of a pharmaceutical preparation, with the dose(s) increasing during the duration of the infusion. This has an objective of overcoming the problem of the sensitivity to a particular drug in a patient when the threshold for this sensitivity is not known prior to the administration of the particular drug. In some embodiments, during the entire duration of the infusion, a full therapeutic dose is provided with a portion of the therapeutic dose being used as one or more test doses. In this manner, there is no need of interrupting the administration of the therapeutic dose by, for example, providing at a first stage, a test dose contained in a particular pharmaceutical preparation; and then, after having confirmed that the patient will have no negative reaction to the drug, continuing to infuse the pharmaceutical preparation to the patient. Thus, according to the first embodiment of the disclosure only a single pharmaceutical preparation is required to provide the full therapeutic dose including any test doses.

The method and system in accordance with a second embodiment of the disclosure also allows the administration to a patient a single pharmaceutical preparation to provide the full therapeutic dose including the test doses. However, as will be explained below, the method and system in accordance with a second embodiment of the disclosure allows the accuracy with which the pharmaceutical preparation is provided to the patient to be increased. It does so by permitting an increase in the initial flow rate of a pharmaceutical preparation driven by an infusion driver 14, when compared to a flow rate of the pharmaceutical preparation when using the method and system in accordance with the first embodiment of the disclosure (the Tansy Method). In some embodiments, the infusion driver 14 may be, a syringe driver or peristaltic pump or similar drug infusion pump. In some embodiments, the infusion driver is in the form of an infusion device. In some embodiments, an infusion device comprises the infusion driver.

Increasing the flow rate with which the pharmaceutical preparation exits the infusion driver 14 when the flow rate is relatively low increases the accuracy of the administration process of the pharmaceutical preparation because it is known that infusion drivers 14 do not deliver accurately pharmaceutical preparations at relative low rates such as those that occur when using the Tansy function.

However, the methods and systems in accordance with a second embodiment of the disclosure use another function (a Sadleir function) to control a rate at which the pharmaceutical preparation is delivered (infused) to the patient. Infusing the pharmaceutical preparation as dictated by the Sadleir function allows the pharmaceutical preparation to be given at a higher initial flow rate (with respect to the Tansy method) as a consequence of the use of a dilution chamber 32 that is located between an active agent chamber and the patient. The pharmaceutical preparation flows through the dilution chamber 32 prior entering the patient. The dilution chamber 32 comprises a diluent for mixing with the pharmaceutical preparation entering the dilution chamber 32. The dilution chamber 32 is adapted to ensure rapid mixing of the pharmaceutical preparation with the diluent in the dilution chamber 32. The mixing is initially done by varying repeatedly the flow rates between lower and higher values during a second priming step (occurring when the initial mixed pharmaceutical preparation is infused from the dilution chamber 32 via the conduit 30b to the patient intravenous access point). Subsequent mixing and dilution occurs within the dilution chamber 32 during the course of the delivery of the Sadleir function infusion program. This may include the use of an injection catheter within the dilution chamber 32 that includes a flexible sleeve to allow dynamic adjustment to resistance according to the flow rates.

In particular, using the Sadleir method permits reducing the concentration of the pharmaceutical preparation entering the patient at the start of the infusion process when compared to the Tansy method. The Sadleir method therefore requires a higher initial flow rate to give a similar dosing profile as that of the Tansy function, and a higher minimum infusion rate. It is important to note that the pharmaceutical drug dosing profile in accordance with the Sadleir method is the same as that delivered by the Tansy method, except that the dose in the Sadleir method at any point in time during the infusion is reduced by a fixed fraction to compensate for an amount of the drug remaining in the dilution chamber 32 at the end of the infusion process. However, it is important to note that using either the Tansy method or the Sadleir method will result in separation of orders of magnitude of cumulative dose of active ingredient of the pharmaceutical preparation.

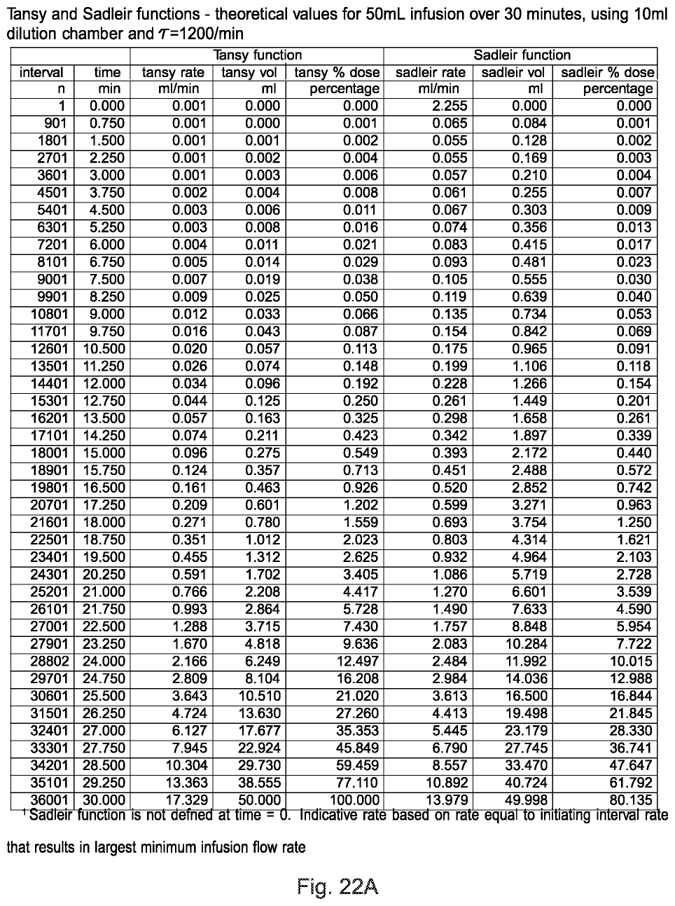

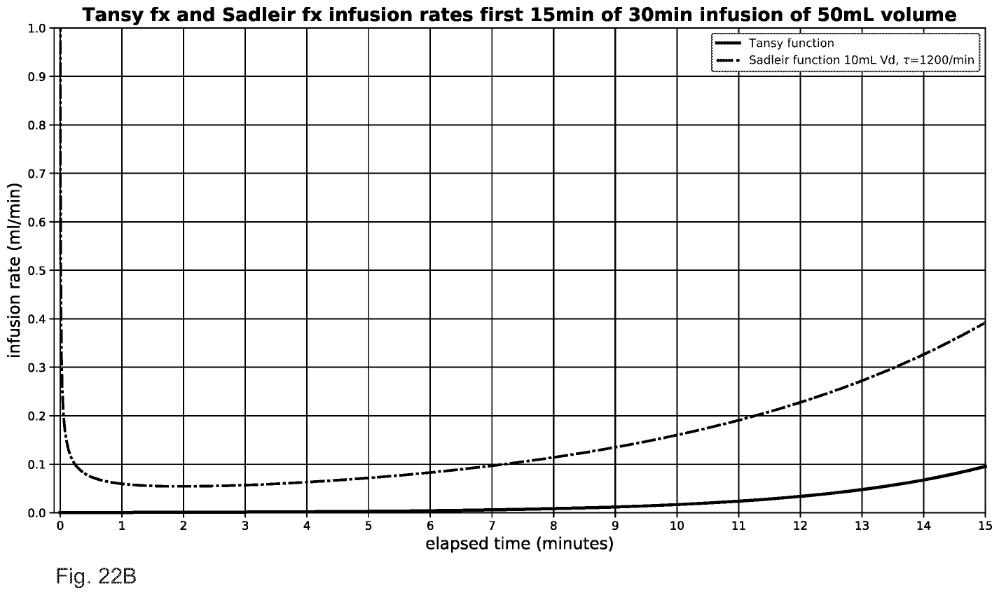

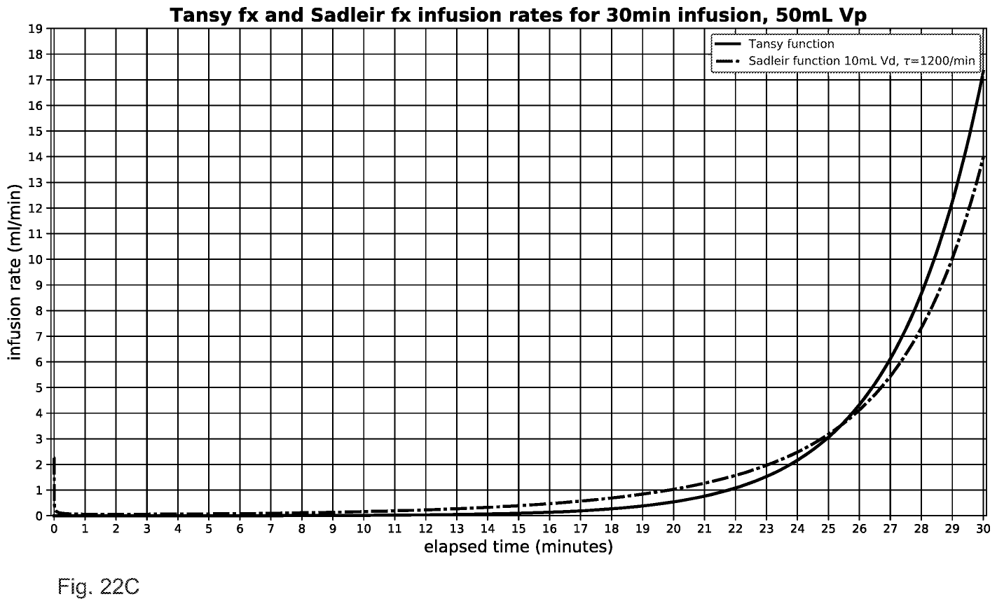

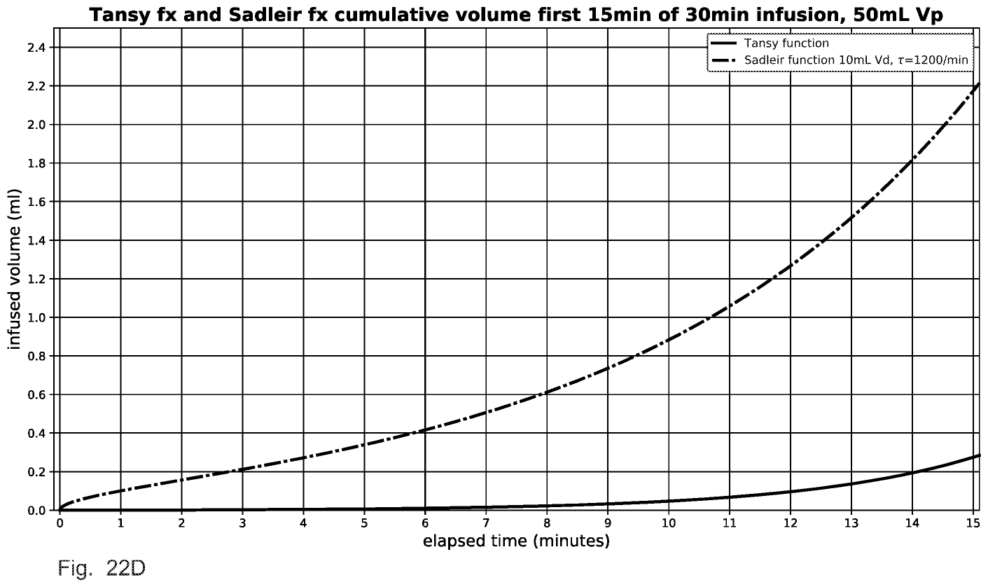

FIGS. 22B and 22C illustrate the difference in pharmaceutical preparation fluid injection or infusion rates (ml/min) when using the first (Tansy) or second (Sadleir with 10 ml dilution chamber) embodiments of the disclosure for a 50 ml infusion over 30 minutes. FIG. 22B illustrates the first 15 minutes of the 30 minute infusion, with the pharmaceutical preparation flow rate (in ml/min) being greater for the Sadleir method early in the infusion, with the Tansy method having a higher flow rate at the end of the infusion.

FIGS. 22D and 22E illustrate the difference in cumulative volume infused from the pharmaceutical preparation fluid syringe or container over the course of a 30 minute infusion when using the first (Tansy) or second (Sadleir with 10 ml dilution chamber) embodiments of the disclosure for a 50 ml infusion. FIG. 22D illustrates the first 15 minutes of a 30 minute infusion. The cumulative volume infused at a point in time is intended to mean the total volume of pharmaceutical preparation that has been infused into the patient since the beginning of the infusion until that point in time.

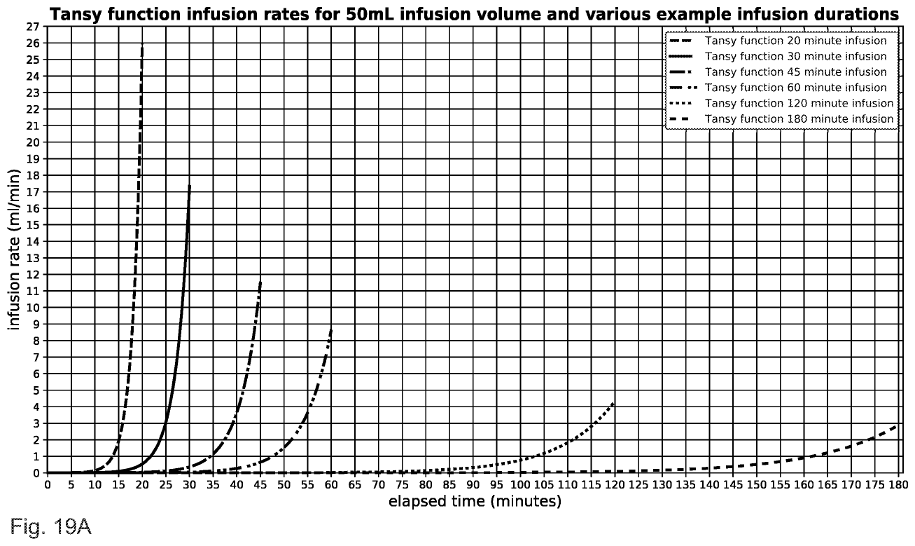

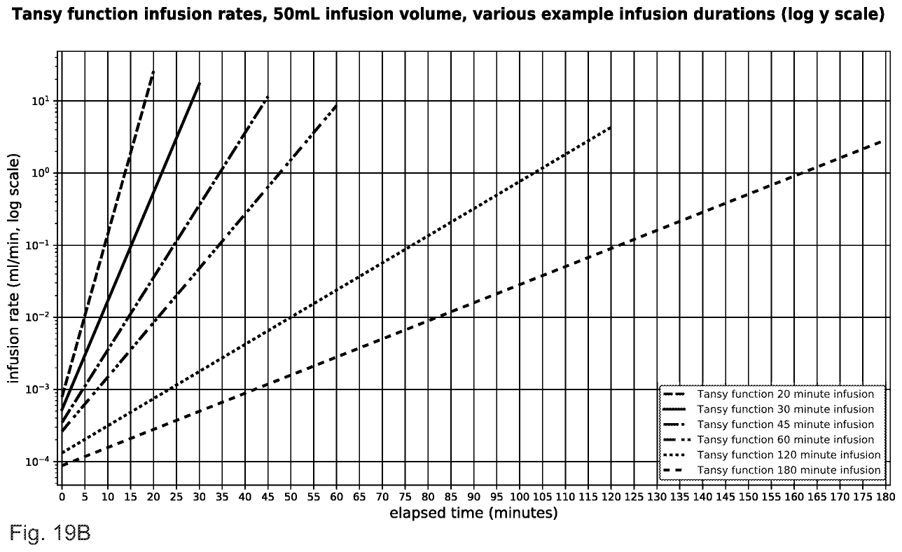

In accordance with the first embodiment of the disclosure, there is provided a method and a system that provide a pharmaceutical preparation to a patient. A flow rate of the pharmaceutical preparation follows a curve of a Tansy function (see FIGS. 19A and 19B). This method (referred to as a Tansy Method) comprises the step of providing the drug at a particular flow rate dictated by the Tansy function.

Part 2—Medication Delivery System

Various examples of medication delivery apparatus and infusion device will now be described.

The medication delivery system 1 comprises a medication delivery apparatus 10 for the provision of the pharmaceutical preparation. The medication delivery apparatus 10 may be referred to herein as an apparatus 10. The medication delivery apparatus 10 is configured to provide a pharmaceutical preparation at, or approximating the flow rate dictated by the Tansy function.

The medication delivery system 1 comprises an infusion device. The infusion device may be in the form of an infusion driver 14. In some embodiments, the apparatus 10 may comprise the infusion driver 14 (such as syringe driver or peristaltic pump or similar drug infusion pump).

The infusion driver 14 comprises a control unit for controlling the flow rate at which the infusion driver 14 delivers the drug (pharmaceutical preparation) from a syringe or bag via a generic length of tubing to the patient. The control unit comprises hardware and software for controlling the infusion driver 14 to deliver the drug at the flow rate established by the Tansy function. The software comprises a plurality of instructions for running an algorithm designed to calculate the flow rate as dictated by the Tansy function.

FIG. 1B show a block diagram of the apparatus 10 for controlling the flow rate at which the infusion driver 14 delivers the drug from a syringe or bag via a generic length of tubing to the patient.

The apparatus 10 comprises a computer system 12. The medication delivery apparatus 10 comprises an infusion driver 14. The infusion driver 14 may be referred to as an infusion device. The infusion driver 14 comprises a syringe 15 and a syringe driver 17. The syringe 15 defines an infusion container 19. The syringe 15 comprises a plunger 21. The infusion container is configured to receive at least a portion of the plunger 21. The plunger 21 and the infusion container together define an active agent chamber 98. The active agent chamber 98 may be referred to as a first chamber. The active agent chamber 98 is configured to receive an active agent. In particular, the active agent chamber 98 is configured to receive a pharmaceutical preparation. The pharmaceutical preparation comprises the active agent.

The active agent chamber 98 comprises an active agent chamber opening. The active agent chamber opening 23 is configured to receive the at least a portion of the plunger 21. The active agent chamber opening 23 may be considered an active agent chamber inlet. The active agent chamber 98 comprises an active agent chamber outlet 25.

The plunger 21 is configured to be displaced with respect to a longitudinal axis of the infusion container. Displacement of the plunger 21 along the longitudinal axis of the infusion container displaces the pharmaceutical preparation in the active agent chamber through the active agent chamber outlet 25. The pharmaceutical preparation is displaced into the conduit 30a.

In some embodiments, the infusion driver 14 comprises the computer system 12 and the syringe driver 17. The infusion driver 14 comprises a driving mechanism. In particular, the syringe driver 17 comprises the driving mechanism. The driving mechanism is controlled by the computer system 12 (the control unit 12). In particular, the control unit 12 is adapted to control the driving mechanism of the syringe driver 17 in order to deliver the drug (contained in the syringe 15) to the patient in a specific manner, for example, in accordance to either the Tansy function or the Sadleir function.

The computer system 12 comprises computer components such as a processor 16, a random access memory (RAM) 18, an external memory drive 20, and a user interface 22 such as a display 24 and a keyboard 26. These computer components are interconnected with respect to each other and the infusion driver 14 via a system bus 28.

In some embodiments, the infusion device comprises at least one infusion device processor in communication with infusion device memory. The at least one infusion device processor may comprise, or be in the form of the processor 16. The infusion device memory may comprise one or more of the random access memory 18 and the external memory drive 20. The at least one infusion device processor is configured to execute infusion device program instructions stored in infusion device memory to cause the infusion device to function as described herein. In other words, the infusion device program instructions are accessible by the at least infusion device processor, and are configured to cause the at least one infusion device processor to function as described herein.

In some embodiments, the infusion device program instructions are in the form of program code. The at least one infusion device processor comprises one or more microprocessors, central processing units (CPUs), application specific instruction set processors (ASIPs), application specific integrated circuits (ASICs) or other processors capable of reading and executing program code.

Infusion device memory may comprise one or more volatile or non-volatile memory types. For example, infusion device memory may comprise one or more of random access memory (RAM), read-only memory (ROM), electrically erasable programmable read-only memory (EEPROM) or flash memory. Infusion device memory is configured to store program code accessible by the at least one infusion device processor. The program code may comprise executable program code modules. In other words, infusion device memory is configured to store executable code modules configured to be executable by the at least one infusion device processor. The executable code modules, when executed by the at least one infusion device processor cause the at least one infusion device to perform certain functionality, as described herein.

The computer system 12 may optionally include a drug library, and database which contains the maximum allowable drug administration rate for each particular drug that may be infused to patients. If the drug delivery rate expected during use of the infusion driver 14 (e.g. during execution of the Tansy or Sadleir method), exceeds the maximum allowable drug administration rate, then the infusion rate will be reduced according to the maximum allowed infusion rate such that the concentration of drug leaving the dilution chamber (C_{d}) does not exceed the maximum allowable drug administration rate. This may result in the infusion time being greater than intended for the infusion, but ensures that the maximum permitted or suggested pharmaceutical drug administration rate is not exceeded.

During the method of infusing the pharmaceutical preparation in accordance with the present methods of the disclosure, the drug library may be accessed by the computer system 12 to confirm whether the drug delivery rate exceeds the maximum allowable drug administration rate; and if it does then, the infusion rate will be reduced according to the maximum allowed infusion rate to give the maximum allowable drug administration rate.

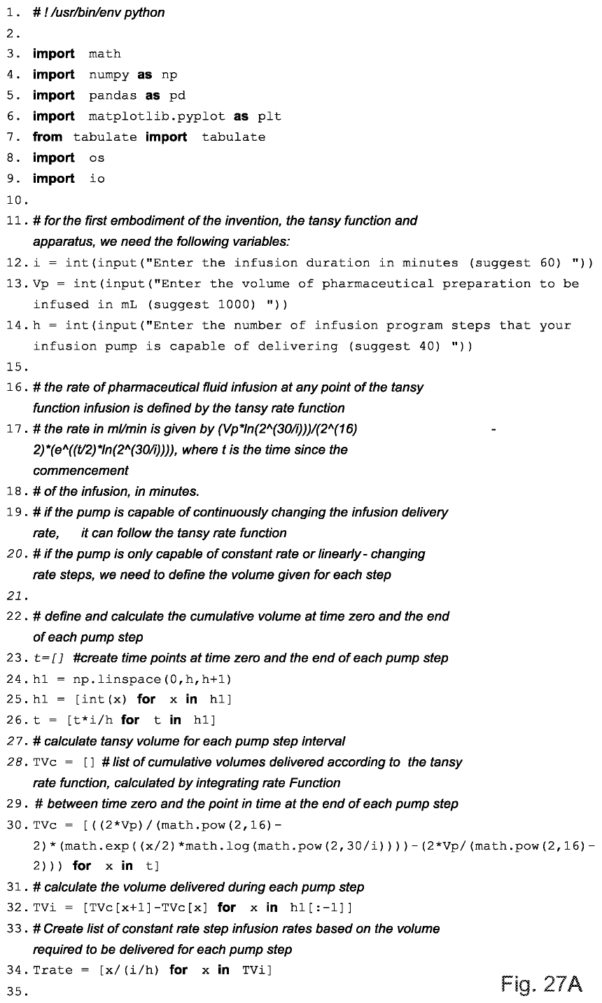

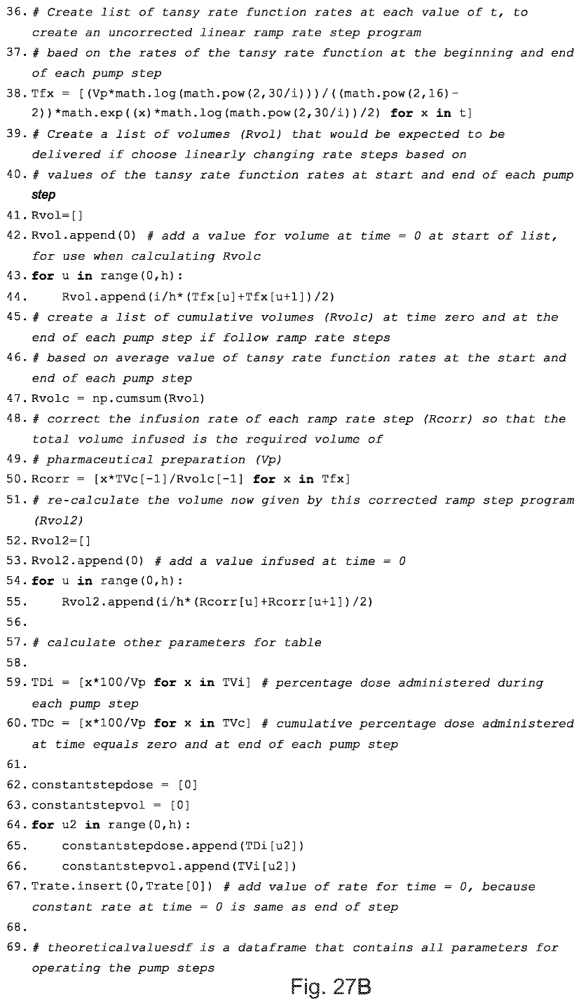

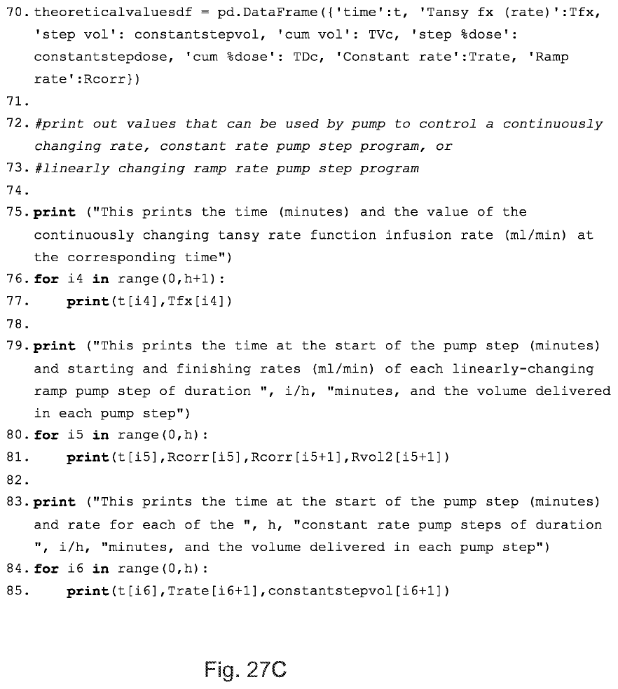

The processor 16 may execute instructions to control the driving mechanism of the syringe driver 17 in order to deliver the drug in accordance to, for example, either the Tansy function or the Sadleir function. The code executed by the processor 16 may be stored in the RAM 18 of the computer system 12 or may be provided from external sources through the external memory drive 20. This software will include the instructions to control the driving mechanism of the infusion driver 14 (e.g. the syringe driver 17) such that the pharmaceutical preparation exits the syringe 15 at a particular flow rate to match, or approximate the infusion rate of the pharmaceutical preparation dictated by the Tansy, Sadleir or another function specifying the rate at which the pharmaceutical preparation will be infused into the patient. In accordance with the first embodiment of the disclosure, the infusion driver 14 directly delivers the drug to the patient via conduit 30a (such as a minimal volume tubing with a three-way-tap to allow priming of the tubing with pharmaceutical preparation prior to commencing the program); and, the processor 16 execute codes for driving of the syringe driver 17 in order to deliver the drug (contained in the syringe 15) to the patient in accordance with the Tansy function. The software code (for example, FIG. 27), executed by the processor 16, comprises instructions for running an algorithm for calculating the infusion rates dictated by the Tansy function to control the flow rate using the syringe driver 17.

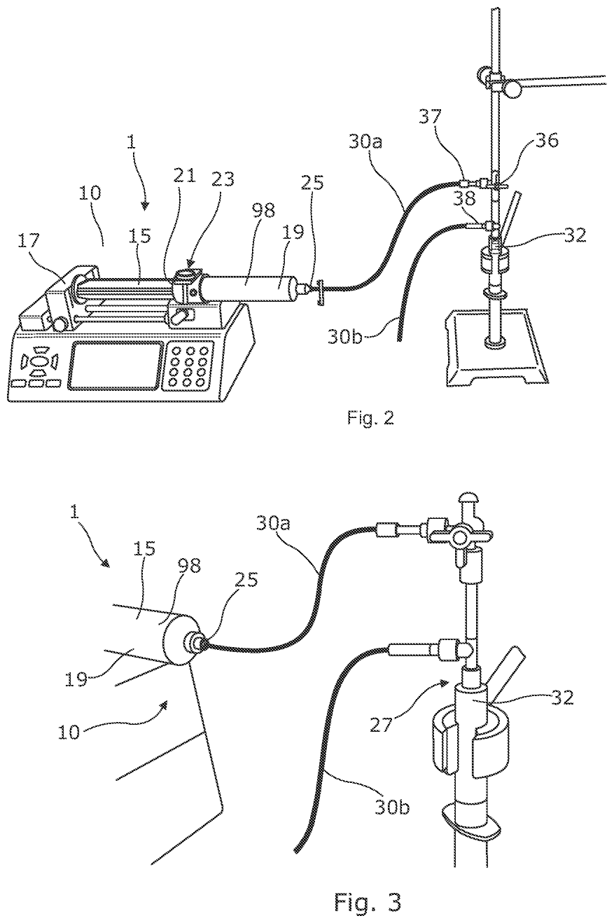

Referring now to FIGS. 2 to 8, FIGS. 2 to 8 show a medication delivery apparatus 10 according to a second embodiment of the disclosure. Again, the medication delivery apparatus 10 may be referred to as an apparatus 10. The apparatus 10 according to the second embodiment is similar to the apparatus 10 according to the first embodiment and similar reference numerals are used to identify similar parts.

As described with reference to FIG. 1, the medication delivery apparatus 10 comprises an infusion container and a plunger 21. The infusion container and the plunger 21 may form at least part of a syringe. The infusion container is configured to receive at least a portion of the plunger 21. The plunger 21 and the infusion container together define an active agent chamber 98. The active agent chamber 98 is configured to receive a pharmaceutical preparation. The pharmaceutical preparation comprises an active agent, as previously described. The active agent chamber 98 comprises an active agent chamber opening 23. The active agent chamber opening 23 is configured to receive at least a portion of the plunger 21. The active agent chamber 98 comprises an active agent chamber outlet 25.

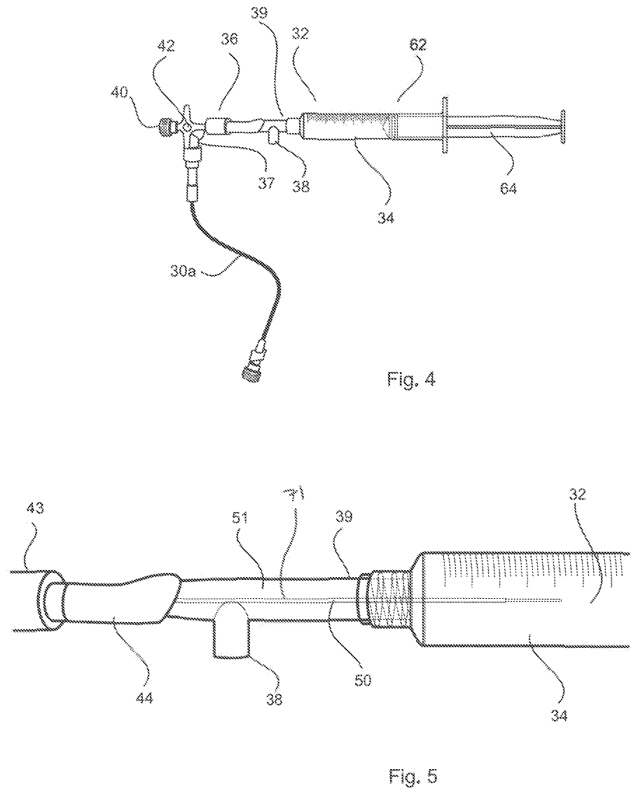

One of the differences of the apparatus 10 of the second embodiment of the disclosure is that the infusion driver 14 delivers the pharmaceutical preparation to a dilution chamber 32 (see, for example, FIGS. 2 and 4), before the pharmaceutical preparation is delivered to the patient. Thus, the medication delivery apparatus 10 comprises the dilution chamber 32. The dilution chamber 32 is fluidly connected to the infusion container. The dilution chamber 32 is configured to receive a diluent. The dilution chamber 32 is configured to receive the pharmaceutical preparation from the active agent chamber 98. In particular, the dilution chamber 32 is configured to receive the pharmaceutical preparation from the active agent chamber outlet 25. The dilution chamber 32 comprises a dilution chamber outlet 27.

The plunger 21 is configured to be displaced with respect to a longitudinal axis of the infusion container. Displacement of the plunger 21 along the longitudinal axis of the infusion container displaces the pharmaceutical preparation in the active agent chamber 98 through the active agent chamber outlet 25. The pharmaceutical preparation is displaced into the conduit 30a. The pharmaceutical preparation is displaced through the conduit 30a into the dilution chamber 32. The pharmaceutical preparation is diluted in the dilution chamber 32. The displacement of the plunger 21 displaces the diluted pharmaceutical preparation from the dilution chamber 32, through a second conduit 30b and to the patient.

The software code, executed by the processor 16, comprises instructions for running an algorithm for calculating the infusion rates dictated by the Sadleir function to control the flow rate of the syringe driver 17. Delivery of the pharmaceutical preparation from the infusion driver 14 (i.e. the active agent chamber 98) to the dilution chamber 32 and subsequently to the patient is conducted via conduits 30a and 30b. The conduits 30a and 30b comprise minimum volume extension tubing. The conduit 30a may be referred to as a first conduit. Conduit 30b may be referred to as a second conduit. The conduit 30a is configured to fluidly connect the active agent chamber outlet 25 and a dilution chamber inlet 29.

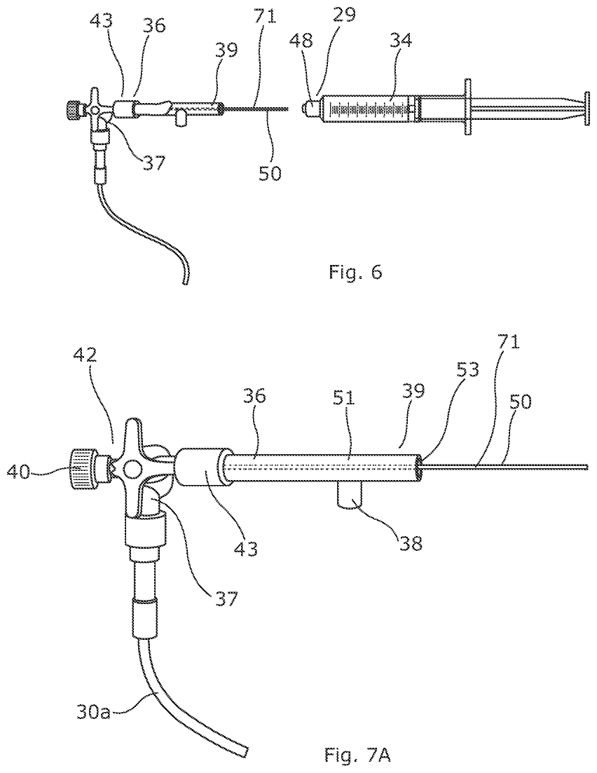

As mentioned before, the apparatus 10 in accordance with the second embodiment of the disclosure comprises a dilution chamber 32. FIGS. 6 to 8 depict a first arrangement of a dilution chamber 32. This particular arrangement of dilution chamber 32 is shown in operation in FIGS. 2 and 3.

As shown in FIGS. 4 and 6, this particular arrangement of medication delivery apparatus 10 comprises a container 34. The container 34 may be referred to as a dilution chamber container. The medication delivery apparatus 10 comprises a manifold 36. In particular, the dilution chamber 32 comprises the manifold 36. The manifold 36 is connected to the container 34 to permit fluid flow (1) from the infusion driver 14 (i.e. the active agent container 98), via the conduit 30a and first inlet 37 of the manifold 36 into the container 34. In other words, the manifold 36 is configured to connect to the dilution chamber 32.

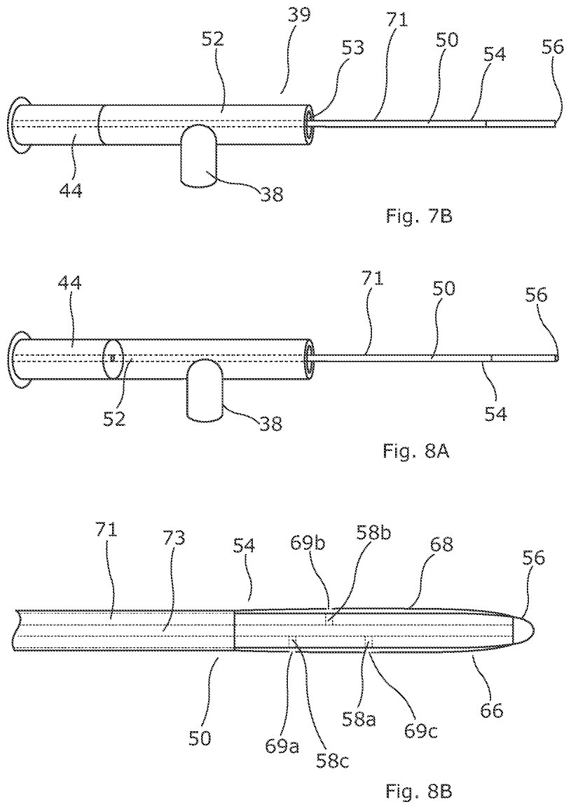

The manifold 36 also enables fluid flow (2) from the container 34, via fluid path 51 (see FIG. 5) and a first outlet 38 of the manifold 36 for delivery of the drug to the patient via conduit 30b as shown in FIG. 3. In a particular arrangement, the manifold 36 may comprise a lower portion 39 for connection to the container 32. The manifold 36 may also comprise an upper portion 43 for connection with the conduit 30a—see, for example, FIG. 7A. In an arrangement, the upper and lower portions 43 and 39 of the manifold 36 may releasably be attached to each other.

Further, the manifold 36 comprises a second inlet 40 (see FIG. 4) to permit delivery of flushing fluid for flushing of the dilution chamber 32 with the objective of delivering any drug remnant inside the dilution chamber 32 into the patient, or priming the apparatus 10 with diluent. The second inlet 40 may be referred to as a flushing inlet. The flushing inlet is configured to receive flushing fluid.

Furthermore, the manifold 36 comprises a multi-way valve 42 (best seen in FIG. 7). The multi-way valve 42 is for controlling fluid flow from the infusion driver 14 (via conduit 30a) and the second inlet 40. In particular, rotation of a valve plug of the multi-way valve 42 (comprising at least one plug port traversing the valve plug) permits selectively displacing the valve plug between a first condition (for opening of the first inlet 37 and closing the second inlet 40), a second condition (for closing of the first inlet 37 and opening the second inlet 40), and a third condition (for opening of the first inlet 37 and opening the second inlet 40, but preventing the flow of pharmaceutical preparation to the container 34). In the first condition fluid flow flows from the infusion driver 14 into the container 34. In the second condition fluid flow flows through the second inlet 40 but is impeded through the first inlet 37. This is particularly useful because it permits setting up (priming with diluent) of the apparatus 10 prior delivering the pharmaceutical preparation to the container 34. In the third condition pharmaceutical preparation flows from the infusion driver 14 and contact is permitted between the conduit 30a and the atmosphere through the second inlet 40 so that the pharmaceutical preparation may reach the manifold 36 for the first time prior the infusion process.

In other words, the multi-way valve 42 is configured to be actuated between a first position and a second position. The multi-way valve 42 is configured to enable flushing fluid from the second inlet 40 into the dilution chamber 32 whilst inhibiting displacement of the pharmaceutical preparation into the dilution chamber 32 when in the first position. The multi-way valve 42 is configured to enable displacement of the pharmaceutical preparation into the dilution chamber 32 and to inhibit flushing fluid from entering the dilution chamber 32 when in the second position. The multi-way valve 42 is also configured to be actuated to a third position. In the third position, the pharmaceutical preparation may flow through the first inlet 37 and the second inlet 40 to atmosphere.

The medication delivery apparatus 10 comprises a one way valve 44. Specifically, the manifold 36 comprises the one way valve 44 (see FIG. 5). The one way valve 44 is configured to allow fluid from the first inlet 37 into the container 34 but to impede fluid flow from the container 34 back into the infusion driver 14 through the first inlet 37. In other words, the one way valve 44 is configured to enable fluid from the active agent chamber 98 to enter the dilution chamber 32 and to inhibit fluid in the displacement chamber 32 from entering the active agent chamber 98. In this manner, any flow exiting the container 34 will necessarily flow through fluid path 51 to outlet 38 for delivery to the patient through conduit 30b.

Referring now to FIGS. 6 to 8, the manifold 36 may be detached from the container 34. For this, a releasable joint is provide between the end 39 of the manifold 36 and the end 48 of the container 34. Detachment of the manifold 36 allows replacement of a catheter 50 extending within and out of the manifold 36 for locating within the container 34. The medication delivery apparatus 10 comprises the catheter 10. The catheter 10 is configured to be at least partially disposed within the dilution chamber 32.

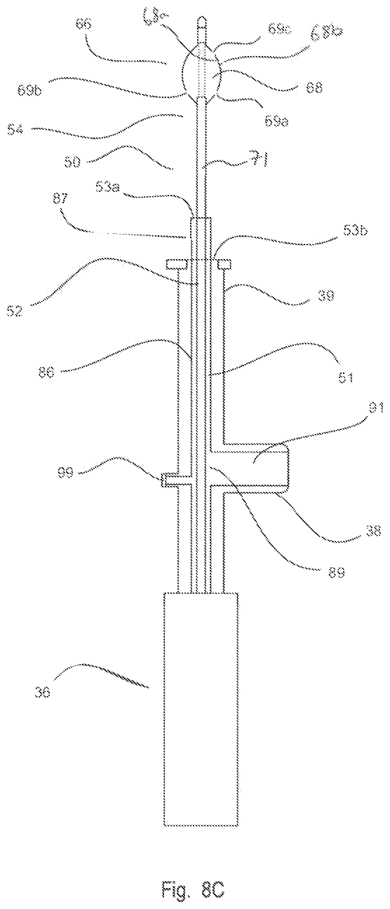

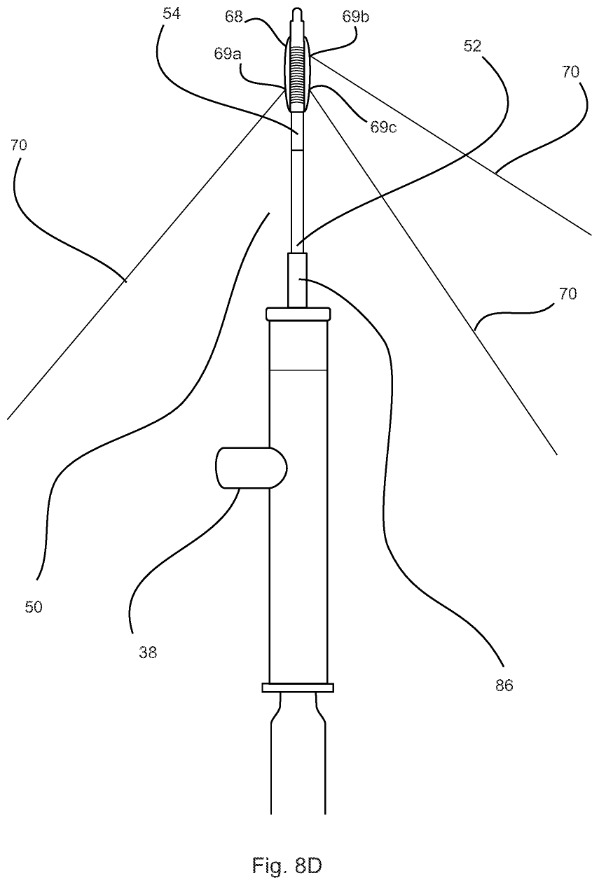

As shown in FIGS. 8, the catheter 50 comprises a catheter body 71. The catheter body 71 defines a hollow core 73 that defines a catheter body fluid flow path. The catheter 50 comprises a plurality of catheter body perforations 58. The catheter body perforations 58 may be referred to as perforations 58. The catheter body perforations 58 are disposed at an end portion of the catheter 50. The catheter 50 comprises proximal end 52 and distal end 54. The distal end 54 may comprise the end portion. That is, the distal end 54 may comprise the catheter body perforations 58. Each catheter body perforation 58 extends between the hollow core 73 and an exterior of the catheter body 71.

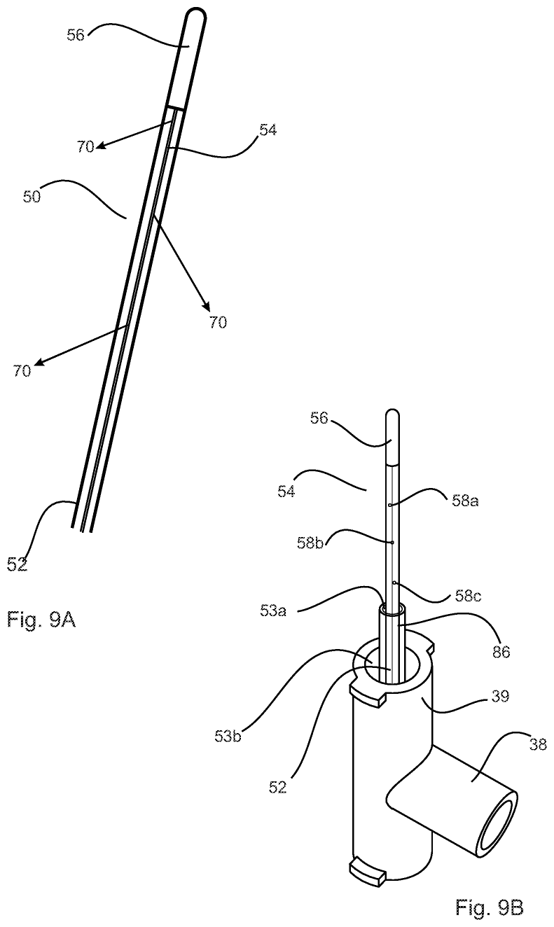

The proximal end 52 is adapted to be fluidly connected to the one way valve 44 to permit fluid from the infusion driver 14 through the catheter 50 and into the container 34. The distal end 54 of catheter 50 comprises (in a particular arrangement) a blind end 56 (seen best in FIGS. 9A and 9B). The blind end 56 impedes fluid flow therethrough. This forces the fluid to flow through perforations 58 traversing the side wall of the distal end 54 of the catheter 50—see FIG. 8b.

The manifold 36 comprises a manifold inlet 53. In particular, the lower portion 39 of the manifold 36 comprises the inlet 53. The manifold 36 comprises a manifold outlet 38. The manifold outlet 38 is configured to connect to the second conduit 30b, thereby enabling the pharmaceutical preparation to be delivered to the patient. The manifold inlet 36 enables the dilution chamber 32 to be fluidly connected with the outlet 38, thereby permitting delivery of the pharmaceutical preparation contained in the dilution chamber 32. As shown best in FIG. 5, a fluid path 51 is formed within the lower portion 39 of the manifold 36 around the proximal end 52 of the catheter 50.

As will be described below with reference to FIGS. 8B to 11E, in accordance with present embodiments of the disclosure, there is provided different types of arrangements of catheters 50.

In accordance with the present embodiments of the disclosure the distal end 54 of the catheter 50 is adapted to deliver the drug received from the infusion driver 14 into the container 34. In the particular arrangement shown in FIGS. 8B to 10, the distal end 54 of the catheter 50 comprises a plurality of perforations 58 (see FIG. 8B). The perforations 58 of the plurality of perforations are arranged in a spaced apart relationship along the length of the catheter 50 and about the outer surface of the catheter 50. The perforations 58 enable the drug (i.e. the pharmaceutical preparation) to exit through the distal end 54 of the catheter 50 in different directions (illustrated by the arrows shown in FIG. 9a or the fluid jets 70). In particular, the perforations 58 enable the drug to exit the catheter 50 as shown in FIG. 8d, with the objective of distributing the drug within the container 34 to ensure proper dilution of the drug in the diluent contained in the container 34.

As shown in FIG. 8B, the catheter 50 may comprise an end location 66. The end location 66 may be on, or part of the distal end 54 of the catheter 50. The end location 66 comprises the perforations 58 referred to previously. The catheter 50 may also comprise a sleeve 68. The sleeve 68 may be flexible. The sleeve 68 surrounds the end location 66. The sleeve 68 is connected to the end portion of the catheter 50. The sleeve 68 comprises a plurality of sleeve perforations 69. The sleeve perforations 69 may be referred to as perforations 69. The sleeve perforations 69 are arranged in a spaced apart relationship along the length of the end location 66 and about the outer surface of the end location 66. The sleeve perforations 69 permit the pharmaceutical preparation to exit through the sleeve 68 in different directions as illustrated in FIG. 8D. In particular instances, during operation, the sleeve 68 expands into a circular or elliptical shape, as can be seen in FIG. 8C.

The sleeve 68 comprises an inner surface 68a and an outer surface 68b. The sleeve perforations 69 extend between the infer surface 68a of the sleeve 68 and the outer surface 68b of the sleeve 68. An active agent catheter flow path is therefore defined between the hollow core 73 and each of the plurality of sleeve perforations 69 via the plurality of catheter body perforations 58.

The catheter 50 is configured to connect to a second end of the first conduit 30a. The end portion of the catheter 50 is configured to be disposed within the dilution chamber 32.

As shown in FIG. 8D, the perforations 69 made in the sleeve 68 are traverse the catheter body 71. In particular, the sleeve perforations 69 angled diagonally in order to encourage the fluid (depicted as jets of fluid 70) exiting the sleeve 68 through the perforations 69 to be directed towards the lower portion 39 of the manifold 36. In a particular arrangement, the flexible sleeve 68 of the catheter 50 (see FIG. 8C) is perforated with three evenly spaced, 30 g (0.25 mm) perforations oriented at 60 degrees above a horizontal.

In an alternative arrangement, the catheter 50 comprises a blind end having plurality of perforations 69. The catheter 50 may be made out, or comprise of a flexible material adapted to be expanded as the flow rate of the active agent increases. Expansion of the catheter 50 results in that the perforations 69 enlarging, reducing resistance to flow rate at high flow rates.

FIGS. 9A and 9B show a second arrangement of the catheter 50 having perforations 58 traversing diagonally of the catheter 50, in order for the fluid flow exiting the distal end 54 of the catheter 50 through the perforations 58 to be directed towards the proximal end 52 of the catheter 50.

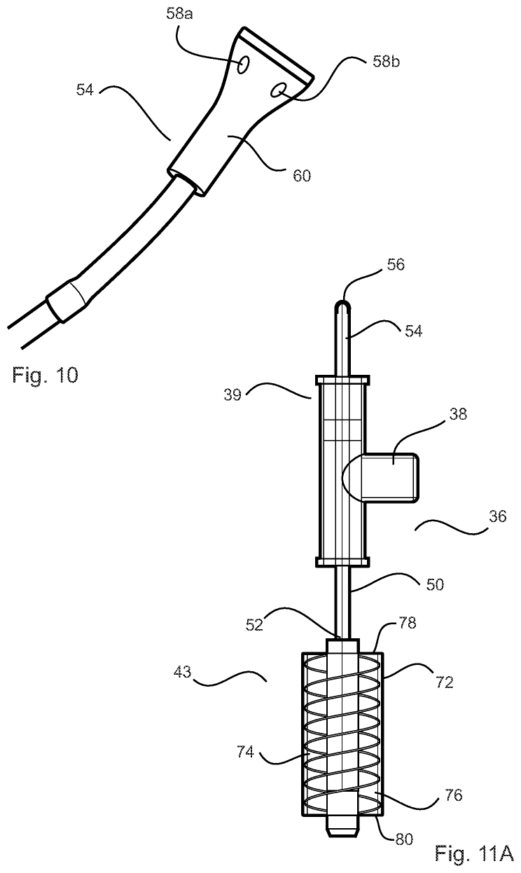

Further, FIG. 10 shows a third arrangement of the catheter 50. In this particular arrangement, the distal end 54 of the catheter 50 comprises a plurality of perforations 58 arranged in a spaced arrangement around side walls of an end 60. In the particular arrangement shown in FIG. 10, the end 60 comprises a conical-like truncated end with an enlarged area of the conical-like truncated end comprising the perforation 58. The distal end 54 may comprise a flexible material.

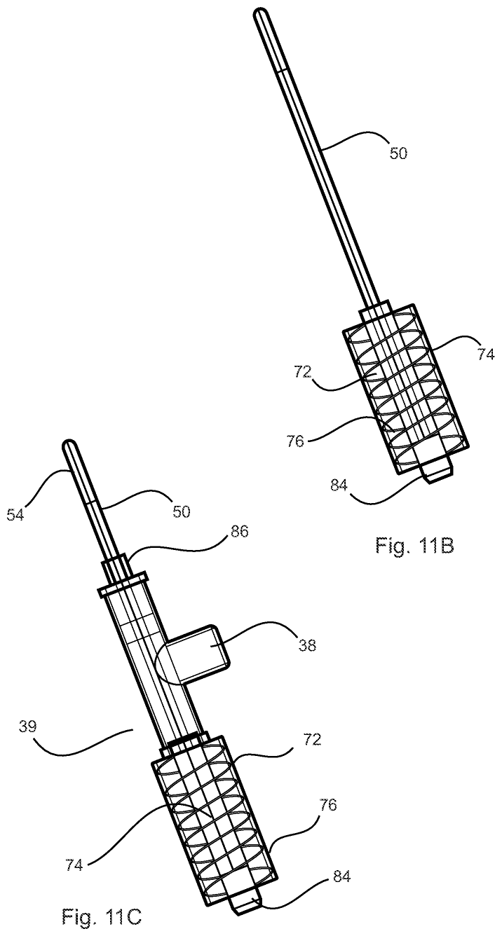

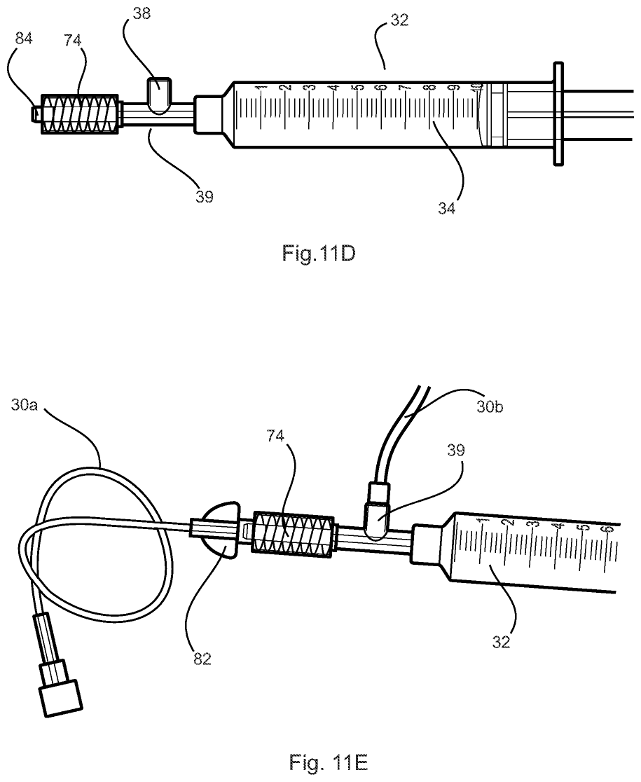

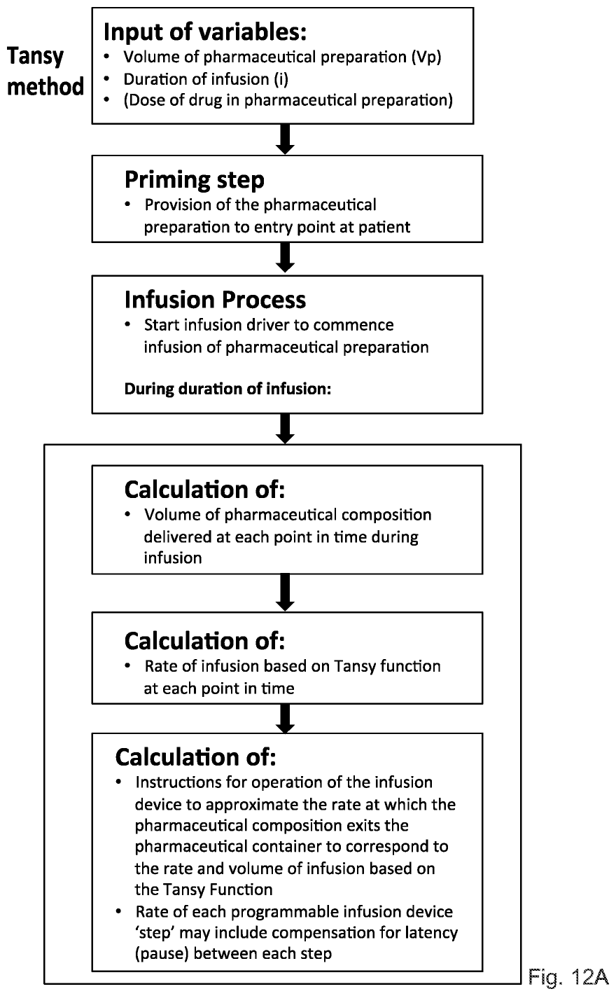

Furthermore, FIGS. 11A-11E show a fourth arrangement of the catheter 50. In the particular arrangement shown in the FIGS. 11A-11E, the catheter 50 comprises a proximal end 52 and distal end 54. In this particular arrangement, the catheter 50 does not have blind end at its end 56. Instead, the end 56 of the catheter 50 is open, permitting exit of fluid flow through the open end 56 of the catheter 50 and allowing the pharmaceutical preparation to enter the container 34 of the dilution chamber 32.

As shown in FIG. 11A, the proximal end of the catheter 50 is attached to a lower end 72 of a connecting body 74. The connecting body 74 has an upper end 76. The connecting body 74 enables the joining together of the upper 43 and lower part 39 of the manifold 36. As shown in FIG. 11B, the lower part 72 of the connecting body 74 is connected to the lower part 39 of the manifold 36.

In the particular arrangement shown in FIGS. 11A-11E, the connecting body 74 comprises a body having two end sections 78 and 80 defining the lower and upper parts 72 and 76 of the connecting body 74. Each end section 78 and 80 comprises an inner thread to permit attachment: (1) of the lower part 39 of the manifold 36 to the lower end 72 of the connecting body 74 as shown in FIG. 11C, and (2) of the upper end 76 of the connecting body 74 to a valve 82 (see FIG. 11E) attached to the conduit 30a. The conduit 30a is fluidly attached to the infusion driver 14 for delivery of the pharmaceutical preparation to the dilution chamber 32 through the catheter 50.

Referring now to FIG. 11D, FIG. 11D shows the lower part 39 of the manifold 36 attached to the container 34 with the catheter 50 inserted in the connecting body 74. As mentioned above, in this arrangement, the pharmaceutical preparation is delivered through the catheter 50 into the container 34. This is done through a one-way valve 84 having a proximal end for attachment of the valve 82 (see FIG. 11E) that is connected to the conduit 30a. Further, the valve 84 at least partially traverses the connecting body 74. The valve 84 has a distal end for attachment to the proximal end 52 of the catheter 50.

During delivery of the pharmaceutical preparation into the container 34, air bubbles may be formed due to the mixing of the pharmaceutical preparation (coming from the infusion driver 14) with the diluent contained in the container 34. The bubbles may reach the conduit 30b which delivers the pharmaceutical preparation (exiting the container 34) to the patients. This should be avoided. FIGS. 8C, 8D and 9B depicts a catheter 50 comprising a bubble trap. The bubble trap is configured to prevent, or minimize the extent to which bubbles reach the conduit 30b.

As shown in FIG. 8C, a particular arrangement of the bubble trap comprises a sleeve 86 at least partially surrounding the proximal end 52 (the first end) of the catheter 50. In particular, the sleeve 86 extends from a particular location within the manifold 36 to a location outside the manifold 36 such that the distal end 87 of the sleeve 86 is located within the container 34 of the dilution chamber 32. A fluid path 51 is defined between the exterior wall of the catheter 50 and the inner wall of the sleeve 86. As will be described below, the fluid path 51 permits delivery of the diluted pharmaceutical preparation (located within the container 34) through the outlet 38 of the manifold 36 and to the patient.

In an arrangement, the particular location within the manifold 36 from which the sleeve 68 extends is where the catheter 50 is attached (within the manifold 36) to an outlet which is fluidly connected to the first inlet 37 of the manifold 36, permitting the delivery of the pharmaceutical preparation flowing through the conduit 30a into the first inlet 37 of the manifold 36 for delivery into the catheter 50.

The fluid path 51 has an open end defined at the distal end 87 of the sleeve 86. The open end is for receiving the diluted pharmaceutical preparation. The fluid path 51 has a sealed end at the particular location within the manifold 36 where the catheter 50 is attached to the outlet. The sealed end is for receiving the pharmaceutical preparation from the first inlet 37. The fact that the fluid path 51 has the sealed end ensures that all the diluted pharmaceutical preparation coming from the dilution chamber 32 is delivered to the outlet 38 for delivery to the patient.

Further, the objective of having the distal end 87 of the sleeve 86 within the container 34 is to permit the diluted pharmaceutical preparation to enter the fluid path 51 for delivery into the outlet 38. For this the fluid path 51 is fluidly connected to the outlet 38. As shown in FIG. 8C, the sleeve 86 comprises an opening 89 fluidly connected to the fluid path 51 defined by the outlet 38.

As shown in FIG. 8C, a first inlet 53a is defined at the distal end 87 of the sleeve 86. This inlet 53a permits the diluted pharmaceutical preparation to enter the fluid path 51 for delivery to the patient via outlet 38. A second inlet 53b is formed at the location where the sleeve 86 exits the manifold 36. This inlet 53b is defined between (1) the particular end (the distal end) of the manifold 36 onto which the container 34 is connected, and (2) the outer wall section of the sleeve 86 that is opposite to the inner wall of the particular end of the manifold 36 onto which the container 34 is connected. Inlets 53a and 53b can be seen in FIG. 9B.

In operation, the pharmaceutical preparation enters the fluid path 51 through the inlet 53a for delivery to the patient.

Further, the sleeve 86 deviates bubbles forming at the distal end of the catheter 50 and floating adjacent the catheter 50 preventing bubbles from entering the fluid path 51 through the inlet 53a. Instead, the bubbles enter the lower portion 39 of the manifold 36 through the inlet 53b (best seen in 9b). In this particular arrangement, a venting means 99 is provided for relieving any excess pressure or removing air bubbles that may be contained in the manifold 36.

In the arrangement shown in the figures (for example, FIG. 4), the dilution chamber 32 comprises a container 34 that is adapted to be selectively displaced between an expanded condition and a contracted condition. In the expanded condition, the container 34 permits storage of the diluent for receiving the drug. In the contracted condition, the container 34 forces any remaining drug contained in the container 34 to be delivered to the patient. In the arrangement shown in the figures, the dilution chamber 32 comprises a syringe 62. The dilution chamber 32 also comprises a plunger 64. The plunger 64 may be referred to as a second plunger. The plunger 64 is adapted to be selectively displaced for displacing the container 34 between the expanded condition and the contracted condition to expel the remnant portion of drug into the patient. The plunger 64 is configured to be selectively displaced along a longitudinal axis of the dilution chamber 32.

There are two different disposable consumable systems particularly suited to clinical use, one with a 10 ml dilution chamber 32, and one with a 20 ml dilution chamber 32, although the method includes arrangements with dilution chambers 32 of other volume sizes (and the example of a method with a chamber volume of 0 mL is equivalent to the Tansy Method). The 20 ml dilution chamber 32 allows for a greater minimum infusion rate and a lower maximum infusion rate that the 10 ml chamber 32, but at a cost. This costs is that the fraction of drug delivered to the patient at any point of the infusion is reduced (by

$$\frac{{V}_{p} - {V}_{d} ( 1 - {e}^{- \frac{Vp}{Vd}} )}{{V}_{p}}$$

where V_{d} is the volume of the dilution chamber 32 and Vₚ is the primary syringe infusion volume; the intention of this is that the drug remaining in the dilution chamber 32 (upon completion of the infusion process) is delivered to the patient as a bolus by emptying the dilution chamber 32 by, for example, depressing the syringe plunger, or by flushing the system with saline.

Alternatively, (1) the concentration of active ingredient in the pharmaceutical preparation can be increased (‘Increased concentration Sadleir method’) or (2) the volume of the pharmaceutical preparation and infusion rates can be increased (‘Increased volume Sadleir method’); any of (1) or (2) is done to deliver the same dose as the equivalent Tansy method at the end of the infusion period (i). In both of these alternative methods, the drug remaining in the dilution chamber 32 upon completion of the infusion process is discarded.

For infusions of duration greater than 25 minutes, a dilution chamber of ⅕ the volume of the infusion volume (i.e. 10 ml for a 50 ml infusion, 20 ml for a 100 ml infusion) is appropriate because approximately 80% of the total dose is given prior to the final bolus. For infusions over 20-25 minutes, a ⅖ (i.e.: a 20 ml dilution chamber for a 50 ml primary infusion volume) ratio ensures that infusion rates do not exceed 20 ml/min for a 50 ml infusion.

Clinically, the 30 minute infusion with 50 ml volume and 10 ml dilution chamber is appropriate in terms of the competing interests of (1) achieving infusion of the full therapeutic dose in a relative short period of time, but also (2) allowing for detection of submaximal adverse reaction in the patient. For infusions that are not witnessed by a doctor (i.e.: given unattended on the ward), it may be more appropriate to use the Sadleir function over 60 to 120 minutes, and with a 100 ml volume and 20 ml dilution chamber.

However, the infusion duration is likely to be limited by several factors. The first factor is the maximum infusion rate tolerated by typical-sized intravenous cannulas (i.e.: 22 g). A second factor is that the maximum infusion rate of 20 ml/hr on most infusion drivers 14 resulting in that the minimum commonly used Sadleir function infusion duration will be 20 minutes for a 50 ml infusion volume and a 20 ml dilution chamber 32.

In accordance with the second embodiment of the disclosure, the infusion driver 14 delivers the drug via conduit 30a to the dilution chamber 32 and then to the patient via conduit 30b fluidly connected to the patient (see FIG. 3). And, the processor 16 executes codes running a particular algorithm for driving of the syringe driver 17 in order to deliver the pharmaceutical preparation (contained in the syringe 15) to the patient as dictated by the Sadleir function.

The apparatus 10 may be used for the administration of all therapeutic doses of any drugs (active ingredients such as medications) diluted in a diluent forming diluted pharmaceutical preparations that can be gradually administered to patients with the objective of decreasing the incidence of severe hypersensitivity reactions and avoid death of any hypersensitivity patients.

In particular, the apparatus 10 in accordance with the first and second embodiment of the disclosure is intended to be used, for example, in one of three scenarios:

Drug Test Dose—in a patient who is not suspected to be hypersensitive to the drug to be administered to the patient, in which case the apparatus 10 is used to administer the therapeutic dose of a drug in a particular manner (for example providing sequentially increasing test doses) that increases the chance that any unexpected hypersensitivity is detected, permitting stopping of the infusion process before a dose that will cause a more serious reaction to the patient has been administered. In this particular scenario, patients, who would otherwise have had an unexpected reaction to the drug, with the particular manner in which the therapeutic dose is administered, a tolerance is induced in the patient and no negative reaction will occur. Thus, this particular scenario generates what is typically referred to as unintentional acute desensitization.

Drug Challenge—in a patient who is suspected of having a hypersensitivity reaction due to a particular drug, and in whom it is deemed advantageous to confirm that the particular drug administered was responsible for the reaction, the apparatus 10 is used to administer the therapeutic dose of a drug in a particular manner that increases the capability or probability that, if a hypersensitivity reaction does occur, the infusion can be stopped before a particular quantity of the drug becomes a dose that will cause a more serious reaction in the patient. This scenario is particularly useful for confirming that the drug administered to the patient was responsible for the patient's hypersensitivity reaction.

Drug Desensitisation—in a patient who is known to be hypersensitive to a particular drug, in which case a therapeutic dose of the particular drug is administered in a particular manner (for example, providing relative low doses at the start of the infusions process) using the apparatus 10 such that tolerance is induced to the drug. This scenario is particularly useful for desensitising the patient to the particular drug.

Part 3—Methods for Delivering a Pharmaceutical Preparation

According to one example of the present disclosure an infusion device is configured to control a medication delivery apparatus to intravenously deliver a pharmaceutical preparation to a patient according to a predetermined dose profile. For example, the infusion device may comprise a processor and a memory storing instructions executable by the processor to cause the medication delivery apparatus to deliver the pharmaceutical preparation to the patient according to a predetermined dose profile.

The predetermined dose profile is designed to deliver a therapeutic dose of the pharmaceutical preparation to the patient over a predetermined infusion time in a manner which facilitates safe detection of an adverse reaction of the patient to the pharmaceutical preparation, or desensitization the patient to the pharmaceutical preparation, during a first stage of administering the therapeutic dose. Various examples of dose profile which match these criteria will now be described.

The predetermined dose profile may be such that delivering the first 0.01% of the therapeutic dose takes longer than 0.01% of the predetermined infusion time. Whereas for a constant dose rate infusion, the 0.01% of the therapeutic dose would in general take 0.01% of the infusion time to deliver, in dose profiles proposed herein, delivering 0.01% of the dose profile takes longer than for a constant rate infusion.

In some examples, the predetermined dose profile is such that, after 56% of the infusion time, or alternatively after 34% of the infusion time, the cumulative dose delivered to the patient is no more than 1% of the therapeutic dose. Furthermore, in some examples, after 14% of infusion time the cumulative dose delivered to the patient is no more than 0.6% of the therapeutic dose. Thus, as at the beginning of the infusion, the dose rate is low and so it takes a relatively long time to achieve these low levels of cumulative dose. This may facilitate detection of a minor adverse reaction before a more serious adverse reaction can occur, or may facilitate desensitization of the patient to the pharmaceutical preparation.

In some examples, the predetermined dose profile is such that there is a first time period between the cumulative dose reaching 0.01% and 0.1% and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction. This may facilitate detection of a minor adverse reaction before a more serious adverse reaction can occur.

In some examples, the predetermined dose profile is such that successive orders of magnitude of cumulative dose, such as 0.01%, 0.1%, 1% and 10% of the therapeutic dose, are separated in time from each other by periods of time; wherein the periods of time are selected from the group comprising: at least 6 minutes, at least 5 minutes, at least 4 minutes, at least 3 minutes between 2 minutes and 10 minutes, and at least the latent period of adverse reaction. Thus, as at the beginning of the infusion, the dose rate is low and so it takes a relatively long time to achieve these low levels of cumulative dose. This may facilitate detection of a minor adverse reaction before a more serious adverse reaction can occur.

In general, the level of exposure at which patients who are sensitive to a particular drug will develop an adverse reaction will vary from patient to patient. For instance, it may be that most patients who will develop an adverse reaction will do so within the first 10% of therapeutic dose, however amongst those some may develop an adverse reaction by the time the cumulative dose has reached 0.01%, of the therapeutic dose, others 0.1%, others by 1% of the therapeutic dose etc. While in conventional dosage regimes, these milestones are reached in quick succession often within the first few seconds of the infusion, by designing a dose profile in which these milestones are spaced out in time by several minutes it becomes possible to detect adverse reactions before serious harm occurs.

The latent period of adverse reaction is the average period of time which takes for an adverse reaction to develop. The latent period of adverse reaction for a given drug may be found by consulting medical literature for that drug. Accordingly, based a dose profile for a particular drug may be designed based on the latent period of adverse reaction for that drug.

The predetermined dose profile may be such as to delivers the therapeutic dose over a predetermined infusion time which is between 20 minutes and 180 minutes. Thus while the initial part of the infusion may be at a slow dose rate, the infusion as a whole may kept within a reasonable frame of time Infusion times over 180 minutes may clinically inconvenient, while for infusion times much under 20 minutes it may be difficult to design a dose profile which delivers the initial part of the dose slowly enough to safely detect adverse reactions.

The predetermined dose profile may be such that the dose rate varies over the predetermined infusion time. In this way the infusion may kept within a reasonable time frame, as the dose rate may start off slow, but increase as the infusion progresses. In some examples, the dose rate may increase over time in the period between 14% of the infusion time and 78% of the infusion time. For instance, in earlier parts of the infusion the dose rate may vary up and or down as part of an initial process and in later parts of the infusion the infusion rate may have reached a steady state.

The predetermined dose profile may be such that the cumulative dose delivered to the patient increases at a rate that increases over time, for at least a portion of the predetermined infusion time. In some examples, the cumulative dose may increase at an exponential rate. This helps to shorten the infusion time while increasing the chance that an adverse reaction will be detected before it becomes serious.

In some examples, the predetermined dose profile is such that the dose rate increases exponentially, or increases at a rate that increases over time, for at least a portion of the predetermined infusion time. For example, the predetermined dose profile may be such that the dose rate increases over the infusion and times at which the dose rate reaches 0.01%, 0.1%, 1% and 10% of the maximum dose rate are separated in time from each other by a period of time selected from the group comprising: at least 6 minutes, at least 5 minutes, at least 4 minutes, at least 3 minutes between 2 minutes and 10 minutes, and at least the latent period of adverse reaction.

The at least a portion of the predetermined infusion time over which the cumulative dose and/or the dose rate increases may be a portion of the predetermined infusion time prior to a maximum drug administration rate for a particular drug being reached. In some examples the at least a portion of the predetermined infusion time may be longer than two minutes. In some examples the at least a portion of the predetermined infusion time may be at least 40% of the predetermined infusion time. In some examples, the at least a portion of the predetermined infusion time may be the between 14% of the infusion time and 78% of the infusion time.

In some examples, the predetermined dose profile includes a maximum dose rate and the predetermined dose profile is such that the maximum dose rate occurs at a time after 50% of the predetermined infusion time has passed.

Where cumulative dose delivered increases exponentially over time, the dose rate may increase steadily or exponentially. Where dose rate increases exponentially over a period of time, the cumulative dose delivered to the patient will also increase exponentially over said period of time. The predetermined dose profile may be described by, or approximate to, a dose function. The inventor has found that dose functions in which both the cumulative dose delivered and the dose rate increase in a similar manner have characteristics which are particularly suitable for facilitating detection of an adverse reaction to a drug and/or drug sensitization. Accordingly, in some examples, the dose profile is such that a time at which the cumulative dose reaches 10% of the therapeutic dose and a time at which the dose rate reaches 10% of the maximum dose rate are the same plus or minus 5% of the predetermined infusion time. In some examples, the dose profile is such that a time at which the cumulative dose reaches 50% of the therapeutic dose and a time at which the dose rate reaches 50% of the maximum dose rate are the same plus or minus 5% of the predetermined infusion time. In some examples, the dose profile is such that a time at which the cumulative dose reaches 1% of the therapeutic dose and a time at which the dose rate reaches 1% of the maximum dose rate are the same plus or minus 5% of the predetermined infusion time. In some examples, the dose profile has a maximum dose rate and the dose profile is such that the time which it takes for the cumulative dose to reach 0.1%, 1% and/or 10% of the therapeutic dose respectively is substantially the same as the time which it takes for the dose rate to reach 0.1%, 1% and/or 10% of the maximum dose rate.

In some examples, the predetermined dose profile is such that the dose rate doubles over a period of time during the infusion, the period of time over which the dose rate doubles being 1.33 minutes to 12 minutes. In some examples, the predetermined dose profile is such that, for at least part of the infusion, the dose rate doubles every X minutes, where X is in the range 1.33 minutes to 12 minutes.

In some examples, the infusion device may be configured to generate a flow rate of pharmaceutical preparation to the patient between 0.005 ml/min and 350 ml/min.

The dose profile may be defined by, or approximate, an exponential dose rate function. The dose rate function may be such that the dose rate doubles every b minutes. Such functions, in which the dose rate doubles every b minutes, may be generally referred to as Tansy Functions. In some examples, a Tansy function may be expressed as an exponential dose rate function having an exponent of the form t/b, for example (t/b)*ln(2\hat{ }(30/i), where t represents the elapsed time, b is a constant and represents the time over which the dose rate doubles, and i is the duration of the infusion in minutes (“i” may also referred to as the “predetermined infusion time”). Dose profiles which have a dose rate defined by a Tansy functions may be particularly useful for controlling an infusion in order to detect an adverse reaction, perform or provocation test, or desensitization infusion.

An example of a Tansy function, in which the doubling time b is equal to 2 minutes, is given below.

$$infusion rate ( ml / min ) = \frac{{V}_{p} \times ln ( {2}^{30 / i} )}{{2}^{16} - 2} {e}^{\frac{t}{2} ln ( {2}^{30 / i} )} ,$$

where t=time elapsed, i=duration of the infusion and Vₚ=volume of the pharmaceutical preparation solution in a primary chamber.

$$dose rate ( mg / min ) = {C}_{p} \times \frac{{V}_{p} \times ln ( {2}^{30 / i} )}{{2}^{16} - 2} {e}^{\frac{t}{2} ln ( {2}^{30 / i} )} ,$$

where Cₚ is the concentration of the active ingredient of the pharmaceutical preparation solution in the primacy chamber

The Tansy cumulative volume function is the integral of the rate function and is:

$$cumulative volume ( ml ) = {\int }_{0}^{t} \frac{{V}_{p} \times ln ( {2}^{30 / i} )}{{2}^{16} - 2} {e}^{\frac{t}{2} ln ( {2}^{30 / i} )} dt$$

Or:

$$cumulative volume ( ml ) = \frac{2 \times {V}_{p}}{{2}^{16} - 2} {e}^{\frac{t}{2} ln ( {2}^{30 / i} )} - \frac{2 \times {V}_{p}}{{2}^{16} - 2}$$

The Tansy cumulative dose function is thus:

$$cumulative dose ( mg ) = {C}_{p} \times ( \frac{2 \times {V}_{p}}{{2}^{16} - 2} {e}^{\frac{t}{2} ln ( {2}^{30 / i} )} - \frac{2 \times {V}_{p}}{{2}^{16} - 2} )$$

However, the above example of a Tansy function, is a special case of a more general series of equations (Tansy Functions) that are particularly suitable for controlling infusions in a manner suitable to detect an adverse reaction, or provocation test, or desensitization infusion.

The general form of the infusion rate Tansy function is:

$$infusion rate ( ml / min ) = \frac{a \times {V}_{p} \times \frac{ln ( {2}^{30 / i} )}{b}}{{2}^{16} - 2} {e}^{\frac{t}{b} ln ( {2}^{30 / i} )} ,$$

where a and b are constants. The constant b expresses time taken for the rate to double. When i=30, b is equal to the number of minutes taken for the rate to double. That is, if i=30 and b=2, then it will take 2 minutes for the dose rate to double.

The general form of the dose rate Tansy function is:

$$dose rate ( mg / min ) = {C}_{p} \times \frac{a \times {V}_{p} \times \frac{ln ( {2}^{30 / i} )}{b}}{{2}^{16} - 2} {e}^{\frac{t}{b} ln ( {2}^{30 / i} )}$$

The general form of the cumulative dose function is:

$$cumulative dose ( mg ) = {C}_{p} \times ( \frac{a \times {V}_{p}}{{2}^{16} - 2} {e}^{\frac{t}{b} ln ( {2}^{30 / i} )} - \frac{a \times {V}_{p}}{{2}^{16} - 2} )$$

Where:

$$a = \frac{{2}^{16} - 2}{{2}^{30 / b} - 1}$$

And the general form of the dose rate Tansy function can be simplified (as can the derived functions above be also in a similar way) to:

$$infusion rate ( mL / min ) = \frac{{V}_{p} \times ln ( {2}^{30 / i} )}{b ( {2}^{30 / b} - 1 )} {e}^{\frac{t}{b} ln ( {2}^{30 / i} - 1 )}$$

The values of b can be chosen to alter the Order Magnitude Delay (OMD). The OMD refers to the period of time which it will take for the delivered cumulative dose to increase from the currently stated dose to 10 times the currently stated dose. For instance the OMD 0.01% is the time taken for the cumulative dose to increase from 0.01% of the therapeutic dose to 0.1% of the therapeutic dose (i.e. to increase by an order of magnitude). The OMD 0.1% is the time taken for the cumulative dose to increase from 0.1% of the therapeutic dose to 1% of the therapeutic dose. The OMD 0.3% is the time taken for the cumulative dose to increase from 0.3% of the therapeutic dose to 3% of the therapeutic dose etc. The OMD of a dose function may vary depending on the current cumulative dose, i.e. it may vary between earlier and later in the infusion.

The inventor has found that by changing the value of b, the OMD of the Tansy dose rate function can be changed. The inventor has further found that values of b in the range 1.5 to 4 provide useful dose profiles for the purposes of detecting an adverse reaction, performing a provocation test or desensitization. The inventor has found that, in general, higher values of b will result in longer OMD for higher cumulative doses (e.g. 1%) but at a cost of lower OMD for lower cumulative doses (e.g. 0.01%). That is, increasing b will decrease the time required to reach lower cumulative doses, but stretch out the time required to reach higher cumulative doses.

Table 1 below shows example values of OMD 0.01%, OMD 0.1%, OMD 1% and OMD 10% for Tansy Functions with values of b between 1.8 and 3.

TABLE 1

| | Equation | | | |
|---|---|---|---|---|
| | OMD 0.01% | OMD 0.1% | OMD 1% | OMD 10% |
| Constant (b) | 0.03 min | 0.27 min | 2.7 min | 27 min |
| 1.8 | 5.87 | 5.97 | 5.98 | 5.97 |
| 2 | 6.28 | 6.61 | 6.64 | 6.64 |
| 2.25 | 6.35 | 7.34 | 7.46 | 7.47 |
| 2.5 | 5.84 | 7.93 | 8.27 | 8.30 |
| 2.75 | 4.97 | 8.32 | 8.27 | 9.12 |
| 3 | 4.02 | 8.45 | 9.78 | 9.93 |

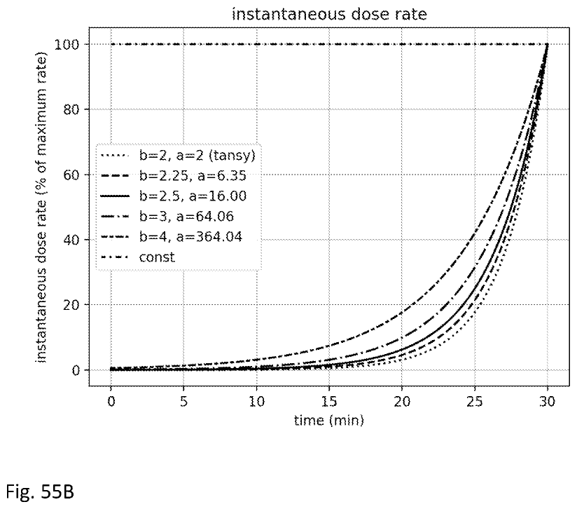

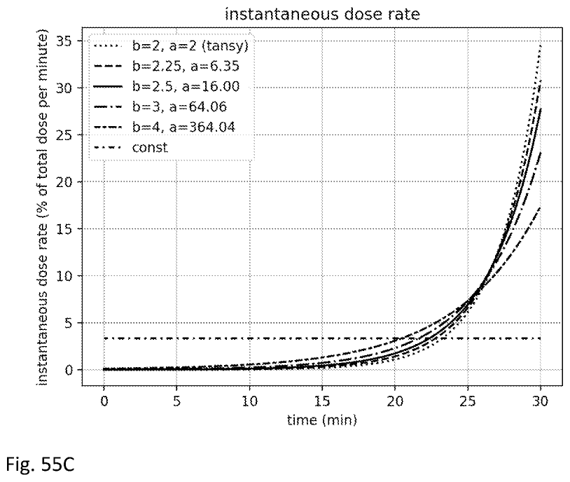

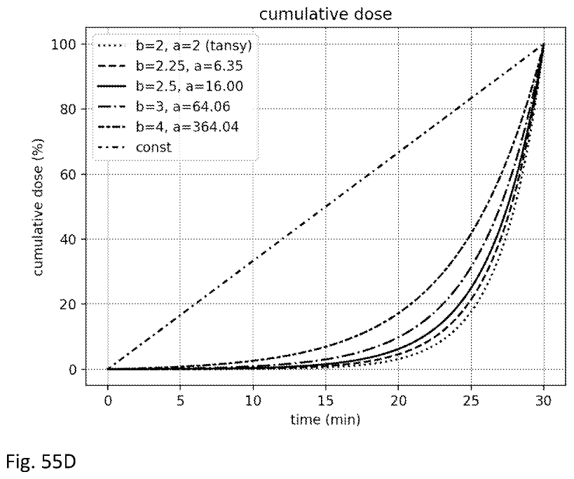

FIG. 55A is a graph showing the variation of OMD against cumulative dose for an infusion having a duration of 30 minutes (i.e. “i” or the “predetermined infusion time” is 30 minutes) for Tansy functions with a value of b between 2 and 4 and corresponding values of a (denoted as “a” in the graph), as well as for a constant infusion rate function.

FIG. 55B is a graph showing the instantaneous dose rate (as a percentage of the maximum dose rate) against time for an infusion having a duration of 30 minutes for Tansy functions with a value of b between 2 and 4 and corresponding values of a (denoted as “a” in the graph), as well as for a constant infusion rate function.

FIG. 55C is a graph showing the instantaneous dose rate (expressed as a percentage of the total dose delivered per minute) against time for an infusion having a duration of 30 minutes for Tansy functions with a value of b between 2 and 4 and corresponding values of a (denoted as “a” in the graph), as well as for a constant infusion rate function.

FIG. 55D is a graph showing the cumulative dose delivered (expressed as a percentage of the therapeutic dose) against time for an infusion having a duration of 30 minutes for Tansy functions with a value of b between 2 and 4 and corresponding values of a (denoted as “a” in the graph), as well as for a constant infusion rate function.

It will be appreciated that while the constant rate infusion function is not suitable for detecting adverse reactions at low dose rates or cumulative doses, the various Tansy functions with b between 1.5 and 4 are suitable for this purpose. It will further be appreciated that as varying b changes the characteristics of the function, certain values of b will be more suitable for certain patient populations or certain drugs, as for certain patient populations or drugs it will be better to have a larger OMD earlier in the infusion and for other patient populations or drugs it will be better to have a larger OMD later in the infusion.

For example, if a patient population has a distribution of drug provocation thresholds for a particular drug were 0.01% of the therapeutic dose is the minimum reactive threshold (minimum threshold at which an adverse reaction may occur), and 5 minutes is the latent period of the adverse reaction, b=2 may work well. This would ensure the OMD at 0.01% and greater is greater than 5 minutes.

For example, if a patient population has a distribution of drug provocation thresholds for a particular drug where 0.1% of the therapeutic dose is the minimum reactive threshold, and 5 minutes is the latent period of the adverse reaction, a dose profile approximating a Tansy function with b=3.5 may work well. This would ensure the OMD at 0.1% and greater is greater than 5 minutes.

For example, if a patient population has a distribution of drug provocation thresholds for a particular drug were 0.1% of the therapeutic dose is the minimum reactive threshold, and 7 minutes is the latent period of adverse reaction, a dose profile approximating a Tansy function in which b=3 may work well. This would ensure the OMD at 0.1% and greater is greater than 7 minutes.

For example, if a patient population has a distribution of drug provocation thresholds for a particular drug were 1% of the therapeutic dose is the minimum reactive threshold, and 10 minutes is the latent period of the drug reaction, a dose profile approximating a Tansy function in which b=4 may work well. This would ensure the OMD at 1% and greater is greater than 10 minutes.

The inventor envisages that Tansy functions with values of b between 2.25 and 4 or between 1.5 and 1.8 may be particularly useful and may in certain circumstances provide superior dose profiles for detecting adverse reactions to certain drugs compared to Tansy functions with a value of b of 2.

While the Tansy functions are exponential functions, other functions such as polynomial functions which approximate an exponential function may over a certain period of time may also be used. The inventor has noticed that, in many cases, one characteristic of some useful functions for detecting an adverse reaction at low doses is that, at certain times during the infusion, the functions have similar values for cumulative dose delivered (as a proportion of therapeutic dose/maximum cumulative dose) and instantaneous dose rate (as a proportion of the maximum dose rate) reached. For instance, the cumulative dose reaching 10% of the therapeutic dose may occur at approximately the same time that the dose rate reaches 10% of the maximum dose rate. This is shown below in Table 2 for Tansy Functions in which b is between 1.8 and 3, but will be the same for non-Tansy functions, for instance polynomial functions, which have similar dose profiles.

In Table 2, the notation t/2 is used to denote a Tansy function in which b=2, t/2.25 to denote a Tansy function in which b=2.25 etc.

TABLE 2

| | t/2 | | t/2.25 | | t/2.5 time | |
|---|---|---|---|---|---|---|
| | % of | t/2 | % of | t/2.25 | % of | |
| % of | infusion | % of | infusion | % of | infusion | |
| maximum | time taken | infusion | time taken | infusion | taken to | t/2.5 |
| cumulative | to reach | time taken | to reach | time taken | reach | % of infusion |
| dose or | stated | to reach | stated | to reach | stated | time taken to |
| dose rate | cumulative | stated | cumulative | stated | cumulative | reach stated |
| reached | dose | dose rate | dose | dose rate | dose | dose rate |
| 0.01% | 14.0% | 11.4% | 7.67% | 0.34% | 4.12% | 0.00% |
| 0.1% | 33.8% | 33.6% | 26.3% | 25.3% | 19.6% | 17.0% |
| 1% | 55.7% | 55.7% | 50.3% | 50.2% | 44.9% | 44.6% |
| 10% | 77.9% | 77.9% | 75.1% | 75.1% | 72.3% | 72.3% |
| 50% | 93.3% | 93.3% | 92.5% | 92.5% | 91.7% | 91.7% |
| | t/2.75 | | t/3 | | t/1.8 | |
| | % of | t/2.75 | % of | t/3 | % of | |
| % of | infusion | % of | infusion | % of | infusion | |
| maximum | time taken | infusion | time taken | infusion | time taken | t/1.8 |
| cumulative | to reach | time taken | to reach | time taken | to reach | % of infusion |
| dose or | stated | to reach | stated | to reach | stated | time taken to |
| dose rate | cumulative | stated | cumulative | stated | cumulative | reach stated |
| reached | dose | dose rate | dose | dose rate | dose | dose rate |
| 0.01% | 2.32% | 0.00% | 7.67% | 0.00% | 21.1% | 20.3% |
| 0.1% | 14.2% | 8.65% | 26.3% | 0.34% | 40.3% | 40.2% |
| 1% | 39.8% | 39.1% | 34.9% | 33.6% | 60.1% | 60.1% |
| 10% | 69.6% | 69.6% | 66.9% | 66.8% | 80.1% | 80.1% |
| 50% | 90.8% | 90.8% | 90.0% | 90.0% | 94.0% | 94.0% |

Thus from Table 2 it can be seen that it takes 14% of the infusion time for the cumulative dose rate of a t/2 Tansy function to reach 0.01% of the therapeutic dose, but only 11.4% of the infusion time for the t/2 Tansy function to reach 0.01% of the maximum dose rate. However, the time taken to reach 10% of the therapeutic dose (77.9% of the infusion time) is approximately the same as the taken to reach 10% of the maximum dose rate (77.9% of the infusion time). Furthermore, this relationship also holds for other values of b, especially for 10% and 50% being reached, but also to a lesser extent for 1% being reached.

Accordingly, in some examples, the dose profile is such that a time at which the cumulative dose reaches 10% of the therapeutic dose and a time at which the dose rate reaches 10% of the maximum dose rate are the same plus or minus 5% of the predetermined infusion time. In some examples, the dose profile is such that a time at which the cumulative dose reaches 50% of the therapeutic dose and a time at which the dose rate reaches 50% of the maximum dose rate are the same plus or minus 5% of the predetermined infusion time.

Various dose profiles, characterized in various different ways, have been described above. While delivering a drug to patient according to the above described dose profiles has various advantages for detecting adverse reactions, controlling a medication delivery apparatus to deliver such a dose profile is not a trivial matter. There are various practical difficulties in implementing such a system. One difficulty is translating the desired dose profile into a succession of flow rates which will deliver a desired dose profile.

Another difficulty is that infusion drivers may not be able to accurately deliver and control low infusion rates. For instance, a syringe driver, may not be able to accurately control a syringe at low infusion rates. However, as discussed above, the most important part of the dose profile is the early parts which have low dose rates.

Accordingly, in some examples, the medication delivery apparatus comprises an active agent chamber for receiving the pharmaceutical preparation and a dilution chamber for receiving pharmaceutical preparation ejected from the active agent chamber and diluting the pharmaceutical preparation with a diluent, the dilution chamber comprising an dilution chamber outlet for delivering the diluted pharmaceutical preparation to the patient. By diluting the pharmaceutical preparation in a dilution chamber, a relatively higher infusion rate may be used to deliver a relatively low dose rate. However, when this approach is used, the concentration of the pharmaceutical preparation may vary over the infusion process, which further complicates the calculation of appropriate flow rates to the patient as the varying concentration needs to be determined, or modelled, in advance and taken into account.

In some examples, the dilution chamber may have a volume of at least 10 ml or at least ⅕ of the volume of the activate agent chamber and the dilution chamber outlet may have a smaller area than a cross sectional area of the dilution chamber in the direction perpendicular the central axis of the dilution chamber.

The Tansy Functions discussed above are developed on the basis of an apparatus which delivers the pharmaceutical preparation directly from the active agent chamber to the patient. Functions have been developed which takes into account the complexity introduced by the dilution chamber and may be used to control an apparatus with a dilution chamber to deliver the same dose rate and dose profile as a Tansy Function. These functions are referred to generally as Sadleir Functions.

In some cases, the infusion device may be configured such that the dilution chamber has a fixed volume in a first portion of the predetermined infusion time and a variable volume in a second portion of the predetermined infusion time. This may example, enable the infusion driver to empty all of the drug (pharmaceutical preparation) out of the dilution chamber in the second time period after the drug chamber (active agent chamber) is empty, so that none of the drug is wasted.

Functions generally referred to as Diocles Functions model this situation with first and second time periods and enable such an arrangement to deliver at a dose rate the same as a Tansy function in the first time period and a constant dose rate in the second time period.

While specific examples of Tansy Function, Sadleir Function and Diocles Function are described in Parts 3A-3C of this application and elsewhere, it is to be understood that these are by way of example only and could be modified to present different Tansy, Sadleir or Diocles Functions by changing the value of b.

The processor of the infusion device may control the medication delivery apparatus to deliver the pharmaceutical preparation according to the predetermined profile by controlling an actuator of the infusion device. For example the actuator may controlled to drive a plunger, or a pump, of the medication delivery apparatus such that the pharmaceutical preparation is delivered according to the predetermined dose profile. For instance, the processor may divide the predetermined infusion time into a number of infusion steps and determine a target flow rate or a target output volume for each infusion step such that the predetermined dose profile is realized when the actuator is controlled according to the target flow rate or target output volume for each infusion step. The target flow rates or target output volumes for the infusion steps for a predetermined dose profile may be determined by referring to a lookup table stored in the memory. The lookup table may be populated according to the techniques described herein, for instance calculating the target flow rate or target output volume for each infusion step based on modelling of the predetermined dose profile. In other examples, target flow rate or target output volume for each infusion step may be calculated by the processor in real time.

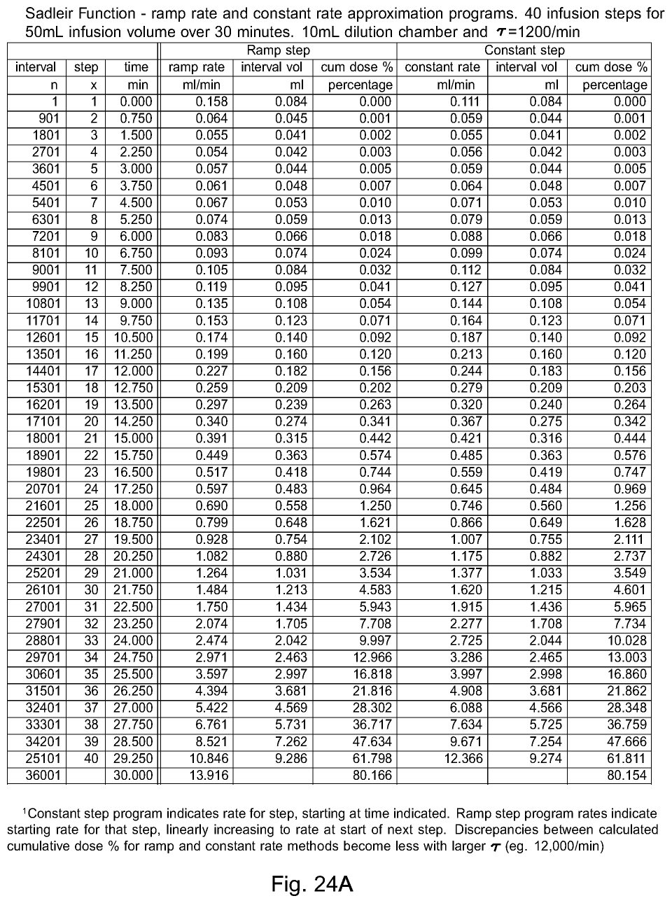

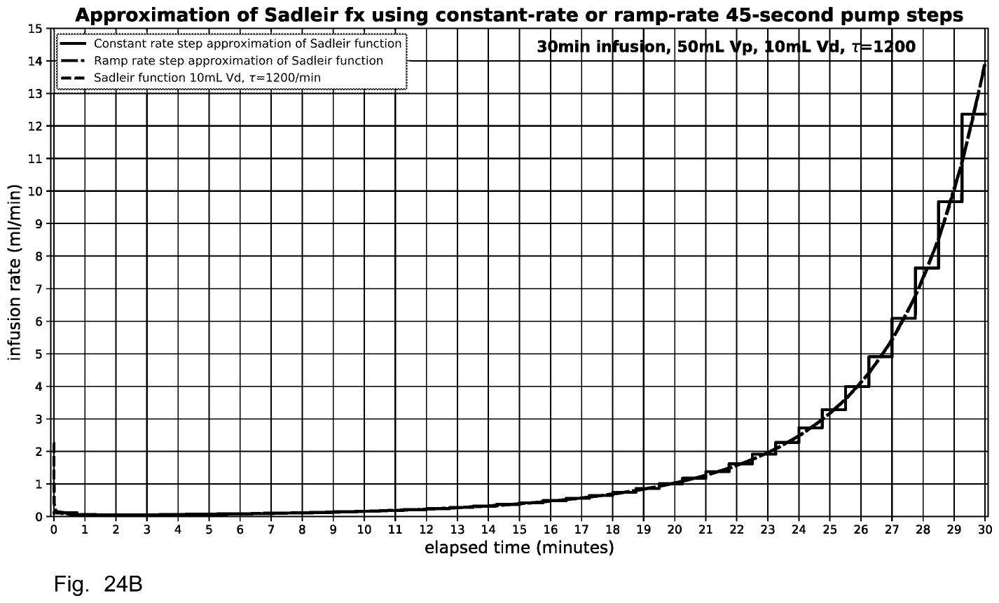

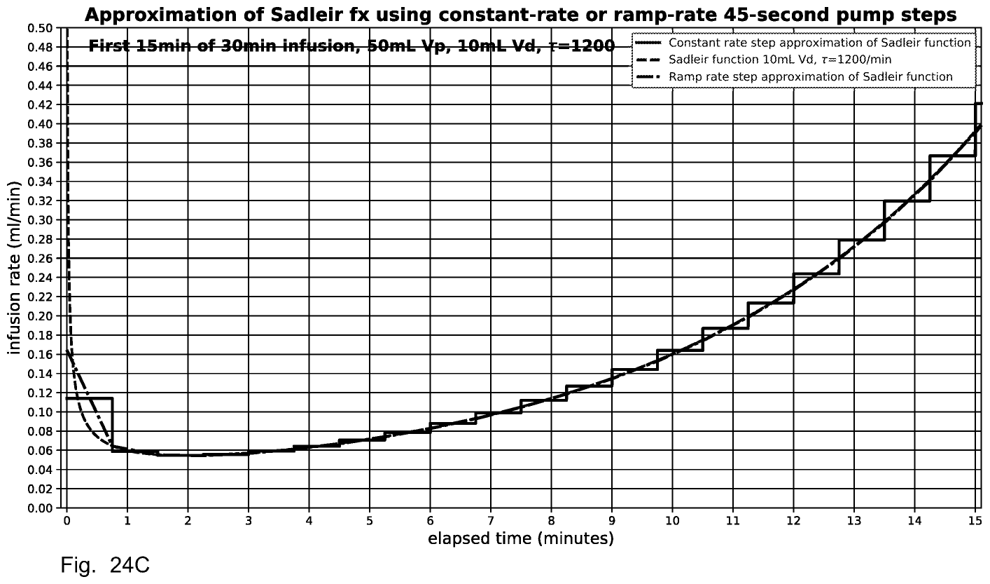

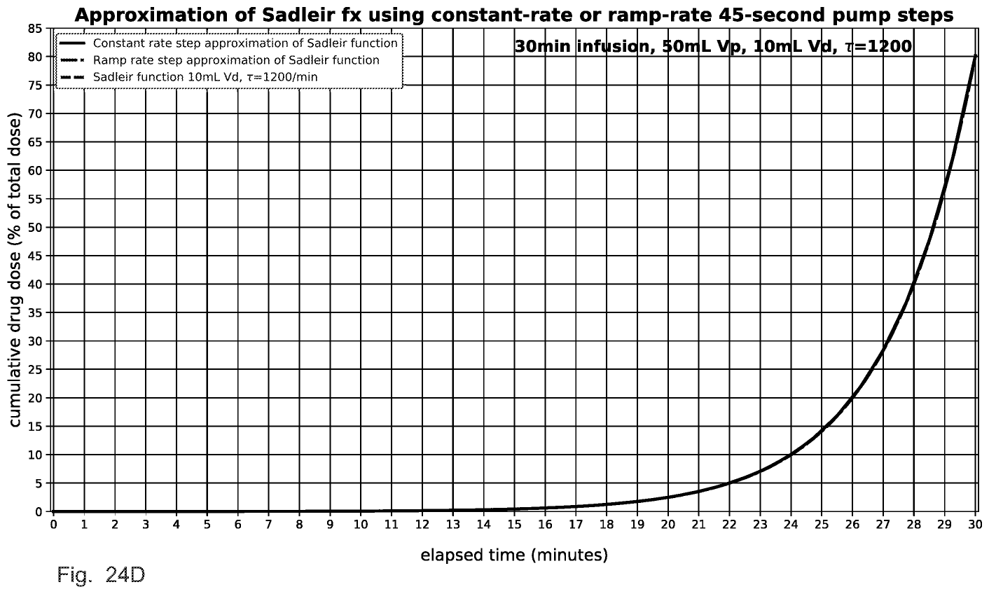

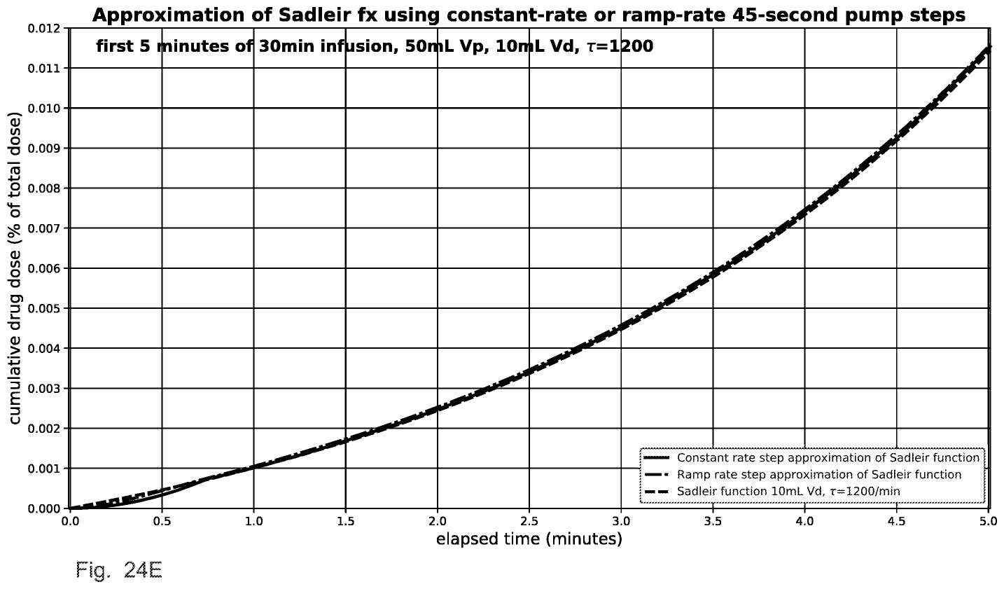

In some examples, the predetermined dose profile is such that the concentration of the pharmaceutical preparation in the dilution chamber increases during the process of infusion to the patient. In some examples, the predetermined dose profile delivers the therapeutic dose over a predetermined infusion time in a manner such that a flow rate of pharmaceutical preparation into the dilution chamber starts at a higher level and decreases during an initial stage to a minimum flow rate and then increases. See for example, FIGS. 24b, 24c, 25b and 29e. In some examples, the minimum flow rate is reached within the first 10% of the predetermined infusion time. See for example, FIG. 24c.

In some examples, the infusion device may comprises a first plunger; a second plunger; and a container configured to receive the second plunger and at least a portion of the first plunger. The dilution chamber may be defined by the container and the second plunger and the dilution chamber outlet comprises a dilution chamber opening defined by the container; wherein the active agent chamber is defined by the first plunger. The container, the second plunger and the active agent chamber may comprises a first active agent chamber opening configured to receive the at least a portion of the first plunger. The second plunger may comprise a valve configured to control a flow of pharmaceutical preparation from the active agent chamber to the dilution chamber in response to applied pressure.

In some examples, the active agent chamber comprises a second active agent chamber opening in a wall of the container; and the active agent chamber is configured to receive the pharmaceutical preparation through the second active agent chamber opening.

In some examples, the instructions executable by the processor of the infusion device may include instructions to:

receive a volume input (Vp) that is indicative of a volume of the pharmaceutical preparation,
receive a time input (i) that is indicative of a time over which the pharmaceutical preparation is to be administered;
determine a number of infusion steps (h) that are to be executed during the time over which the pharmaceutical preparation is to be administered;
determine a pharmaceutical preparation output volume for each of the infusion steps of the number of infusion steps, each pharmaceutical preparation output volume corresponding to a volume of the pharmaceutical preparation that is to be output by the medication delivery apparatus during the respective infusion step;
determine a target flow rate of each infusion step, each target flow rate being indicative of a target flow rate of the pharmaceutical preparation to be output by the medication delivery apparatus during the respective infusion step, wherein each target flow rate is determined based at least in part on the pharmaceutical preparation output volume of the respective infusion step; and
actuate an infusion device actuator to displace the first plunger such that the pharmaceutical preparation is output by the medication delivery apparatus at the respective target flow rate during each infusion step.

In some examples, the include may include instructions to:
receive:
   a concentration input (C_p) that is indicative of a concentration of the pharmaceutical preparation in the active agent chamber;
   a volume input (V_p) that is indicative of a volume of the pharmaceutical preparation that is to be infused,
   a dilution chamber volume input (V_d) that is indicative of a volume of the dilution chamber;
   a time input (i) that is indicative of a time window over which the pharmaceutical preparation is to be administered;
determine a number of infusion steps (h) that are to be executed during the time window;
model the infusion over the time window based on an infusion modelling function wherein modelling the infusion comprises:
determining a target flow rate of the pharmaceutical preparation to the patient and a concentration of the pharmaceutical preparation in the dilution chamber for each of the number of infusion steps;
determine an infusion volume for each of the number of infusion steps (h), based at least in part on said infusion modelling, the infusion volumes being indicative of a volume of the pharmaceutical preparation that is to be output by the medication delivery apparatus during the respective infusion step; and
actuate an infusion device actuator to displace the first plunger such that the determined infusion volume for each infusion step is output by the medication delivery apparatus during the respective infusion step.

While various apparatus have been described above, the present disclosure also includes various methods for delivering an active ingredient into a patient in accordance with dose profiles such as those described above. The method may comprise preparing a pharmaceutical preparation having a particular volume, the pharmaceutical preparation comprising a solvent and therapeutic dose of the active ingredient and intravenously administering the pharmaceutical preparation to the patient, wherein the pharmaceutical preparation is intravenously administered to the patient in in accordance with a predetermined dose profile over a predetermined infusion time in a manner such that at a first stage of administration of the pharmaceutical preparation at least one portion of the therapeutic dose is administered to the patient for detection of a negative reaction in the patient. The predetermined dose profile may be in accordance with any of the dose profiles discussed above.

The method may comprise controlling and/or programming an infusion device to deliver the pharmaceutical preparation according to the predetermined dose profile.

The method may comprise checking the patient for an adverse reaction after 0.01%, 0.1% and 1% of the therapeutic dose has been delivered.

The method may uses any of the infusion devices disclosed herein.

In some examples, the pharmaceutical preparation may be intravenously administered to the patient using an infusion device and the method may comprise attaching a tubing defining a conduit of a predetermined volume to the dilution chamber outlet and wherein the method further comprises controlling the infusion device to perform a priming process to prepare a first volume of diluted pharmaceutical preparation in the tubing prior to connecting the tubing to the patient for intravenous delivery to the patient, wherein the first volume of diluted pharmaceutical preparation forms a first part of the predetermined dose profile.

In some examples, the infusion device may be configured to deliver the diluted pharmaceutical preparation intravenously to a patient through a tubing attached to the dilution chamber outlet, wherein the tubing defines a conduit of a predetermined volume to the dilution chamber outlet and wherein the infusion device is configured to perform a priming process to prepare a first volume of diluted pharmaceutical preparation in the tubing, wherein the first volume of diluted pharmaceutical preparation forms a first part of the predetermined dose profile.

Part 3A—the Tansy Method

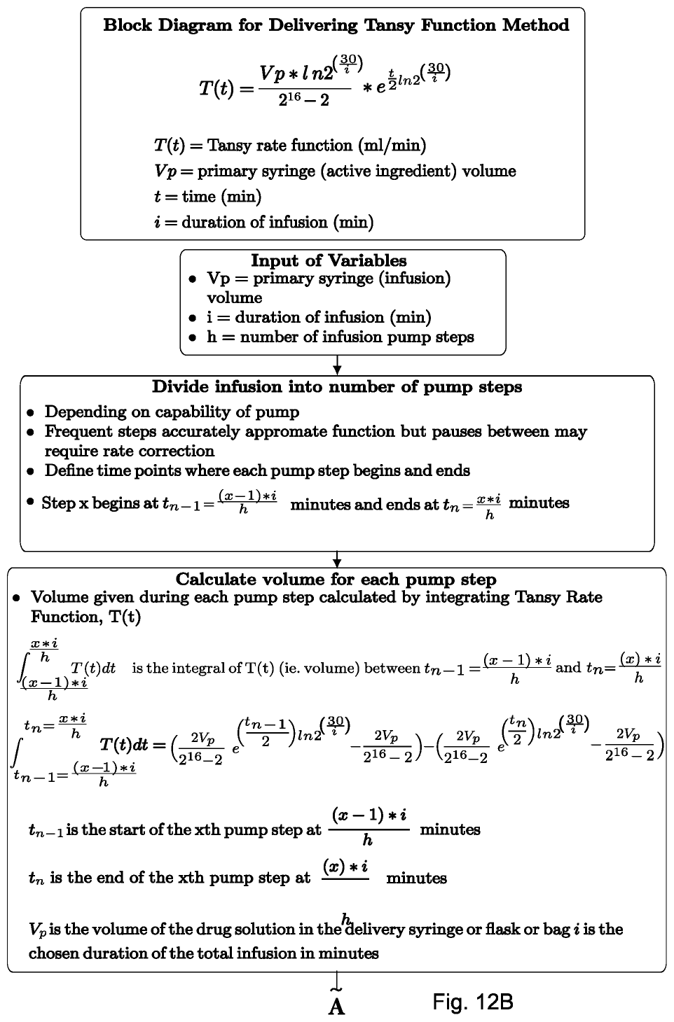

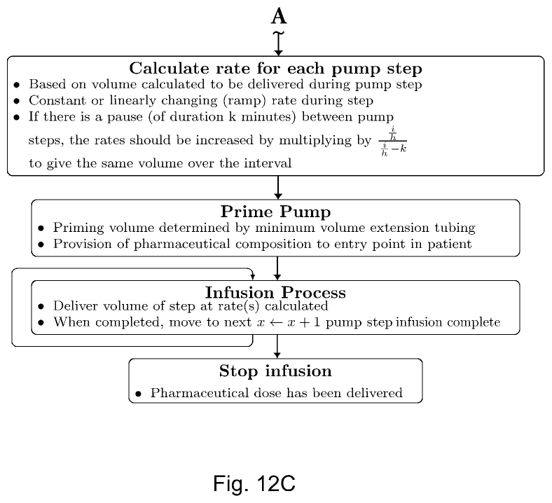

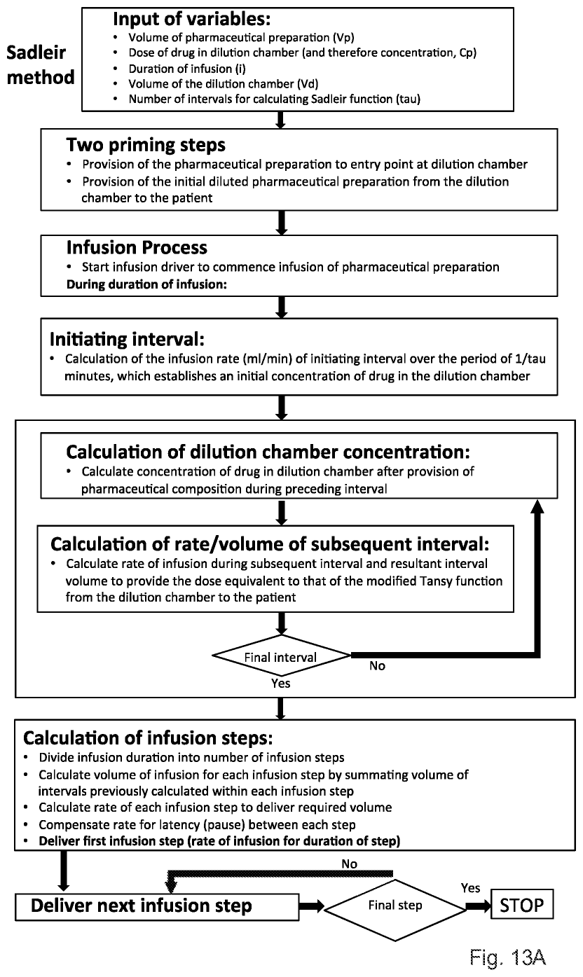

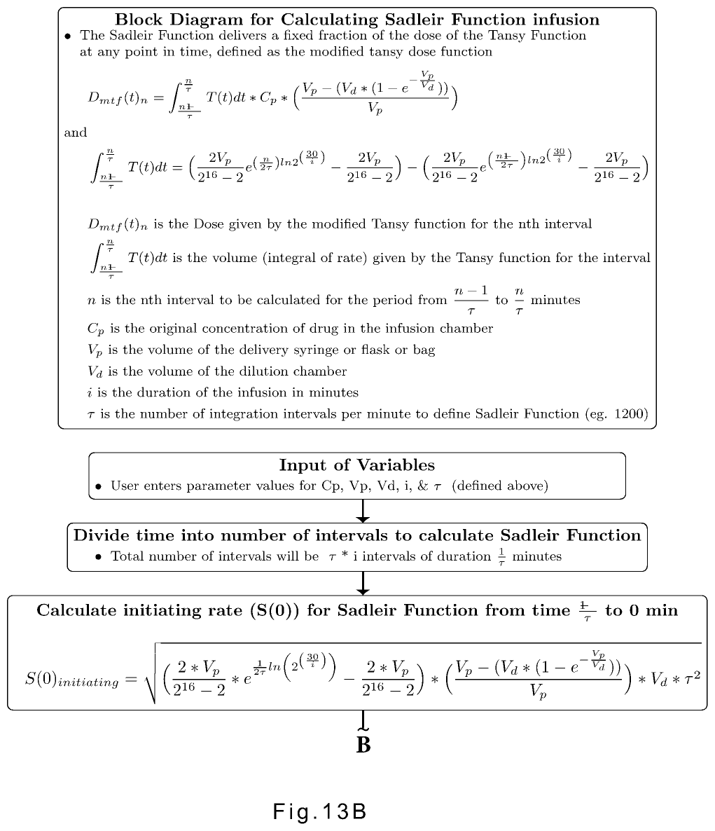

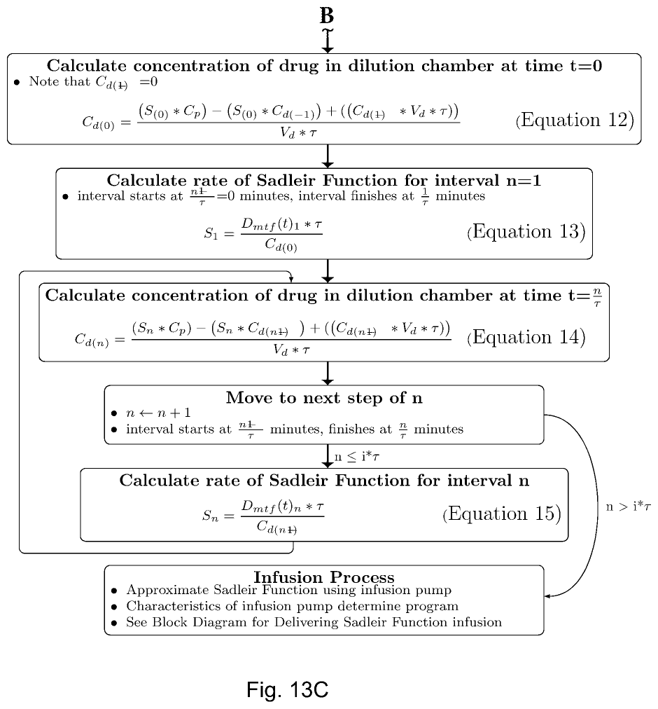

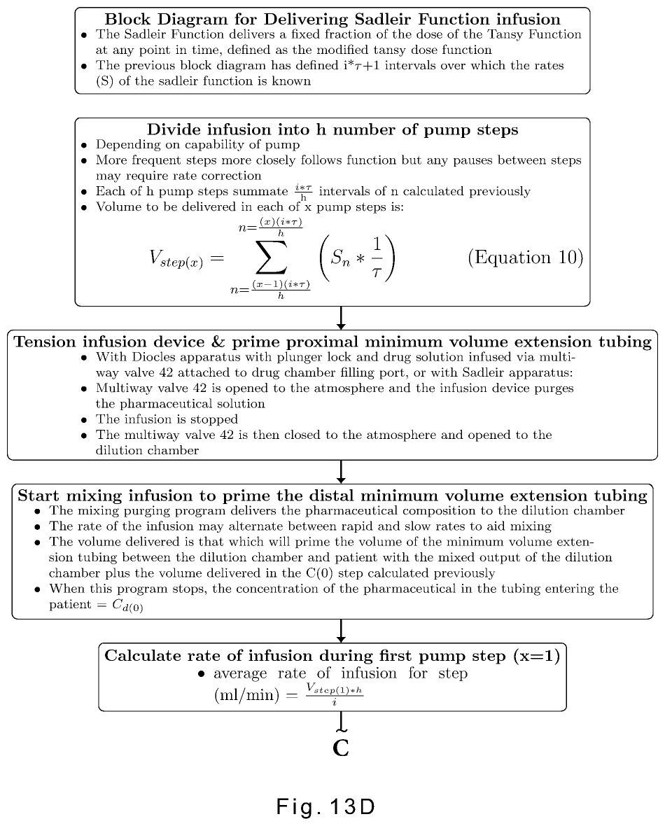

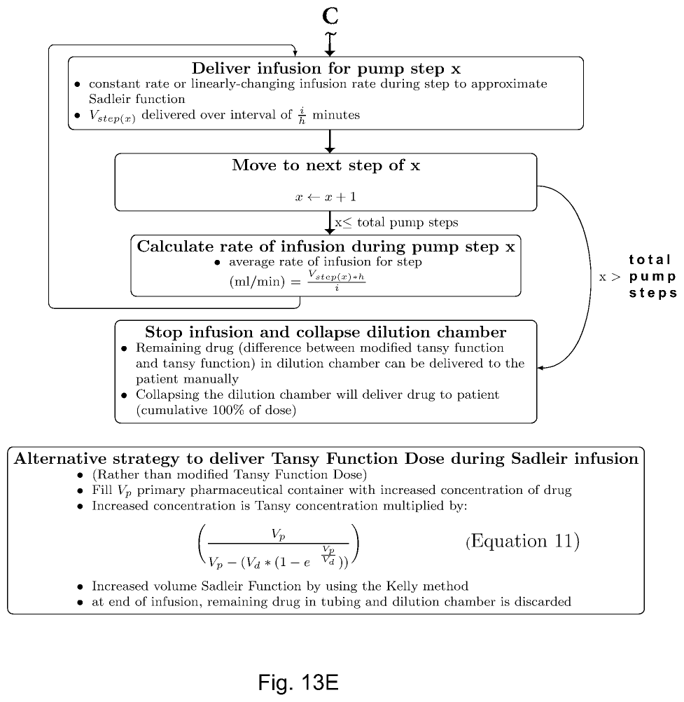

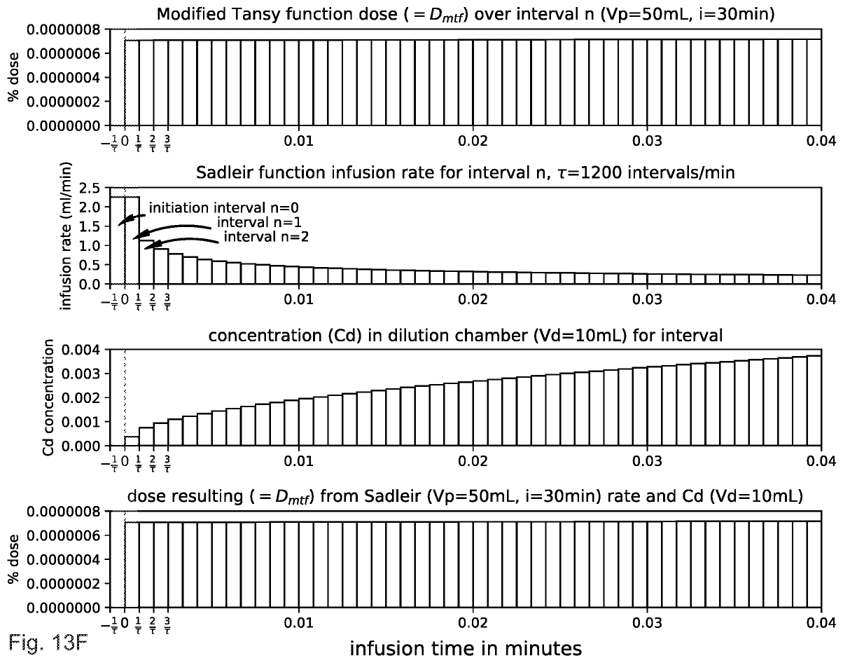

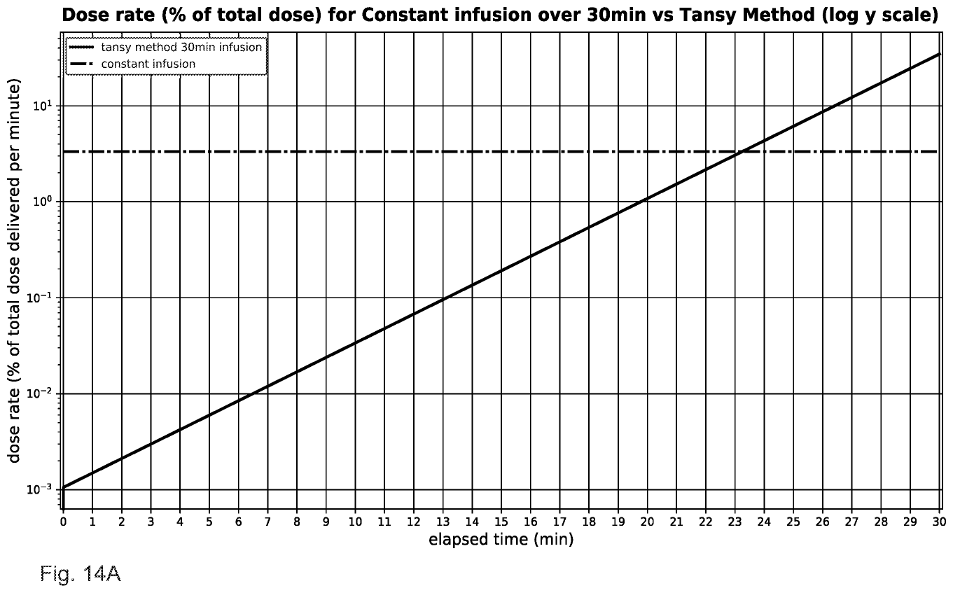

FIGS. 12A and 13A broadly illustrate steps for delivery of a therapeutic dose of the drug contained in the pharmaceutical preparation to be delivered by the infusion driver 14.

FIGS. 12A, 12B and 12C illustrate a method in accordance with a first embodiment of the disclosure. In the first embodiment of the disclosure, there is provided a method of delivering a pharmaceutical preparation to a patient. The pharmaceutical preparation is delivered directly to the patient in accordance with a flow rate as dictated by a Tansy function per equation (1) to be introduced below. In some embodiments, the pharmaceutical preparation is delivered in accordance with an infusion modelling function. In some embodiments, the Tansy function is the infusion modelling function.

In accordance with the first embodiment of the disclosure, there is provided a method for delivering a therapeutic dose of a particular drug to a patient using the apparatus 10 in accordance with the first embodiment of the disclosure, and depicted in FIG. 1. This method is referred to as the Tansy Method.

As mentioned before, the apparatus 10 in accordance with the first embodiment of the disclosure uses the Tansy function to control the flow rate to deliver the therapeutic dose of a particular drug directly (without using the dilution chamber 32) to a patient.

The particular drug to be administered is prepared in the syringe 15 containing a solvent (sterile water or saline), and delivered via the infusion driver 14 to the patient.

As shown in FIG. 12A, the operator inputs via the keyboard 26 of the infusion driver 14:

a) volume of pharmaceutical preparation (Vₚ) to be administered to the patient in ml, comprising an amount of drug (active ingredient in units of mass) and volume of solvent for mixing with the drug (the active ingredient); and

b) time over which the pharmaceutical preparation is to be administered in minutes (also referred to as the duration of infusion),

c) optionally, the identity of the particular drug (drug name), dose of drug, and/or maximum drug administration rate (dose/min) for the particular drug to ensure that the maximum drug administration rate is not exceeded during the infusion process.

Subsequently, the operator provides the pharmaceutical preparation to the entry point of the patient. This step is referred to as the priming step.

Then, the operator starts the infusion driver 14 via instructions through the keyboard 26.

The processor 16 of the infusion driver 14 than executes corresponding instructions for calculating the flow rate (ml/min) of the pharmaceutical preparation at each point in time during the duration of the infusion as dictated by the Tansy function per equation (1) below:

$$T ( t ) = \frac{Vp * ln ( {2}^{( \frac{30}{i} )} )}{{2}^{16} - 2} * {e}^{\frac{t}{2} ln ( {2}^{( \frac{30}{i} )} )} T ( t ) = Tansy rate function ( ml / min ) Vp = primary syringe ( infusion ) volume t = time ( min ) i = duration of infusion ( min ) \qquad ( 1 )$$

The Tansy method for a duration of infusion of 30 minutes has the following original features:

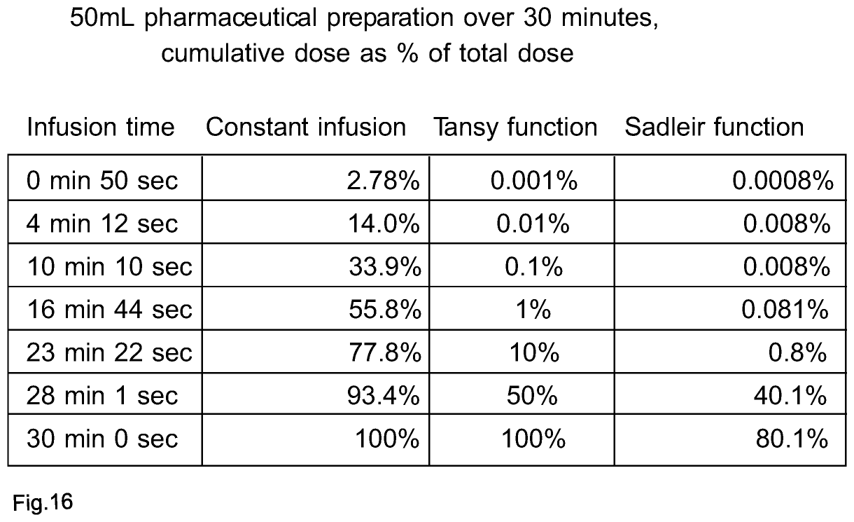

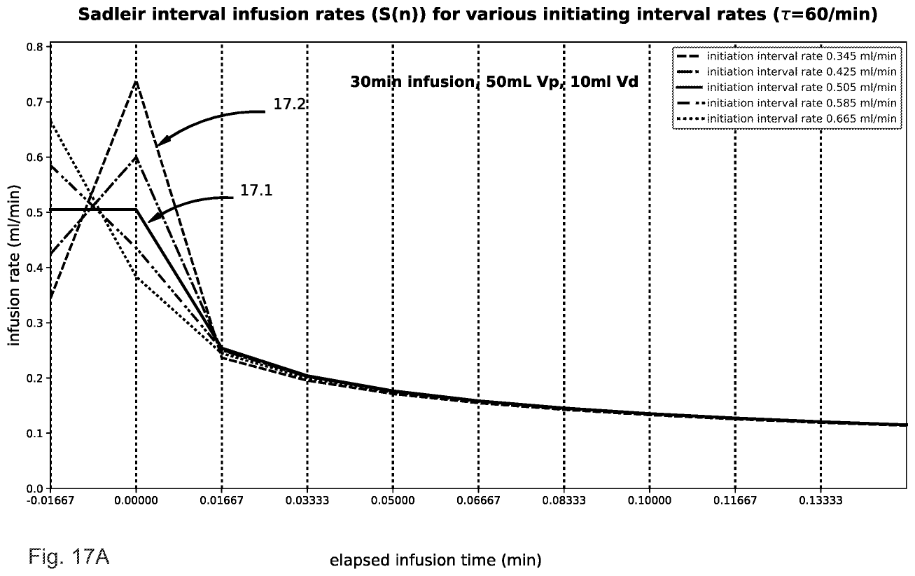

a) the Tansy method will deliver 0.01% of the dose after 14%, 0.1% after 34%, and 1% after 56% of the time period corresponding to the duration of the infusion process (see FIGS. 15 and 16). This increases the likelihood that a negative reaction will be detected and the infusion process may be stopped before a more serious negative reaction occurs. (In contrast, when using a conventional method based on a constant infusion 0.01%, 0.1% and 1% of the total dose will be all administered within the first 1% of the infusion process).

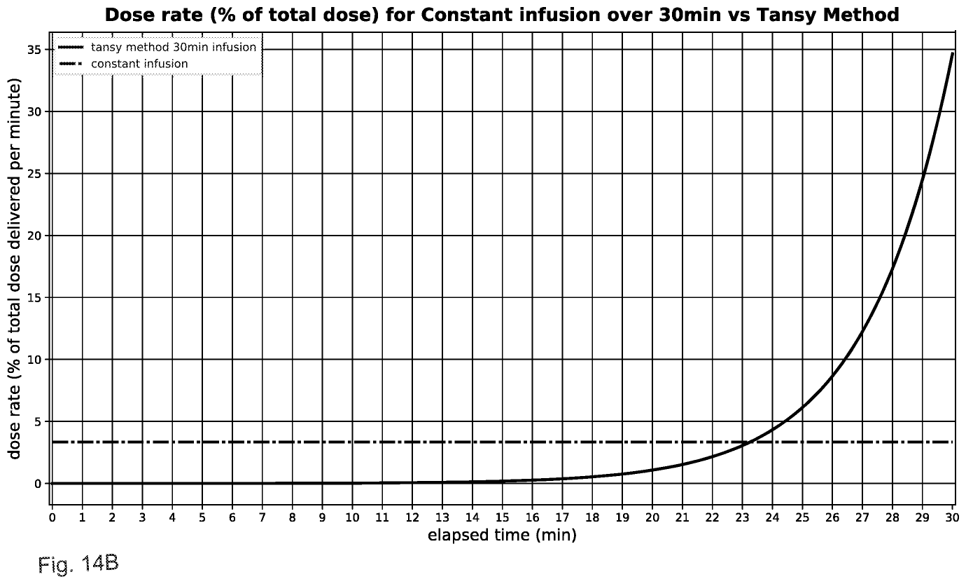

b) the flow rate increases continuously throughout the infusion, doubling every 2 minutes for a 30 minute infusion—see FIGS. 14A and 14B.

In relation to the original feature (a.) mentioned above, FIG. 15 shows the difference in cumulative dose administered over a period of a 30 minute infusion for the Tansy method versus the conventional Constant Infusion method. The total dose delivered over 30 minutes is the same in both methods (Tansy and Conventional (constant infusion over 30 minutes) methods).

Further, FIGS. 15Aa and 15B illustrate the clear separation in time of clinically-relevant magnitudes of cumulative drug administration when using the Tansy method.

However, as shown in FIG. 15, using the Constant Infusion method over 30 minutes will result in 0.01%, 0, 1% and 1% of the dose to have been administered over only the first 18 seconds of the infusion. When using the Constant Infusion method, if a patient was to have a minor reaction at 0.01% of the dose, and a maximal reaction at 10× or 100× times the 0.01% dose, the clinician is unlikely to recognize that the patient is hypersensitive to the drug and will not stop the infusion process before the dose that will induce a maximal reaction has been administered resulting in injury and potential death of the patient.

In contrast, the Tansy method starts at a relative low infusion rate and continuously increases the infusion rate. In particular, using the Tansy Method will result in a patient being administered 0.01% of the dose at 4.18 minutes, and 0.1% of the dose 5.97 minutes later. This almost 6-minute interval will increase the ability of a reaction being detected and permit ceasing of the infusion prior to the patient receiving a supramaximal dose, therefore minimizing any complications. Similarly, a cumulative 1% dose is achieved after another 6 minutes, as is the 10% cumulative dose. The approximately 6 minute separation of orders of magnitude of cumulative dose (for a 30 minute infusion) is a particular feature of the apparatus 10 in accordance with the first and second embodiment of the disclosure. This is illustrated in FIGS. 15 and 16.

In relation to the original feature (b.) mentioned above, FIG. 14A illustrates rate of drug administration, comparing the conventional Constant Infusion method and the Tansy method, using a logarithmic scale. This demonstrates that the rate of drug administration varies (in this particular arrangement it doubles) every two minutes when using the Tansy method for a 30-minute infusion. In particular, the Tansy method has the properties that the rate of drug administration is 0.01% of final infusion rate at 3.425 minutes into the infusion, 0.1% of maximal at 10.07 minutes, 1% at 16.71 minutes, 10% at 23.36 minutes, and 100% at 30 minutes. The total drug administered at is 0.01% after 4.18 minutes, 0.1% after 10.15 minutes, 1% after 16.72 minutes, 10% after 23.35 minutes, and 100% after 30 minutes (see FIG. 16).

As mentioned above, for a 30 minutes infusion the flow rate doubles every two minutes. However, the variation in flow rate is adjustable by changing the infusion duration (see FIGS. 19A and 19B). As shown in FIG. 19B, as the duration of infusion increases the variation in rate is reduced and as the duration of infusion decreases the variation in flow rate is increased.

Below is outlined the general equation for the cumulative volume of pharmaceutical preparation provided at each point in time during infusion in accordance with the first embodiment (i.e.: using the Tansy method).

$$V ( t ) = \frac{2 * Vp}{{2}^{16} - 2} * {e}^{\frac{t}{2} ln ( {2}^{( \frac{30}{i} )} )} - \frac{2 * Vp}{{2}^{16} - 2} \qquad ( 2 )$$

$$V ( t ) = Tansy volume function , cumulative volume ( ml / min ) at time t ( min )$$

$$Vp = primary syringe ( infusion ) volume$$

$$t = time ( min )$$

$$i = duration of infusion ( min )$$

As previously described, the medication delivery system 1 may comprise the above described medication delivery apparatus 10. The medication delivery system 1 may also comprise the infusion device. The infusion device comprises the at least one infusion device processor and infusion device memory storing program instructions accessible by the at least one infusion device processor. The program instructions are configured to cause the at least one infusion device processor to actuate an infusion device actuator (e.g. infusion driver 14) to control the medication delivery apparatus 10 to deliver medication in accordance with the Tansy method.

In particular, the program instructions are configured to cause the at least one infusion device processor to receive a volume input (Vₚ) that is indicative of a volume of the pharmaceutical preparation. This may be a volume of the pharmaceutical preparation in the active agent chamber. The volume input (Vₚ) may be received via an input provided by a user. For example, the volume input (Vₚ) may be input using the user interface 22. Alternatively, the volume input (Vₚ) may be retrieved from the infusion device memory. Throughout this disclosure, the volume input (Vₚ) may correspond to the volume of pharmaceutical preparation.

The program instructions are further configured to cause the at least one infusion device processor to receive a time input (i) that is indicative of a time over which the pharmaceutical preparation is to be administered. The time input (i) may be received via an input provided by a user. For example, the time input (i) may be input using the user interface 22. Alternatively, the time input (i) may be retrieved from the infusion device memory.

The program instructions are further configured to cause the at least one infusion device processor to determine a number of infusion steps that are to be executed during the time over which the pharmaceutical preparation is to be administered. Although referred to as “infusion steps” herein, it will be understood that an infusion step may be considered, or referred to as a pump step. Determining the number of infusion steps may comprise receiving an infusion step input that is indicative of the number of infusion steps. Determining the number of infusion steps may comprise retrieving the number of infusion steps from the infusion device memory.

The program instructions are further configured to cause the at least one infusion device processor to determine a pharmaceutical preparation output volume for each of the infusion steps of the number of infusion steps. Each pharmaceutical preparation output volume corresponds to a volume of the pharmaceutical preparation that is to be output by the medication delivery apparatus during the respective infusion step. Determining the pharmaceutical preparation output volume for each of the number of infusion steps may comprise integrating the Tansy function between a first time that corresponds to a start of the relevant infusion step, and a second time that corresponds to an end of the relevant infusion step.

The Tansy function T(t) may be defined by:

$$T ( t ) = \frac{{V}_{p} \times ln {2}^{( \frac{30}{i} )}}{{2}^{16} - 2} {e}^{\frac{t}{2} l n {2}^{( \frac{30}{i} )}}$$

Where Vₚ is the volume input, t is the time and i is the time input.

Determining the pharmaceutical preparation output volume for each of the number of infusion steps comprises calculating:

∫ₙ₋₁ⁿT(t)dt

The program instructions are further configured to cause the at least one infusion device processor to determine a target flow rate of each infusion step. Each target flow rate is indicative of a target flow rate of the pharmaceutical preparation to be output by the medication delivery apparatus during the respective infusion step. Each target flow rate is determined based at least in part on the pharmaceutical preparation output volume of the respective infusion step. Determining the target flow rate of each infusion step may comprise dividing the pharmaceutical preparation output volume of a respective infusion step by a length of that infusion step. Determining the target flow rate of each infusion step may comprise determining an initial target flow rate and a final target flow rate for each infusion step. The initial target flow rate of a respective infusion step may be equal to the final target flow rate of a preceding infusion step. The final target flow rate of the respective infusion step may be equal to the initial target flow rate of the following infusion step.

The program instructions are further configured to cause the at least one infusion device processor to receive a pharmaceutical preparation input. The pharmaceutical preparation input is indicative of one or more of: an identity of the pharmaceutical preparation, a dose of the pharmaceutical preparation, and a maximum pharmaceutical preparation administration rate. The target flow rate may be limited at the maximum pharmaceutical preparation administration rate, such that the target flow rate does not exceed the maximum pharmaceutical preparation administration rate during infusion.

The program instructions are further configured to cause the at least one infusion device processor to actuate an infusion device actuator to displace the plunger 21 within the active agent chamber 98 such that the pharmaceutical preparation is output by the medication delivery apparatus 10 at the respective target flow rate during each infusion step.

Part 3B—the Sadleir Method

In accordance with the second embodiment of the disclosure there is provided a method for delivering a therapeutic dose of a particular drug to a patient using the apparatus 10 in accordance with the second embodiment of the disclosure.

As mentioned before, the apparatus 10 in accordance with the second embodiment of the disclosure uses the Sadleir function to control the flow rate of pharmaceutical preparation leaving the infusion driver 14 for delivery of the pharmaceutical preparation to the dilution chamber 32 and, from the dilution chamber 32, to the patient.

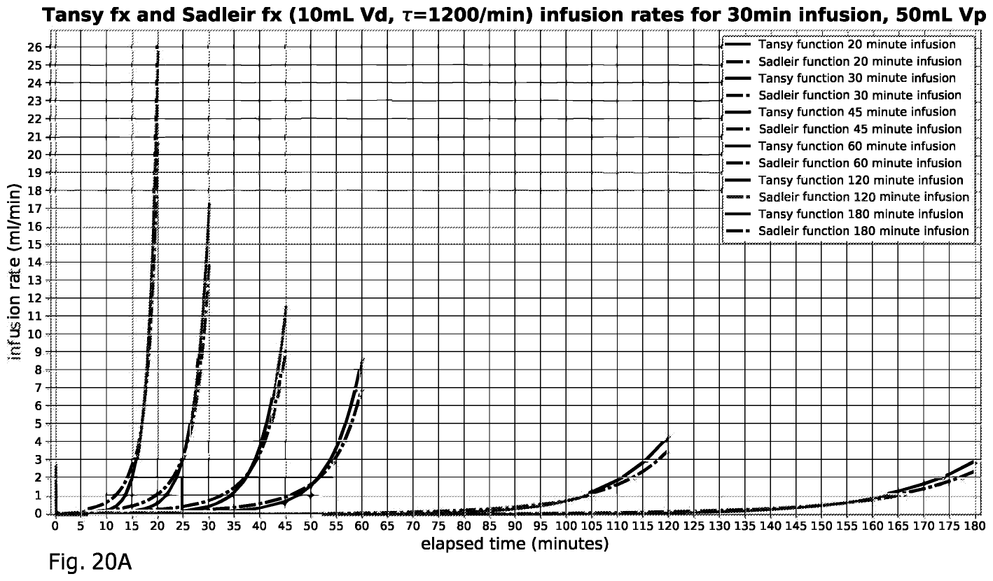

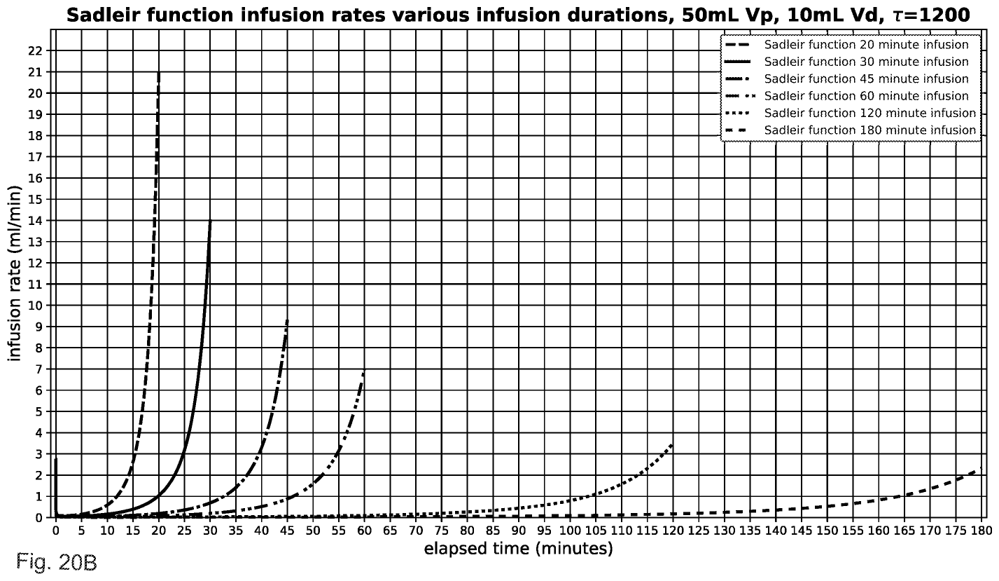

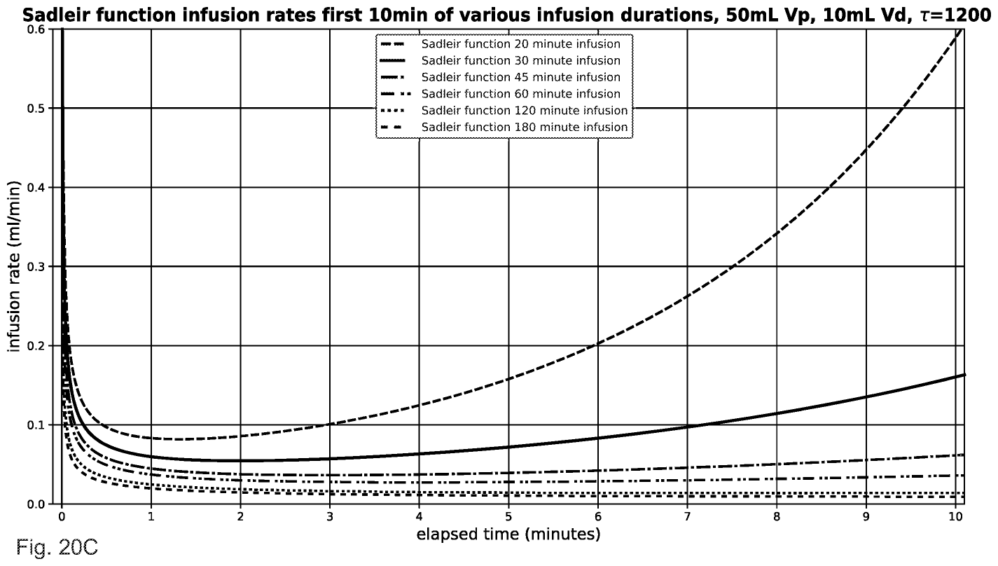

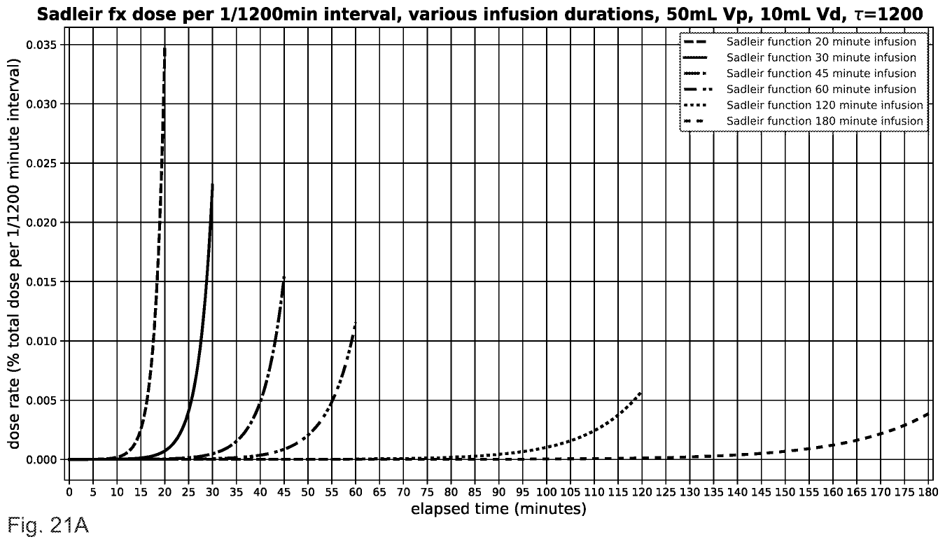

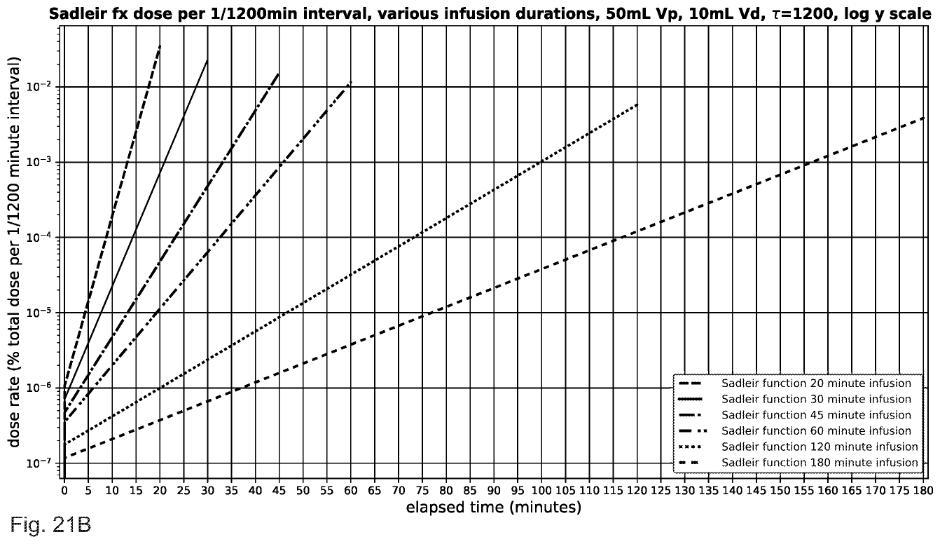

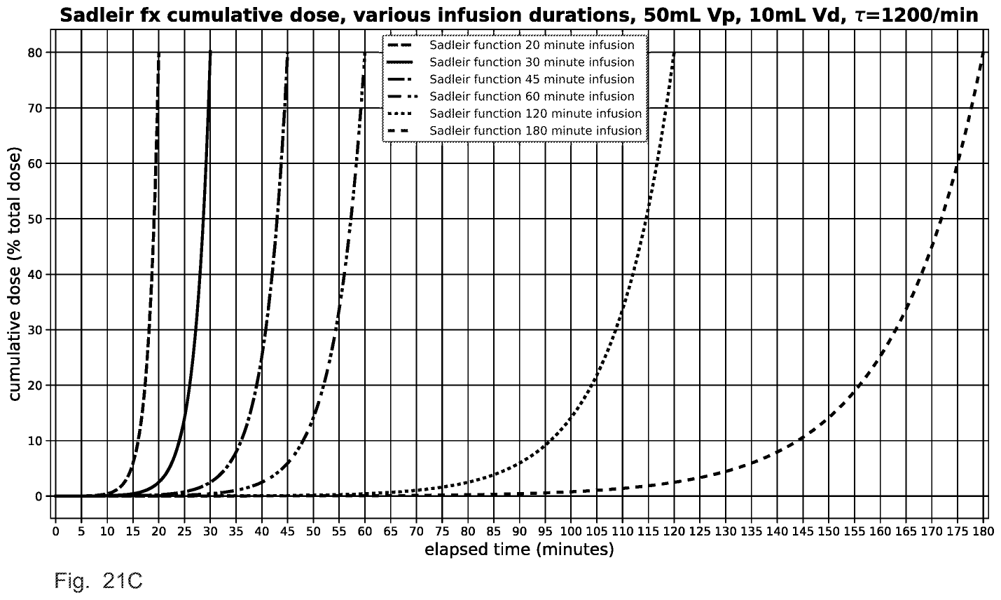

The method in accordance with the second embodiment of the disclosure improves the accuracy of the manner in which the drug is delivered by delivering the drug at similar variations of rate as the first embodiment of the disclosure but, in contrast with the first embodiment of the disclosure, the drug when using the second embodiment of the disclosure is delivered at (1) a minimum flow rate that is greater than the minimum flow rate of the first embodiment of the disclosure, and (2) at a maximum infusion rate that is lower than the maximum rate of the first embodiment of the disclosure. See FIGS. 20A, 22B and 22C.

The improvement in accuracy (i.e.: being able to deliver a higher flow rate of the pharmaceutical preparation during the early phase of the infusion process) is achieved by delivering the pharmaceutical preparation to the dilution chamber 32. The dilution chamber 32 contains a fixed volume of diluent (saline or similar) to which the pharmaceutical preparation will mix during the course of the infusion. Therefore, by directing the pharmaceutical preparation into the dilution chamber 32, diluted pharmaceutical preparation is provided.

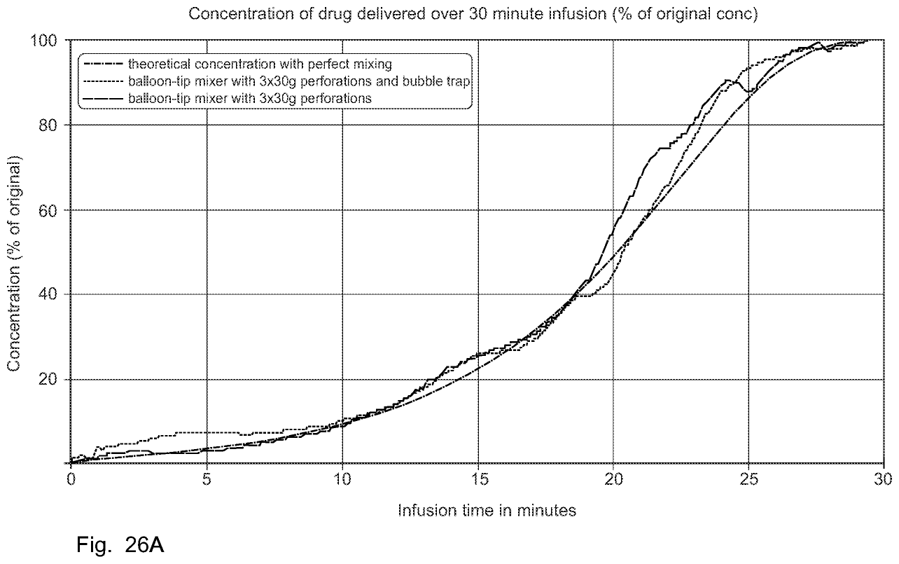

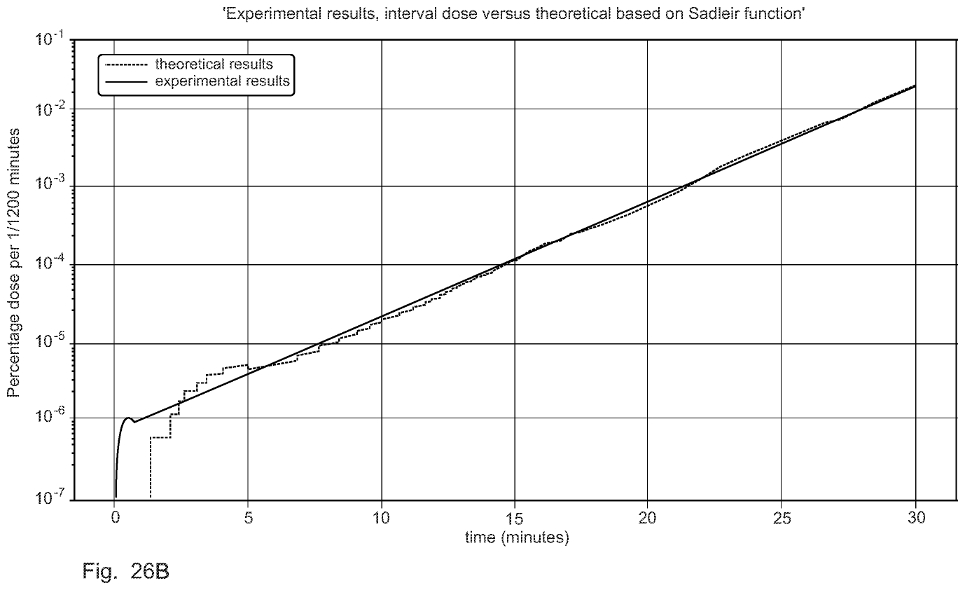

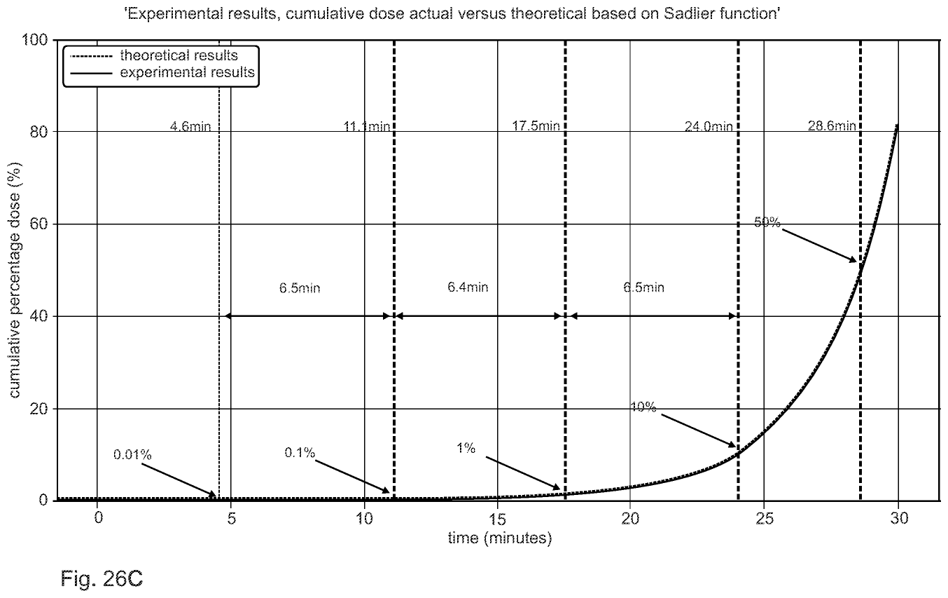

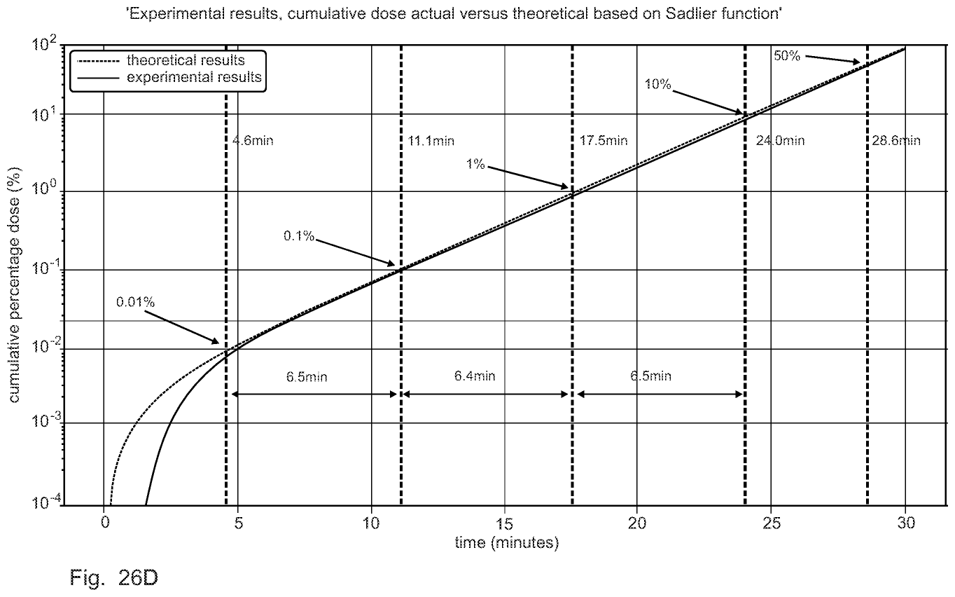

However, the fact that the pharmaceutical preparation is diluted in the dilution chamber 32 results in a reduction in the drug concentration within the dilution chamber 32 as compared to the drug concentration of the pharmaceutical preparation contained in the syringe 15 (i.e. the active agent chamber 98). This results in the pharmaceutical preparation exiting the dilution chamber 32 having a lower concentration than the pharmaceutical preparation contained in the syringe 15 (the active agent chamber 98) of the infusion driver 14. The concentration of pharmaceutical preparation leaving the dilution chamber 32 will be lowest at the beginning of the infusion, and increase throughout the duration of the infusion (see FIG. 26C for an example using a 10 ml dilution chamber with 50 mL infusion over 30 minutes). The flow rate of the pharmaceutical preparation is adjusted to a higher rate in order to compensate for the reduction in pharmaceutical preparation (drug) concentration (due to having been diluted in the dilution chamber 32) compared to that provided by the first embodiment of the disclosure (the Tansy Method),

Further, due to the pharmaceutical preparation being delivered not directly to the patient but instead to the dilution chamber 32, at the end of the process of administering the pharmaceutical preparation, a remainder of the pharmaceutical preparation will remain in the conduits 30 and the dilution chamber 32. The remainder of the pharmaceutical preparation (contained in the dilution chamber 32) may be administered by either, for example, decreasing the volume of the dilution chamber 32 or by flushing conduits 30 and the dilution chamber 32 with saline or other appropriate solution. For this, as described before in accordance with the second embodiment of the disclosure, in the arrangement shown in the figures, the dilution chamber 32 comprises a syringe permitting reduction of the volume of the dilution chamber 32 by pressing the plunger of the syringe. The dilution chamber 32 may comprise a second plunger (i.e. part of the syringe).

The quantity of remainder of the dose (Vᵣ) in the dilution chamber 32 at the end of the infusion process is dependent on the ratio of the volume of the drug to be administered (Vₚ) and volume of the dilution chamber (V_{d}). In particular, the volume of the remainder of the dose (Vᵣ) in dilution chamber 32 at end of the drug administration process is given by:

$$\frac{{V}_{p} - {V}_{d} ( 1 - {e}^{- \frac{{V}_{p}}{{V}_{d}}} )}{{V}_{p}} = {V}_{r} {V}_{p} = volume of drug ‐ containing infusion container {V}_{d} = volume of dilution chamber \qquad ( 3 )$$

Comparing the Tansy and Sadleir methods, the particular quantity of drug that remains (at the end of the infusion process) in the dilution chamber 32 and is not delivered to the dose delivered via the Sadleir method, is less than the full therapeutic dose or the dose that is delivered by the Tansy method. In particular, at any point in time during the drug administration process the dose delivered using the Sadleir function is obtained using equation 3 below

$$\frac{{V}_{p} - {V}_{d} ( 1 - {e}^{- \frac{{V}_{p}}{{V}_{d}}} )}{{V}_{p}} \qquad ( 3 )$$

by multiplying the dose that is delivered by the Tansy method by equation 3 above. Equation 3 is referred to as the ‘correction factor’.

The variation in the rate of administration of drug (active ingredient) for the Tansy method and the Sadleir method is similar, but the amount per unit time and the total dose (of the drug) delivered to the patient is reduced by a fixed fraction (by multiplying by the ‘correction factor’) that depends on the volume of the dilution chamber 32 relative to that of the total infusion volume, see FIG. 22A.

In particular, for a 10 ml dilution chamber with a 50 ml primary drug infusion (or 20 ml dilution chamber with 100 ml primary drug infusion), 19.865% of the dose remains in the dilution chamber 32 at the end of the infusion, and therefore only 80.135% of the full therapeutic dose is administered to the patient.

The volume of dose remaining in the dilution chamber 32 may be delivered to the patient by reducing the volume of the dilution chamber 32 in order that the final 19.865% of dose can be given to the patient as a push (by depressing the plunger in the dilution chamber), or by flushing the system with saline solution and deliver it to the patient.

The advantage of the Sadleir method, which is used in conjunction with the apparatus 10 incorporating the dilution chamber 32, is that the minimum flow rate of the pharmaceutical preparation exiting the infusion driver 14 is orders of magnitude greater than that of the Tansy method, and so the ability to accurately administer the drug is improved, and the total volume of the pharmaceutical preparation can be reduced. As mentioned before, infusion drivers 14 are not able to provide proper infusion rates at relative low flow rates such as the initial rates of infusion using the Tansy method. The Sadleir method also reduces the maximum flow rates required, reducing the required size of patient intravenous cannula size and improving patient tolerance.

The Sadleir method accomplishes this by using the dilution chamber 32 of the apparatus 10 in accordance with the second embodiment of the disclosure.

The precision of the estimate for the volume administered in the first minute of the Sadleir function achieves 3 significant figures when the algorithm used for calculating the calculate the volume operates on a time interval of 1/600th of a minute or shorter intervals (see FIG. 16 for volume in first minute for a 30 minute infusion from a 50 ml syringe with a 10 ml dilution chamber).

The Sadleir method, when using the same pharmaceutical preparation concentration, delivers a known fraction of the Tansy protocol dose, increasing proportionally at a similar rate. The Sadleir function is calculated by numerical approximation of a nonlinear function and this calculation is detailed below.

FIGS. 13A, 13B, 13C, 13D and 13E illustrates the method in accordance with the second embodiment of the disclosure where the pharmaceutical preparation is delivered via the dilution chamber 32 to the patient in accordance with the variation of the flow rate as dictated by the Sadleir function per equation (6) to be introduced below. FIG. 13D illustrates for each interval n (with an interval duration of 1/1200 min) the value of: the flow rates of as dictated by the Sadleir function, the concentration in dilution chamber and the % dose.

In accordance with the second embodiment of the disclosure the method for delivering a therapeutic dose of a particular drug to a patient uses the apparatus 10 in accordance with the second embodiment of the disclosure and depicted in FIGS. 2 and 3. This method is referred to as the Sadleir Method.

As mentioned before, the apparatus 10 in accordance with the second embodiment of the disclosure uses the Sadleir function to indicate to the syringe driver 17 at which flow rate the pharmaceutical preparation will be delivered to a patient using the dilution chamber 32.

The particular drug to be administered to the patient is prepared in, the syringe 15 containing a diluent (sterile water or saline), and delivered via the infusion driver 14 to the patient. The diluent may also be referred to as a solvent.

Referring to FIG. 13A, the operator inputs via the keyboard 26 of the infusion driver 14:

a) Volume of the pharmaceutical preparation (Vₚ) in mL to be delivered to the patient, comprising of the volume of solution to give the correct therapeutic dose of drug (active ingredient);

b) Volume of dilution chamber 32;

c) Concentration of drug in primary syringe (e.g. percent of therapeutic dose/ml);

d) Time (i) over which the pharmaceutical preparation is to be administered in minutes (also referred to as the duration of infusion);

e) Number of intervals per minute (t). (As will be explained below, the infusion process is divided into intervals over which the algorithm (run by the processor 16 of the infusion driver 14 and used for calculating the flow rate values as dictated by the Sadleir function) will be iterated.); and

f) Optionally, the identity of the particular drug (drug name), dose of drug, and/or maximum drug administration rate (dose/min) for the particular drug to ensure that the maximum drug administration rate is not exceeded during the infusion process.

Subsequently, the processor 16 of the infusion driver 14 calculates the parameters required for calculating the flow rate at which the infusion driver 14 needs to drive the pharmaceutical preparation with the syringe driver 17 from the syringe 15 (the pharmaceutical preparation) in order to comply with the Sadleir function; these parameters are:
1. the number of intervals during the infusion process over which the values of the dilution chamber concentration is calculated (the number of intervals per minute (t) multiplied by the duration of the infusion in minutes (i)); and
2. the flow rate S(0)_{initiating} of the pharmaceutical preparation that establishes a particular concentration of drug in the dilution chamber 32. This interval occurs prior to the delivery of drug to the patient and it begins at 1/τ minutes prior to the infusion, is of duration 1/τ minutes, and finishes at time 0. The equation below provides the rate of the initiating dose in ml/min:

$$\sqrt{( ( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) ) * {τ}^{2} * {V}_{d}} \qquad ( 4 )$$

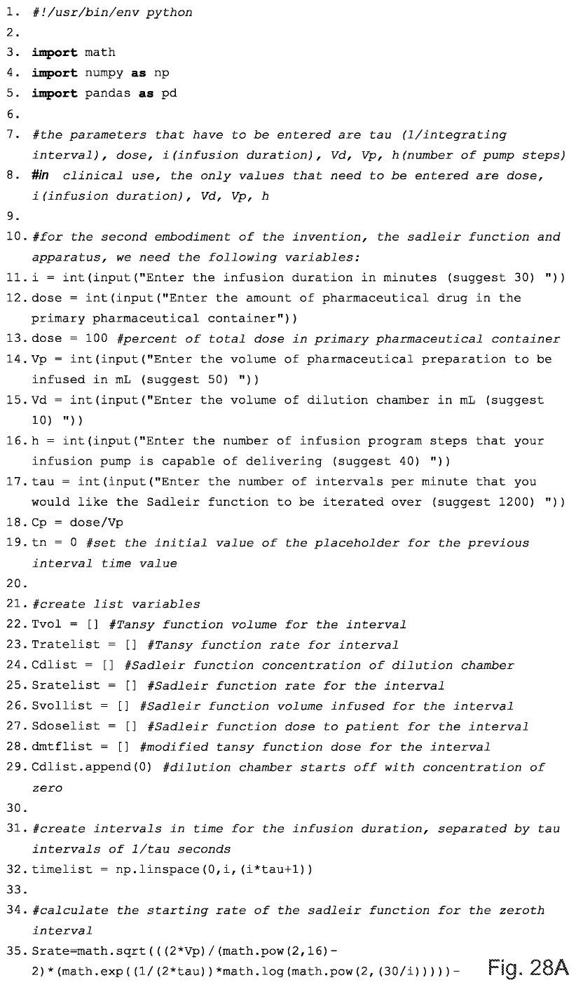

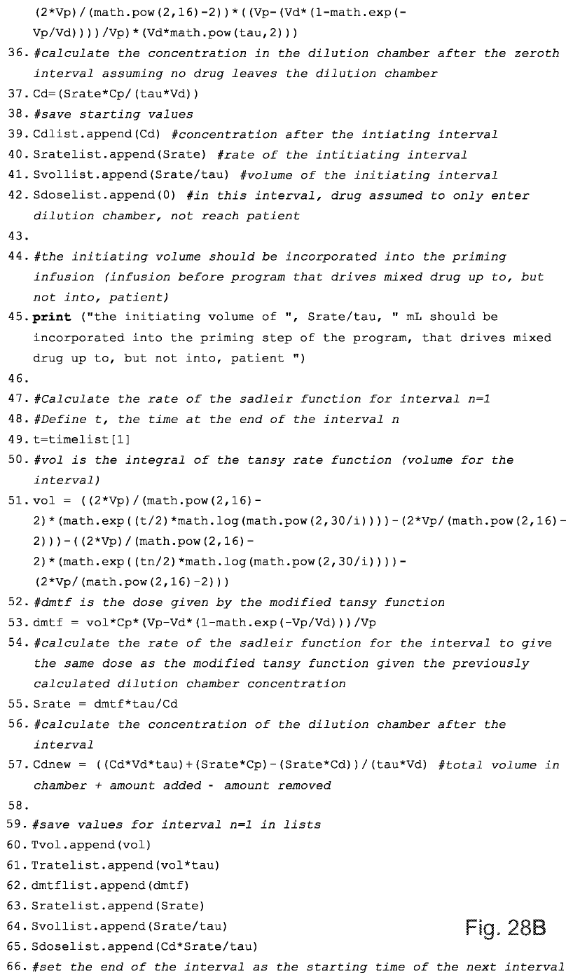

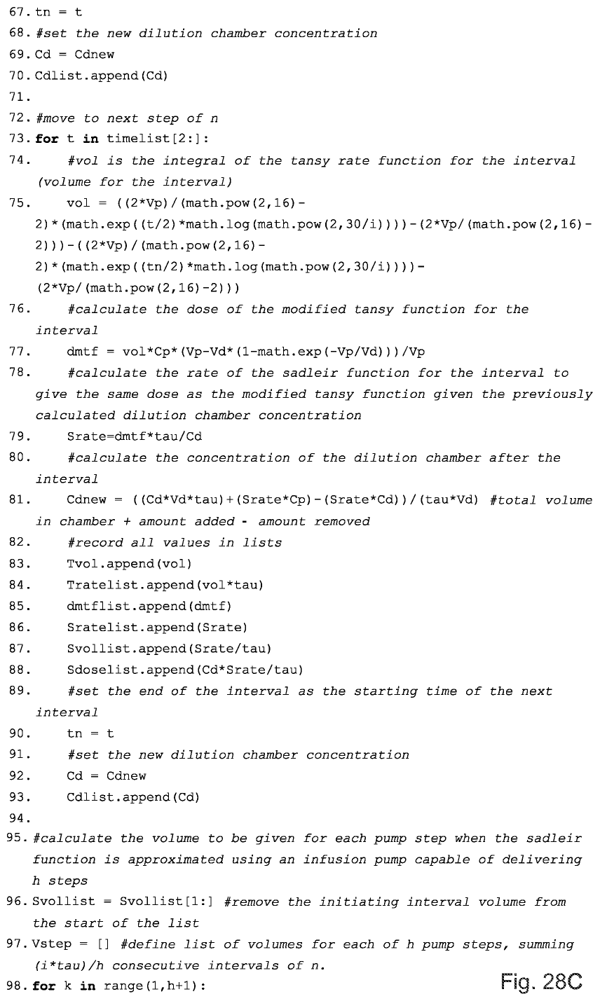

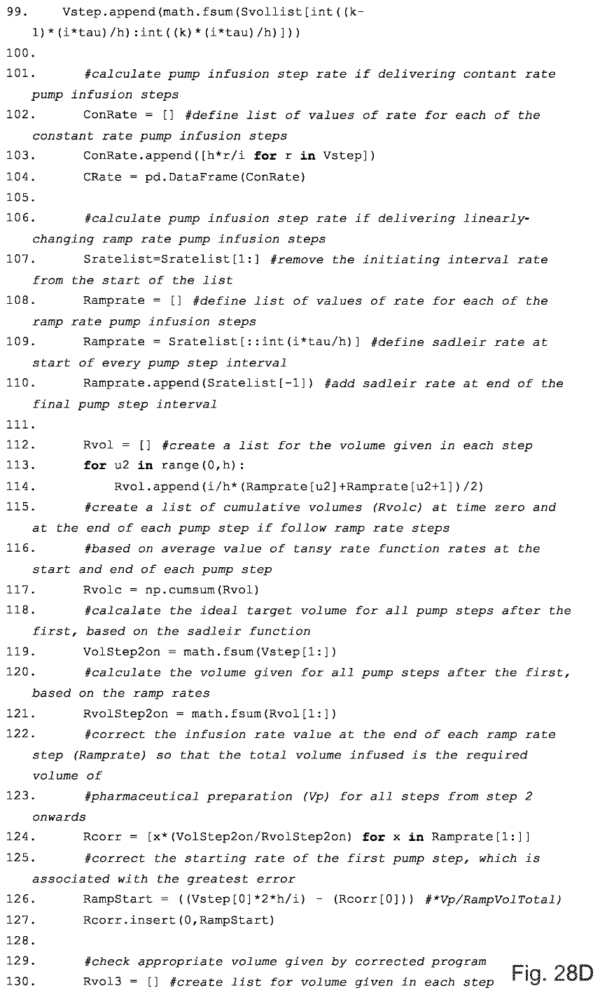

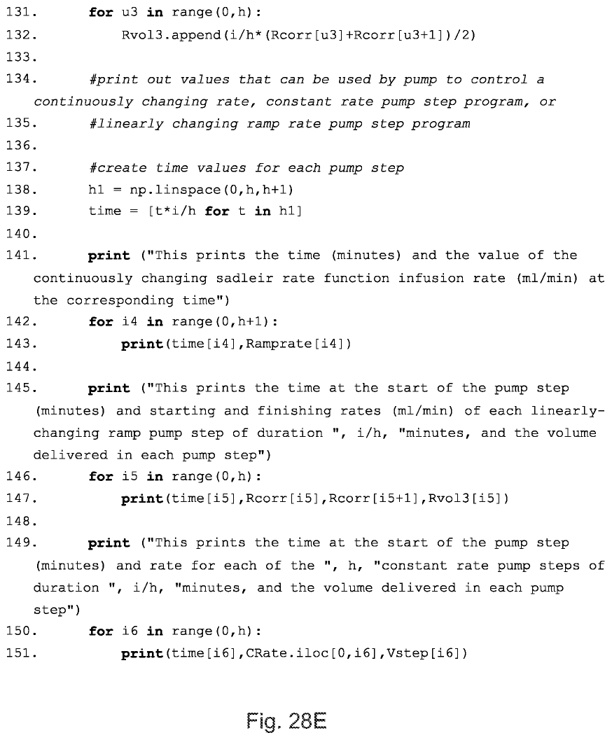

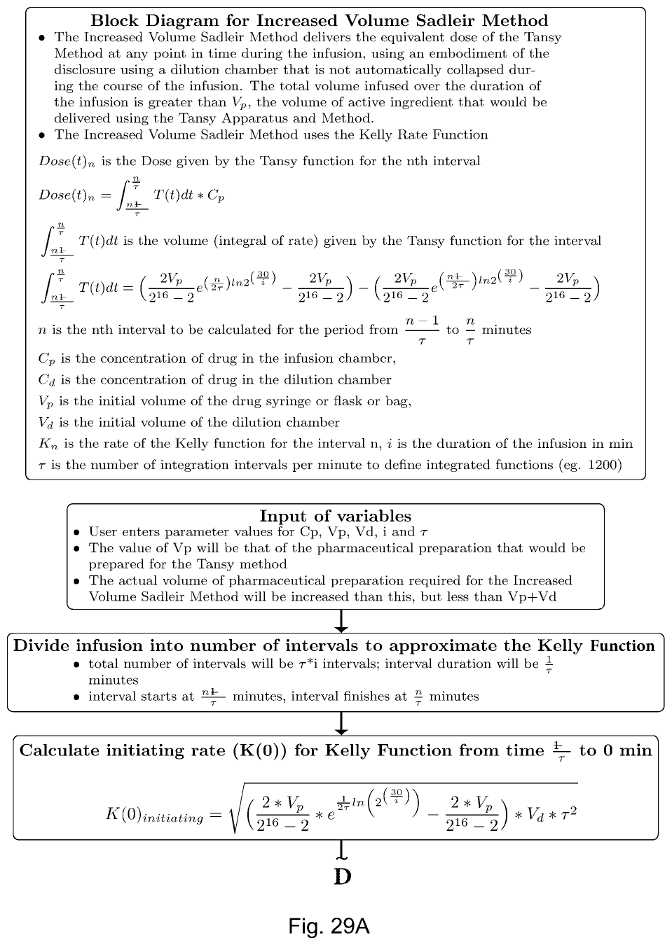

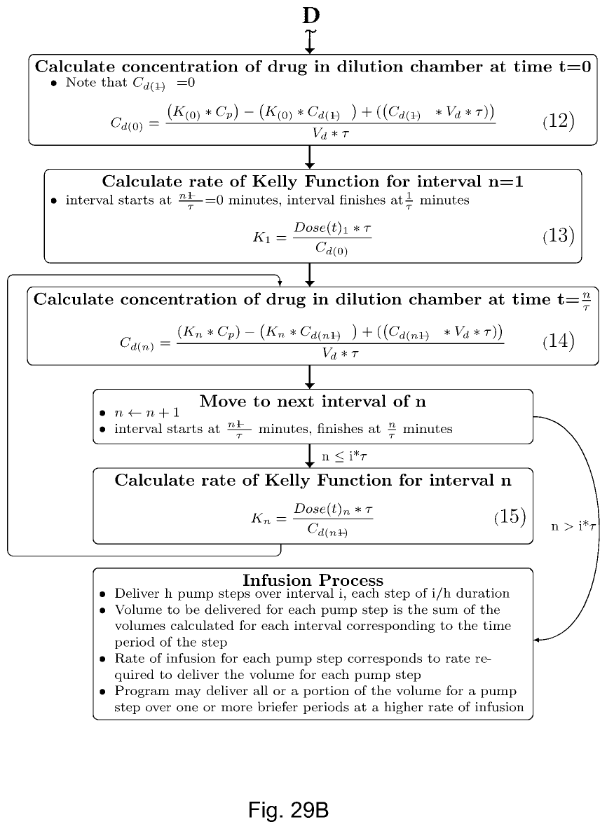

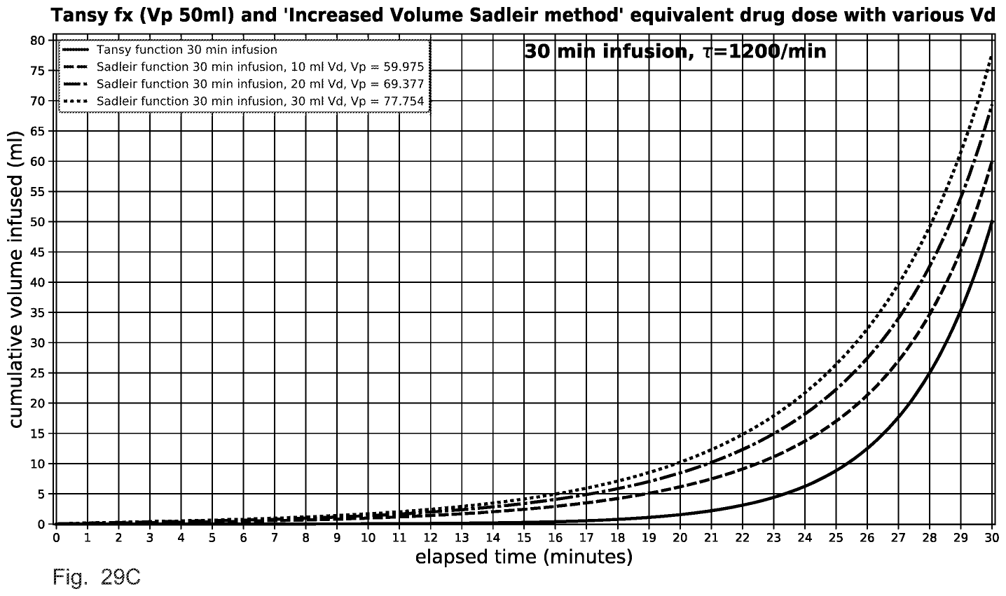

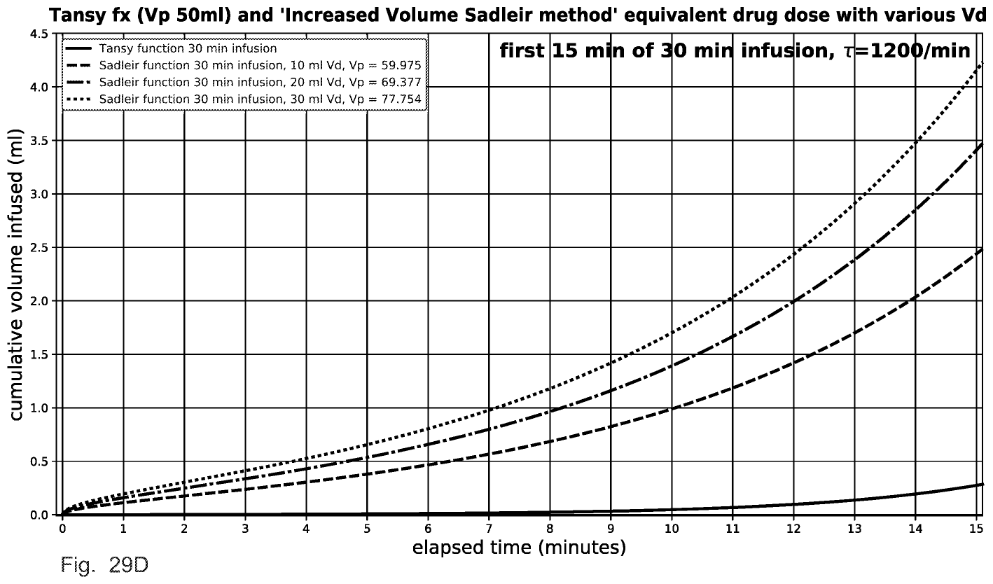

The processor 16 executes instructions to run an algorithm for calculating the rate or volume of the initiating interval, and that of the τ* i intervals during the infusion process according to the algorithm illustrated in FIG. 13b conducted by the python 3 software instructions (software) shown in FIG. 28.

The processor 16 executes instructions to run an algorithm for calculating the initiating interval rate using the equation (4) above for delivering of the pharmaceutical preparation during the time period from −1/τ to 0 to the dilution chamber 32. Deduction of the equation 4 is shown at a later stage below.

The initiating step occurs during the time period from −1/τ to 0 and during this step a concentration of active ingredient is established within the dilution chamber 32.

To calculate the flow rate that the pharmaceutical preparation needs to exit the syringe driver 17 in accordance with the Sadleir method during each subsequent interval after the initiating interval, it is necessary to calculate the concentration of the dilution chamber 32 prior to each subsequent interval.

For example, at time 0 and prior to commencement of the infusion process, it is necessary to calculate the concentration of the pharmaceutical preparation contained in the dilution chamber 32 in order to calculate the flow rate for the first subsequent interval occurring after the initiating step. Equation 12 shown in FIG. 13B provides the concentration of the dilution chamber 32 at time 0.

Once the concentration of the dilution chamber 32 at time 0 is calculated, the flow rate during the first interval (n=1) is calculated by the processor 16 via equation 13 shown in FIG. 13B. This requires calculation of the dose of drug (active ingredient) that is administered using the Tansy function for the equivalent interval of time for an infusion with the same pharmaceutical preparation characteristics in the infusion driver syringe (concentration of drug, volume of pharmaceutical preparation to be administered (Vₚ), and total duration of the infusion (i)). This particular dose (as it would be administered using the Tansy function) is then reduced by multiplying its value by the correction factor

$$\frac{{V}_{p} - {V}_{d} ( 1 - {e}^{- \frac{{V}_{p}}{{V}_{d}}} )}{{V}_{p}} .$$

The dose obtained by this multiplication is referred to as the dose of the modified Tansy function, or D_{mtf}, and is defined in FIG. 13B.

After the flow rate for the interval n=1 is calculated by the processor 16, the concentration of drug in dilution chamber 14 at the end of this interval (at time 1/τ minutes) is calculated using equation 14 of FIG. 13b. This equation estimates the concentration of drug in the dilution chamber 14 at the end of the interval n (in this example n=1) by dividing the amount of drug in the dilution chamber by the volume of the dilution chamber 32. The amount of drug in the dilution chamber 32 is estimated from the amount of drug present in the dilution chamber 32 at the start of the previous interval (n−1, at this point n=0 or the initiating interval), the particular dose that has entered the dilution chamber 32 during the interval n, and the particular dose has exited the dilution chamber 32 during the interval, n.

At this stage, the flow rate during each subsequent interval n, after the first interval that occurred between time 0 to 1/τ minutes, is calculated by the processor 16 by calculating in sequence the flow rate for each interval n via equation 15 shown in FIG. 13B, and then the concentration of drug in the dilution chamber at the end of each interval n via equation 14 shown in FIG. 13B.

In particular, the flow rate (Sₙ) during each subsequent interval is such that the same dose is given to a patient as when using the Tansy method but modified by reducing the rate of the Tansy function to account for the amount of drug remaining inside the dilution chamber 32 at the end of the infusion.

The infusion rate is calculated per equation:

$${S}_{n} = \frac{{D}_{m t {f ( t )}_{n}} * τ}{{C}_{{d}_{( n - 1 )}}}$$

Deduction of the equation of the Starting Rate for Priming Dose

The initial rate of the theoretical Sadleir Function is undefined (as the dilution chamber concentration is zero, the initial rate is equal to the Tansy Function dose (0), divided by the concentration (0), i.e. 0/0).

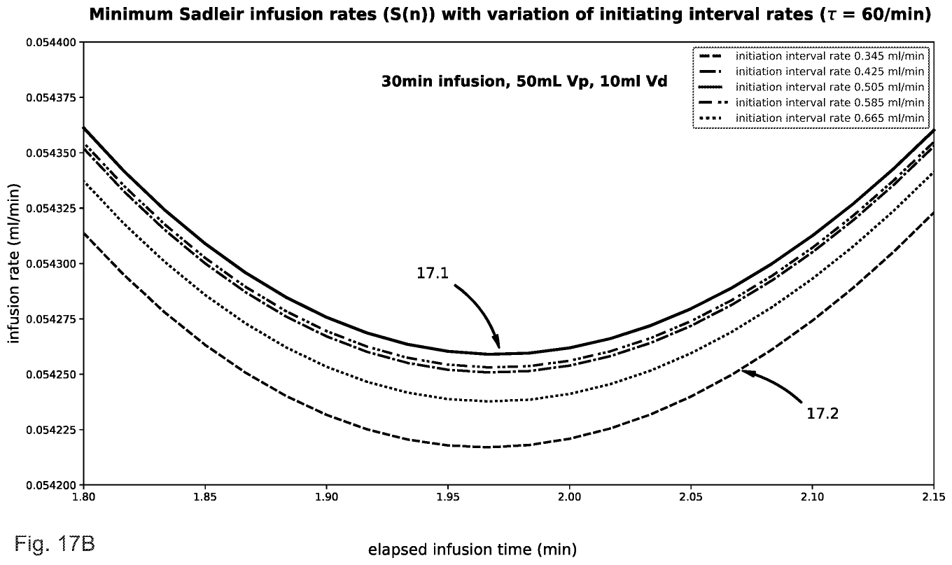

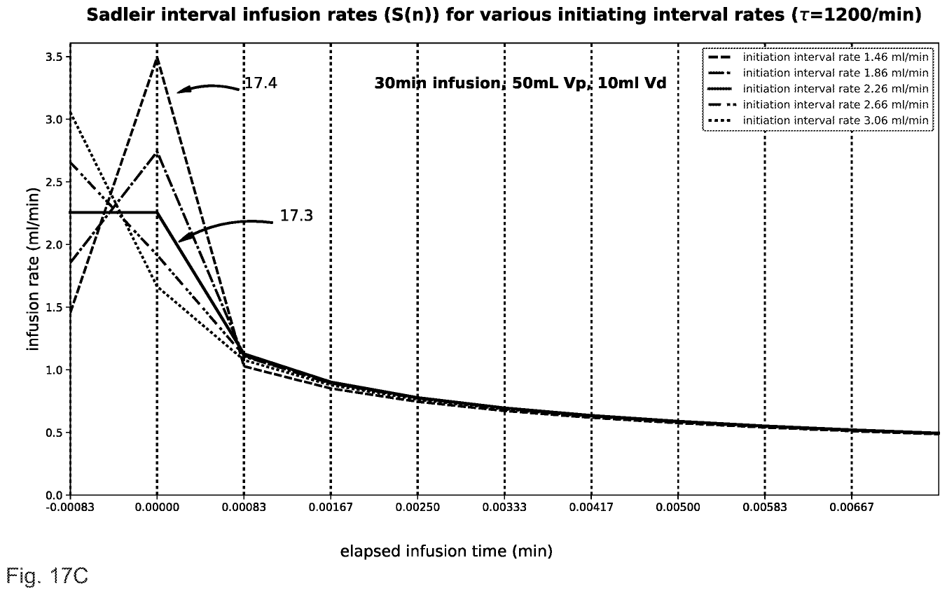

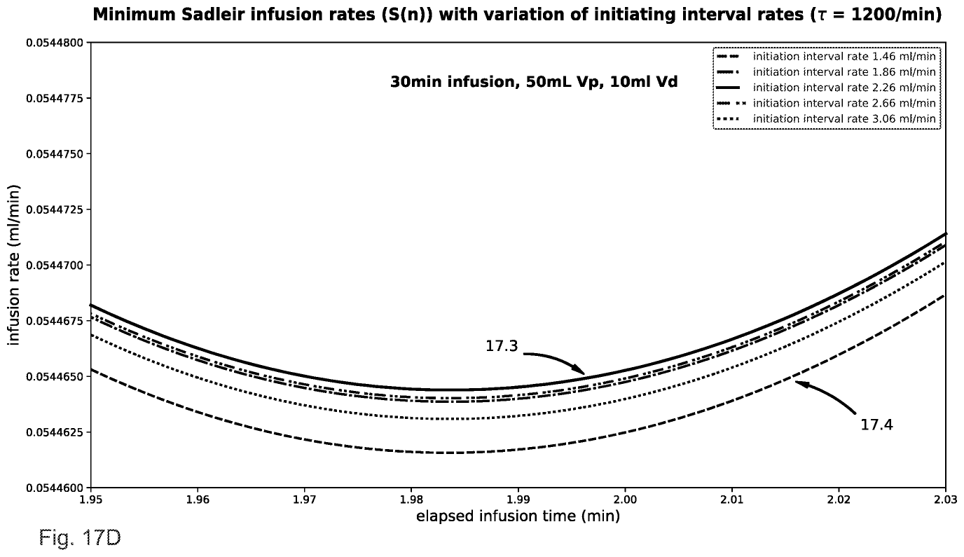

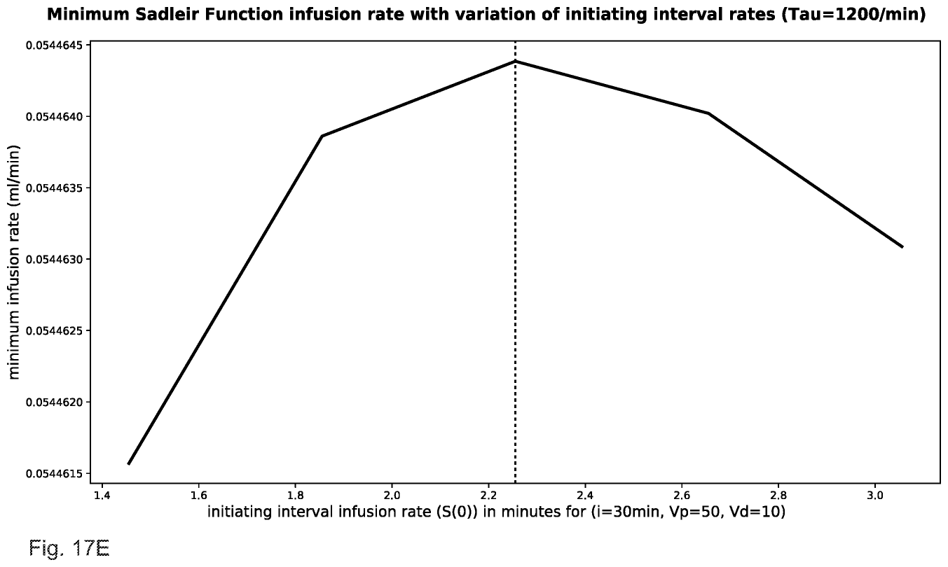

The Sadleir Function follows a concave curve starting from a particular value at t=0, reducing to a minimum value, and, after reaching the minimum value, increasing to a final value. FIGS. 17, in particular FIGS. 17B and 17D, illustrate the flow rate as dictated by the Sadleir function over a particular period of time for a 30 minute infusion duration, for different values of τ (60 and 1200, respectively).

As shown in, for example, FIGS. 17A and 17B, the flow rate starts at a particular flow rate and slows down until reaching a minimum flow rate at which thereafter the flow rate increases continuously until completion of the infusion process.

The optimal initiating flow rate (for infusion processes in accordance with the Sadleir method) is that particular flow rate that will result in the maximum minimum flow rate over the course of the infusion process. The reason that this particular flow rate is the optimal flow rate is that, as mentioned before, increasing the flow rate with which the pharmaceutical preparation exits the infusion driver 14 (i.e. the active agent chamber 98) increases the accuracy of the administration process of the pharmaceutical preparation because it is known that infusion drivers 14 do not deliver accurately pharmaceutical preparations at relative low rates as occurs when using the Tansy function.

As can be seen from FIGS. 17A (tau=60, i=30 min, Vₚ=50 mL, V_{d}=10 mL), the lowest initiating interval rate (17.2) results in a lower concentration in the dilution chamber at the end of this interval, resulting in a higher rate for S₁, but lower subsequent rates. It can be seen in FIG. 17B that the initiating rate that results an equal S₁ rate will result in the maximum flow rate minimum (17.1).

FIGS. 17C and 17D show a graph plotting the flow rates as dictated by the Sadleir function over particular periods of time for a multitude of instances having different initiating flow rates as for 17A and 17B, but with tau=1200. As shown in FIG. 17D, line 17.1 has a starting flow rate of approximately 2.26 ml/min and (as shown in FIG. 17D) the maximum minimum flow rate, and line 17.2 has the minimum flow rate compared to all other instances.

FIG. 17e shows a graph plotting the value of minimum flow rates for each particular flow rate of a multitude of flow rates from FIGS. 17C and 17D. As shown in FIG. 17E, the maximum minimum flow rate occurs with an initiating flow rate of approximately 2.26 ml/min. This particular flow rate will be chosen as the starting flow rate due to having maximum minimum flow rate.

The ideal priming (initiating) dose will be the one having as flow rate the starting flow rate of line 17.3; due to the fact that this line 17.3 has the maximum minimum flow rate as can be seen in FIG. 17E. The ideal initiating dose or rate, prior commenced of the infusion process, is that which results in the infusion rate of the initiating step (S(0)initiating) to be equal to the infusion rate of the first interval (S(1st interval)), such that the rate of S(0)=S(1).

The sensitivity of the Sadleir function to the variations in the flow rate of the initiating step is increased when the size of the interval (1/τ) over which, the Sadleir function is iterated, is greater; FIGS. 17A and 17B, and FIGS. 17C and 17D demonstrate the above. In fact, in FIGS. 17A and 17B, a value of τ of 60/min is used and the change in minimum flow rate is greater. And, as shown in FIGS. 17C and 17D, if a value of 1200/min for τ is chosen, the change in minimum flow rate is less. Decreasing the size of the intervals (increasing τ) decreases the sensitivity to changes in the initiating interval rate.

Further, after the initiating interval, the infusion process will commence.

The flow rate during the first interval of the Sadleir Function is such that the dose given during the first interval is D_{mtf}(t)₁ (as defined above) calculated based on the concentration inside the dilution chamber 32 after occurrence of the initiating dose.

The infusion time is divided up into τ* i intervals, where i is the number of minutes over which the infusion is delivered, and τ is the number of intervals per minute. Each interval is of duration 1/τ minutes.

The volume given by the modified Tansy function for interval n (between time=(n−1)/τ and n/τ minutes), is given by the integral of the tansy rate function multiplied by a correction factor which accounts for the amount of drug remaining in the dilution syringe at the end of the Sadleir Infusion (the second embodiment of the disclosure), or:

$${{V}_{m t f} ( t )}_{n} = {\int }_{\frac{n - 1}{τ}}^{\frac{n}{τ}} T ( t ) d t * ( \frac{{V}_{p} - ( {V}_{d} * ( 1 - {e}^{- \frac{{V}_{p}}{{V}_{d}}} ) )}{{V}_{p}} )$$

The rate of the Tansy function at any point in time (t) is defined as:

$$T ( t ) = \frac{{V}_{p} l n ( {2}^{\frac{30}{i}} )}{{2}^{16} - 2} {e}^{\frac{t}{2} l n ( {2}^{\frac{30}{i}} )}$$

therefore, the volume of the modified tansy function for any interval n is:

$${{V}_{m t f} ( t )}_{n} = {\int }_{\frac{n - 1}{τ}}^{\frac{n}{τ}} \frac{{V}_{p} ln ( {2}^{\frac{30}{i}} )}{{2}^{16} - 2} {e}^{\frac{t}{2} ln ( {2}^{\frac{30}{i}} )} d t * ( \frac{{V}_{p} - ( {V}_{d} * ( 1 - {e}^{- \frac{{V}_{p}}{{V}_{d}}} ) )}{{V}_{p}} )$$

or expanded to:

$${{V}_{mtf} ( t )}_{n} = ( ( \frac{2 {V}_{p}}{{2}^{16} - 2} {e}^{\frac{n}{2 τ} ln ( {2}^{\frac{30}{i}} )} - \frac{2 {V}_{p}}{{2}^{16} - 2} ) - ( \frac{2 {V}_{p}}{{2}^{16} - 2} {e}^{\frac{n - 1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} )$$

The dose of the modified tansy function (Dmtf(t)n) for interval n is given by multiplying (1) the volume given over the interval (Vmtf(t)n) by (2) the concentration of drug from the primary pharmaceutical container (Cₚ), or:

D_{mtf}(t)ₙ=V_{mtf}(t)ₙ*Cₚ
Therefore, the dose of the modified Tansy function for the interval n can be defined:

$${{D}_{m t f} ( t )}_{n} = {\int }_{\frac{n - 1}{τ}}^{\frac{n}{τ}} \frac{Vp * ln ( {2}^{\frac{30}{i}} )}{{2}^{16} - 2} * {e}^{\frac{1}{2} l n {2}^{( \frac{30}{i} )}} dt * ( \frac{{V}_{p} - ( {V}_{d} * ( 1 - {e}^{- \frac{{V}_{p}}{{V}_{d}}} ) )}{{V}_{p}} ) * {C}_{p}$$

Or:

$${{D}_{m t f} ( t )}_{n} = ( ( \frac{2 {V}_{p}}{{2}^{16} - 2} {e}^{\frac{n}{2 τ} l n ( {2}^{\frac{30}{i}} )} - \frac{2 {V}_{p}}{{2}^{16} - 2} ) - ( \frac{2 {V}_{p}}{{2}^{16} - 2} {e}^{\frac{n - 1}{2 τ} l n ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) * {C}_{p}$$

The rate of the initiating interval (S(0)) should equal that of the first interval, (S(1)), as has been explained previously.

The rate of the first interval (S(1)) is determined by the dose of the modified Tansy function for the equivalent interval of the infusion (from time zero to time 1/τ minutes) and concentration C_{d(0)} in the dilution chamber V_{d} at the start of this interval. The rate is equal to the volume given divided by the interval of time, and the volume is determined by the dose divided by the concentration, or:

$$S ( 1 ) = \frac{volume}{time interval} = \frac{( \frac{dose}{concentration} )}{( \frac{1}{τ} )} = \frac{dose * τ}{concentration}$$

Or:

$$S ( 1 ) = \frac{{{D}_{m t f} ( t )}_{1} * τ}{{C}_{d ( 0 )}} \qquad ( 16 )$$

The initial concentration of the dilution chamber is given by the dose given during the initiation step (n=0), divided by the volume of the dilution chamber, V_{d}. The dose given during the initiation step is equal to the volume (V₀) given during the initiation step, multiplied by the concentration in the primary pharmaceutical syringe (Cₚ). The volume given during the initiation step is equal to the initiation step rate (S(0)), multiplied by the duration of the interval (1/τ minutes) or:

$${C}_{d ( 0 )} = \frac{{V}_{0} * {C}_{p}}{{V}_{d}} = \frac{S ( 0 ) * \frac{1}{τ} * {C}_{p}}{{V}_{d}} \qquad ( 17 )$$

from equation 16 above, giving the rate of the interval (S1), we substitute C_{d(0)} to give:

$$S ( 1 ) = \frac{{{D}_{mtf} ( t )}_{1} * τ}{\frac{S ( 0 ) * \frac{1}{τ} * {C}_{p}}{{V}_{d}}} \\ S ( 1 ) * \frac{S ( 0 ) * \frac{1}{τ} * {C}_{p}}{{V}_{d}} = {{D}_{mtf} ( t )}_{1} * τ$$

Rearranged:
Or

$$S ( 1 ) * S ( 0 ) * \frac{\frac{1}{τ} * {C}_{p}}{{V}_{d}} = {{D}_{mtf} ( t )}_{1} * τ$$

As S(0)=S(1), therefore S(0)*S(1)=S(0)²:

$${S ( 0 )}^{2} = \frac{{{D}_{mtf} ( t )}_{1} * τ}{\frac{\frac{1}{τ} * {C}_{p}}{{V}_{d}}}$$

Or

$${S ( 0 )}^{2} = \frac{{{D}_{mtf} ( t )}_{1} * τ * {V}_{d}}{\frac{1}{τ} * {C}_{p}}$$

Or

$${S ( 0 )}^{2} = \frac{{{D}_{mtf} ( t )}_{1} * τ * {V}_{d}}{\frac{1}{τ} * {C}_{p}}$$

and as D_{mtf}(t)₁=V_{mtf}T(1)₁*Cₚ

$${S ( 0 )}^{2} = \frac{{V}_{mtf} T ( t ) * {C}_{p} * τ * {V}_{d}}{\frac{1}{τ} * {C}_{p}}$$

Cancel out the Cₚ values and multiply the right hand side by τ/τ:
to give S(0)²=V_{mtf}(t)₁*τ²* V_{d}
Or:

S(0)=\sqrt{V_{mtf}(t)₁*τ²*V_{d}}

As V_{mtf}(t)₁ is the integral of the modified tansy (rate) function between 0 minutes and 1/τ minutes:

$$S ( 0 ) = \sqrt{{\int }_{\frac{0}{τ}}^{\frac{1}{τ}} T ( t ) dt * ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

substitute in tansy rate function:

$$S ( 0 ) = \sqrt{{\int }_{\frac{0}{τ}}^{\frac{1}{τ}} \frac{{V}_{p} ln ( {2}^{\frac{30}{i}} )}{{2}^{16} - 2} {e}^{\frac{t}{2} l n ( {2}^{\frac{30}{i}} )} dt * ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

which expands to:

$$S ( 0 ) = \sqrt{\begin{matrix}( ( \frac{2 {V}_{p}}{{2}^{16} - 2} {e}^{\frac{1}{2 τ} l n ( {2}^{\frac{30}{i}} )} - \frac{2 {V}_{p}}{{2}^{16} - 2} ) - ( \frac{2 {V}_{p}}{{2}^{16} - 2} {e}^{\frac{0}{2 τ} l n ( {2}^{\frac{30}{i}} )} - \frac{2 {V}_{p}}{{2}^{16} - 2} ) ) * \\ ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}\end{matrix}}$$

which equals:

$$S ( 0 ) = \sqrt{( \frac{2 {V}_{p}}{{2}^{16} - 2} {e}^{\frac{1}{2 τ} l n ( {2}^{\frac{30}{i}} )} - \frac{2 {V}_{p}}{{2}^{16} - 2} ) * ( \frac{{V}_{p} - ( {V}_{d} * ( 1 - {e}^{- \frac{{V}_{p}}{{V}_{d}}} ) )}{{V}_{p}} ) * {τ}^{2} * {V}_{d}}$$

Notes
The initiation step or interval (n=0) is the dose that establishes the concentration in the dilution chamber, prior to the patient being administered the pharmaceutical in the first interval of the Sadleir method (n=1) The initiation step occurs prior to the infusion, from time

$$- \frac{1}{τ}$$

to time 0 minutes
Subsequent intervals span from

$$\frac{n - 1}{τ}$$

minutes to

$$\frac{n}{τ}$$

minutes
The first interval (n=1) spans from 0 minutes to

$$\frac{1}{τ}$$

minutes of the infusion
i is the duration in minutes of the infusion
τ is the number of intervals per minute calculated for the Sadleir function

$$\frac{1}{τ}$$

is the duration of each interval in minutes
n is the nth interval, spanning from

$$\frac{n - 1}{τ}$$

to

$$\frac{n}{τ}$$

minutes of the infusion
for example, a 30 minute infusion with τ=1200 intervals per minute will have 36,000 intervals in total, and the 1801st interval (n=1801) will start at time=1800/1200 minutes and finish at time=1801/1200 minutes

In particular, for a 30 minute infusion from a 50 ml syringe 15, with 10 ml dilution chamber 32 and 1/600 minute steps, the initial infusion rate is:

$$Rate = \sqrt{( \frac{2 {V}_{p}}{{2}^{16} - 2} * {e}^{\frac{1}{2 τ} l n ( {2}^{( \frac{30}{i} )} )} - \frac{2 {V}_{p}}{{2}^{16} - 2} ) * ( \frac{{V}_{p} - ( {V}_{d} * ( 1 - {e}^{- \frac{{V}_{p}}{{V}_{d}}} ) )}{{V}_{p}} ) * {V}_{d} * {τ}^{2}}$$

therefore:

$$Rate = \sqrt{( \frac{2 * 50}{{2}^{16} - 2} * {e}^{\frac{1}{2 * 600} l n ( {2}^{( \frac{30}{30} )} )} - \frac{2 * 50}{{2}^{16} - 2} ) * ( \frac{50 - ( 10 * ( 1 - {e}^{- \frac{50}{10}} ) )}{50} ) * 10 * {600}^{2}}$$

simplified to:

$$Rate = \sqrt{( 0.0015259 * 1.0005778 - 0.0015259 ) * ( 0.8013476 * 10 * 60 {0}^{2} )}$$

simplified to:
Rate=1.5948 ml/hr

$$\frac{1}{τ}$$

S(0)_{initiating} is the rate of infusion for the initiating interval which is of duration minutes
τ is the number of iterated intervals per minute
Vₚ is the volume of the drug solution in the delivery syringe or flask or bag
i is the chosen duration of the total infusion in minutes
V_{d} is the volume of the dilution chamber

For the same configuration, but τ= 1/1200 minutes, the priming rate (for 1/1200 minutes duration) is =2.25526 ml/min).

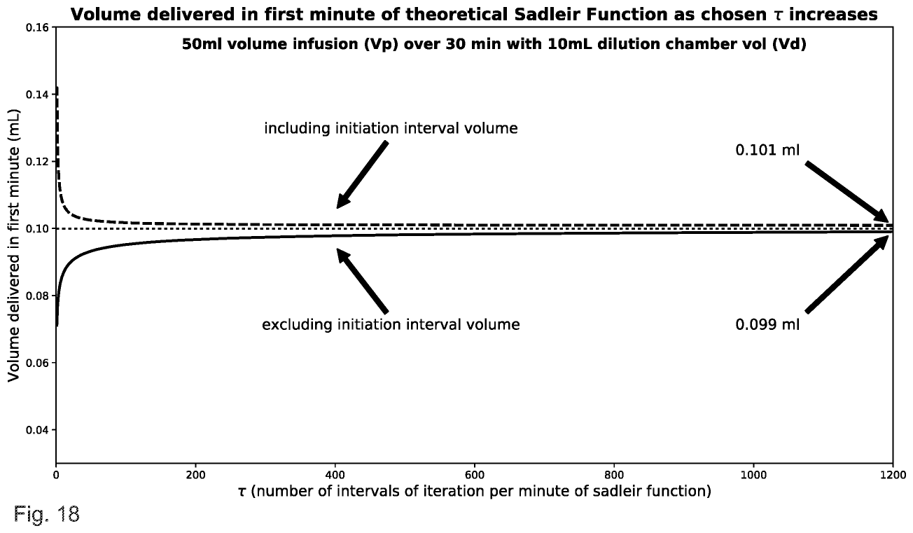

FIG. 18 illustrates the volume administered in the first minute using the Sadleir method using a 30 minute infusion from a 50 ml syringe, with a 10 ml dilution chamber.

The precision of the estimate for the volume administered in the first minute of the Sadleir function achieves 3 significant figures when iterating to a time interval of 1/1200^{th} of a minute (see FIG. 18 for the volume in first minute for a 30 minute infusion from a 50 ml syringe with a 10 ml dilution chamber).

Calculation of Rate of Subsequent Interval

As mentioned before, to calculate the value of the infusion rate for each subsequent interval occurring after the initiating interval, first requires an estimate of the concentration of drug in the dilution chamber 32 at the end of the interval the occurred prior the particular subsequent interval for which its infusion rate (the subsequent infusion rate) is being calculated. The subsequent infusion rate is calculated as that required to deliver a volume of fluid in the dilution chamber 32 that contains the equivalent dose (D_{mtf}) that would be given by the modified Tansy function (that is, the dose given by the Tansy function in the corresponding interval that is reduced by multiplying by the ‘correction factor’, see FIG. 13B and equation 6a below) assuming the concentration of drug calculated by equation 9 below (equation 14 in FIG. 13B) Thus:

The concentration in the dilution chamber 32 at the end of a particular subsequent interval n is approximated as the amount of drug in the dilution chamber 32 at the end of that subsequent interval n divided by the volume of the dilution chamber 32. The amount of drug in the dilution chamber 32 at the end of the subsequent interval n is approximated by:

the amount of drug in the dilution chamber 32 at the start of the interval (dilution chamber volume multiplied by dilution chamber drug concentration at the end of the previous interval (C_{d(n-1))});

added to the amount of drug that entered the dilution chamber during the interval (infusion rate (Sₙ) multiplied by interval duration (1/tau)) multiplied by the concentration of drug in the pharmaceutical preparation Cᵣ);

and subtracting the amount of drug that exited the dilution chamber 32 during the interval (interval infusion rate (Sₙ) multiplied by interval duration (1/tau)) multiplied by the concentration of drug in the dilution chamber at the end of the previous interval (C_{d(n-1)})).

Thus:

$${C}_{d ( n )} = \frac{( {C}_{d ( n - 1 )} * {V}_{d} ) + ( {S}_{n} * {C}_{p} * \frac{1}{τ} ) - ( {S}_{n} * {C}_{d ( n - 1 )} * \frac{1}{τ} )}{{V}_{d}}$$

The dilution chamber concentration (C_{d(n)}) can be simplified to:

$${C}_{d ( n )} = \frac{( {C}_{d ( n - 1 )} * {V}_{d} * τ ) + ( {S}_{n} * {C}_{p} ) - ( {S}_{n} * {C}_{d ( n - 1 )} )}{{V}_{d} * τ}$$

The infusion rate (Sₙ) of the subsequent interval (n) is then equal to the volume of pharmaceutical preparation to be delivered to the dilution chamber 32 divided by the duration of that interval n. The volume is equal to the dose of active ingredient dictated by the modified Tansy function divided by the concentration in the dilution chamber 32 at the end of the previous interval. The rate of the subsequent interval n is equal to the volume divided by the duration of the interval in minutes, or alternatively the volume multiplied by the number of intervals per minute, or:

$${S}_{n} = \frac{{D}_{{mtf ( t )}_{n}} * τ}{{C}_{{d}_{( n - 1 )}}}$$

As mentioned before, using the Sadleir function instead of the Tansy function, results in administration of a dose that is less than the dose administered at any point in time during the Tansy function. The dose per the Sadleir function is reduced by multiplying the dose as dictated by the Tansy function by the correction factor:

$$\frac{{V}_{p} - {V}_{d} ( 1 - {e}^{- \frac{{V}_{p}}{{V}_{d}}} )}{{V}_{p}}$$

Reducing the dose ensures that the duration of the infusion is equal to that of that provided by the Tansy function for the same volume of infusion and given that at the end of the infusion, an amount of drug remains in the dilution chamber 32.

The number of subsequent intervals is divided by the duration of infusion (in minutes) to give the number of intervals per minute (t), giving a total of (i*τ) intervals over the infusion period (each interval from a time (n−1)/τ to a time n/τ minutes, see FIG. 13D.

The volume administered by the Tansy function infusion for each interval is calculated by integrating the Tansy function over the time period of each interval; extending from (n−1)/τ to a time n/τ minutes.

The integral of the tansy function is calculated as:

$${\int }_{\frac{n - 1}{τ}}^{\frac{n}{τ}} T ( t ) dt = ( \frac{2 {V}_{p}}{{2}^{16} - 2} {e}^{( \frac{n}{2 τ} ) l n ( {2}^{( \frac{30}{i} )} )} - \frac{2 {V}_{p}}{{2}^{16} - 2} ) - ( \frac{2 {V}_{p}}{{2}^{16} - 2} {e}^{( \frac{n - 1}{2 τ} ) l n ( {2}^{( \frac{20}{i} )} )} - \frac{2 {V}_{p}}{{2}^{16} - 2} ) \qquad ( 5 )$$

$${\int }_{\frac{n - 1}{τ}}^{\frac{n}{τ}} T ( t ) dn is the integral of the \\ Tansy function ( ie . volume ) between \frac{n - 1}{τ} and \frac{n}{τ} minutes \\ {V}_{p} is the volume of the drug \\ solution in the delivery syringe or flask or bag \\ n is the iteration interval \\ τ is the number of iterated intervals per minute \\ i is the chosen duration of the total infusion in minutes$$

The volume administered for each interval (as calculated above) is converted into a dose by multiplying it by the concentration of the drug in the syringe 15. The calculated numerical value of the dose is then reduced to account for the fact that the total dose administered to the patient using the apparatus 10 using the Sadleir method is less than that infused from the syringe 15 due to the fact that a portion of the drug will remain in the dilution chamber 32 at the end of the infusion. Reduction of the numerical value of the dose is done by multiplying each the dose to be infused during each interval by

$$\frac{{V}_{p} - {V}_{d} ( 1 - {e}^{- \frac{{V}_{p}}{{V}_{d}}} )}{{V}_{p}}$$

which is 0.80135 for a 10 ml or 20 ml dilution chamber 32 with 50 ml or 100 ml syringes 15, respectively.

The dose administered by the modified Tansy function (the Sadleir Function) for each interval is therefore given by:

$${{D}_{mT} ( t )}_{n} = {\int }_{\frac{n - 1}{τ}}^{\frac{n}{τ}} T ( t ) dt * {C}_{p} * ( \frac{{V}_{p} - ( {V}_{d} * ( 1 - {e}^{- \frac{{V}_{p}}{{V}_{d}}} ) )}{{V}_{p}} ) \\ {{D}_{mT} ( t )}_{n} is the modified Tansy dose for the interval \\ {\int }_{\frac{n - 1}{τ}}^{\frac{n}{τ}} T ( t ) dt is the volume ( integral ) of the modified tansy \\ function for the interval \\ {C}_{p} is the original concentration of drug in the delivery \\ syringe or flask or bag \\ {V}_{p} is the volume of the delivery syringe or flask or bag \\ {V}_{d} is the volume of the dilution chamber \qquad ( 6 a )$$

As mentioned before, prior administering the pharmaceutical preparation to the patient, it is necessary establish a concentration of drug in the dilution chamber 32 by filling the dilution chamber 32 with the pharmaceutical preparation. This is done via the initiating step mentioned earlier and occurs prior to infusion of the pharmaceutical preparation to the patient. As mentioned earlier, the initiating interval (n=0, see FIG. 13D), is of the same duration as the first subsequent interval (interval n=1, see FIG. 13D) and ideally has the same flow rate and volume as the first subsequent interval n=1; using equation (4), the flow rate for the initiating interval (the starting rate S(0)_{initiating}) is given by solving:

$${S ( 0 )}_{initiating} = \sqrt{( \frac{2 {V}_{p}}{{2}^{16} - 2} * {e}^{\frac{1}{2 τ} l n ( {2}^{( \frac{30}{i} )} )} - \frac{2 {V}_{p}}{{2}^{16} - 2} ) * ( \frac{{V}_{p} - ( {V}_{d} * ( 1 - {e}^{- \frac{{V}_{p}}{{V}_{d}}} ) )}{{V}_{p}} ) * {V}_{d} * {τ}^{2}} \qquad ( 6 b )$$

$${S ( 0 )}_{initiating} is the rate of infusion for the initiating period \\ which is of duration \frac{1}{τ} minutes \\ τ is the number of iterated intervals per minute \\ {V}_{p} is the volume of the drug solution in the delivery syringe or \\ flask or bag \\ i is the chosen duration of the total infusion in minutes \\ {V}_{d} is the volume of the dilution chamber$$

This infusion occurring during the initiating interval, results in delivery of a dose to the dilution chamber of volume V_{d}. The resulting concentration in the dilution chamber 32 after the initiating interval is given by:

$${C ( 0 )}_{initiating} = \frac{{S ( 0 )}_{i} * {C}_{p}}{τ * {V}_{d}} \\ {C ( 0 )}_{initiating} = concentration of drug in the dilution chamber \\ after the initiating interval \\ {S ( 0 )}_{i} = rate of the sadleir function during the initiating step , \\ in ml / min \\ τ is the number of iterated intervals per minute , \\ and \frac{1}{τ} is the duration of each interval \\ {V}_{d} is the volume of the dilution chamber \\ {C}_{p} is the original concentration of drug in the drug delivery \\ flask or syringe or container \qquad ( 7 )$$

The rate for the first subsequent interval, n=1, after the initiating interval is then calculated using C₍₀₎ as the initial dilution chamber concentration (Cₙ₋₁). This is calculated:

$${S ( t )}_{n} = \frac{{{D}_{mT} ( t )}_{n} * τ}{{C}_{n - 1}} \\ {S ( t )}_{n} is the Sadleir function rate for interval n ( between \\ \frac{n - 1}{τ} and \frac{n}{τ} min ) in ml / min \\ {{D}_{mT} ( t )}_{n} is the dose given by the modified tansy function \\ between time \frac{n - 1}{τ} and \frac{n}{τ} \\ {C}_{n - 1} is the concentration of drug in the dilution \\ chamber at the end of interval n - 1 \\ τ is the number of intervals per minute \qquad ( 8 )$$

The concentration of drug in the dilution chamber 32 at the end of interval n is then calculated using the equation below:

$${C}_{n} = \frac{( {V}_{d} * τ * {C}_{n - 1} ) + {S ( t )}_{n} * {C}_{p} ) - ( {S ( t )}_{n} * {C}_{n - 1} )}{τ * {V}_{d}} \\ {C}_{n} is the concentration of drug in the dilution chamber at \\ the end of the nth step \\ {C}_{n - 1} is the concentration of drug in the dilution chamber \\ at the start of the nth step \\ {V}_{d} is the volume of the dilution chamber \\ {C}_{p} is the concentration of drug in the delivery syringe \\ or flask or bag \\ τ is the number of intervals per minute \qquad ( 9 )$$

The flow rate of each particular subsequent interval n is calculated from the last two equations (8) and (9), using the appropriate modified Tansy dose for each particular subsequent interval. In particular, the flow rate of each particular subsequent interval as dictated by the Sadleir function is calculated to give the volume that will result in the same dose as the Tansy function multiplied by the correction factor.

$$\frac{{V}_{p} - {V}_{d} ( 1 - {e}^{- \frac{{V}_{p}}{{V}_{d}}} )}{{V}_{p}}$$

which reduces the rate of drug administration at all stages of the Sadleir method by a constant fraction equal to the fraction of drug remaining in the dilution chamber compared to that of the total therapeutic drug dose.

Then, the concentration of the dilution chamber 32 is calculated for the next subsequent intervals based on the amount of pharmaceutical preparation that entered the dilution chamber 32 during the particular subsequent interval preceding each next subsequent intervals.

It is important to note that the above described process (as illustrated in FIGS. 13B and 13D) provides the values of rate as dictated by the Sadleir functions providing a curve (Sadleir theoretical curve) as shown for a particular example (for a 50 mL pharmaceutical preparation, with a 10 mL dilution chamber) in FIGS. 20B (for various infusion durations) and 20C (first 10 minutes of a 30 minute infusion) and FIGS. 22A, 22B and 22C (for a 30 minute infusion). Once the Sadleir theoretical curve has been calculated, the apparatus 10 in accordance with the second embodiment of the disclosure is programmed accordingly in order to administer the drug to the patient using the infusion driver 14. (In FIGS. 20B and 20C, the rate refers to the flow rate (ml/min) at which the infusion driver 14 infuses the pharmaceutical preparation out of the syringe 15 into the conduits 30a, dilution chamber 32, conduit 30b of the Sadleir apparatus 10).

The process for administering the drug using the infusion driver 14 in accordance with the Tansy or Sadleir function requires approximating the Tansy or Sadleir function with a series of ramp infusion steps (linearly changing infusion rate from beginning of the step to end) or constant infusion steps occurring sequentially during the duration of the infusion. Each step need to be adjusted to give the same or approximate volume of pharmaceutical preparation for the summation of corresponding intervals of the infusion driver 14 controlled by the Sadleir function. This particular process of approximation will be described at a later stage.

In operation, the process of setting up the apparatus 10 in accordance with the second embodiment of the disclosure for administering the drug requires two ‘priming’ steps and the drug-dosing infusion sequence for delivering the drug in accordance with the Sadleir function, as follows:

a) a first priming step to ensure the infusion driver 14 is free of slack and primes the conduit 30a, and

b) a second priming step to move the diluted pharmaceutical preparation from the exit of the dilution chamber 38 to the point of intravenous access in the patient.

In the first priming step, the conduit 30a is filled with the pharmaceutical preparation. This is done by opening the multi-way valve 42 to the atmosphere and operating the infusion driver 14 to purge the drug to the multi-way valve 42. The infusion driver 14 is stopped and the multi-way valve 42 is moved to impede contact between the conduit 30a and the atmosphere, and to open the dilution chamber 32 for delivery of the pharmaceutical preparation into the container 34 of the dilution chamber 32.

In the second priming step, the container 34 of the dilution chamber 32 and the catheter 50 plus its distal end 54 are filled with the pharmaceutical preparation. This will result in the pharmaceutical preparation entering the dilution chamber 32. During this second priming step, the infusion driver 14 is programmed to generate alternating rapid and slow flow rates to allow mixing of the drug and diluent contained in the container 34 of the dilution chamber 32. The second priming step continues until the first initial portion of mixed drug and diluent entering the first outlet 38 is advanced the length of conduit 30b and up to the point of entry into the patient. In this step, no drug is administered to the patient; thus, the alternating flow rate does need to be taking into account when calculating patient dosing.

Subsequently, the Sadleir method (for example, using ramp-step or constant-step approximation) is then started, resulting in infusion of the pharmaceutical preparation into the patient at the flow rate as dictated by the Sadleir function.

The functions used in Tansy or Sadleir methods (referred to as Tansy Functions and Sadleir functions) define the flow rate of the pharmaceutical preparation to administer the pharmaceutical preparation active ingredient (drug) to the patient at an initially low rate, with the flow rate varying as the infusion continues.

Approximations of the Tansy or Sadleir function may be used if the infusion driver 14 is only capable of delivering a finite number of infusion steps. The approximation may be done using a constant-infusion profile over each infusion step, or a linearly increasing or decreasing infusion rate over each step.

In fact, typically, programmable infusion devices (such as syringe drivers or peristaltic pumps or similar drug infusion pumps) are not capable of providing in a continuous manner the pharmaceutical preparation (with infinitely small steps). Instead the infusion devices provide either a series of constant steps, or a series of ‘ramp’ steps. The “ramp steps” start at one rate and linearly increase or decrease to another rate over the interval of the step. There may be a finite number of steps, either because of memory limits, or due to the unfavourable effect of latency between each step (an interruption to the infusion between each step). It should be noted that with the Sadleir method, even a series of constant rate or ramp rate infusion steps will result in a continuously changing active ingredient (drug) administration rate due to the continuously changing concentration of pharmaceutical preparation leaving the dilution chamber 32.

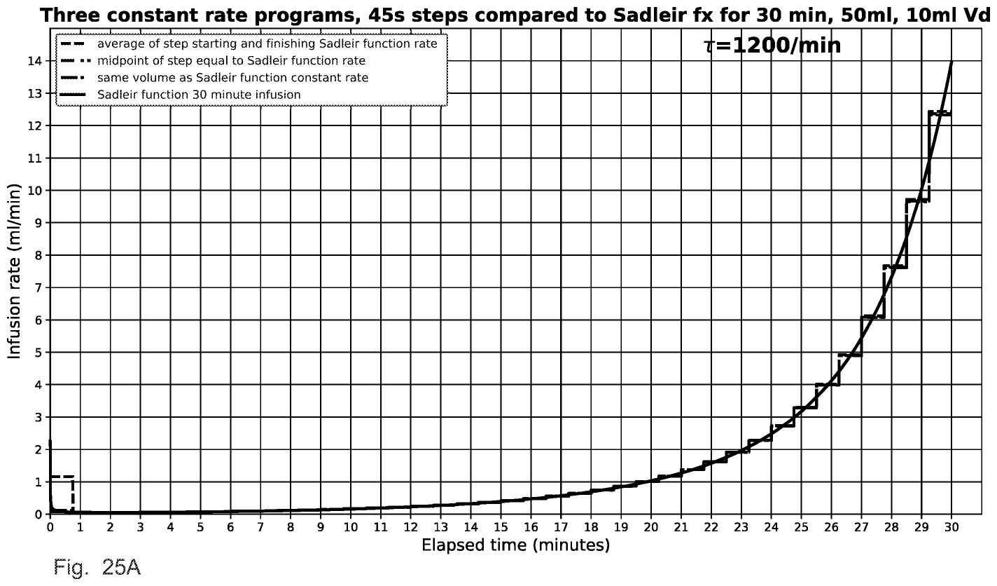

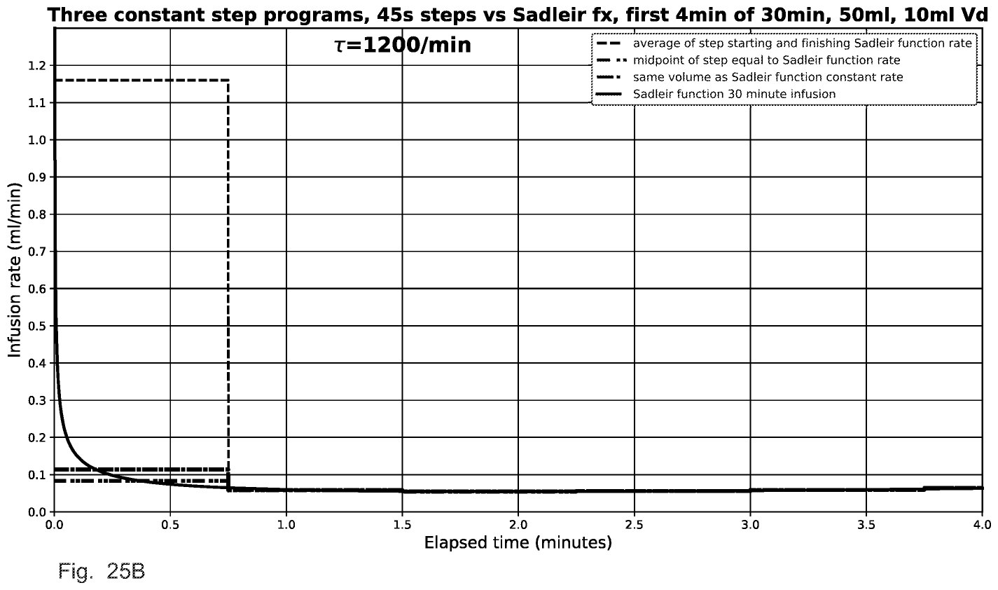

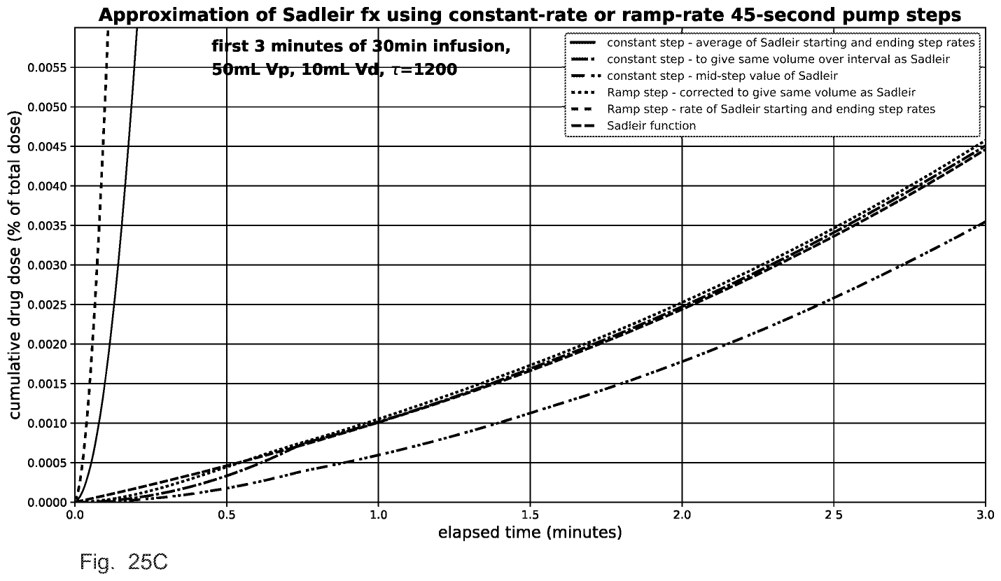

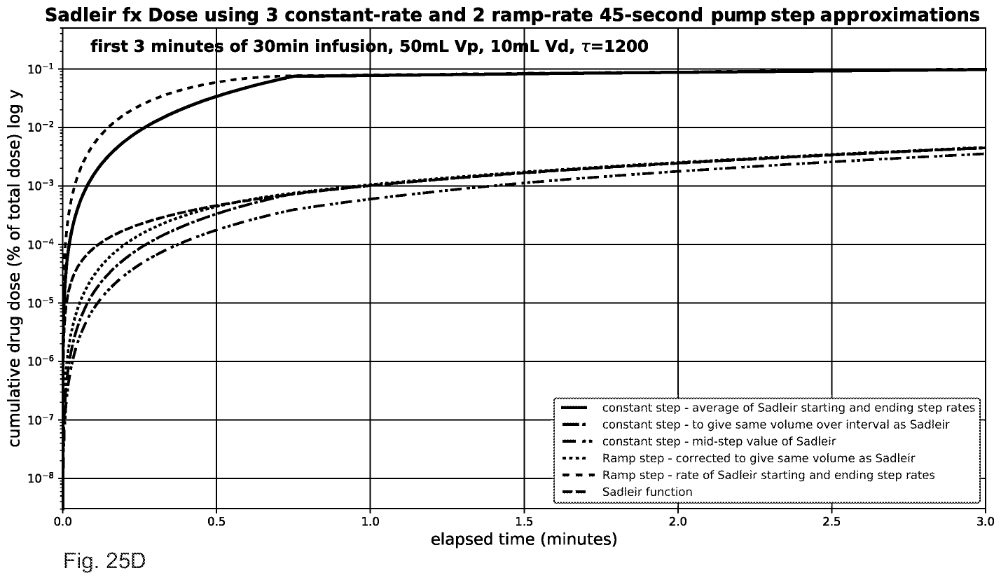

In accordance with the present embodiments of the disclosure there are provided several methods of approximating the Tansy or Sadleir function with a series of constant steps or ramp steps, and an improved method for each. FIGS. 25C and 25D illustrate the dose of active ingredient administered to the patient that results from the approximation process for the Sadleir function using a constant infusion method or ramp infusion method for the first 4 minutes of a 30 minute infusion, using 40 steps of 45 seconds duration.

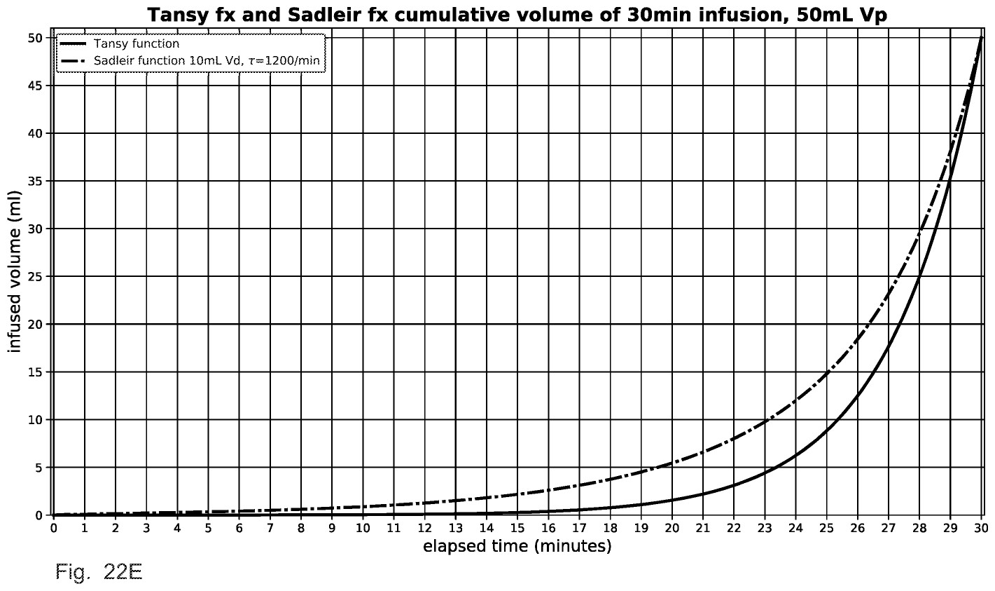

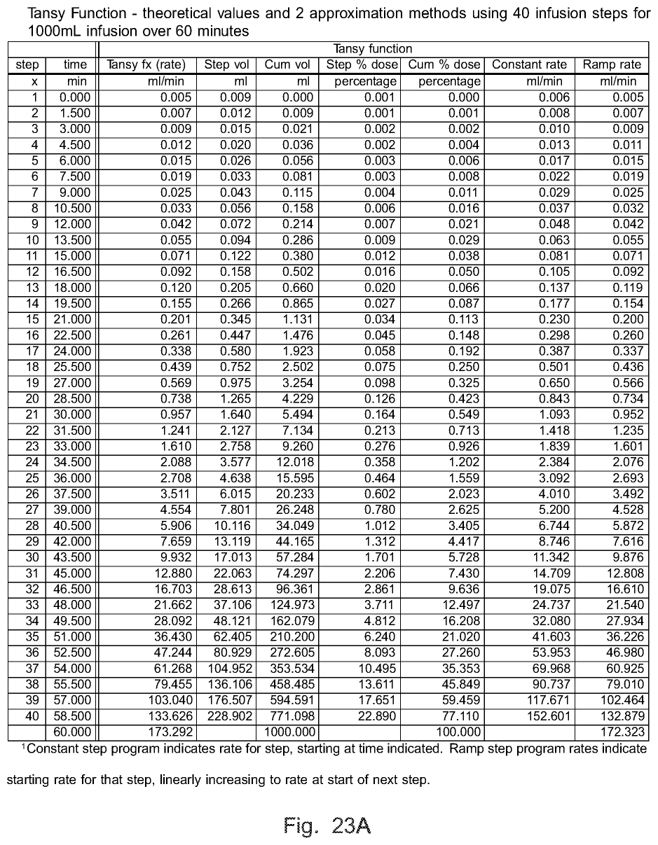

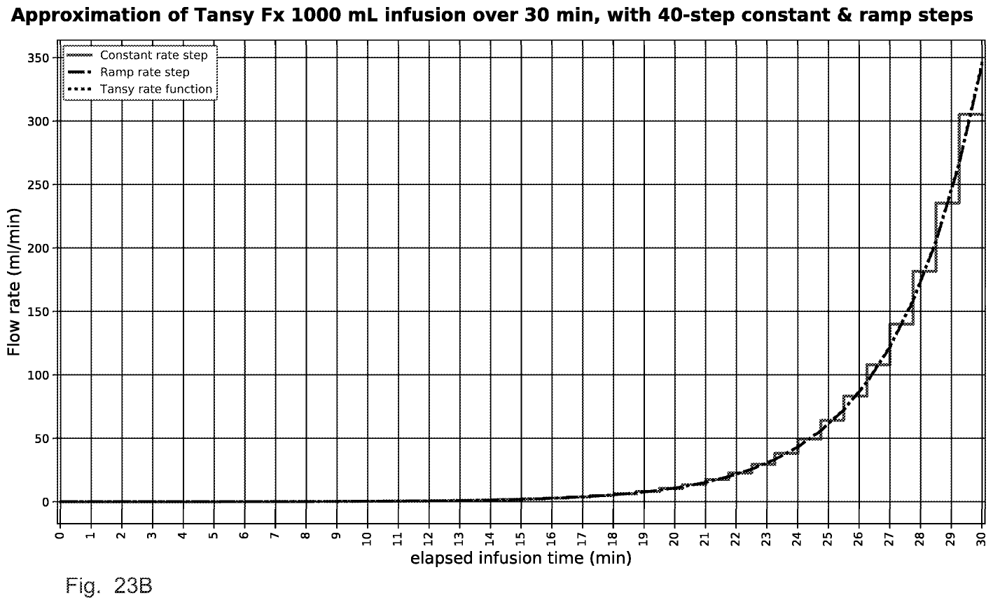

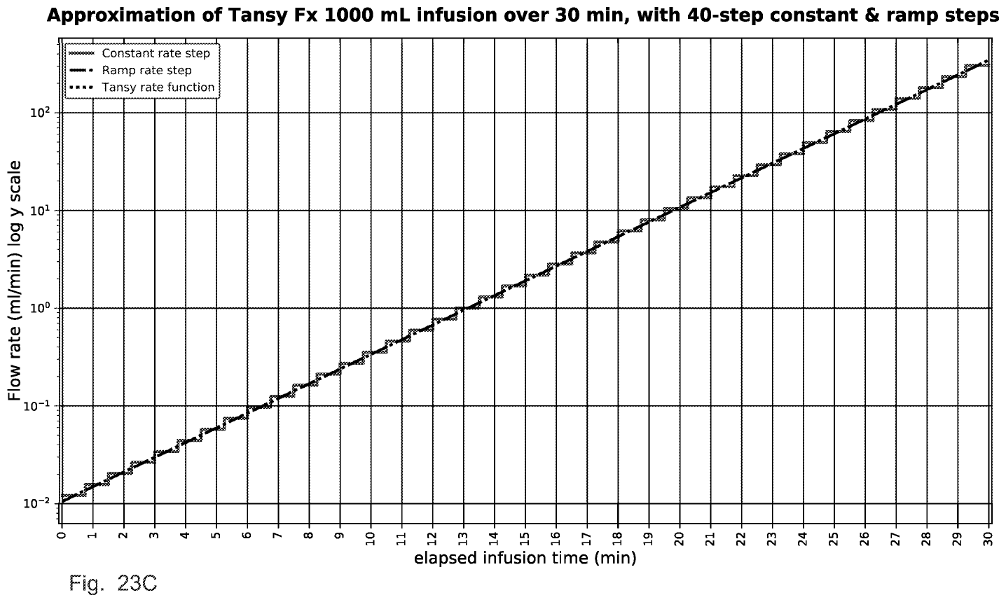

As shown in the FIGS. 12 and 13, each of the Tansy (FIGS. 23B and 23C) and Sadleir (FIGS. 24B, 24C, 25A and 25B) methods include defining the quantity of infusion steps that will sequentially occur during the duration of the infusion. Each step has a specific duration during which a particular quantity of pharmaceutical preparation will be provided. In a particular arrangement, these steps will deliver a similar volume as the Tansy or Sadleir function over the equivalent time interval of the infusion.

As mentioned above, during each of these steps there will be provided a particular quantity of the pharmaceutical preparation. The particular quantity of the pharmaceutical preparation that will be provided during each particular step will depend on the particular quantity of the pharmaceutical preparation that the Tansy or the Sadleir functions dictate that must be provided during the time interval of the particular infusion interval; in particular, as will be described below, this particular quantity is calculated using the quantity dictated for each particular interval at the corresponding particular moments of time during the infusion process as dictated by the Tansy (see FIG. 12B) or the Sadleir (see FIG. 13D) functions.

FIGS. 12B, 12C and 13C respectively illustrate the methods of approximating the Tansy and Sadleir function and delivering the pharmaceutical preparation to the patient.

As shown in FIGS. 12B and 12C in relation the Tansy method, after having calculated the actual amount (volume) of pharmaceutical preparation that will be delivered at the particular period of time of each step, it is decided whether the flow rate will be kept constant or increased linearly over each infusion step, depending on the capabilities of the infusion driver 14. The volume delivered in each step will be based on the volume of pharmaceutical preparation that has been calculated to be delivered over the corresponding interval of the Tansy function (see FIG. 12B).

Subsequently, the priming step will commence by delivering enough pharmaceutical preparation to the patient to fill the conduits 30a with the pharmaceutical preparation up to the point of the intravenous access point of the patient. At this stage, the infusion process may start by delivering to the patient, during each step, the amount of pharmaceutical preparation calculated for each step. Upon expiry of the infusion period, the infusion process is stopped.

As shown in FIG. 13D in relation to the Sadleir method, after having calculated the actual amount of pharmaceutical preparation that will be infused at the particular period of time of each step, the first priming step will commence by delivering enough pharmaceutical preparation to fill the conduits 30a with the pharmaceutical preparation. Subsequently, the second priming step will commence for filing the dilution chamber 32 and the conduit 30b for the diluted pharmaceutical preparation to reach the patient.

The infusion process then may start by (1) calculating the flow rate during the first step and (2) then delivering the pharmaceutical preparation to the patient at the calculated rate. At this stage, the pharmaceutical preparation may be delivered during each step to the patient until culmination of the infusion process.

With reference to the Sadleir method, as shown in FIGS. 13D and 13E, after delivery of the pharmaceutical preparation during each step, it is necessary to calculate the flow rate that is required for delivering the required amount of pharmaceutical preparation during the subsequent step. Finally, upon expiry of the infusion period, the infusion process is stopped and the remaining pharmaceutical preparation is delivered to the patient by, for example, collapsing the dilution chamber 32 as was described before in relation to the apparatus 10 depicted in FIGS. 1 to 11.

Part 3C—Variations of Sadleir Method

Alternative arrangements of the Sadleir method to approximate the active ingredient dosing rates of the Tansy method.

In an alternative arrangement of the Sadleir method, the apparatus may comprise a container 34 (comprising the dilution chamber 32) with the container 34 not having the ability to be selectively displaced between an expanded condition and a contracted condition (i.e. is of fixed volume). In order to compensate for the reduced total dose of drug administered compared to the equivalent Tansy function which results as a consequence of drug being present in the dilution chamber 32 at completion of the infusion, the concentration or volume of pharmaceutical preparation can be increased so that the active ingredient dosing rates of the equivalent Tansy method is provided. Particularly we can increase the:

a) concentration of the drug in the syringe 15 of the infusion driver 14 prior to commencement of the infusion process. The concentration will be equal to the original concentration (the concentration that would be required in order to provide the prescribed dose of active ingredient) multiplied by the inverse of the ‘correction factor’, or

$$\frac{{V}_{p}}{{V}_{p} - {V}_{d} ( 1 - {e}^{- \frac{{V}_{p}}{{V}_{d}}} )} .$$

The method (‘Increased concentration Sadleir method’) will deliver the equivalent drug dosing as for the Tansy function, rather than the modified Tansy function, see FIGS. 13D and 13E. For example, if the Tansy method is used to deliver 2 g of cephazolin as a 50 mL pharmaceutical preparation over 30 minutes (concentration 0.04 g/ml) the Sadleir method that will deliver the same dosing profile will be programmed to deliver 2.496 g of cephazolin in 50 mL of pharmaceutical preparation (concentration approximately 0.05 mg/ml) over 30 minutes, and a pharmaceutical preparation of this concentration (0.05 mg/ml) with sufficient volume to allow for priming the apparatus 10 (total volume of preparation approximately 53 mL) will be required; or

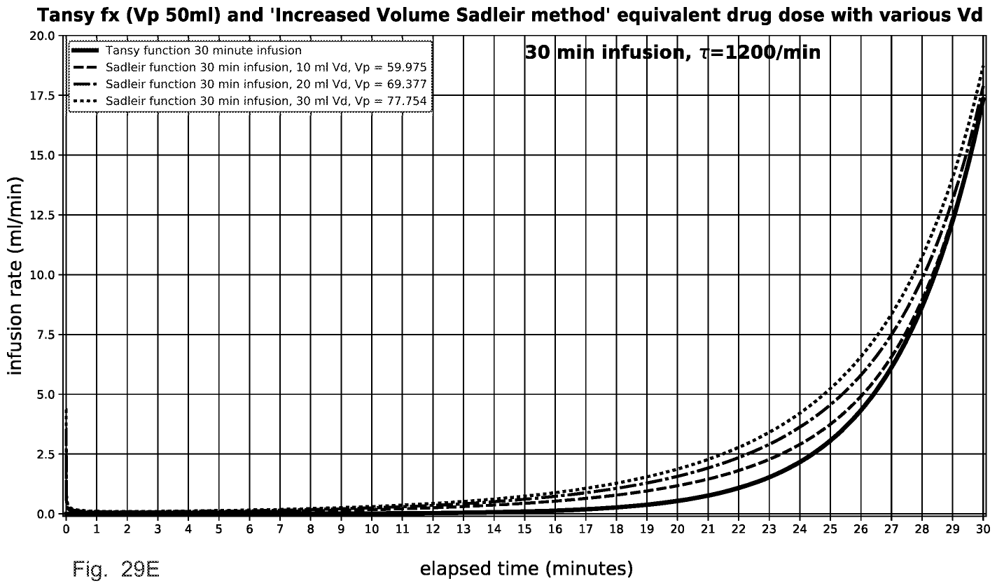

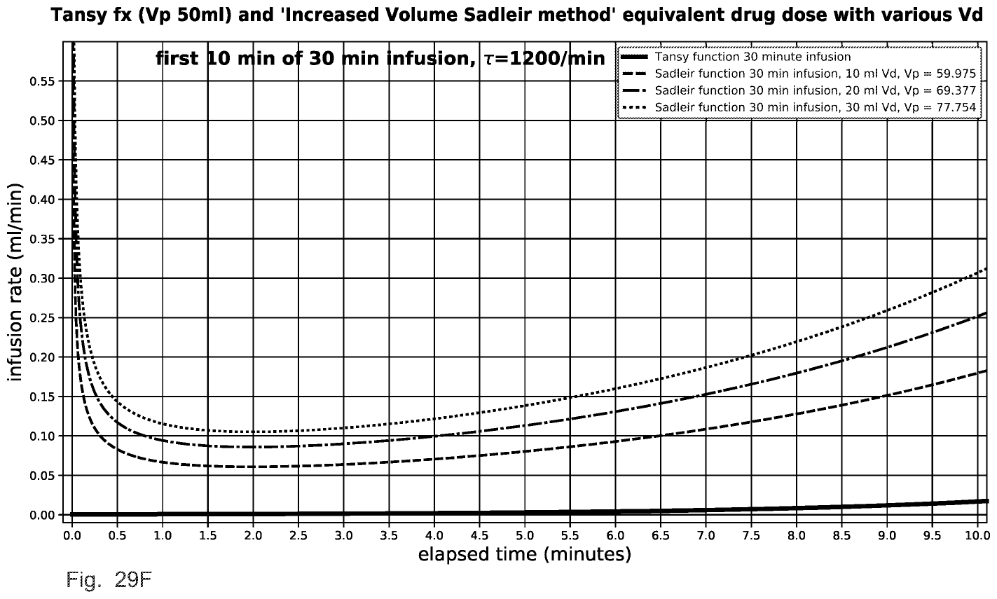

b) volume of pharmaceutical preparation, whereby an increased total volume of pharmaceutical preparation with the same concentration as that of the pharmaceutical preparation for the equivalent Tansy method is delivered over the same duration of the infusion. This increased volume of pharmaceutical preparation is determined by determining the volume that would be delivered in executing the method of the Kelly function over the infusion duration. That is, this is determined by applying the Kelly function to the duration of the infusion interval, and will be an amount less than Vₚ+V_{d}. This total volume is then delivered over the duration of the infusion according to the Kelly function (see FIGS. 29A and 29B). This alternate version delivers to the patient a dose of active ingredient that is equal to the equivalent Tansy method at each point in time during the infusion, rather than a fraction of the amount as occurs in the Sadleir method. For example, if the Tansy method is used to deliver 2 g of cephazolin as a 50 mL pharmaceutical preparation over 30 minutes (concentration 0.04 g/ml), this method to deliver the same dosing profile will be programmed to deliver 59.98 mL of infusion of pharmaceutical preparation of the same concentration as the Tansy function (2.4 g of cephazolin in 59.98 mL, concentration 0.04 g/ml) and over the same infusion duration, using the algorithm in FIGS. 29A and 29B. This is illustrated in FIGS. 29C, 29D and 29E and referred to as the ‘Increased volume Sadleir method’. The total volume of pharmaceutical preparation in the syringe in the syringe driver may need to be increased further to allow for the volume required for the priming steps prior to the infusion.

The ‘Increased concentration Sadleir method’ comprises the use of the second embodiment of the disclosure with an increased concentration of active ingredient in the pharmaceutical preparation compared to that of the comparable (same Vp and i) Tansy Method. The active ingredient concentration in the pharmaceutical preparation equals the equivalent Tansy method concentration multiplied by:

$$\frac{{V}_{p}}{{V}_{p} - {V}_{d} ( 1 - {e}^{- \frac{{V}_{p}}{{V}_{d}}} )} .$$

For the example of a 50 mL infusion volume with a 10 mL dilution chamber, choosing a pharmaceutical preparation with a drug concentration that is

$$\frac{50}{50 - 10 ( 1 - {e}^{- \frac{50}{10}} )} = 1.2479$$

times the concentration in the equivalent Tansy Method will result in the same active ingredient (drug) dosing profile. The infusion rates and volumes delivered during the ‘Increased concentration Sadleir method’ are the same as for the previously described Sadleir method. At completion of the infusion, the contents of the dilution chamber are discarded.

If an increased volume of infusion is not contraindicated, a further alternative arrangement of the second embodiment of the disclosure that provides the same active ingredient dosing profile as the equivalent Tansy method is the ‘Increased volume Sadleir method’. In the Increased volume Sadleir method, when compared to the equivalent Tansy method, the same infusion duration and pharmaceutical preparation active ingredient concentration is used. However, a larger volume of pharmaceutical infusion and higher rate of infusion is used to deliver the same dosing of active ingredient as for the equivalent Tansy method. The higher infusion volume is calculated by an iterative function described below, and the higher infusion rates are calculated using a modification of the Sadleir function. Any solution in the dilution chamber 32 at the end of the infusion period is discarded. For the equivalent Tansy function using a 50 mL infusion over 30 minutes, the ‘Increased volume Sadleir method’ comprises an infusion of 59.98 mL when using a 10 mL dilution chamber 32, 69.38 mL when using a 20 mL dilution chamber 32 or 77.75 ml when using a 30 mL dilution chamber 32 (see FIGS. 29C, 29D, 29E and 29F for the infusion rates and infusion volumes over the duration of a 30 minute infusion time). The same active ingredient (drug) dosing over the duration of the infusion is delivered to the patient compared to the equivalent Tansy method, as illustrated in FIG. 29F.

The algorithm (for calculating the required total infusion volume of pharmaceutical preparation at the same concentration as that of the equivalent Tansy method infusion, but delivering the same dose of active ingredient at any point in time during the ‘Increased volume Sadleir method’) is an iterative process. The values Vₚ refers to the volume of pharmaceutical preparation used in the equivalent Tansy method infusion, and V_{d} refers to the dilution chamber volume of the apparatus. The volume of active ingredient infused into the dilution chamber during the infusion will be greater than the value Vₚ entered into the algorithm (the Kelly function).

The algorithm for calculating the infusion rate for the ‘Increased Volume Sadleir Method’ is depicted in FIGS. 29A and 29B. The infusion rates are higher than for the equivalent (same Cp, Vd, i) Sadleir function, and the rate of increase in the dilution chamber drug concentration is greater with respect to time due to this. Over each interval n of the infusion, the volume of diluted pharmaceutical preparation that will give the same dose as the equivalent (same Cp, i) Tansy function is delivered, rather than the same dose as the modified Tansy function. As a result, infusion rates are higher and a larger total volume (ξ) is delivered over the infusion period. At the completion of the infusion process, drug remaining in the dilution chamber is discarded.

FIGS. 29A and 29B depicts a flowchart illustrating the modified Sadleir function which is applied to calculate the infusion flow rates for the ‘Increased Volume Sadleir method’. This is similar to the Sadleir function and method except that the equation for determining the starting rate omits the ‘correction factor’, and equations 13 and 15 use the Dose of the Tansy function rather than the Dose of the modified Tansy function to calculate the rates of infusion.

The rates and volumes delivered over the course of a 30 minute infusion using the ‘Increased Volume Sadleir Method’ using various dilution chamber volumes and that will administered the similar dosing of active ingredient over the infusion period as the equivalent Tansy method are illustrated in FIGS. 29C, 29D, 29E, 29F and 29G.

FIGS. 29C and 29D illustrate the cumulative volume infused using the alternative embodiment (‘Increased Volume Sadleir method’) of the second embodiment of the disclosure with various dilution chamber volumes (10 mL, 20 mL and 30 mL), wherein FIG. 29D illustrate the first 15 minutes of a 30 minute infusion. A larger volume of pharmaceutical preparation is used compared to the equivalent Tansy method, with an equivalent dose of drug administered to the patient at any time during the infusions;

FIGS. 29E and 29F illustrate the infusion rates using the alternative embodiment (‘Increased Volume Sadleir method’) of the second embodiment of the disclosure with various dilution chamber volumes (10 mL, 20 mL and 30 mL), wherein FIG. 29F illustrate the first 10 minutes of a 30 minute infusion. The infusion rates are higher for the alternative embodiment than the Tansy method, resulting in the equivalent dose of drug being administered to the patient at any time during the infusions.

FIG. 29G illustrates the similar dosing of active ingredient over the infusion period when using the ‘Increased volume Sadleir method’ with various dilution chamber volumes, compared to the equivalent Tansy method.

If the Sadleir method is used with an increased pharmaceutical preparation concentration or increased infusion volume as described above, an alternative arrangement may comprise a container 34 (having a dilution chamber 32 (without the ability to be selectively displaced between an expanded condition and a contracted condition (i.e. is of fixed volume).

Approximation of infusions using infusion pumps capable of discrete infusion steps (‘pump steps’)

In operation, the processor 16 will run instructions of code (e.g. similar to FIG. 27 (Tansy method) or FIG. 28 (Sadleir method)) for obtaining the particular quantity of the pharmaceutical preparation that will be provided during each particular step; in particular, running the instructions will calculate the amount (the theoretical amount) of pharmaceutical preparation to be delivered at the particular period of time of each interval as dictated by the Tansy or the Sadleir functions; and, this theoretical amount of pharmaceutical preparation will be used for calculating the actual amount of pharmaceutical preparation that will be delivered at the particular period of time of each step. The actual amount of pharmaceutical preparation to be delivered during each step may be, as will be explained below, the average of the theoretical amount of pharmaceutical preparation to be delivered over the periods of time as dictated by the Tansy or the Sadleir functions.

A first method is to use an infusion driver 14 that is capable of infusing a series of constant-rate infusion steps (see FIGS. 25A and 25B). This method is referred to as the constant step methods.

A first arrangement of the constant step methods (the “average constant step” is to set the flow rate of pharmaceutical preparation out of the syringe driver during each step as the average of the value at the beginning of the step and the value at the end of the counterpart step as dictated by the Tansy or Sadleir function.

A second arrangement of the constant step methods (the “middle-value constant step” method) is to set the flow rate of pharmaceutical preparation to be delivered during each step equal to the flow rate at the mid-point (mid-way between the start and end of the step) of the counterpart time period as dictated by the Tansy or Sadleir function.

A third arrangement of the constant step methods (“corrected constant step”) is to set the flow rate of the pharmaceutical preparation as that which will deliver the same volume as delivered during the duration of the time period by counterpart Tansy or Sadleir function.

The second method is to use an infusion driver 14 that is capable of delivering a series of ramp-step infusions. This method is referred to as the ramp-step method.

FIG. 24A is a table of values of two examples of approximating the Sadleir function for a 50 mL infusion of pharmaceutical preparation over 30 minutes, using a 10 mL dilution chamber and τ of 1200/min. The first column lists the integration interval (n) at the boundary of each step, the second column the infusion step commencing at that point, and the third column the elapsed infusion time at that point. The starting rate of a ramp-rate program infusion step is indicated (‘ramp rate’), linearly increasing or decreasing in rate until it reaches the starting rate of the subsequent step is given in the fourth column. Values for this ramp-rate approximation for volume delivered over each step (‘interval vol’), and percentage of the total dose of the equivalent Tansy function (‘cum dose %’), are given in the fifth and sixth columns, respectively. In column 7, the step rate for approximation of the Sadleir function with a constant-rate infusion step of 90 seconds is given for each step (‘constant rate’), as is volume delivered over each step (interval vol) and percentage of total dose of the equivalent Tansy function (‘cum dose %’) in columns 8 and 9.

In a first arrangement of the ramp-step method is to use a pump that is capable of delivering a series of ramp-steps, where each step begins at one first rate and linearly decreases or increases to a second rate at the end of each step, see FIGS. 24B, 24C and 24D.

The actual amount of pharmaceutical preparation to be delivered at the start of each step is defined as the amount of pharmaceutical preparation dictated by the Tansy or Sadleir function at the start of each counterpart interval. To calculate the total volume for each step it is assumed that the variation of rate between the end and the start of each step varies linearly (either decreases or increases).

However, the total volume calculated for each step using (1) the actual rate of infusion of pharmaceutical preparation to be delivered at the start and end of each step, and (2) assuming that the variation in flow rate is linear, does not correspond to the theoretical volume as dictated by the Tansy or Sadleir function—this is because the variation in flow rate between the start and end of each interval as dictated by the Tansy and Sadleir function is not a linear variation; instead, the curve representing this particular variation in flow rate is concave in shape. Thus, the flow rate for each step is reduced to match: (1) the actual volume to be delivered by the infusion driver 14 during each step with (2) the volume to be delivered during each interval as dictated by the Tansy or Sadleir function.

A second arrangement of the ramp step method (the “corrected ramp step”) is to define the rates at the starting and finishing rates at each step as the rate of the Tansy or Sadleir function, as above; and then, to calculate the volume to be delivered as dictated by the Tansy or Sadleir function for all intervals excluding the first interval as the majority of the error for the Sadleir function occurs in the first interval. All rates from the start of the second interval are reduced by the proportion of volume that is given in excess of the intended volume over this period due to discrepancy arising due to assuming a linear variation instead than a variation that follows a concave curve as dictated by the Tansy or Sadleir function.

The rate for the end of the first step is defined as this corrected starting rate for the second step. The rate for the start of the first step is then defined as the rate that will result in the ramp-function for this first step delivering the same volume that would be given by the Sadleir function over the first interval (see FIGS. 24B and 24C).

The different rates over time for the three constant step approximations of the Sadleir function are illustrated in the FIGS. 25A and 25B, and the percentage of cumulative dose administered vs infusion time for the five approximation methods of the Sadleir function and compared to the theoretical Sadleir curve are given for the first 3 minutes of a 30 minute infusion in FIGS. 25C and 25D. (The five different approximations of the flow rate include 2 different approximations using an infusion with ramp-rate steps or and 3 different approximations using constant-rate steps, with an infusion step occurring every 0.75 minutes over a 30-minute infusion for: total infusion volume 50 ml, 40 steps, and τ=1200. The pharmaceutical dose administered by the Sadleir apparatus using these protocols is dependent on the concentration of drug leaving the dilution chamber 32 and entering the patient, and the speed at which the infusion driver 14 drives the diluted pharmaceutical preparation from the dilution chamber 32 into the patient.

Part 4—Examples

Referring now to FIGS. 23, these figures refer to a realisation of the first embodiment of the disclosure.

FIG. 23A is a table of the instantaneous rates of the Tansy function at various points in time of a 60 minute infusion of 1000 mL of pharmaceutical preparation, and the values for approximating this function with an infusion device using forty constant-rate steps or forty ramp-rate steps methods. The two methods demonstrated comprise 40 constant-rate steps or 40 ramp-rate steps. The table includes the program values for 40 infusion steps of 45 seconds duration involving a constant-rate step (‘Constant rate’ column), or rate that changes linearly from a rate at the start of the step to the rate at the start of the next step (‘Ramp rate’ column). The volume delivered over each step interval (‘Step vol’ column), cumulative volume delivered (‘Cum vol’) and dose over each step interval (percentage of pharmaceutical preparation, ‘Cum % dose’) is also given;

FIG. 23B (linear y axis scale) and 23C (logarithmic y axis scale) illustrate the flow rate of the two approximations of the Tansy function using 40 infusion steps over a 30-minute infusion of 1000 ml. One (‘Constant rate step’) approximation uses 40 constant rate infusion steps, the other (‘Ramp rate step’) uses 40 infusion steps in which the rate linearly increases over the duration of each step to give the equivalent volume of the tansy function with the starting and finishing rates proportional to the tansy function rate at those points in time.

This particular realisation related to the first embodiment of the disclosure comprises:

a) a therapeutic dose of a pharmaceutical preparation over an appropriate time frame (in this case 30 minutes) is administration to a patient;

b) a large volume of solution (1000 ml) is used so as to reduce inaccuracy in the early stages of the infusion;

The equipment used for conducting this particular realisation is a relative large volume computer controlled peristaltic pump acting as the infusion driver 14 having a 1000 ml syringe 15 containing the pharmaceutical preparation to be administered to the patient via conduits 30a to a three-way tap which is attached to the intravenous access (conduit 30b) to the patient.

An example of the software instructions in python 3 language for use with the computer system 12 to calculate variables for operating the infusion driver 14 is presented in FIG. 27. FIG. 27 is the software code, written in python 3, to calculate the values that can be sent to the infusion device to realise the Tansy method (first embodiment of the disclosure). These can be manually entered into the infusion device for either constant infusion steps or ramp infusion steps, or they can be sent to microprocessor through various means. This software will generate the infusion step rates and volumes which can be manually entered via the keypad 26, or stored in the external memory drive 20 with additional software instructions depending on the characteristics of the computer system 12.

For this particular realisation the initial variable to be keyed in the infusion driver 14 by the operator are:

a) infusion duration (i)=30 minutes

b) infusion volume (Vₚ)=1000 ml

c) 40 steps for a 30 minutes infusion

The particular infusion driver 14 used for this realisation is capable of linearly-changing rate throughout each infusion step (Ramp-step program) or a constant rate throughout each infusion step (Constant-step program). If there is a period between infusion steps where fluid is not administered (a pause), this is defined as a latent period and the duration of this period is noted and accounted for as was described earlier when explaining the method for approximating the Tansy curve.

In the present realisation, the ramp-step program and the constant-step program are conducted and compared against each other in graphs illustrated in FIG. 23b.

The volume delivered by the Tansy function is then calculated for each interval as explained with reference to FIGS. 12A, 12B and 12C. The flow rate for each programmed infusion step of the infusion driver 14 is then calculated for each constant-step program and ramp-step program.

For the Constant-step program, the infusion rate (ml/min) is equal to the rate over the interval that will result in the same volume being delivered that would be delivered by the calculated Tansy function over the same infusion period.

For a Ramp-step program, the infusion driver 14 is capable of delivering flow over the infusion step that begins at one rate and linearly decreases or increases to a finishing rate. The next infusion step will then begin at this finishing rate and linearly increase or decrease until the finishing rate of that step. This process is reiterated over all infusion steps.

The Ramp-step program is initially calculated such that the starting values of each infusion step correspond to the same flow rate as the Tansy function interval at that point of the infusion process. This program will calculate the volume of the pharmaceutical preparation during each step to be greater than the volume dictated by the Tansy function as a consequence that the variations in flow rates are linear rather than non-linear as is in the Tansy function.

A process of correcting the ramp rates to more closely follow the Tansy function is explained below:

a) calculate the volume of pharmaceutical preparation that all the ramp rates (in this particular example there are 40 ramp steps) will over-deliver volume (this is referred to as V₂) when compared to volume V₁ dictated by the Tansy function over the duration of the infusion process;

b) multiply the starting and finishing flow rates by the quotient: V₁/V₂; and

c) correct for the latent period between each infusion step—this is only applicable if flow rate pauses between infusion steps. In particular, if the pump has a latent interval (pause) of 0.250 sec between each step (each step having a duration 45 seconds), correction is required by increasing the flow rate at all times by multiplying the value of each flow rate by 45/(45−0.250) to ensure a similar volume is given as dictated by the Tansy function.

In operation, the 3-way tap (receiving the conduit 30a coming from the infusion driver 14 and extending to a three-way-tab attached to the intravenous access of the patient) is open to the atmosphere for priming of the conduit 30a by starting the first priming program (e.g.: 0.5 ml over 30 seconds) for provision of the pharmaceutical composition to the entry point of patient.

The infusion driver 14 is then stopped and the 3-way-tap is closed to direct the pharmaceutical preparation to the patient.

Subsequently, the infusion driver 14 is restarted and the infusion process as described with reference to FIGS. 12A, 12B and 12C; upon, completion of the infusion process the infusion driver 14 is stopped.

Upon inspection of the graphs of FIGS. 23B and 23C, it can be seen that the flow rates as dictated by the Tansy function that even though a relative large volume of intravenous fluid has been used to dilute the drug, the infusion rates are very low compared to that delivered by the second embodiment of the disclosure (the Sadleir Function; see FIGS. 22B and 22C). In particular, it is not until after about 14 minutes that 1 ml of the pharmaceutical preparation has been delivered to the patient. The flow rates at the later part of the infusion process are relative high; these relative high flow rates may be addressed (reduced) by, for example, either (1) choosing a longer duration of infusion (60 or 120 minutes, for example) or (2) smaller volume of pharmaceutical preparation (e.g. 250 or 500 ml).

The above described realisation administers a therapeutic dose of a pharmaceutical preparation over an appropriate time frame and uses a large volume of solution (1000 ml) so as to reduce inaccuracy in the early stages of the infusion process.

Further, as mentioned above, the infusion rates are relative low during the first half of the infusion process; this allows (during the infusion process) administering to the patient a wide range of test doses that may detect a negative reaction in the patient (who was not known to be allergic to the pharmaceutical drug) resulting in the identification that the patient is allergic to the drug that is being infused into the patient. The present infusion processes are also particularly useful (1) in the circumstances where it is suspected that the patient may be allergic to the drug (a drug challenge), or (2) to induce desensitization in a patient who is allergic to the drug but that which may or may not be suspected prior (a drug desensitization).

Referring now to FIGS. 26, these figures refer to a realisation of the second embodiment of the disclosure.

The equipment used for conducting this particular realisation is a Chemyx 200 syringe driver acting as the infusion driver 14 having a 60 ml syringe 15 containing 53 ml of the pharmaceutical preparation (e.g. Simacid Blue dye in this case used as a spectroscopic marker). The pharmaceutical preparation is to be administered to the patient via conduits 30a (minimum volume extension tubing having a volume of 0.3 ml) extending from the syringe 15 to the multiway-tap attached to the dilution chamber 32 attached to conduits 30b (minimum volume extension tubing of 2.0 ml volume) attached to the patient's intravenous access.

An example of the software instructions in python 3 language for use with the computer system 12 to calculate variables for operating the infusion driver 14 is presented in FIG. 28. FIG. 28 is the software code, written in python 3, to calculate the values that can be sent to the infusion device to realise the Sadleir method (second embodiment of the disclosure). These can be manually entered into the infusion device for either constant infusion steps or ramp infusion steps, or they can be sent to microprocessor through various means. This software will generate the infusion step rates and volumes which can be manually entered via the keypad 26, or stored in the external memory drive 20 with additional software instructions depending on the characteristics of the computer system 12.

The dilution chamber 32 in this particular realization is configured with a catheter 50 having three evenly spaced perforations of 0.25 mm diameter (items 58a to 58c in FIG. 9B) around the upper aspect of the sleeve 68 that expands when in use forming an elliptical balloon at the end of the catheter. The perforation are oriented at 60 degrees above the horizontal towards the inlet 53 and outlet 38 of the manifold 36. The arrangement comprises a dilution chamber 32 orientated in a vertical manner as shown in FIGS. 2 and 3.

For this particular realization, the initial variables to be entered in the infusion driver 14 by the operator are:

a) infusion duration (i)=30 minutes

b) infusion volume (Vₚ)=50 ml

c) dilution chamber (V_{d})=10 ml

d) number of intervals per minute (t)=1200 giving a total of 36,000 intervals during the infusion process having a duration of 30 minutes

e) concentration of drug in syringe 15 (C_{d})=2% of total therapeutic dose/ml

The particular infusion driver 14 used for this realisation is capable of linearly-changing rate throughout each infusion step (Ramp-step program) or a constant rate throughout each infusion step (Constant-step program). For this demonstration, a Ramp-step program was used, although equivalent dosing can be achieved with a Constant-step program (see FIG. 25C). The infusion driver 14 provides 250 ms pause (latent period) between infusion steps.

For the approximation process of the Sadleir function, 40 steps for a 30 minutes infusion were chosen (each 0.745833 min long due to the 0.250 sec pause between each step).

Further, the Tansy function for the 50 ml pharmaceutical preparation over a 30 minute infusion duration was calculated to determine the dose that deeds to be provided at each point of the infusion process. The 30 minutes infusion period was divided into 36,000 intervals of 0.0008333 minutes ( 1/1200 of a minute). Then, the volume dictated by the Tansy function was calculated for each of the intervals. This volume was used to calculate the dose of drug given in each interval (interval volume multiplied by concentration of drug in the dilution chamber (Cd)).

Subsequently, the dose for each interval of the infusion is modified by a correction factor to calculate the modified tansy function (D_{mtf}) for each interval of the infusion is calculated. In particular, the dose given in each interval for the Tansy function infusion protocol is then reduced by multiplying each dose by a ‘constant fraction’. This constant fraction is (1) the total dose of active ingredient minus the amount of active ingredient remaining in the dilution chamber at the end of the infusion process using the Sadleir method, divided by (2) the total dose of active ingredient. This can be simplified to

$$\frac{{V}_{p} - {V}_{d} ( 1 - {e}^{- \frac{Vp}{Vd}} )}{{V}_{p}} \\ {V}_{p} = volume of \text{drug-containing} infusion container \\ {V}_{d} = volume of dilution chamber$$

For a 50 ml infusion dose with a 10 ml dilution chamber, the fraction of the total dose remaining in the dilution chamber at the completion of 50 mL of infusion is 0.1987. The ‘constant fraction’ is 0.80135 (equation 3). Reducing the dose given in each interval ensures that the infusion per the Sadleir method runs for the same duration as would be dictated by the Tansy method.

The flow rate as dictated by the Sadleir function is calculated over each of the 36,000 intervals (occurring during the 30 minutes infusion process) to determine the required flow rate to ensure the patient receives the same dose as calculated for the D_{mtf}.

At this stage, the infusion driver 14 is programed so as to approximate the flow rate to be delivered by the infusion driver 14 to the flow rate as dictated by the Sadleir function calculated above.

To approximate the Sadleir function with the infusion driver 14 capable of providing a finite number of infusion steps, the volume of pharmaceutical preparation to be delivered over each programmed infusion step is calculated. In particular, as shown in FIG. 13D with respect to Equation (10), the volume of pharmaceutical preparation for each infusion step is equal to the sum of the volume of pharmaceutical preparation delivered during 900 corresponding intervals of the Sadleir function. The number 900 is obtained by dividing (a) the total number of intervals (36,000) used during calculation of the value of the flow rate dictated by the Sadleir Function by (b) the number of infusions steps (40), i.e.: 36000/40=900. Thus, the number of intervals (used during calculation of the value of the flow rate dictated by the Sadleir Function) per infusion step is 900.

The flow-rate for each programmed infusion step of the infusion driver 14 (the Chemyx 200 infusion pump) is then calculated.

For a Constant-step program, the flow rate (ml/min) for each infusion step (occurring during a particular time period) is such that the volume of pharmaceutical preparation delivered during each infusion step is equal to the total volume of pharmaceutical preparation delivered during the corresponding 900 intervals (occurring during the particular time period of each step) as calculated using the Sadleir function.

For a Ramp-step program, the infusion driver 14 is capable of delivering a flow rate over the infusion step (occurring during a particular time period) that begins at a first flow rate and linearly decreases or increases to a second rate. The next infusion step will then begin at the second rate and linearly increase or decrease to reach the final rate of that infusion step. This process continues for each step of the infusion process.

The Ramp-step program is initially calculated such that the first flow rate of each infusion step (occurring during a particular time period) is equal to the starting flow rate of the 900 intervals (occurring during the particular time period) used for calculating the flow rates as dictated by the Sadleir function; and the second flow rate of each infusion step is equal to the starting rate of the next 900 intervals (occurring at the subsequent infusion step time period). The variation of flow rate occurring during the particular time period of each infusion step will, from the first flow rate, either linearly decrease or increase to reach the second flow rate. This approximation is only approximate because the Sadleir function is not a linear function; thus, the volume of pharmaceutical preparation to be delivered during the particular time period as dictated by the Sadleir function will not be equal to the volume to be delivered by the infusion driver 14 during the particular time period.

In particular, the volume to be delivered during the particular time period by the infusion driver 14 is greater than the volume dictated by the Sadleir function during the particular time period. The difference between both volumes will be greatest for the first infusion step.

A process of correcting the above mentioned inaccuracy is:

a) Calculate the volume (V₂) administered by the ramp step program from infusion step 2 until the final infusion step (step 40).

b) Calculate the (V₁) volume associated with the Sadleir infusion for the corresponding intervals of the infusion (intervals 901 to 36000).

c) Multiply the rate at the end of each ramp step by V₁/V₂ (and also therefore the starting rate of the subsequent intervals 2 to 40).

d) Calculate the volume associated with the Sadleir function for the intervals corresponding to the 1^{st} ramp step (intervals 1-900). Set the starting rate of the 1^{st} ramp step to deliver the same volume over the step as for the Sadleir function (i.e. adjust from 2.255 ml/min to 0.158 ml/min).

e) correct for the latent period (where the delivery of pharmaceutical preparation is paused) between neighbouring infusion steps; in particular, as the infusion driver 14 of the present realisation has a latent period of 0.250 sec between infusion steps (each infusion step of duration 45 seconds), to correct for this particular latent period the flow rate at all times is multiplied by 45/(45−0.250) to ensure a similar volume of pharmaceutical preparation is delivered the infusions process as dictated by the Sadleir function over each infusion step.

In operation, the multiway tap is open to the atmosphere for priming of the conduit 30a and the first priming step is started (e.g.: 0.5 ml over 30 seconds).

Then the multiway-tap is moved to direct the pharmaceutical preparation into the dilution chamber 32 to start second priming step that delivers mixed, diluted pharmaceutical preparation from the dilution chamber 32 up to the patient and then stops. In the present realization, this requires infusion of 1.96 ml of the pharmaceutical preparation, dependent on the volume of the conduits 30b between the dilution chamber 32 and the patient. To enhance mixing within the dilution chamber 32 of the diluent originally contained within the dilution chamber 32 and the delivered pharmaceutical preparation the flow rate is varied alternatively between rapid flow rate (e.g.:1 ml/min) and a slow flow rate (e.g.: 0.1 ml/min). As mentioned before, these variations in flow rate with not affect the amount of pharmaceutical preparation provided to the patient due the mixing occurring prior the infusion process.

At this stage, the ramp-step program is started for commencing of the infusion process to deliver the pharmaceutical preparation to the patient. At the end of the infusion process the infusion driver 14 is stopped and the pharmaceutical composition remaining within the dilution chamber 32 is delivered to the patient by collapsing of the dilution chamber.

Demonstration of the efficacy of mixing of drug in the dilution chamber using the Sadleir method with two arrangements (with an without a bubble trap) of the dilution chamber 32 with manifold 36 and catheter 50 (illustrated in FIGS. 6, 7 and 8) is presented in FIGS. 26C and 26D for a 50 mL infusion over 30 minutes, using a ramp-step method with 40 infusion steps of 45 seconds each to approximate the Sadleir function. The flexible sleeve 68 of the catheter 50 (see FIG. 8C) in these examples was perforated with three evenly spaced, 30 g (0.25 mm) perforations angle at 60 degrees above the horizontal.

Demonstration of the desired dosing profile over the infusion period for the realization, ensuring a separation in time of orders of magnitude of cumulative dose and dosing rate, is illustrated in FIGS. 26B and 26C, and FIG. 26D.

As mentioned earlier, the Sadleir and Tansy the infusion rates are relatively low during most of the start of the infusion process. This allows, concurrently with the actual process of infusing the pharmaceutical preparation, administering a wide range of test doses that may recognize a negative reaction in the patient (who was not known to be allergic to the pharmaceutical drug). This may result in the identification that the patient is allergic to the drug that is being infused into the patient and allows the infusion to be stopped before the patient is administered a dose that would result in a more serious or lethal reaction. The present infusion processes are also particularly useful (1) in the circumstances where it is suspected that the patient may be allergic to the drug (a drug challenge), or (2) to induce desensitization in a patient who is allergic to the drug but that which may or may not be suspected prior (a drug desensitization).

Part 5—Example Medication Delivery System with Two Plungers Medication Delivery System

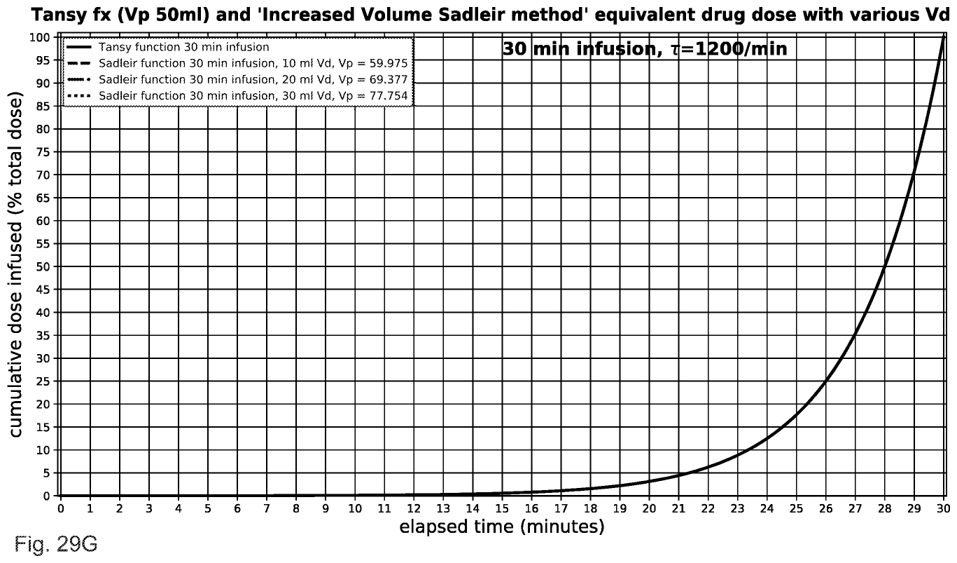

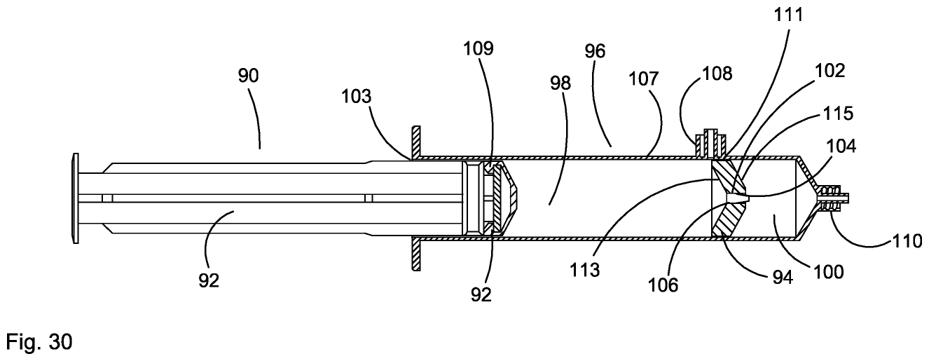

Referring now to FIGS. 30 to 47, FIGS. 30 to 47 show particular arrangements of a medication delivery system 91 comprising a medication delivery apparatus 90 in accordance with particular embodiments of the disclosure.

As shown in FIG. 30, in some embodiments, the medication delivery system 91 comprises the medication delivery apparatus 90 and an infusion device 93. The infusion device 93 is illustrated in the form of a syringe driver 17. The infusion device 93 may be similar to, or the same as the previously described infusion device 14. In some embodiments, the medication delivery apparatus 90 comprises a first plunger 92 (which may also be referred to as a primary plunger) and a second plunger 94 (which may also be referred to as a separating plunger). The medication delivery apparatus 90 also comprises a container 96 for receiving the second plunger 94 and at least a portion of the first plunger 92. This may be a distal portion of the first plunger 92.

The presence of the separating plunger 94 within the container 96 defines two chambers within the container 96, in particular: a first chamber 98 (the active agent chamber) and a second chamber 100 (the mixing chamber). In particular, the container 96 and the second plunger 94 together define a dilution chamber 100 that is configured to receive a diluent. The dilution chamber 100 may be similar to, or the same as the dilution chamber 32 previously described. The first plunger 92, the container 96 and the second plunger 94 together define an active agent chamber 98. The active agent chamber 98 is configured to receive a pharmaceutical preparation.

Further, as will be described with the method of operation of the medication delivery apparatus 90, the separating plunger 94 is adapted to permit flow of the fluid (e.g. the active agent) contained in the active agent chamber 98 into the dilution chamber 100. The dilution chamber 100 may also be referred to as a mixing chamber. The mixing chamber 100 comprises the diluent for mixing with the pharmaceutical preparation (or active agent) flowing from the active agent chamber 98 into the mixing chamber 100, for preparation of the pharmaceutical composition (diluted pharmaceutical preparation) to be delivered to the patient.

In accordance with the present embodiments of the disclosure, the second plunger 94 comprises valve means 102 (which may also be referred to as a valve 102) adapted to control flow the active agent entering the mixing chamber 100. In other words, the second plunger 94 comprises a valve 102 configured to control a flow of pharmaceutical preparation from the active agent chamber 98 to the dilution chamber 100. The valve 102 may be configured to control the flow of the pharmaceutical preparation in response to applied pressure. The pressure may be applied by the first plunger 92. Alternatively, the pressure may be applied via the first plunger 92. In the particular arrangement shown in FIGS. 30 to 34A, the valve means 102 comprises a duckbill valve 104. The duckbill valve 104 comprises a plurality of flaps 106 that, as pressure is applied to the first plunger 92, separate with respect to each other opening the duckbill valve 104. Upon removal of the pressure, that is being applied to the first plunger 92, the flaps 106 return to their original condition closing the duckbill valve 104 and impeding backflow of the pharmaceutical preparation back into the active agent chamber 98.

The valve 102 (or valve means 102) comprises an inlet side 113 and an outlet side 115. The valve 102 (or valve means 102) is configured to move from a closed position to an open position upon application of pressure to the inlet side 113. Pressure may be applied to the inlet side 113 of the valve 102 (or valve means 102) by longitudinally displacing (or actuating) the first plunger within the chamber 96 to displace the pharmaceutical preparation. The valve 102 (or valve means 102) is configured to move from the open position to the closed position upon removal of the pressure applied to the inlet side. The valve 102 (or valve means 102) may be configured to move from the closed position to the open position when a pressure applied to the inlet side 113 exceeds a pressure threshold. The valve 102 (or valve means) may be configured to move from the open position to the closed position when the pressure applied to the inlet side 113 is below a pressure threshold. The valve 102 (or valve means 102) is biased towards the closed position. The valve 102 (or valve means 102) comprises the plurality of flaps 106. The plurality of flaps 106 are configured to separate upon application of pressure to the inlet side 113. The first plunger 92 is configured to contact the second plunger 94 once all, or most of, the pharmaceutical preparation in the active agent chamber 98 has been transferred to the dilution chamber 100. Further actuation of the first plunger 92 will also result in movement of the second plunger 94. Thus, actuation of the first plunger 92 causes movement of the second plunger 94, and causes the pharmaceutical preparation in the dilution chamber 100 to be output by the medication delivery apparatus 90.

Further, the container 96 comprises at least one first port 108 (the inlet port) and a second port 110 (the outlet port). The inlet port 108 allows filling of the container 96 with active agent and the second port 110 allows either (1) filing the mixing chamber with diluent or (2) permitting exit of the mixture of the active agent and diluent (the pharmaceutical composition) from the container 96 (in particular, from the mixing chamber 100) for delivery to the patient. The container 96 comprises a first active agent chamber opening 103 that is configured to receive at least a portion of the first plunger 92. In particular, the active agent chamber 98 comprises the active agent chamber opening 103. The inlet port 108 may be considered a second active agent chamber opening that is configured to receive the pharmaceutical preparation. In other words, the active agent chamber 98 may be said to comprise a second active agent chamber opening that is configured to receive the pharmaceutical preparation. The second active agent chamber opening (the inlet port 108) is defined in the wall of the container 96. The active agent chamber 98 may be filled with pharmaceutical preparation by introducing the pharmaceutical preparation into the active agent chamber 98 via the second active agent chamber opening (i.e. the first port 108). The first port 108 may therefore be referred to as an active agent chamber inlet. The dilution chamber 100 comprises a dilution chamber opening 110 that is defined by the container 96. The dilution chamber opening 110 may be referred to as the outlet port of the container 96.

In the arrangement shown in figures, the inlet and outlet ports 108 and 110 (as well as inlet and outlet ports 118 and 120) are shown as male luer-lock connector; however, in alterative arrangements, for example, the inlet ports, such as 108 and 118, may comprise female luer-lock connectors.

The first plunger 92 and the second plunger 94 are each configured to be displaced with respect to a longitudinal axis of the container 96. The second plunger 94 is disposed between the first plunger 92 and the dilution chamber opening 110 (i.e. the outlet port 110). The second plunger 94 is disposed between the inlet port 108 (the second active agent chamber opening) and the dilution chamber opening 110.

The container 96 defines a inner container surface 107. The first plunger 92 comprises a first plunger sealing surface 109. The first plunger 92 is configured to seal with the inner container surface 107. In particular, the first plunger sealing surface 109 is configured to seal with the inner container surface 107 to inhibit fluid flow between the inner container surface 107 and the first plunger sealing surface 109.

The second plunger 94 comprises a second plunger sealing surface 111. The second plunger 94 is configured to seal with the inner container surface 107. In particular, the second plunger sealing surface 111 is configured to seal with the inner container surface 107 to inhibit fluid flow between the inner container surface 107 and the second plunger sealing surface 111.

The medication delivery apparatus 91 comprises a conduit 30a. The conduit 30a is configured to be fluidly connected to the dilution chamber opening 110. The conduit 30a is of a predetermined volume. That is, a length and an internal surface area of the conduit 30a are sized so that the conduit 30a defines a predetermined volume. The conduit 30a can therefore hold or store a volume of the diluted pharmaceutical preparation prior to the diluted pharmaceutical preparation being delivered to the patient. The conduit 30a may be referred to as a minimum volume extension tube. The conduit 30a is configured to retain a first volume of infusion to be delivered to the patient. The first volume of infusion can be prepared by the priming process at a rate that will result in effective mixing in the dilution chamber 110. This is possible because during this time, no pharmaceutical preparation is delivered to the patient. Thus, a different flow rate can be used for the first volume when priming, while driving the mixed fluid exiting the dilution chamber 100 to the end of the conduit 30a. Although the conduit 30a of the medication delivery apparatus 91 is described to be of a predetermined volume, it will be understood that a conduit of a predetermined volume could be used with any of the medication delivery apparatuses disclosed herein to achieve similar functionality and benefits.

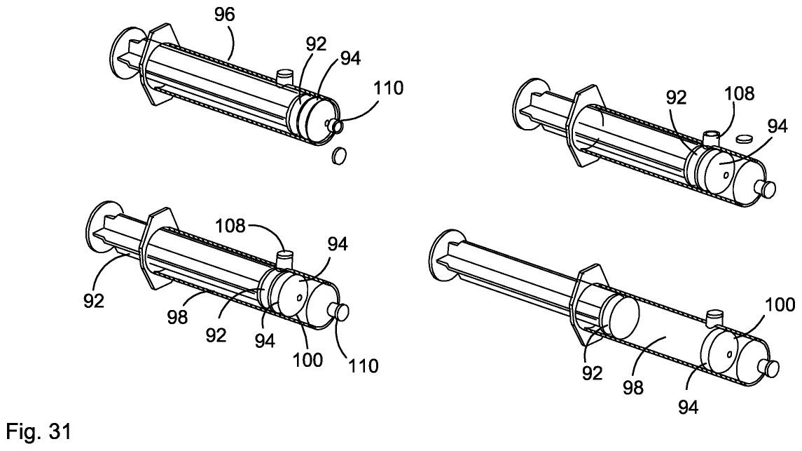

FIG. 31 shows the process for filing the container 96 of the medication delivery apparatus 90 with active agent and diluent.

As shown in FIG. 31, the process for filing the container 96 comprises the step of delivering the diluent into the mixing chamber 100 by opening the outlet 110 and delivering the diluent into the mixing chamber 100. Due to the entrance of the diluent into the mixing chamber 100, the separating plunger 94 is displaced away from the outlet 110 permitting entrance of the diluent and carrying with it the primary plunger 92.

Once the mixing chamber 100 is filled with the corresponding quantity of diluent, the outlet 110 is closed permitting filling of the active agent chamber 98.

Filling the active agent chamber 98 comprises the step of opening the inlet port 108 for delivery of the pharmaceutical preparation into the active agent chamber 98. Filling of the active agent chamber 98 displaces the primary plunger 92 further from the outlet 110 until all of the corresponding quantity of pharmaceutical preparation is delivered into the active agent chamber 98.

At this stage, the inlet 108 is closed and the medication delivery apparatus 90 may be prepared for delivery of the pharmaceutical composition to the patient.

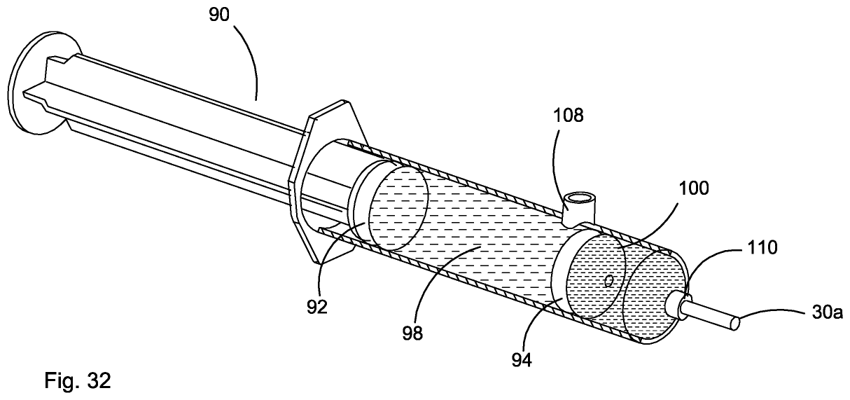

Preparation of the medication delivery apparatus 90 comprises the step of attaching the conduit 30a to the outlet 110 as is shown in FIG. 32. The conduit 30a comprises a minimal volume tubing adapted to be attached to the outlet 110 and to an infusion device for delivering the pharmaceutical composition into the patient's blood stream.

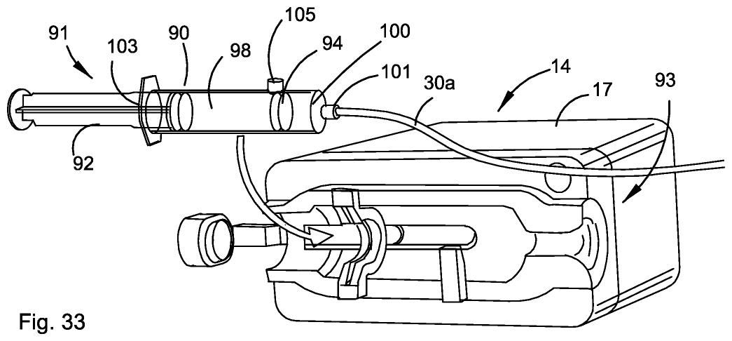

Subsequently, as shown in FIG. 33, the medication delivery apparatus 90 is mounted on the infusion device 14, thereby forming a medication delivery system 91. The infusion device of FIG. 33 is in the form of a syringe driver 17. The medication delivery apparatus 90 is mounted to the syringe driver 17 in order to: (1) prepare the pharmaceutical composition by mixing the pharmaceutical preparation and the diluent, and (2) deliver the pharmaceutical composition (i.e. the diluted pharmaceutical preparation, or the pharmaceutical preparation—when the diluent is consumed) into the conduit 30a for infusion into the patient.

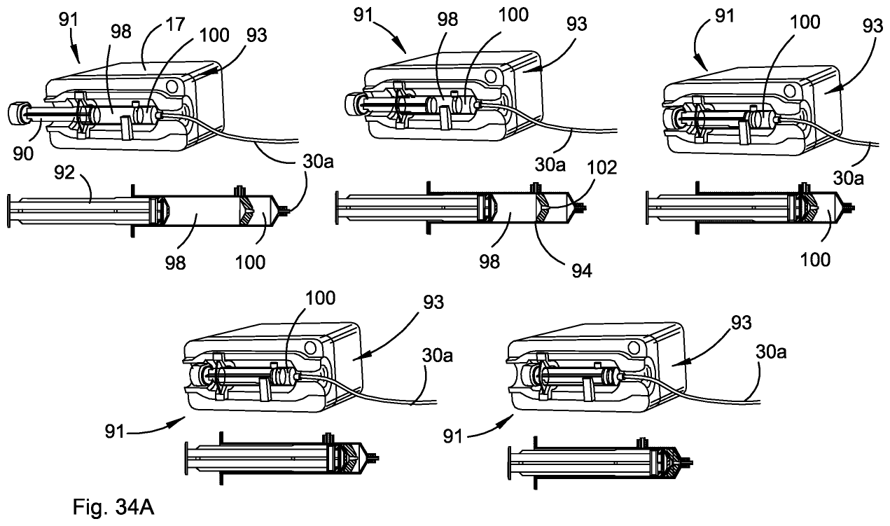

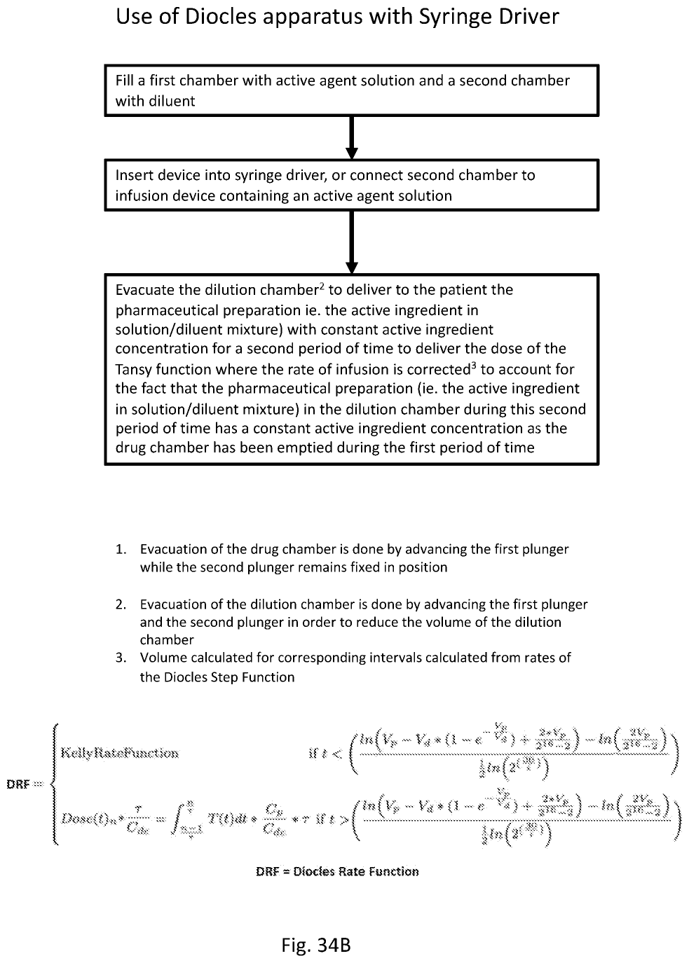

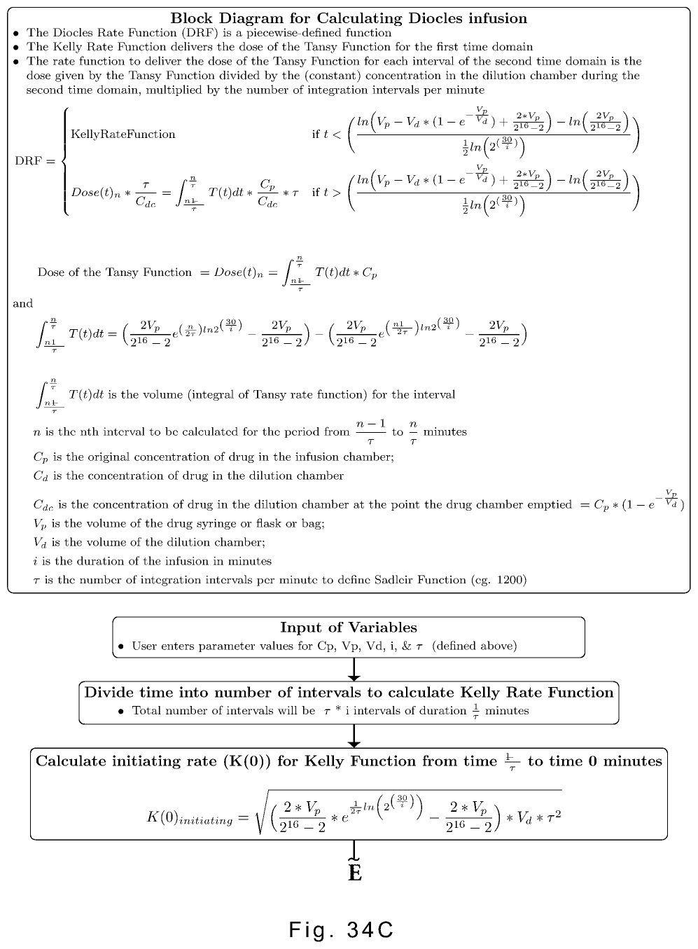

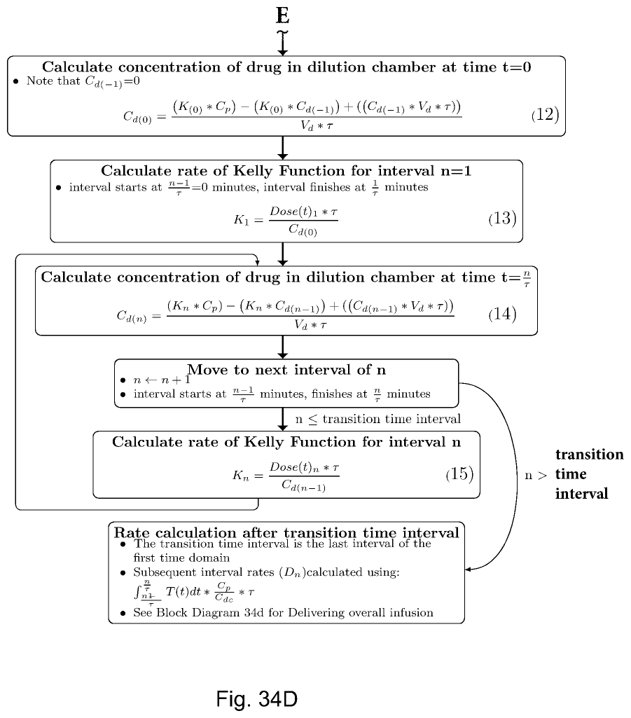

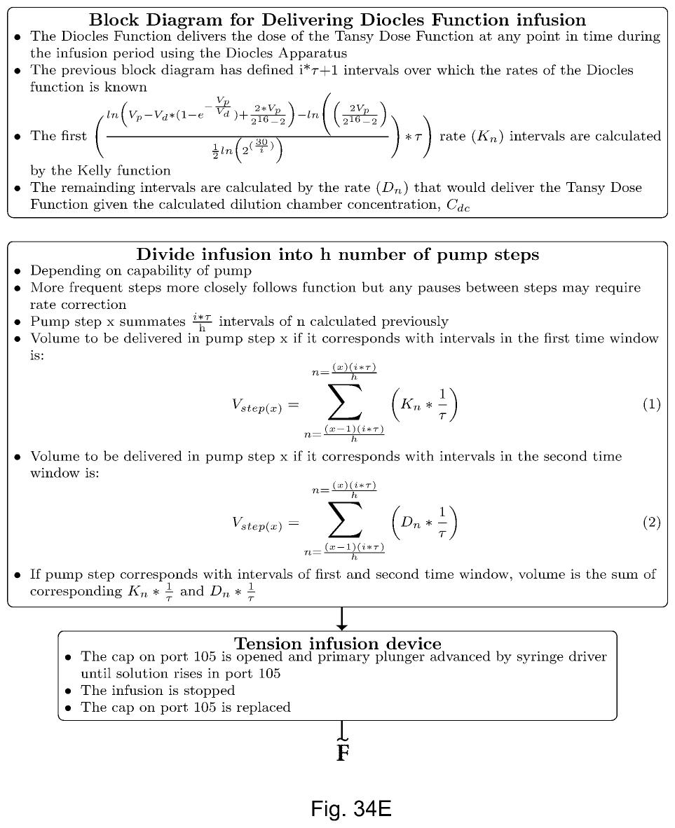

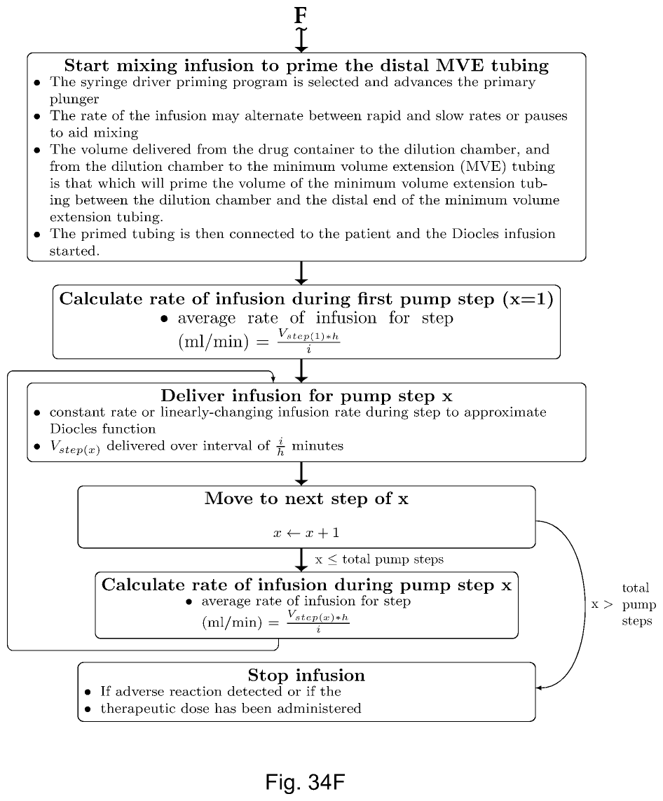

As shown in FIG. 34A, preparation of the pharmaceutical composition comprises the step of pushing the primary plunger 92 in order that the pharmaceutical preparation contained in the active agent chamber 98 is delivered into the dilution chamber 100 for mixing with the diluent contained in the diluent chamber 100. The primary plunger 92 is pushed by the syringe driver 17 in such a manner that the pharmaceutical preparation is delivered into the mixing chamber 100 in order to provide, in conjunction with the valve means 102, a particular mixing profile within the mixing chamber 100 to allow proper mixing of the pharmaceutical preparation with the diluent.

As the pharmaceutical preparation contained in the active agent chamber 98 is delivered into the dilution chamber 100, mixing occurs for generating the pharmaceutical composition (in this case, the diluted pharmaceutical preparation), which is then delivered into the conduit 30a for infusion into the patient. As the pharmaceutical composition is delivered into the conduit 30a, the concentration of active agent within the dilution chamber 100 will increase as the active agent is delivered into the dilution chamber 100 during the infusion. For delivery of the pharmaceutical composition to the patient, the primary plunger 92 (with the separating plunger 94 abutting the primary plunger 92) is pushed in such a manner that the pharmaceutical composition is delivered in accordance with a particular profile. In particular, the primary plunger 92 is driven based on particular algorithms.

Initially, before the primary plunger 92 is driven based on the particular algorithms and the conduit 30a is fluidly connected to the patient, the syringe driver 17 is operated to drive the primary plunger 90 in such a manner to fill (i.e. to prime) the conduit 30a to be fluidly connected to the patient for delivery of the pharmaceutical composition.

One advantage of priming the conduit 30a (as described in the previous paragraph) is that the conduit 30a will be filled with pharmaceutical composition (i.e. diluted active agent) prior delivering the pharmaceutical composition to the patient; thus, ensuring that the patient will immediately receive the pharmaceutical composition comprising diluted active agent.

Another advantage of priming the conduit 30a is that during priming of the conduit 30a (prior delivering any pharmaceutical composition to the patient) the active agent may be driven at an arbitrarily fast rate into the dilution chamber 100 to allow good mixing before any of the pharmaceutical composition is delivered to the patient; this ensures proper mixing of the pharmaceutical preparation and the diluent within the dilution chamber 100 prior delivering any pharmaceutical composition to the patient.

The syringe driver 17 is adapted to drive the primary plunger 92 in a particular manner. For example, the syringe driver 17 may comprise processing means for running of algorithms for driving the primary plunger 92 in a particular manner to obtain a particular mixing profile as well as a delivery profile of the pharmaceutical composition.

Part 6—Example with the Sadleir Method

The medication delivery system 91 previously described may be controlled to deliver a pharmaceutical preparation to a patient according to the Sadleir method. As previously described, the medication delivery system 91 comprises the medication delivery apparatus 90 and the infusion device 93. The infusion device 93 comprises the at least one infusion device processor and infusion device memory as previously described. The infusion device memory stores program instructions accessible by the at least one infusion device processor. The program instructions are configured to cause the at least one infusion device processor to actuate an infusion device actuator (e.g. syringe driver 17) to control the medication delivery apparatus 90 to deliver medication in accordance with the Sadleir method.

In particular, the program instructions are configured to cause the at least one infusion device processor to receive a concentration input (Cₚ) that is indicative of a concentration of the pharmaceutical preparation in the active agent chamber. The concentration may be a concentration of active agent in the pharmaceutical preparation. The concentration input (Cₚ) may be received via an input provided by a user. For example, the concentration input (Cₚ) may be input using the user interface 22. Alternatively, the concentration input (Cₚ) may be retrieved from the infusion device memory. Throughout this description, the concentration input (Cₚ) may be a concentration of a drug in, or delivered from the active agent chamber.

The program instructions are further configured to cause the at least one infusion device processor to receive a volume input (Vₚ) that is indicative of a volume of the pharmaceutical preparation. This may be a volume of the pharmaceutical preparation in the active agent chamber. The volume input (Vₚ) may be received via an input provided by a user. For example, the volume input (Vₚ) may be input using the user interface 22. Alternatively, the volume input (Vₚ) may be retrieved from the infusion device memory.

The program instructions are further configured to cause the at least one infusion device processor to receive a dilution chamber volume input (V_{d}) that is indicative of a volume of the dilution chamber 100. The dilution chamber volume input (V_{d}) may be received via an input provided by a user. For example, the dilution chamber volume input (V_{d}) may be input using the user interface 22. Alternatively, the dilution chamber volume input (V_{d}) may be retrieved from the infusion device memory. Throughout this disclosure, the dilution chamber volume input (V_{d}) may correspond to volume of the relevant dilution chamber.

The program instructions are further configured to cause the at least one infusion device processor to receive a time input (i) that is indicative of a time window over which the pharmaceutical preparation is to be administered. The time input (i) may be received via an input provided by a user. For example, the time input (i) may be input using the user interface 22. Alternatively, the time input (i) may be retrieved from the infusion device memory.

The program instructions are further configured to cause the at least one infusion device processor to receive an infusion number input (T) that is indicative of a number of infusion intervals per minute over which an infusion modelling function is to be numerically approximated over the time window. The infusion number input (T) may be received via an input provided by a user. For example, the infusion number input (T) may be input using the user interface 22. Alternatively, the infusion number input (T) may be retrieved from the infusion device memory. Throughout this disclosure, the infusion number input (T) may correspond to the number of infusion intervals per minute over which the relevant function (e.g. the Sadleir function) is calculated.

Throughout this disclosure, it will be understood that an infusion interval is an interval over which the infusion is approximated via a numerical approximation. This may differ from infusion steps. Infusion steps are the actual infusion steps delivered by the relevant infusion device. The number of infusion intervals may exceed the number of pump steps for a given period of time. For example, a 30 s pump step may be numerically approximated by 600 infusion intervals. These infusion intervals are used to improve the accuracy of numerical approximations when using infusion modelling functions. The volumes, concentrations and/or flow rates determined with respect to infusion intervals during the numerical approximations are targeted when executing the lower resolution infusion steps that are actually executed by the infusion devices disclosed herein.

The program instructions are further configured to cause the at least one infusion device processor to receive a number of infusion steps (h) that are to be executed during the time window. Receiving the number of infusion steps (h) that are to be executed during the time over which the pharmaceutical preparation is to be administered may comprise receiving an infusion step input that is indicative of the number of infusion steps. Alternatively, receiving the number of infusion steps that are to be executed during the time over which the pharmaceutical preparation is to be administered may comprise retrieving the number of infusion steps from the infusion device memory. Receiving the number of infusion steps that are to be executed during the time over which the pharmaceutical preparation is to be administered may comprise multiplying the time input (i) and the infusion number input (τ). There are h infusion steps of

$$\frac{i}{h}$$

duration during the allusion process.

The program instructions are further configured to cause the at least one infusion device processor to receive a pharmaceutical preparation input. The pharmaceutical preparation input is indicative of one or more of an identity of the pharmaceutical preparation, a dose of the pharmaceutical preparation and a maximum pharmaceutical preparation administration rate.

The program instructions are further configured to cause the at least one infusion device processor to numerically approximate the infusion modelling function over the time window. The at least one infusion device processor may approximate the infusion modelling function over the time window as described in FIGS. 13A to 13C.

The program instructions are further configured to cause the at least one infusion device processor to determine the infusion rate of an infusion step. This is determined by summating a plurality of infusion interval volumes calculated by numerical approximation over which the infusion step will occur, and then determining the infusion rate that will deliver this volume across the duration of the infusion step.

The program instructions are configured to cause the at least one infusion device processor to take the user inputs and create a theoretical program of infusion rate versus time or infusion cumulative volume versus time where time is the duration over which the pharmaceutical preparation is to be administered. Alternatively, the program instructions may be configured to cause the at least one infusion device processor to look up a theoretical program stored in the device memory. The theoretical program may be the numerical approximation described herein.

Numerically approximating the infusion modelling function comprises determining a number of infusion intervals within the time window. That is, the at least one infusion device processor determines a number of infusion intervals within the time window.

Numerically approximating the infusion modelling function comprises determining an initiating target flow rate parameter (S(0)_{initiating}) The initiating target flow rate parameter (S(0)_{initiating}) is indicative of a target flow rate of the pharmaceutical preparation to be output by the medication delivery apparatus 90 during an initiating infusion interval of the numerical approximation.

Determining the initiating target flow rate (S(0)_{initiating}) comprises calculating:

$$\sqrt{( ( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) ) * {τ}^{2} * {V}_{d}}$$

The program instructions are further configured to cause the at least one infusion device processor to determine an initiating pharmaceutical preparation concentration. The initiating pharmaceutical preparation concentration is indicative of an approximated concentration of the pharmaceutical preparation in the dilution chamber after the initiating infusion interval of the numerical approximation. The at least one infusion device processor determines the initiating pharmaceutical preparation concentration by calculating:

$${C}_{{d}_{( o )}} = \frac{( {S ( 0 )}_{initiating} \times {C}_{p} ) - ( {S ( 0 )}_{initiating} \times {C}_{{d}_{( - 1 )}} ) + ( {C}_{{d}_{( - 1 )}} \times {V}_{d} \times τ )}{{V}_{a} \times τ}$$

where C_{d}_{_{(−1)}}=0 and C_{d}_{₍ₒ₎} is the initiating pharmaceutical preparation concentration.

The program instructions are further configured to cause the at least one infusion device processor to determine a subsequent target flow rate and a subsequent pharmaceutical preparation concentration for each of a plurality of subsequent infusion intervals of the numerical approximation. The subsequent target flow rates are each indicative of a target flow rate of the pharmaceutical preparation to be output by the medication delivery apparatus 90 during a respective subsequent infusion interval of the numerical approximation. The subsequent pharmaceutical preparation concentrations are each indicative of a subsequent approximated concentration of the pharmaceutical preparation in the dilution chamber after the respective subsequent infusion interval.

Each of the subsequent target flow rates is determined based at least in part on the subsequent pharmaceutical preparation concentration of a previous infusion interval of the respective infusion interval. That is, each of the subsequent target flow rates is determined based at least in part on the subsequent pharmaceutical preparation concentration of the infusion interval that occurred immediately before the infusion interval of that subsequent target flow rate. Each of the subsequent pharmaceutical preparation concentrations is determined based at least in part on the subsequent target flow rate of the respective subsequent infusion interval.

Determining a subsequent target flow rate for one of the plurality of subsequent infusion intervals of the numerical approximation comprises determining a flow rate parameter Sₙ where n is the number of the relevant infusion interval. Determining the flow rate parameter Sₙ comprises determining a dose parameter D_{mtf}(t)ₙ. Determining the dose parameter D_{mtf}(t)ₙ comprises calculating:

$${{D}_{mtf} ( t )}_{n} = {\int }_{\frac{n - 1}{τ}}^{\frac{n}{τ}} T ( t ) dt \times {C}_{p} \times ( \frac{{V}_{p} - ( {V}_{d} \times ( 1 - {e}^{- \frac{{V}_{p}}{{V}_{d}}} ) )}{{V}_{p}} )$$

where:
T(t) is a Tansy rate function;
Cₚ is the concentration input;
Vₚ is the volume input;
V_{d} is the dilution chamber volume input;
n is the number of the relevant infusion interval; and
τ is the infusion number input.

Determining the flow rate parameter Sₙ comprises calculating:

$${S}_{n} = \frac{{{D}_{mtf} ( t )}_{n} \times τ}{{C}_{d ( n - 1 )}}$$

where n is the number of the relevant infusion interval, C_{d(n-1)} is a subsequent pharmaceutical preparation concentration of a previous infusion interval of the nth infusion interval and D_{mtf}(t)ₙ is the dose parameter.

In some embodiments, determining the subsequent pharmaceutical preparation concentrations of the numerical approximation comprises calculating:

$${C}_{d ( n )} = \frac{( {S}_{n} \times {C}_{p} ) - ( {S}_{n} \times {C}_{d ( n - 1 )} ) + ( {C}_{d ( n - 1 )} \times {V}_{d} \times τ )}{{V}_{d} \times τ}$$

where C_{d(n)} is the subsequent pharmaceutical preparation concentration for the nth infusion interval of the numerical approximation and C_{d(n-1)} is the subsequent pharmaceutical preparation concentration for the n−1 th infusion interval of the numerical approximation. In other words, n is the number of the relevant infusion interval and C_{d(n-1)} is a subsequent pharmaceutical preparation concentration of a previous infusion interval of the nth infusion interval.

This calculation may be performed for each subsequent pharmaceutical preparation concentration of the iteration.

In some embodiments, determining the initiating target flow rate (S(0)_{initiating}) comprises calculating:

$$\sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) \times {V}_{d} \times {τ}^{2}}$$

In some embodiments, determining the dose parameter comprises determining a dose of the Tansy function, by calculating

$${\int }_{\frac{n - 1}{τ}}^{\frac{n}{τ}} T ( t ) dt \times {C}_{p} .$$

Refer, for example, to FIG. 29A.

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} )$$

The subsequent target flow rate is indicative of a target flow rate of the pharmaceutical preparation to be output by the medication delivery apparatus 90 during a subsequent infusion step. The subsequent target flow rate is determined based at least in part on the subsequent pharmaceutical preparation concentration. The subsequent target flow rate is limited at the maximum pharmaceutical preparation administration rate. Therefore, the subsequent target flow rate does not exceed the maximum pharmaceutical preparation administration rate during infusion. Determining the subsequent target flow rate comprises determining a flow rate parameter Sₙ where n is the number of the relevant infusion step. Determining the flow rate parameter Sₙ comprises determining a dose parameter D_{mtf}(t)ₙ. Determining the dose parameter D_{mtf}(t)ₙ comprises calculating:

$${{D}_{mtf} ( t )}_{n} = {\int }_{\frac{n - 1}{τ}}^{\frac{n}{τ}} T ( t ) dt \times {C}_{p} \times ( \frac{{V}_{p} - ( {V}_{d} \times ( 1 - {e}^{- \frac{{V}_{p}}{{V}_{d}}} ) )}{{V}_{p}} )$$

where:
T(t) is a Tansy function;
Cₚ is the concentration input;
Vₚ is the volume input;
V_{d} is the dilution chamber volume input;
n is the number of the relevant infusion step; and
τ is the infusion number input.

Determining the initiating target flow rate (S(0)initiating) may comprise calculating:

$$\sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) \times {V}_{d} \times {τ}^{2}}$$

Determining the dose parameter may comprise determining a dose of the Tansy function, by calculating:

$${\int }_{\frac{n - 1}{τ}}^{\frac{n}{τ}} T ( t ) dt$$

Which may be equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} )$$

The program instructions are further configured to cause the at least one infusion device processor to determine an infusion volume for each of the number of infusion steps (h). The at least one processor determines the infusion volume for each of the number of infusion steps (h) based at least in part on the numerical approximation. Each infusion volume is indicative of a volume of the pharmaceutical preparation that is to be output by the medication delivery apparatus 90 during the respective infusion step.

In some embodiments, determining the infusion volume for one of the infusion steps comprises calculating:

$${V}_{step ( x )} = {\sum }_{n = \frac{( x - 1 ) ( i \times τ )}{h}}^{n = \frac{( x ) ( i \times τ )}{h}} ( {S}_{n} \times \frac{1}{τ} )$$

where Vₛₜₑₚ₍ₓ₎ is the infusion volume of the xth infusion step.

In some embodiments, the program instructions are further configured to cause the at least one infusion device processor to determine an infusion rate for each of the infusion steps. Determining the infusion rate for one of the infusion steps comprises calculating

$$\frac{{V}_{step ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator to displace the first plunger within the chamber such that the determined infusion volume for each infusion step is output by the medication delivery apparatus 90 during the respective infusion step.

In some embodiments, the program instructions are further configured to cause the at least one infusion device processor to actuate the infusion device actuator such that the determined infusion volume for each infusion step is output by the medication delivery apparatus 90 during the respective infusion step at the determined infusion rate.

In some embodiments, the program instructions are further configured to cause the at least one infusion device processor to actuate the infusion device actuator such that the determined infusion volume for each infusion step is delivered according to a constant-rate profile or a linearly-changing rate profile. The constant-rate profile may be as described herein. The linearly-changing rate profile may be as described herein.

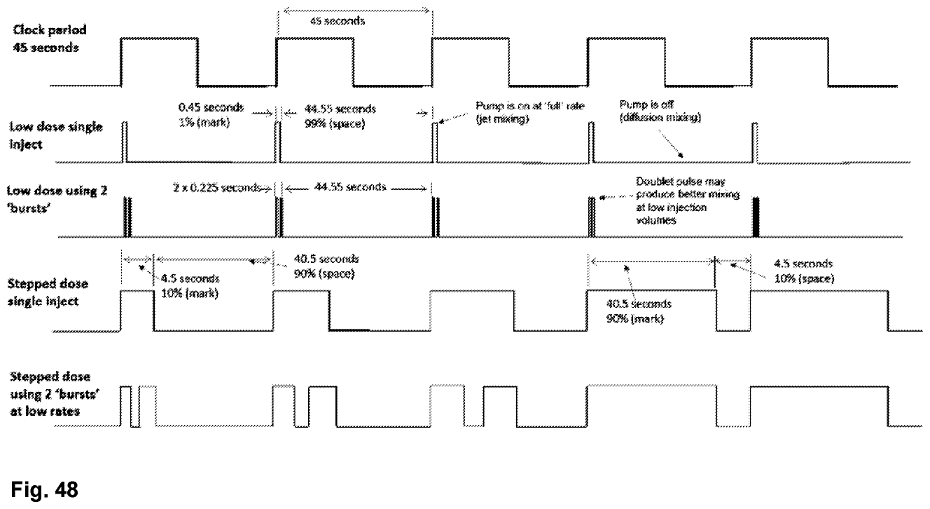

In some embodiments, the program instructions are further configured to cause the at least one infusion device processor to actuate the infusion device actuator such that the determined infusion volume for each infusion step is output by the medication delivery apparatus 90 during the respective subsequent infusion step in bursts. The bursts may be as described herein, for example, with reference to FIG. 48. The volume of infusion given during any infusion step in the Sadlier method may be given by constant infusion or linearly-varying infusion rates (‘ramp’). It may also be given by a single brief injection at a higher injection rate but lower duration such that the same volume is given but that the velocity of injection is greater and there is also a period where there is no advancement of the first plunger. There may be more than one cycle of advancement and no advancement during an infusion step (eg. ‘double burst’). The period of no advancement of the first plunger may allow the valve means 102 to close and resumption of advancement may result in opening and enhanced mixing.

In some embodiments, the concentration input Cₚ is increased by a factor of

$$( \frac{{V}_{p}}{{V}_{p} - ( {V}_{d} ( 1 - {e}^{- \frac{{V}_{p}}{{V}_{d}}} ) )} ) .$$

In some embodiments, the infusion modelling function is a Sadleir function.

The program instructions are further configured to cause the at least one infusion device processor to actuate the infusion device actuator to displace the first plunger 92 within chamber 96 such that the pharmaceutical preparation is output by the medication delivery apparatus 90 at subsequent flow rates for the remaining infusion steps until a total of h infusion steps have been delivered and the infusion is complete.

The volume of infusion given during any pump step in the Diocles method may be given by constant infusion or linearly-varying infusion rates (‘ramp’). It may also be given by a single brief injection at a higher injection rate but lower duration such that the same volume is given but that the velocity of injection is greater and there is also a period where there is no advancement of the first plunger. There may be more than one cycle of advancement and no advancement during an infusion pump step (e.g. ‘double burst’). The period of no advancement of the first plunger 92 may allow the valve means 102 to close and resumption of advancement may result in opening and enhanced mixing.

After completion of the infusion, the active ingredient remaining in the dilution chamber can be administered to the patient by collapsing the dilution chamber.

Part 7—Example with the Diocles Method

FIGS. 34B, 34C, 34D and 34E show a particular arrangement of the method of operation of the medication delivery system 91 depicted in FIGS. 30 to 41. That is, FIGS. 34B, 34C, 34D and 34E show a particular arrangement of the method of operation of the medication delivery apparatus 90 while being mounted on the syringe driver 17 (which may also be referred to as an infusion driver or an infusion device).

In particular, the rate of drug administration is controlled by a particular function (referred to as the Diocles function) in accordance with the present embodiments of the disclosure. The Diocles function may be referred to as an infusion modelling function. The Diocles is a piecemeal function with two time periods to deliver to the patient the same dose of drug over time as the Tansy function using the medication delivery apparatus 90 depicted in FIGS. 30 to 41. The first time period (when the volume of the active agent chamber 98 is greater than zero and is reducing) uses a Kelly function (see FIGS. 34C and 34D). The Kelly function is a numerically integrated algorithm to determine the volumes over time to be delivered to the patient so that the dose delivered to the patient after mixing in the dilution chamber 100 approximates that of the Tansy Function. The second time period is controlled by the Tansy function corrected for the concentration of active agent in the dilution chamber 100, which is constant once the active agent chamber 98 has been emptied.

The Diocles method is used to actuate the infusion device in order to deliver the pharmaceutical preparation to the patient by means of a medication delivery apparatus, in order to give the patient the dose of pharmaceutical preparation over time that is defined by a Tansy Dose Function. The Diocles method provides the pharmaceutical preparation in accordance with a step function with two time windows, as there are two physically different stages in the use of the medication delivery apparatus (changing drug chamber volume, constant dilution chamber volume vs constant (empty) drug chamber volume, changing dilution chamber volume).

The dilution chamber 100 is filled with diluent and a cap placed on the exit from the dilution chamber 100. The active agent chamber 100 is filled with the pharmaceutical preparation as a solution and cap placed on the filling port. The medication delivery apparatus 90 is placed in the syringe driver (i.e. the infusion driver). The cap is removed from the filling port and the syringe driver advances the first plunger 92 until fluid rises up the filling port (slack removed from system). The cap is replaced on the filling port.

The cap is removed from the exit of the dilution chamber 100. A minimum volume extension tubing is attached to the exit of the dilution chamber 100. The infusion driver advances the first plunger 92, injecting the pharmaceutical preparation into the dilution chamber 100 and fluid from the dilution chamber 100 into the minimum volume extension tubing until the mixed fluid reaches the end of the tubing, then the infusion is stopped.

The tubing is attached to a patient intravenous access. The program is started and the first of h infusion steps begins. Once the first infusion step has completed, the subsequent infusion step begins. Once the final infusion step has completed, the infusion stops.

FIGS. 34C to 34F are a flow diagrams that illustrates a method for delivering a pharmaceutical preparation to a patient. The method is the Diocles method.

As previously described, a medication delivery system 91 comprises the medication delivery apparatus 90 and the infusion device 93. The infusion device 93 may be as previously described. That is, the infusion device 93 comprises at least one infusion device processor and infusion device memory. The infusion device memory stores program instructions accessible by the at least one infusion device processor.

The program instructions are configured to cause the at least one infusion device processor to receive a concentration input (Cₚ) that is indicative of a concentration of the pharmaceutical preparation in the active agent chamber 98. The concentration may be a concentration of active agent in the pharmaceutical preparation. The concentration input (Cₚ) may be received via an input provided by a user. For example, the concentration input (Cₚ) may be input using the user interface 22. Alternatively, the concentration input (Cₚ) may be retrieved from the infusion device memory.

The program instructions are further configured to cause the at least one infusion device processor to receive a volume input (Vₚ) that is indicative of a volume of the pharmaceutical preparation. This may be a volume of the pharmaceutical preparation in the active agent chamber 98. The volume input (Vₚ) may be received via an input provided by a user. For example, the volume input (Vₚ) may be input using the user interface 22. Alternatively, the volume input (Vₚ) may be retrieved from the infusion device memory.

The program instructions are further configured to cause the at least one infusion device processor to receive a dilution chamber volume input (V_{d}). The dilution chamber volume input (V_{d}) is indicative of a volume of the dilution chamber 100. The dilution chamber volume input (V_{d}) may be received via an input provided by a user. For example, the dilution chamber volume input (V_{d}) may be input using the user interface 22. Alternatively, the dilution chamber volume input (V_{d}) may be retrieved from the infusion device memory.

The program instructions are further configured to cause the at least one infusion device processor to receive a time input (i). The time input (i) is indicative of a time window over which the pharmaceutical preparation is to be administered. The time input (i) may be received via an input provided by a user. For example, the time input (i) may be input using the user interface 22. Alternatively, the time input (i) may be retrieved from the infusion device memory. The time window comprises a first time window and a second time window.

The program instructions are further configured to cause the at least one infusion device processor to receive an infusion number input (T). The infusion number input (T) is indicative of a number of infusion intervals per minute over which an infusion modelling function is to be numerically approximated over the first time window. The infusion modelling function may be the Kelly function. The infusion number input (T) may be received via an input provided by a user. For example, the infusion number input (T) may be input using the user interface 22. Alternatively, the infusion number input (T) may be retrieved from the infusion device memory.

The program instructions are further configured to cause the at least one infusion device processor to receive a number of infusion steps (h) that are to be executed during the time window. A first number of infusion steps (h₁) are to be executed during the first time window. A second number of infusion steps (h₂) are to be executed during the second time window. Receiving the number of infusion steps (h) that are to be executed during the time window may comprise receiving an infusion step input that is indicative of the number of infusion steps (h). Alternatively, determining the number of infusion steps (h) that are to be executed during the time window may comprise retrieving the number of infusion steps (h) from the infusion device memory. Receiving the number of infusion steps (h) that are to be executed during the time window may comprise multiplying the time input (i) and the infusion number input (τ).

The program instructions may further be configured to cause the at least one infusion device processor to determine a current time (t). The current time (t) may indicate the time within the time window.

The at least one infusion device processor numerically approximates the infusion modelling function. In particular, the at least one infusion device processor numerically approximates the infusion modelling function over the first time window. To numerically approximate the infusion modelling function over the first time window, the at least one infusion device processor may perform the functionality described below. That is, numerically approximating the infusion modelling function may comprise the functionality described below.

The at least one processor determines a number of infusion intervals of the first time window. Determining the number of infusion intervals within the first time window of the numerical approximation comprises multiplying the time input (i) and the infusion number input (T). As previously described, the infusion device is capable of executing a certain number of infusion ‘events’ per minute (i.e. infusion steps). It might be that the infusion device, for example, can deliver an infusion at a particular rate for an interval of 20 seconds at a certain constant rate, then 20 seconds at another constant rate, then 20 seconds for another constant rate. So there would be three infusion ‘events’ per minute (i.e. three infusion steps per minute). Some infusion devices, for example are limited to 99 programmable ‘events’ during the course of the infusion and so a 30 minute infusion with 3 events per minute would be close to the limit of programmability of this infusion device. The particular characteristics of the infusion device will vary, what is important is that they can be programmed and that the infusion device is able to approximating the ‘ideal’ infusion program by means of a series of ‘steps’ of infusion at a particular rate.

The at least one processor determines an initiating target flow rate parameter (K(0)_{initiating}) The initiating target flow rate parameter is indicative of a target flow rate of the pharmaceutical preparation to be output into the dilution chamber 100 during an initiating infusion interval of the numerical approximation.

Determining the initiating target flow rate parameter (K(0)_{initiating}) comprises calculating:

$${K ( 0 )}_{initiating} = \sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) \times {V}_{d} \times {τ}^{2}}$$

The at least one processor determines an initiating pharmaceutical preparation concentration. The initiating pharmaceutical preparation concentration is indicative of an approximated concentration of the pharmaceutical preparation in the dilution chamber 100 after the initiating infusion interval of the numerical approximation. Determining the initiating pharmaceutical preparation concentration comprises calculating:

$${C}_{{d}_{( o )}} = \frac{( {K ( 0 )}_{initiating} \times {C}_{p} ) - ( {K ( 0 )}_{initiating} \times {C}_{{d}_{( - 1 )}} ) + ( {C}_{{d}_{( - 1 )}} \times {V}_{d} \times τ )}{{V}_{d} \times τ}$$

where C_{d}_{_{(−1)}}=0 and C_{d}_{₍ₒ₎} is the initiating pharmaceutical preparation concentration.

The at least one processor iteratively determines a subsequent target flow rate and a subsequent pharmaceutical preparation concentration for each of a plurality of subsequent infusion intervals of the numerical approximation. The subsequent target flow rates are each indicative of a target flow rate of the pharmaceutical preparation to be output by the medication delivery apparatus during a respective subsequent infusion interval of the numerical approximation. The subsequent pharmaceutical preparation concentrations are each indicative of a subsequent approximated concentration of the pharmaceutical preparation in the dilution chamber 100 after the respective subsequent infusion interval. Each of the subsequent target flow rates is determined based at least in part on the subsequent pharmaceutical preparation concentration of a previous infusion interval of the respective infusion interval. Each of the subsequent pharmaceutical preparation concentrations is determined based at least in part on the subsequent target flow rate of the respective subsequent infusion interval.

Determining the subsequent target flow rates comprises determining a flow rate parameter Kₙ for each of the subsequent target flow rates. The at least one infusion device processor determines Kₙ by calculating:

$${K}_{n} = \frac{Dose {( t )}_{n} * τ}{{C}_{d ( n - 1 )}}$$

where n is the number of the relevant infusion interval, C_{d(n-1)} is a subsequent pharmaceutical preparation concentration of a previous infusion interval of the nth infusion interval and Dose(t)ₙ is a target dose of the respective infusion interval of the first time window. The target dose is described in more detail herein. For example, the target dose Dose(t)ₙ may be similar to the previously described dose parameter.

In particular, determining the target dose Dose(t)ₙ comprises determining a dose of a Tansy function T(t). That is, determining the target dose Dose(t)ₙ comprises calculating:

$${\int }_{\frac{n - 1}{τ}}^{\frac{n}{τ}} T ( t ) d t \times {C}_{p}$$

where T(t) is the Tansy function.

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} )$$

In some embodiments, determining the subsequent pharmaceutical preparation concentrations of the first numerical approximation comprises calculating:

$${C}_{d ( n )} = \frac{( {K}_{n} \times {C}_{p} ) - ( {K}_{n} \times {C}_{d ( n - 1 )} ) + ( {C}_{d ( n - 1 )} \times {V}_{d} \times τ )}{{V}_{d} \times τ}$$

where C_{d(n)} is the subsequent pharmaceutical preparation concentration for the nth infusion interval and C_{d(n-1)} is the subsequent pharmaceutical preparation concentration for the n−1 th infusion interval. In other words, n is the number of the relevant infusion interval and C_{d(n-1)} is a subsequent pharmaceutical preparation concentration of a previous infusion interval of the nth infusion interval.

This calculation may be performed for each subsequent pharmaceutical preparation concentration of the iteration.

The at least one infusion device processor determines a first infusion volume for each of the first number of the infusion steps (h₁). In particular, the at least one infusion device processor determines the first infusion volume for each of the first number of the infusion steps (h₁) based at least in part on the numerical approximation. The infusion volume is indicative of a volume of the pharmaceutical preparation that is to be output by the medication delivery apparatus during the respective infusion step.

The at least one infusion device processor determines the first infusion volume f or one of the first number of the infusion steps (h₁) by calculating:

$${V}_{step ( x )} = {\sum }_{n = \frac{( x - 1 ) ( i \times τ )}{h}}^{n = \frac{( x ) ( i \times τ )}{h}} ( {K}_{n} \times \frac{1}{τ} )$$

where Vₛₜₑₚ₍ₓ₎ is the infusion volume of the xth infusion step of the first number of the infusion steps (h₁).

The at least one infusion device processor determines a number of infusion intervals of the second time window.

The at least one infusion device processor determines a target dose Dose(t)ₙ for each of the number of infusion intervals of the second time window. This may be as described in FIGS. 34A to 34D.

The at least one infusion device processor determines a target flow rate Dₙ for each of the number of infusion intervals of the second time window, based at least in part on the target dose for the respective infusion interval. Determining the target flow rate for each of the number of infusion intervals of the second time window comprises calculating:

$${\int }_{\frac{n - 1}{τ}}^{\frac{n}{τ}} T ( t ) dt \times \frac{{c}_{p}}{{c}_{dc}} .$$

where C_{dc} is a concentration of the pharmaceutical preparation in the dilution chamber at a point when the active agent chamber is empty.

The at least one infusion device processor determines determine a second infusion volume for each of the second number of infusion steps (h₂) based at least in part on the target flow rate. Determining the second infusion volume for one of the second number of the infusion steps (h₂) comprises calculating:

$${V}_{step ( x )} = {\sum }_{n = \frac{( x - 1 ) ( i \times τ )}{h}}^{n = \frac{( x ) ( i \times τ )}{h}} ( {D}_{n} \times \frac{1}{τ} )$$

where Vₛₜₑₚ₍ₓ₎ is the infusion volume of the xth infusion step of the second number of the infusion steps (h₂) and Dₙ is the target flow rate for one of the number of infusion intervals of the second time window.

The at least one infusion device processor actuates an infusion device actuator to displace the first plunger such that the determined infusion volume for each infusion step (h) is output by the medication delivery apparatus 90 during the respective infusion step.

In some embodiments, the program instructions are further configured to cause the at least one infusion device processor to determine an infusion rate for each of the infusion steps (h), and wherein determining the infusion rate for one of the infusion steps comprises calculating

$$\frac{{V}_{s t e p ( x )} \times h}{i} ,$$

where

Vₛₜₑₚ₍ₓ₎ is the infusion volume of the xth infusion step.

In some embodiments, the program instructions are further configured to cause the at least one infusion device processor to actuate the infusion device actuator such that the determined infusion volume for each infusion step is output by the medication delivery apparatus 90 during the respective infusion step at the determined infusion rate.

In some embodiments, the program instructions are further configured to cause the at least one infusion device processor to actuate the infusion device actuator such that the determined infusion volume for each infusion step is delivered according to a constant-rate profile or a linearly-changing rate profile.

In some embodiments, the program instructions are further configured to cause the at least one infusion device processor to actuate the infusion device actuator such that the determined infusion volume for each infusion step is output by the medication delivery apparatus during the respective subsequent infusion step in bursts.

In some embodiments, receiving the number of infusion steps that are to be executed during the time window comprises receiving an infusion step input that is indicative of the number of infusion steps. In some embodiments, receiving the number of infusion steps that are to be executed during the time window comprises retrieving the number of infusion steps from the infusion device memory

In some embodiments, the program instructions are further configured to cause the at least one infusion device processor to receive a pharmaceutical preparation input. The pharmaceutical preparation input may be indicative of one or more of an identity of the pharmaceutical preparation; a dose of the pharmaceutical preparation; and a maximum pharmaceutical preparation administration rate. In some embodiments, the subsequent target flow rates are limited at the maximum pharmaceutical preparation administration rate, such that the subsequent target flow rates do not exceed the maximum pharmaceutical preparation administration rate.

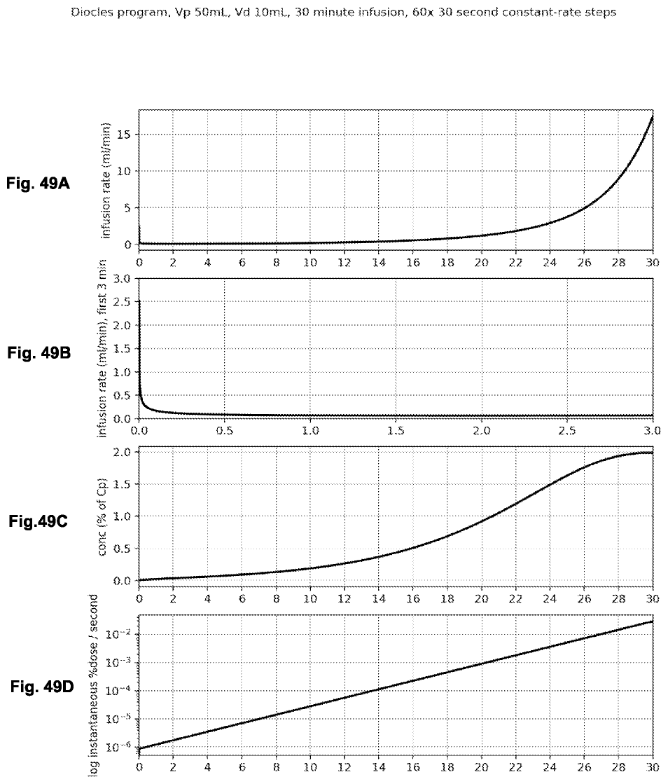

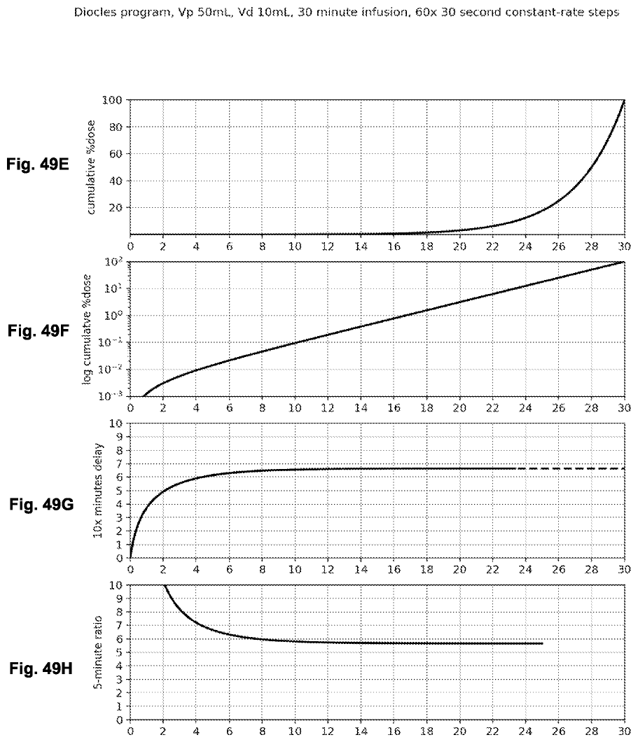

FIGS. 49A to 49G show theoretical results provided by implementation of the Diocles method for a particular value of Vₚ, V_{d} and i. FIG. 49A is a chart illustrating a fluid injection rate (y-axis) in mL/min of the Diocles method against an infusion time (x-axis) in minutes. FIGS. 49A and 49B (first 3 minutes of a 30 minute infusion) are charts illustrating the infusion flow rate vs time. FIG. 49C is a chart illustrating a concentration of the pharmaceutical preparation delivered to the patient (x-axis), with the units of the x-axis being percentage of total dose in the active agent chamber 98 (or therapeutic dose per mL) against an infusion time (x-axis) in minutes. FIG. 49D is a log chart of the instantaneous percentage dose of the pharmaceutical preparation delivered per second (y-axis) against infusion time (x-axis) in minutes. FIG. 49E is a chart illustrating the cumulative percentage dose (y-axis) against infusion time (x-axis) in minutes. FIG. 49F is a log chart illustrating the cumulative percentage dose (y-axis) against infusion time (x-axis) in minutes. FIG. 49G is a chart showing the number of minutes until the cumulative dose delivered is 10 times that at the time indicated on the x-axis. For example, at the point in time 2 minutes in the infusion, it will be another 5 minutes before the cumulative dose is 10 times that which the cumulative dose was at 2 minutes, and at the point in time 14 minutes into the infusion, it will be another 6.7 minutes before the cumulative dose administered is 10× what the cumulative dose was at 14 minutes. FIG. 49H is a chart showing the ratio of cumulative dose at each point in time during the infusion at that point compared to the cumulative dose 5 minutes later in the infusion. For example, at 2 minutes into the infusion the cumulative dose 5 minutes later will be approximately 10 times the cumulative dose at 2 minutes, and at 14 minutes into the infusion the ratio of cumulative dose 5 minutes later will be approximately 5.7 times greater than it was at 14 minutes. These graphs indicate the interval likely to be available to wait for the emergence of an adverse reaction before a dose that would cause a more severe reaction is given.

The volume of infusion given during any pump step in the Diocles method may be given by constant infusion or linearly-varying infusion rates (‘ramp’). It may also be given by a single brief injection at a higher injection rate but lower duration such that the same volume is given but that the velocity of injection is greater and there is also a period where there is no advancement of the first plunger 92. There may be more than one cycle of advancement and no advancement during an infusion step (e.g. ‘double burst’). The period of no advancement of the first plunger 92 may allow the valve means 102 to close and resumption of advancement may result in opening and enhanced mixing.

Part 8—Method of Delivery where a Maximum Delivery Rate is Exceeded or a Maximum Tolerable Dose is Exceeded

The maximum rate of delivery of drug may be exceeded during the infusion process as a consequence of user settings. In order to ensure this does not happen, the medication delivery system can check that each infusion step does not exceed the maximum allowable dosing rate by estimating the dilution chamber drug concentration and the fluid infusion rate. The dilution chamber drug concentration as a function of cumulative drug volume infused (V) is given by the following equation:

$${C}_{d} = {C}_{p} * ( 1 - {e}^{- \frac{V}{{V}_{d}}} )$$

$${C}_{d} is the concentration of drug in the dilution chamber$$

$${C}_{p} is the original concentration of drug in the drug delivery flask or syringe or container$$

$${V}_{d} is the volume of the dilution chamber$$

$$V is cumultive volume infused into the dilution chamber or patient$$

Cisplatin Dosing

For example, current dosing for a man is 40 mg/m2 over 1 hour in 1000 mL of diluent. This protocol (i.e. the medication delivery system) will deliver 72 mg of Cisplatin in 1000 mL over 60 minutes, which is a fluid injection rate of 16.7 ml/min, and a dose rate of 1.2 mg/min.

If this is delivered using the previously disclosed medication delivery apparatus 90, one can prepare the 72 mg of Cisplatin in 1000 mL of diluent in a flask, connected to the medication delivery apparatus 90 by a peristaltic fluid pump. The dilution chamber 100 can be set at 50 mL volume. The Diocles algorithm can be used as the infusion duration will be limited by the maximum dosing rate in both described instances (rather than using the Sadleir algorithm which is chosen when the dilution chamber 100 is not automatically collapsed and where the automatic program prior to manually collapsing the dilution chamber is desired to be of the duration set).

The duration of infusion can be set to 60 minutes using 120 constant infusion steps of 30 seconds. Using this arrangement, the dose rate increases exponentially over the duration of the infusion. The minimum infusion flow rate will be 0.306 ml/min (18.4 ml/hr). The maximal allowable dose rate (1.2 mg/min) is reached at 46 minutes and 29 seconds, when the cumulative volume administered has been 143 mL, the dilution chamber concentration is 0.0.679 mg/mL, and the infusion rate is 17.7 mL/min. For the subsequent infusion step, the infusion rate is limited to 17.7 ml/min and the cumulative volume at the end of that step is 161 mL. The dilution chamber concentration is then estimated at 0.0691 mg/mL. The following step will have the infusion rate reduced to 17.4 mL/min to ensure that the maximal allowable dose rate (1.2 mg/min is not exceeded). This adjustment of each step infusion rate will continue until the infusion is completed. The duration of the infusion will be extended to a total infusion duration of approximately 98 minutes. The final step infusion rate will be approximately 16.7 mL/min and the dilution chamber concentration 0.072 mg/ml. After completion of the infusion, either the dilution chamber can be collapsed to deliver the final 50 mL of solution, or an additional 50 mL of drug infusion can be delivered from the drug flask via the dilution chamber.

The duration of infusion can be set to 180 minutes using 360 constant infusion steps of 30 seconds. Using this arrangement, the dose rate increases exponentially over the duration of the infusion. The minimum infusion flow rate will be 0.1 mL/min (6 ml/hr). The maximum allowable infusion dose rate (1.2 mg/min) is exceeded at 158 minutes and 29 seconds, therefore the infusion will be limited to the infusion rate for the subsequent interval (starting at 158 minutes and 30 seconds). The cumulative volume delivered is 338.5 mL and the infusion rate 16.7 ml/min. The dilution chamber concentration is estimated at 0.0719 mg/mL and so the allowable infusion rate is 16.7 mL/min for all subsequent intervals. The remaining 661.5 mL infusion will complete in a further 40 minutes (total infusion duration approximately 198 minutes). After 1000 mL of the drug infusion has been infused, the dilution chamber can be collapsed to deliver the final 50 mL of solution, or an additional 50 mL of drug infusion can be delivered from the drug flask via the dilution chamber.

Rocuronium Dosing

Rocuronium is a non-depolarising neuromuscular blocking agent and is chosen as an example of a drug that can only have a part of its therapeutic dose administered slowly (the rest having to be administered either quickly or slowly when anaesthetized). It is administered intravenously in a dose of 0.6 mg/kg (50 mg for an 80 kg patient). This is usually administered as a push after anaesthesia is induced.

Rocuronium may be administered to an awake patient up to a dose of approximately 0.03 mg/kg (2.4 mg in an 80 kg patient). This will cause minor, tolerable side-effects (blurred vision).

Test doses or desensitisation may be administered by diluting 50 mg of rocuronium in an infusion volume Vp of 50 mL, with a 10 mL dilution chamber, infused over 30 minutes, but pausing the infusion for induction of anaesthesia once 0.03 mg/kg has been administered. Then the remainder of the infusion can either be given as a push (if relaxation is required immediately at induction) or by continuing the remainder of the infusion.

Using the medication delivery system 91 with this protocol, 2.4 mg is administered after 21 minutes and 14 seconds. The infusion rate at this point is 1.43 ml/min and 7.83 ml of solution has been infused.

Method of calculating infusion rate and cumulative volume delivered using the medication delivery system 91

As described, the dilution chamber drug concentration as a function of cumulative drug volume infused (V) is given by the following equation:

$${C}_{d} = {C}_{p} * ( 1 - {e}^{- \frac{V}{{V}_{d}}} )$$

$${C}_{d} is the concentration of drug in the dilution chamber$$

$${C}_{p} is the original concentration of drug in the drug delivery flask or syringe or container$$

$${V}_{d} is the volume of the dilution chamber$$

$$V is cumultive volume infused into the dilution chamber or patient$$

This relationship can hold until the dilution chamber volume is reduced by the advancing plunger, beyond which point the dilution chamber concentration remains constant.

Part 9—Further Examples of Diocles Method

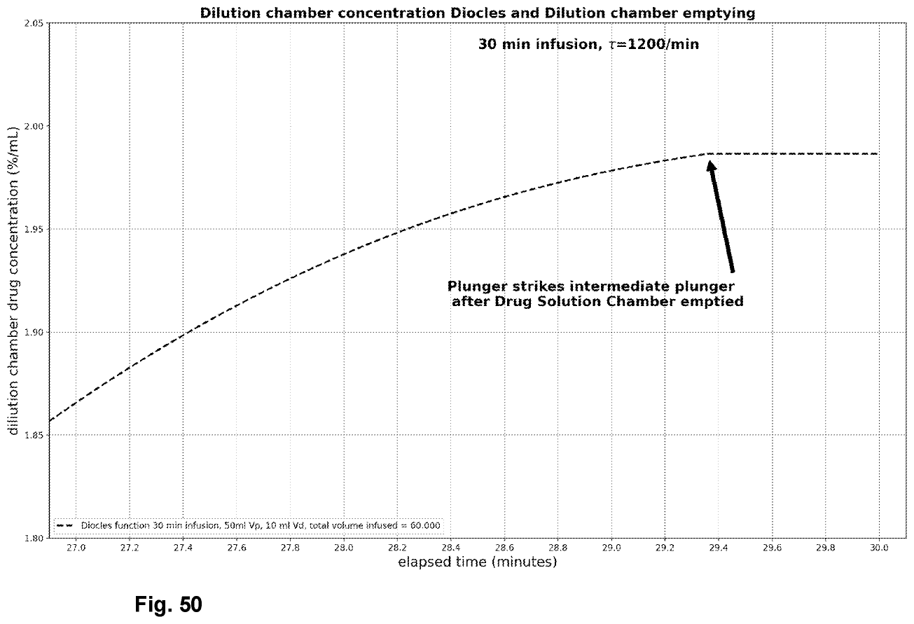

Example infusions in accordance with the Diocles method and the medication delivery system 91 FIG. 50 illustrates the dilution chamber drug concentration of an infusion delivered using the Diocles method (y-axis) against infusion time (x-axis) in minutes.

FIG. 51 illustrates a comparison of the cumulative dose infused as a percentage of the total dose and the cumulative volume infused in mL of infusions delivered according to the Tansy method and the Diocles method.

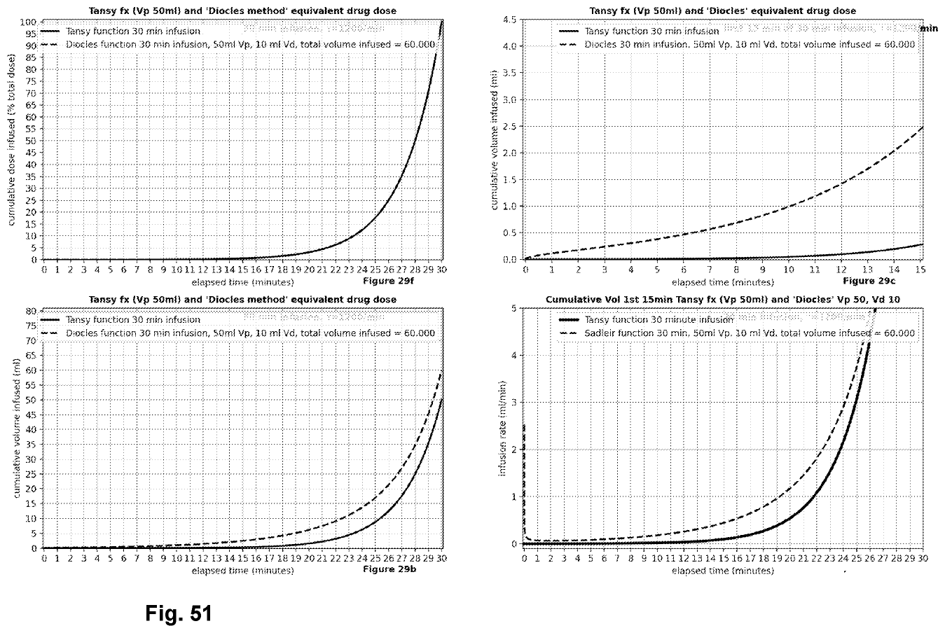

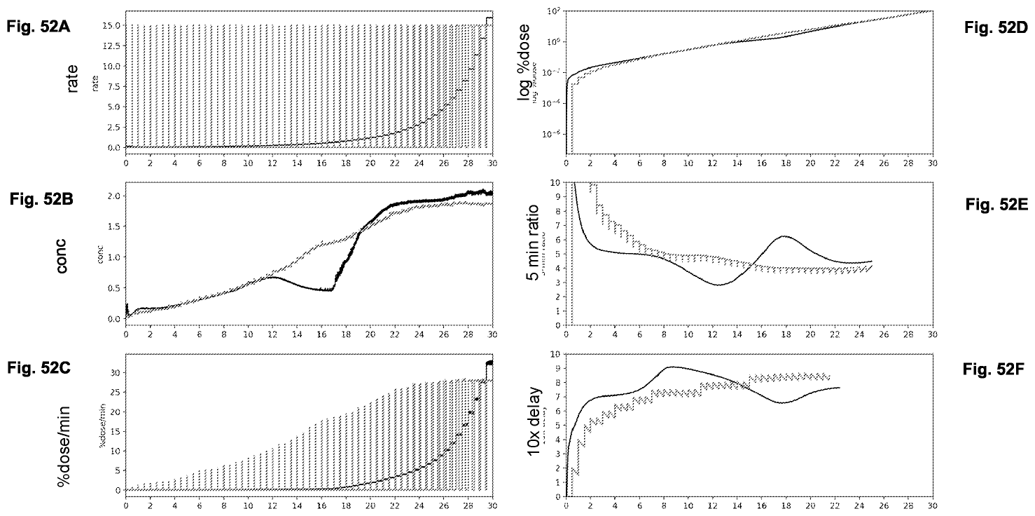

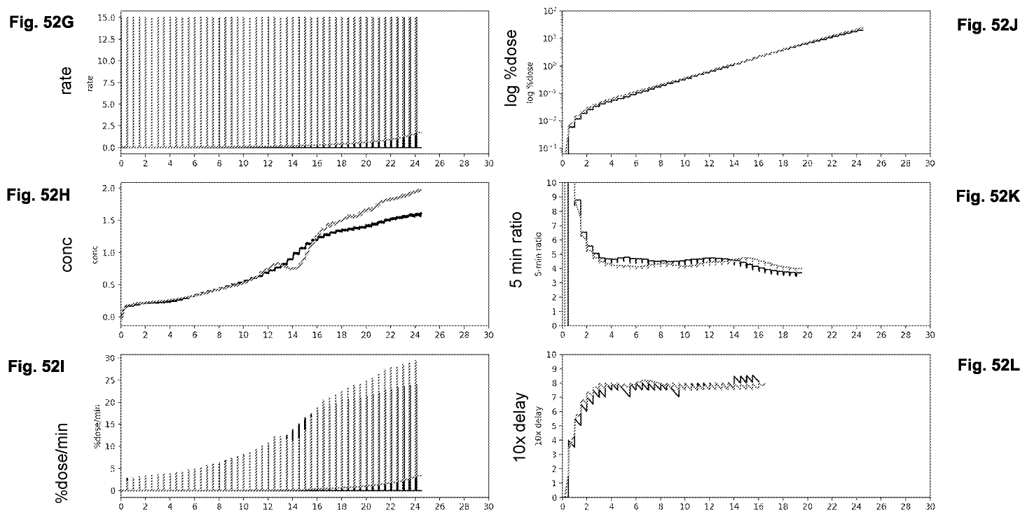

FIGS. 52A to 52F illustrate a test comparison of a 30 minute infusion using 60 30 second steps, with each step being a constant infusion and a 30 minute infusion using 60 bursts at a higher infusion rate. As is illustrated in these figures, the method involving bursts provides for better mixing of the pharmaceutical preparation and the diluent.

FIGS. 52G to 52L illustrate a test comparison of a 30 minute infusion using 60 30 second steps with a double burst at 15 mL/min separated by 1 second (darker colour) compared to a single burst at 15 mL/min with the volume of the second burst spread throughout the interval (i.e. no closure of the valve and no cracking (lighter colour). The former has improved mixing.

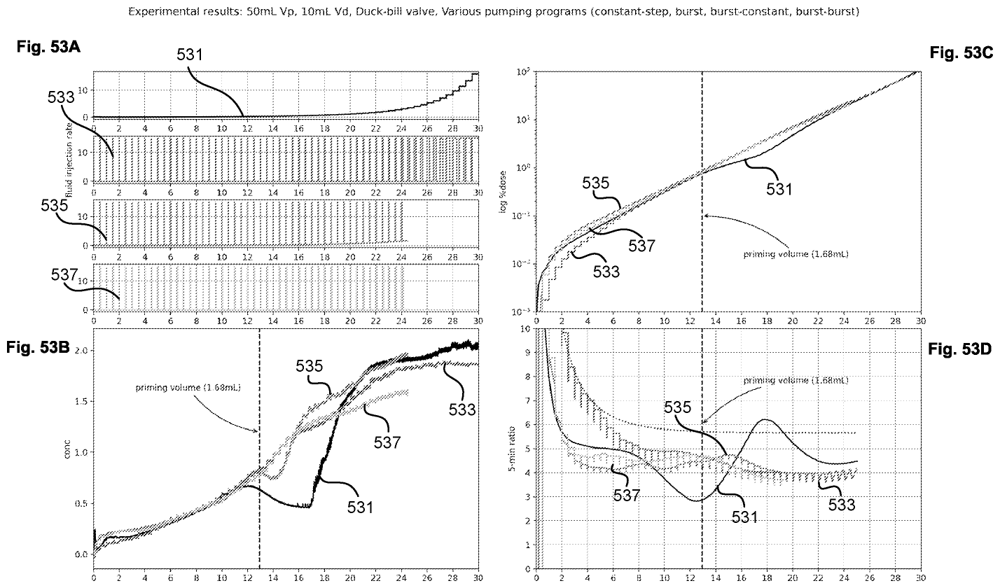

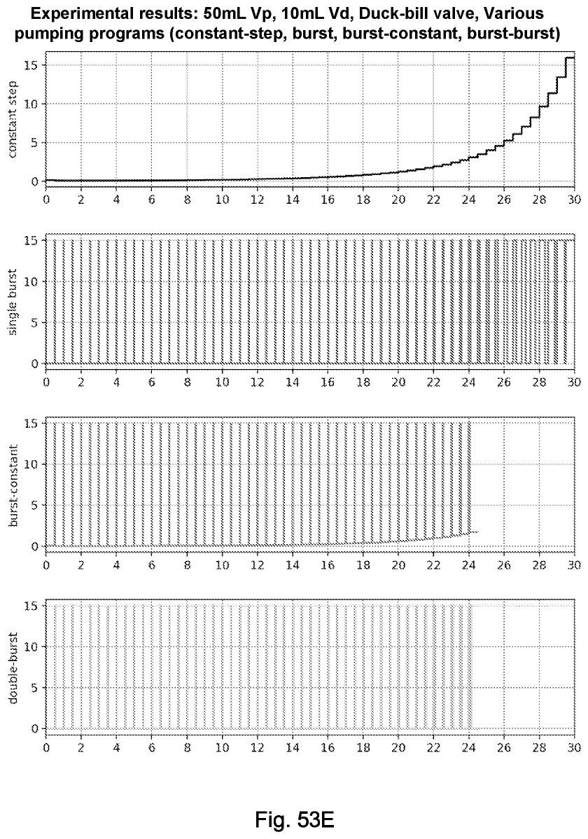

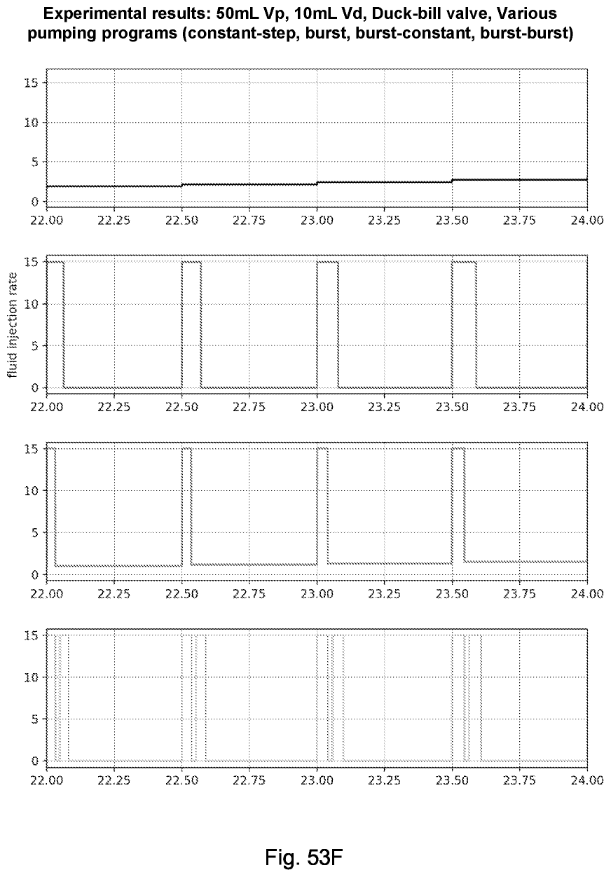

FIGS. 53A to 53D illustrate constant step, burst, burst-constant and burst-burst infusion delivery programs and resultant pharmaceutical preparation delivery results. FIG. 53A illustrates a plurality of infusion profiles. The infusion driver of the described medication delivery systems may actuate the infusion driver actuator according to an infusion profile (which may also be referred to as an infusion program). FIG. 53A illustrates a constant step infusion program 531, a burst infusion program 533, a burst-constant infusion program 535 and a burst-burst infusion program 537.

In the constant step delivery program, the infusion device actuates the infusion device actuator at a constant rate. In the burst program, the infusion device actuates the infusion device actuator in bursts (i.e. rapid relatively larger actuations over a shorter period of time). In the burst-constant program, the infusion device actuates the infusion device actuator in bursts, with an underlying constant actuation also applied. That is, the burst-constant program may be considered to be a superposition of a constant-rate infusion and a burst infusion. In the burst-burst program, two bursts are provided in rapid succession. That is, two rapid, relatively larger actuations are provided over a relatively short period of time.

FIG. 53B illustrates the concentration of the pharmaceutical preparation output from the medication delivery system for each of the infusion programs. FIG. 53C illustrates the percentage of the pharmaceutical preparation output from the medication delivery system (y-axis) according to a logarithmic scale, against infusion time for each of the infusion programs. FIG. 53D illustrates the ratio of cumulative dose given 5 minutes after the time indicated on the x-axis, to the cumulative dose given at the time indicated on the x-axis.

FIGS. 53E and 53F illustrates a constant step, single burst, burst-constant and double-burst infusion step programs, according to some embodiments.

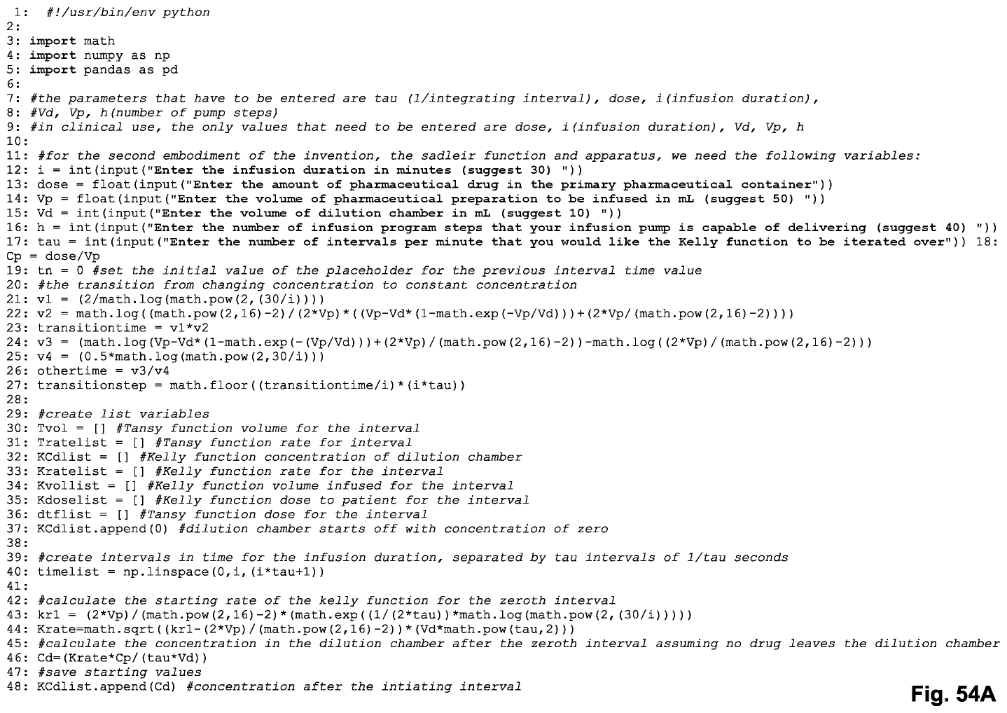

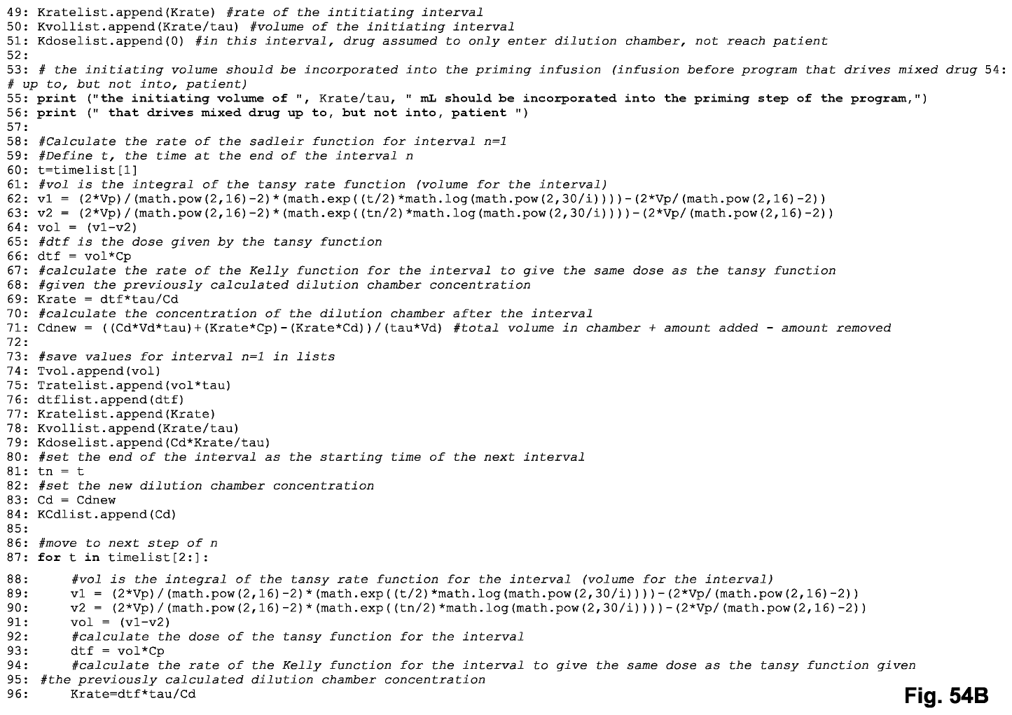

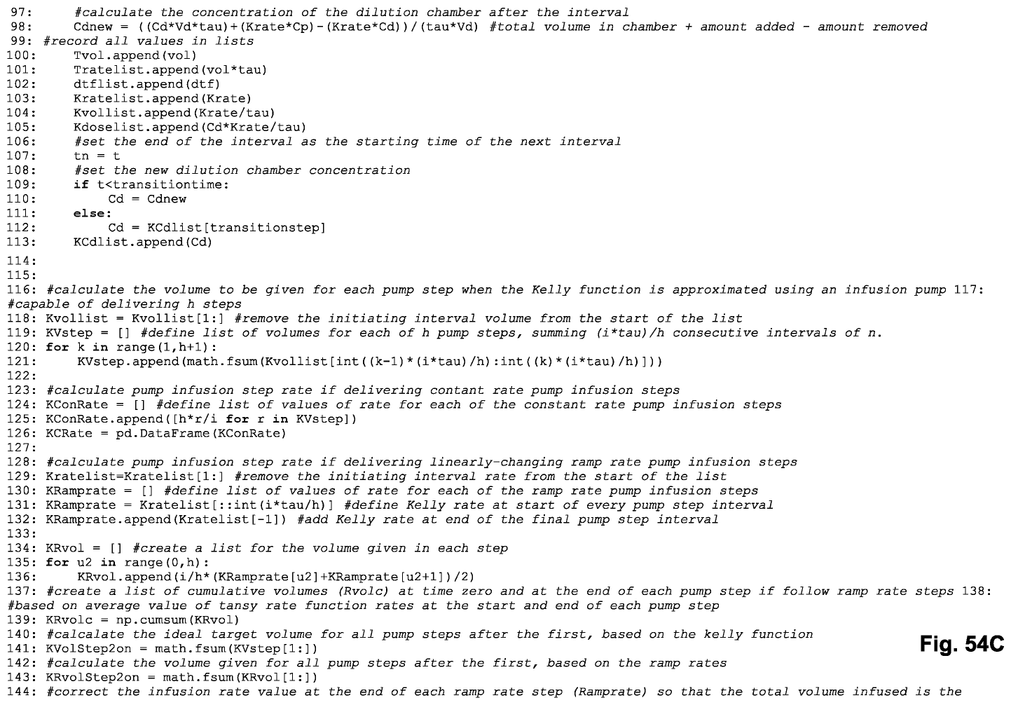

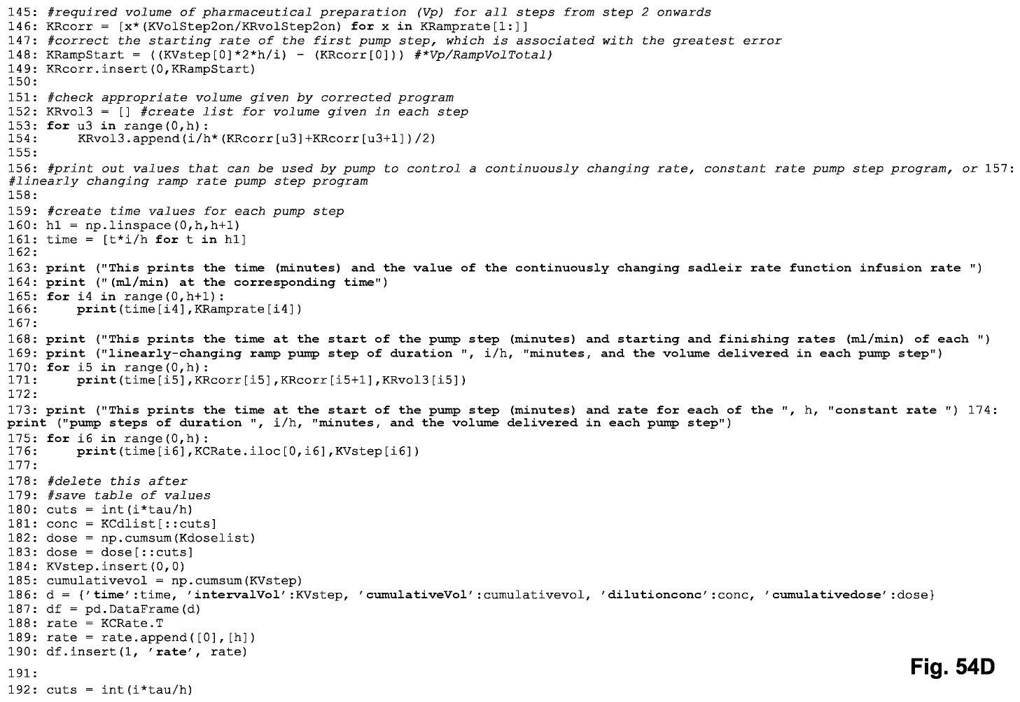

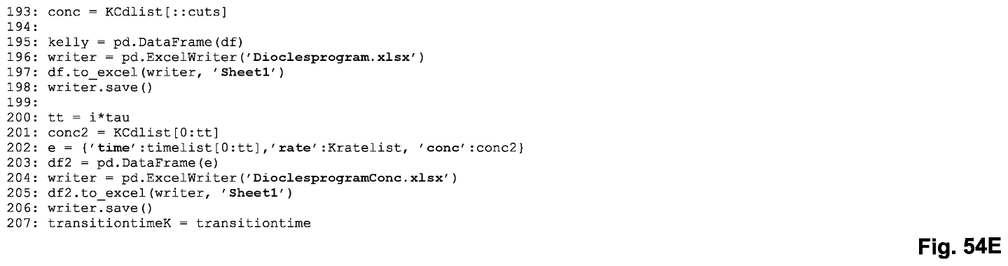

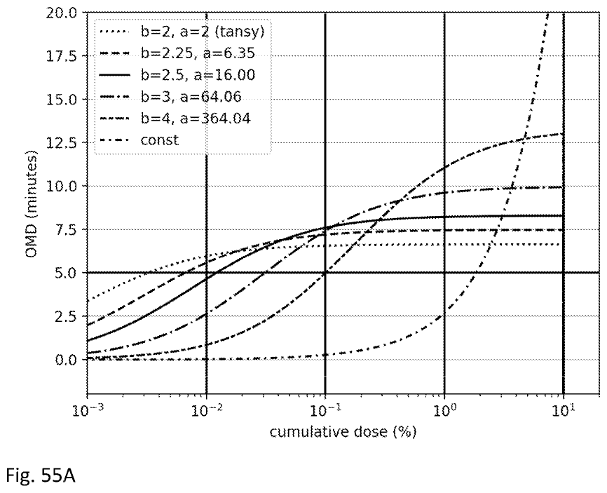

FIGS. 54A to 54E show software code, written in Python 3, to calculate the values that can be sent to the infusion device to realise the Diocles method, according to some embodiments.

Part 10—Further Example Medication Delivery Systems

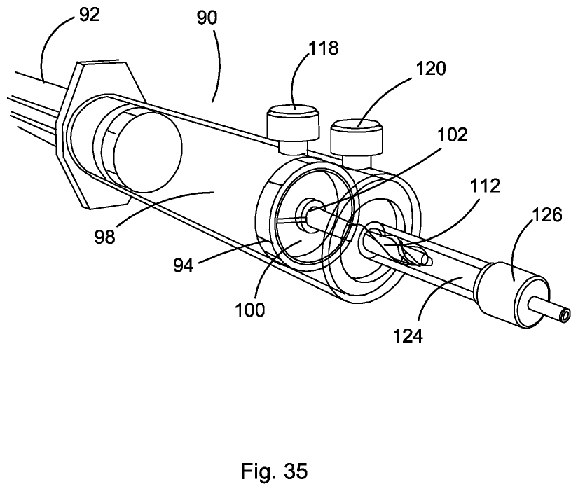

Referring now to FIGS. 35 to 39, FIGS. 35 to 39 show an alternative arrangement of a medication delivery apparatus 90. The medication delivery apparatus 90 may be referred to as a dilution chamber 90. Again, the medication delivery apparatus 90 may form part of a medication delivery system 91 comprising an infusion device as previously described.

The medication delivery apparatus 90 shown in FIGS. 35 to 39, differ with respect to the medication delivery apparatus 90 depicted in FIGS. 30 to 34, in that it comprises a separating plunger 94 having a particular arrangement of valve means 102 and a particular arrangement of outlet 110 that differs from the separating plunger 94, valve means 102 and outlet 110 of the medication delivery apparatus 90 depicted in FIGS. 30 to 34.

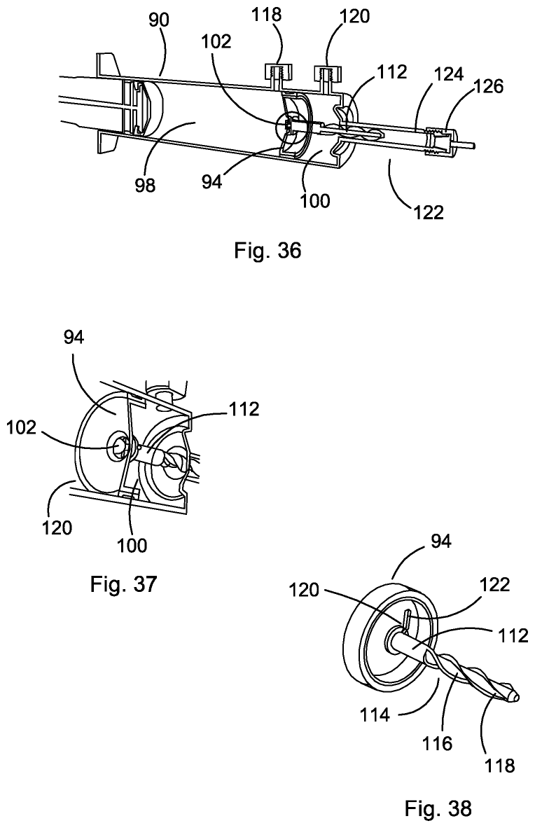

In particular, as shown in FIGS. 36 to 38, the separating plunger 94 comprises a valve means 102 having stirring means 112 for mixing the diluent and the active agent (entering the mixing chamber 100). As shown in particular in FIG. 37, the valve means 102 provides communication between the active agent chamber 98 and the mixing chamber 100 to permitting flow of the active agent into the mixing chamber 100. Flow of the active agent drives rotational movement of the stirring means 112.

As shown in FIG. 38, the stirring means 112 comprise a screw member 114. The screw member 114 is rotatably attached to the valve means 102. The screw member 114 comprises a cylindrical shaft 116 and a helical structure 118 surrounding the cylindrical shaft 116. Further, the stirring means 12 also comprises a stirrer 120 having an extension 122 extending outwards from the stirring means 112. The stirrer 120 aids in the stirring process during rotation of the stirring means 112 as the active agents flows into the mixing chamber 100.

Further, referring back to FIG. 36, the medication delivery apparatus 90 comprises one inlet port 118 for filling the active agent chamber 98 and another inlet 120 for filing the mixing chamber 100. The process for filing the active agent chamber 98 and the mixing chamber 100 is substantially identical to the process explained above with respect to FIG. 31.

Furthermore, the medication delivery apparatus 90 comprises an outlet port 122. The particular arrangement of medication delivery apparatus 90, shown in FIG. 36, comprises an outlet port 122 having a tube section 124 extending from the mixing chamber 100 defining in this manner a space for receiving at least the distal portion of the stirring means 112 comprising the screw member 114—see FIG. 36. During operation of the medication delivery apparatus 90, and in particular when the separating plunger 94 is displaced for discharging the pharmaceutical composition out the mixing chamber 94, the screw member 114 is inserted into the tube section 124 as shown, for example, in FIG. 36 ensuring proper mixing of the active agent and the diluent for production of the pharmaceutical composition.

In order to deliver the pharmaceutical composition to the patient, the tube section 124 is adapted to receive the proximal end of the conduit 30a with its distal end in fluid communication with the infusion device (fluidly connected to the patient's blood stream) that delivers the pharmaceutical composition into the patient. As shown in FIG. 36 a hollow cap 126 is attached to the tube section 124 for fluidly connecting the medication delivery apparatus 90 to the conduit 30a.

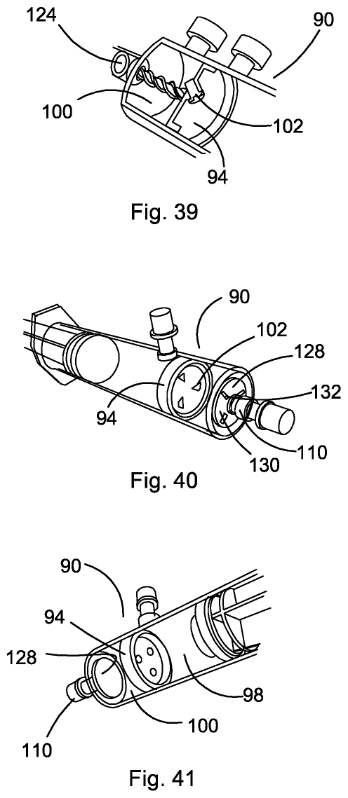

Further, FIGS. 39 to 41 shows alternative arrangements of valve means 102 incorporated in the separating plunger 94 of the dilution chambers 90 depicted in FIGS. 39 to 41.

Furthermore, as shown in FIGS. 40 and 41, the particular arrangement of medication delivery apparatus 90 shown in FIGS. 40 and 41 comprises stirring means 128 in the shape of a disc 130 having a helix-like groove structure 132 indenting into the face of the disc 130 that faces the outlet 110 of the medication delivery apparatus 90.

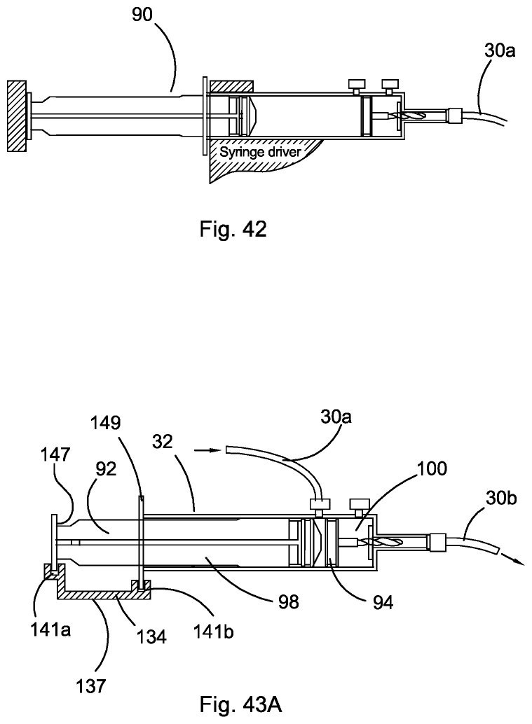

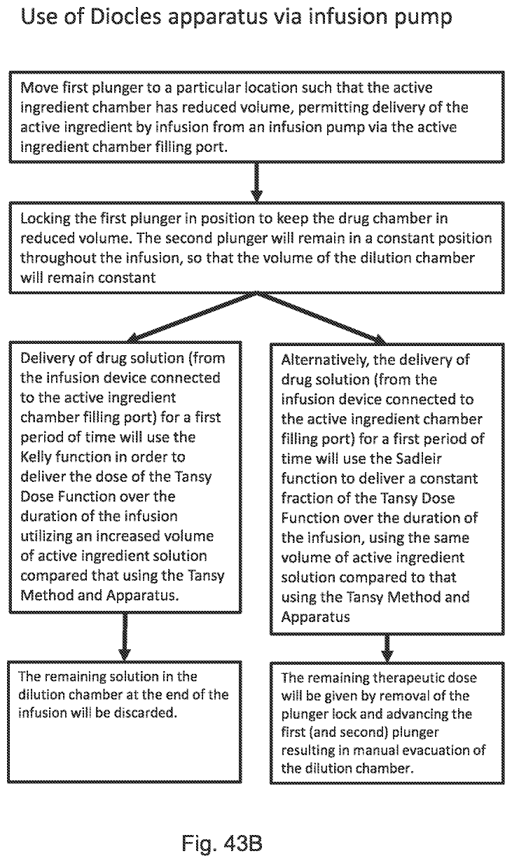

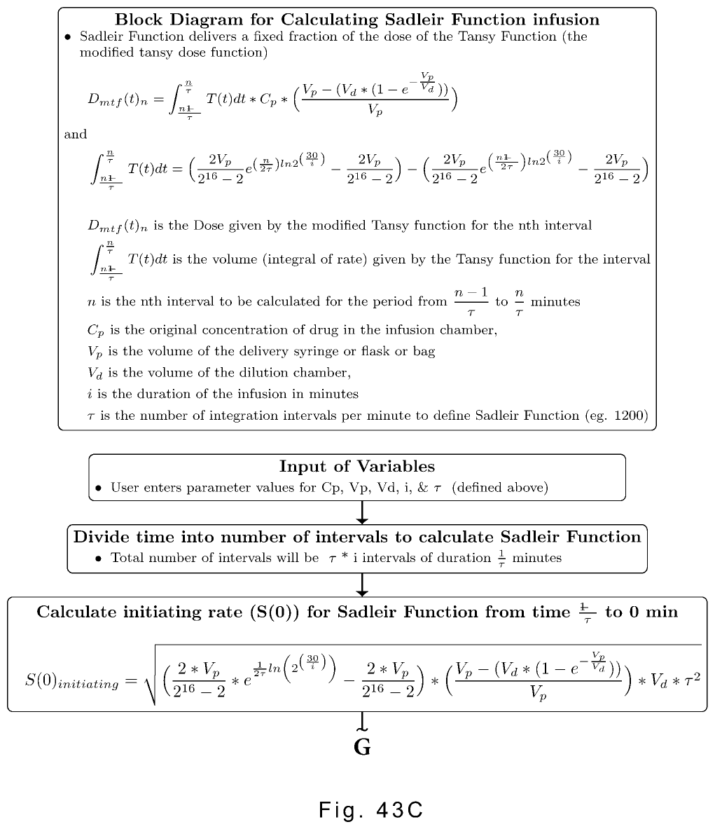

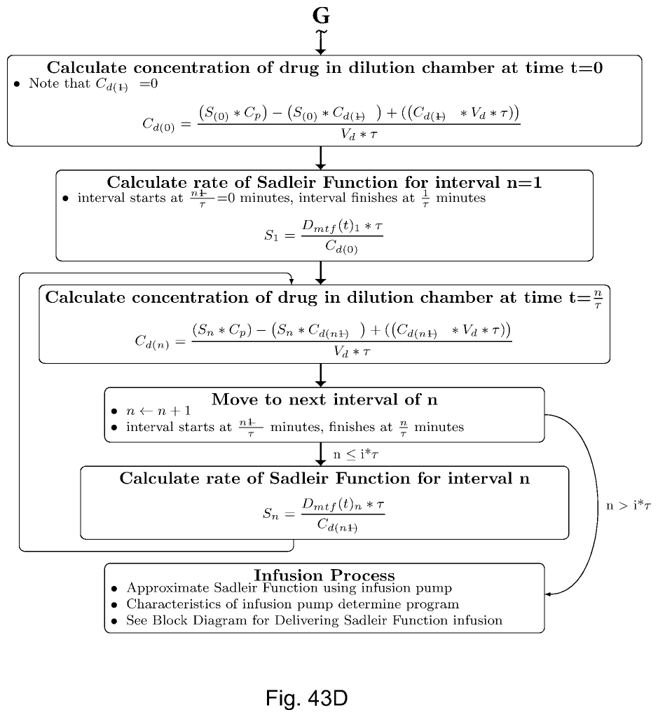

Referring now to FIGS. 42 and 43, FIGS. 42 and 43 depict the particular arrangement of the medication delivery apparatus 90 shown in FIG. 35.

The dilution chambers 100 depicted in FIGS. 42 and 43 as well as in FIGS. 30 to 39 are adapted to operate either as (1) a syringe for mounting on a syringe driver 17 or (2) the dilution chamber 32 described in relation to FIGS. 2 to 7 in which the dilution chamber 32 is located at a remote location of the syringe driver 17 having a syringe 15 solely filled with active agent.

FIGS. 30 to 35 and 40 show the medication delivery apparatus 90 operating as a syringe for mounting on a syringe driver 17 for delivering the pharmaceutical composition (i.e. mixture of active agent and diluent). This particular arrangement of medication delivery apparatus 90 is particularly useful (when compared against the dilution chamber 32 described with reference to FIGS. 2 to 7) because it permits omitting the dilution chamber 32 (located at a remote location of the syringe driver 17) which is used for mixing the active agent and diluent prior delivering the pharmaceutical composition (containing active agent and diluent) to the patient.

However, in alternative arrangements (see FIG. 43), the dilution chambers 100 may function as a dilution chamber 32 being located at a remote location of the syringe driver 17 as depicted in FIGS. 2 to 7.

As shown in FIG. 43A, the medication delivery apparatus 90 comprises a plunger lock 134 in order to fix the primary plunger 92 at a particular location permitting to deliver the active agent (coming from the syringe driver 17) into the active agent chamber 98 for delivery of the drug, through the separation plunger, into the mixing chamber 100 for delivery into the patient via the conduit 30b.

The plunger lock 134 shown in FIG. 43A comprises a body having a lower surface 137 for resting on a support surface and an upper surface 139 having spaced apart grooves 141a and 141b for receiving the flanges 145 and 147 of the primary plunger 92 and the active agent chamber 98. In this manner, the primary plunger 92 is fixed at a particular location not being able to move within the active agent chamber 98.

As shown in FIG. 43A, the primary plunger 92 is located in a particular location such that the active agent chamber 98 has a relatively small volume. The fact that the primary plunger 92 is locked in position due to the plunger lock 134, impedes the primary plunger 92 from moving, thus maintaining the relative small volume of the active agent chamber 98 constant as active agent is delivered from the syringe driver 17 via conduit 30a into the active agent chamber 98.

In operation, as active agent is delivered into the active agent chamber 98 of constant volume, the pharmaceutical preparation is forced to flow through the separating plunger 94 into the mixing chamber 100 for mixing of the pharmaceutical preparation and the diluent to prepare the pharmaceutical composition for delivery into the blood stream of the patient via conduit 30a. FIGS. 43B and 43C illustrates the method of operation of the medication delivery apparatus 90 when operating remotely from the syringe driver 17 comprising a syringe 15 solely filled with active agent.

In particular, the rate of active agent administration is governed by the Sadleir function. The Sadleir function is a numerically-integrated function to determine the volumes over time to be delivered to the patient so that the dose delivered to the patient after mixing in the medication delivery apparatus 90 approximates that of a fixed fraction of the Tansy Function at that time using the Sadleir embodiment. This is because at the end of the infusion some pharmaceutical composition still remains in the mixing chamber 100 that will be delivered to the patient by moving the primary plunger 92 towards the outlet 110.

In an alternative arrangement and as mentioned before, it is also possible to increase the concentration of the active agent in the dilution chamber 100 to deliver the same dose as the Tansy function over time, and then discard the remaining pharmaceutical composition in the dilution chamber 100 instead than delivering it to the patient.

Alternative Medication Delivery System

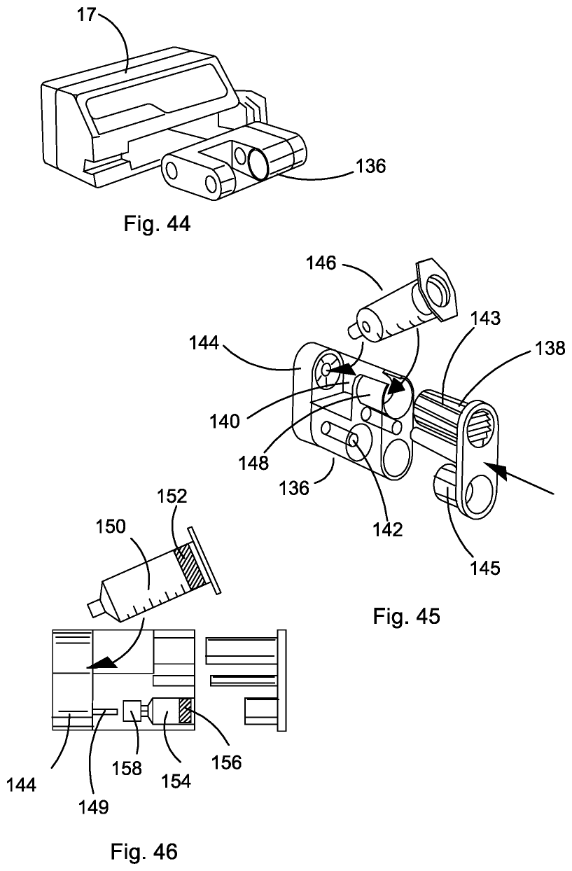

Referring now to FIGS. 44 to 47, FIGS. 44 to 47 depict a particular arrangement of a medication delivery system 91, according to some embodiments. The medication delivery system may comprise an infusion device 14, as previously described. The infusion device 14 may be in the form of a syringe driver 17. The medication delivery system 91 also comprises a medication delivery apparatus 136 in accordance with an alternative embodiment of the present embodiment of the disclosure.

As shown in FIG. 44, the medication delivery apparatus 136 is adapted to be mounted on a syringe driver 17 (which may be the previously described infusion driver). By mounting the medication delivery apparatus 136 onto the syringe driver 17, the syringe driver 17 drives a piston assembly 138 (see FIG. 45) for mixing of the active agent with the diluent in order to prepare the pharmaceutical composition to be delivered to the patient.

The medication delivery apparatus 136 comprises a first plunger 92. The medication delivery apparatus 136 comprises a second plunger 94.

The medication delivery apparatus 136 comprises a body having first and second chambers 140 and 142 for containment, respectively, of the active agent and the diluent. The first and second chambers 98 and 100 are adapted to receive pistons 143 and 145 of the piston assembly 138 in order to apply a pushing force to the active agent and diluent contained in the first and second chambers 98 and 100.

The first chamber 98 is an active agent chamber 98. The active agent chamber 98 may be as previously described. The second chamber 100 is a dilution chamber 100. The dilution chamber 100 may be as previously described.

The medication delivery apparatus 136 comprises a first container 101. The first container 101 is configured to receive at least a portion of the first plunger 92. The medication delivery apparatus 136 comprises a second container 97. The second container 97 is configured to receive at least a portion of the second plunger 94. That is, each of the first container 101 and the second container 97 is configured to receive at least a portion of the piston assembly 138.

The pistons 143 and 145 are slideably received by the first and second chambers 98 and 10 such that when the medication delivery apparatus 136 is mounted on the syringe drive 17, the piston assembly 138 is moved in order that the pistons 143 and 145 enter slideably into the first and second chambers 98 and 100 for applying the pushing force to the pharmaceutical preparation and the diluent.

As shown in FIG. 45, the piston 143 (that pushes the active agent contained in the first chamber 98) is longer than the second piston 145 (that pushes the diluent contained in the second chamber 100). The fact that the first piston 143 is of greater length than the second piston 145 results in that the first piston 143 applies the pushing force to the active agent before the second piston 145 applies the pushing force to the diluent. This allows for the active agent to flow in direction to the first chamber 98 (via a manifold assembly 144) before the diluent is pushed out of the second chamber 100, permitting entrance of the active agent into the second chamber 100 for mixing with the diluent.

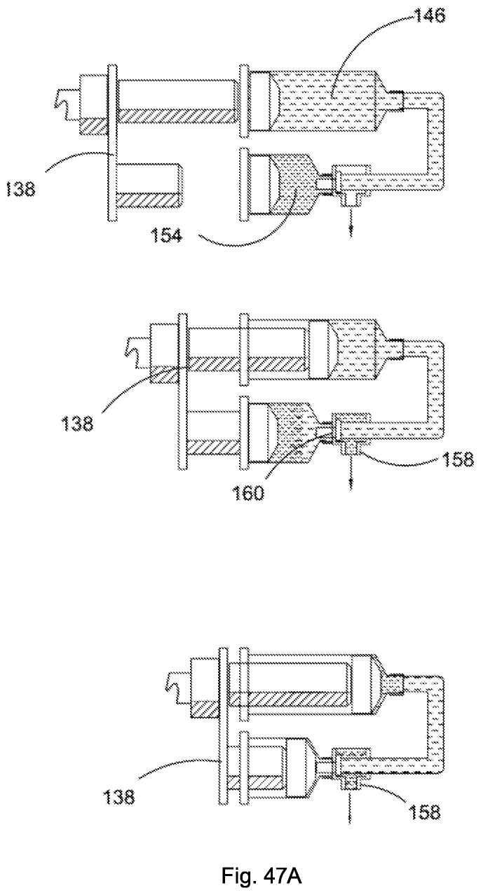

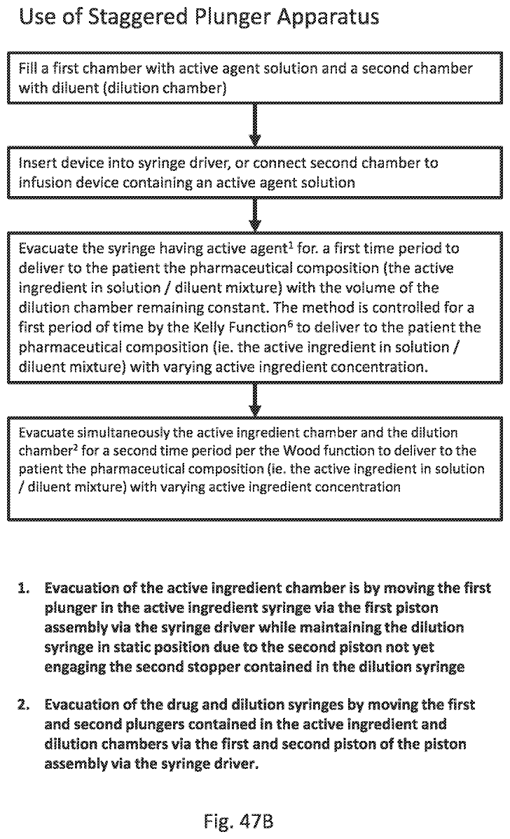

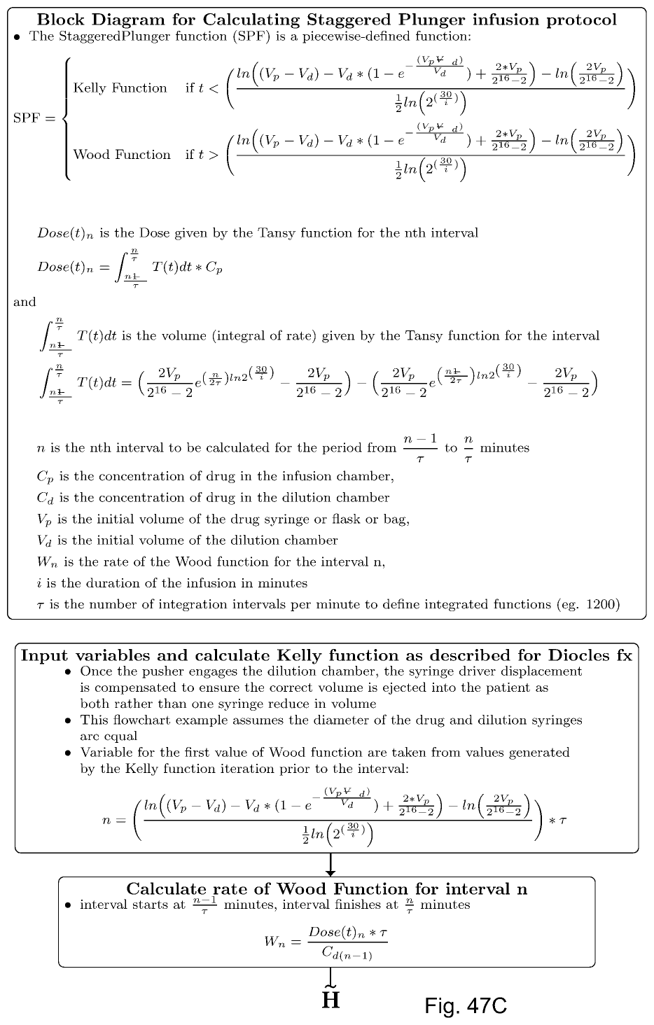

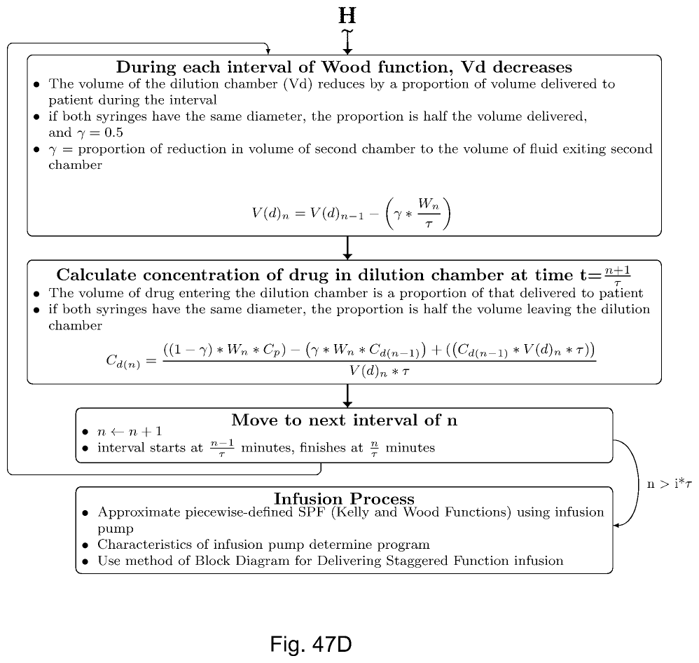

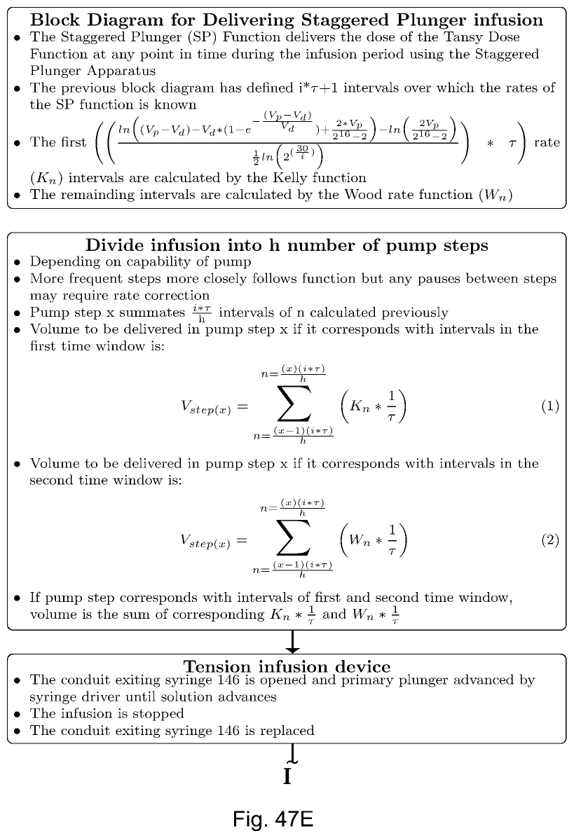

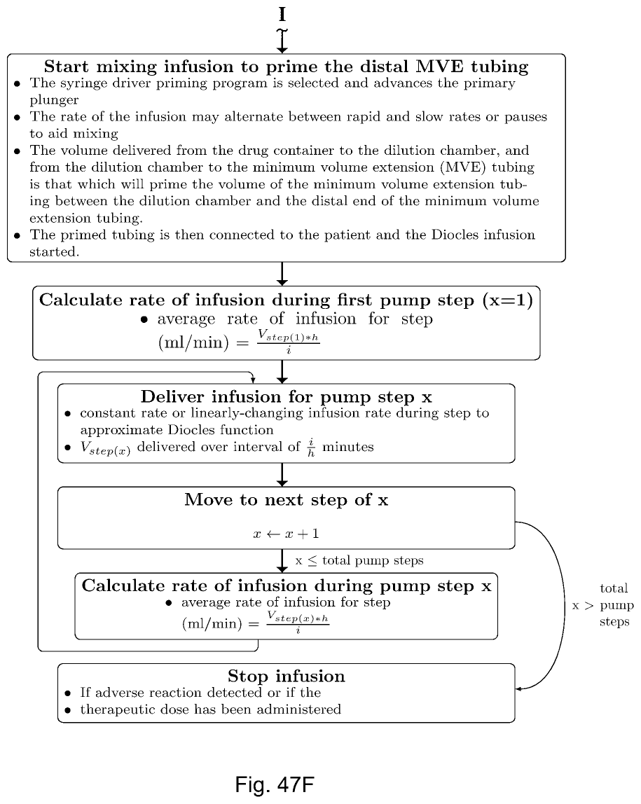

The manifold assembly 144 is adapted to be fluidly connected to the first and second chambers 98 and 100 in order to mix the active agent and the diluent—see FIG. 47. The manifold assembly 144 is adapted to (1) receive the front portion of the syringe 146 to permit the active agent to enter the manifold assembly 144, and (2) being in fluid communication via a conduit 149 with the second chamber 142 for mixing of the active agent with the diluent.

In the particular arrangement shown on FIGS. 44 to 47, the medication delivery apparatus 136 is adapted to receive a syringe 146 containing the active agent. In particular, as shown in FIG. 45, the medication delivery apparatus 136 comprises a snap-on section 148 for receiving a portion of the syringe 146 in order to secure the syringe 146 to the medication delivery apparatus 136.

Further, the syringe 146 comprises a barrel 150 and first seal 152 slideably received within the barrel 150. The barrel 150 may correspond to the first container 101. The first seal 152 may correspond to the first plunger 92. Alternatively, the first plunger 92 may correspond to the first seal 152 and another portion of the piston assembly 138 (e.g. an elongate portion). The first seal 152 impedes the active agent from exiting the syringe 146 and is adapted to receive the pushing force applied by the piston 143 during operation of the dilution chamber 136.

The second chamber 100 is configured as a syringe integrated within the body of the medication delivery apparatus 136. In particular, as shown in FIG. 46, the second chamber 100 comprises a barrel-like space 154 for containment of the diluent having a second seal 156 contained within the space 154 to receive the pushing force applied by the piston 141 during operation of the medication delivery apparatus 136. The second seal 156 may correspond to the second plunger 94. Alternatively, the second plunger 94 may correspond to the second seal 156 and another portion of the piston assembly 138 (e.g. an elongate portion).

Further, the second chamber 100 comprises an outlet 158 for delivering the mixture of the active agent and diluent to the patient. As shown in FIG. 46, the outlet 158 is fluidly connected to the space 154 of the second chamber 142 and the manifold assembly 144; in this manner, the active agent (exiting the syringe 146 and delivered via the manifold assembly 144 to the second chamber 142) may flow into the space 154 for mixing with diluent.

Furthermore, as shown in FIG. 47A, between the space 154 and the outlet 156 of the second chamber 14 there is provided a check valve 160 for controlling entrance of the active agent into the space 154 and exit of the pharmaceutical composition (the mixture of active agent and diluent) from the space 154.

The first container 101 and the first plunger 92 together define the active agent chamber 98. The active agent chamber 98 is configured to receive the pharmaceutical preparation. The active agent chamber comprises an active agent chamber opening. The active agent chamber opening is configured to facilitate the transfer of the pharmaceutical preparation to the dilution chamber 100.

The second container 97 and the second plunger 94 together define the dilution chamber 100. The dilution chamber 100 is configured to receive the diluent. The dilution chamber 100 comprises a dilution chamber opening 121.

The medication delivery apparatus 136 comprises a conduit outlet 95. The conduit outlet 95 is configured to facilitate the transfer of the pharmaceutical preparation from the active agent chamber 98 to the dilution chamber 100 via a conduit 119. The dilution chamber opening 121 is coaxial with the conduit outlet 95. A diameter of the dilution chamber opening 121 is larger than a diameter of the conduit outlet 95. Therefore, the conduit outlet 95 enables outgoing fluid flow from the dilution chamber 100 simultaneously with incoming fluid flow via the conduit opening 121.

The first plunger 92 is configured to be actuated to apply a pushing force to the pharmaceutical preparation within the first container 101 to deliver the pharmaceutical preparation to the second container 97. The second plunger 94 is configured to be actuated to apply a pushing force to the pharmaceutical preparation within the second container 100 to push the pharmaceutical preparation through a medication delivery apparatus outlet 158.

The medication delivery apparatus 136 comprises a valve 123. The valve 123 may define, comprise and/or be in fluid communication with the dilution chamber opening 121. The valve 123 may define, comprise and/or be in fluid communication with the conduit outlet 95. The valve 123 is configured to enable fluid from the active agent chamber 98 to enter the dilution chamber 100 and to inhibit fluid in the dilution chamber 100 from entering the active agent chamber 98. As previously described, the first container 101 (and therefore the active agent chamber 98) and the second container 97 (and therefore the dilution chamber 100) are connected by the conduit 119.

FIGS. 47A to 47F illustrate a method of operation of the dilution chamber 136.

Initially, before the piston assembly 138 is driven based on a particular algorithm and the conduit 30a is fluidly connected to the patient, the syringe driver 17 is operated to drive the piston assembly 138 in such a manner to fill (i.e. to prime) the conduit 30a to be fluidly connected to the patient for delivery of the pharmaceutical composition. As described before, the advantage of priming the conduit 30a is that proper mixing is ensured and that, once the piston assembly 138 is driven based on a particular algorithm and the conduit 30a is fluidly connected to the patient, it is certain that diluted active agent is delivered to the patient.

In particular, the rate of active agent administration is governed by a piecemeal function with two time periods to deliver to the patient the same dose of active agent over time as the Tansy function using the dilution chamber 136.

The first time period (when the piston 145 has not yet engaged the second seal 156 leaving the volume of diluent constant) uses the Kelly function, which is a numerically-integrated function to determine the volumes over time to be delivered to the patient such that the dose of active agent delivered to the patient after mixing in the dilution chamber approximates that of the Tansy Function.

The second time period is controlled by the Wood function. The Wood function is a numerically-integrated function that ensures that the dose of active agent delivered to the patient during the period when the piston 145 has also engaged the second seal 156 is the same as that delivered by the Tansy function. The Wood function compensates the rate of plunger advancement to take into account the fact that over each time interval the space 154 volume diminishes, and that the volume of active agent-containing fluid entering the space 154 is a proportion (related to the relative diameters of the drug and dilution syringes) of diluted active agent containing pharmaceutical composition leaving the space 154 through the outlet 158 for infusion into the patient. The rate of advancement of the piston 156 is compensated so that the volume of pharmaceutical composition infused into the patient for distance of pusher advancement is different (of greater magnitude) prior to the pusher engaging the dilution chamber plunger compared to after (when a lesser magnitude of advancement will result in the same volume of drug entering the patient) engagement. Execution of the Wood function may be referred to as a Wood method

Execution of the Wood Method

The medication delivery system 91 of FIGS. 44 to 47A previously described may be controlled to deliver a pharmaceutical preparation to a patient according to the Wood method of FIGS. 47C to 47F. As previously described, the medication delivery system 91 comprises the medication delivery apparatus 136 and the infusion device (not shown). The infusion device may be similar of the same as a previously described infusion device. The infusion device comprises the at least one infusion device processor and infusion device memory as previously described. The infusion device memory stores program instructions accessible by the at least one infusion device processor. The program instructions are configured to cause the at least one infusion device processor to actuate an infusion device actuator (e.g. syringe driver 17) to control the medication delivery apparatus 136 to deliver medication in accordance with the Wood method.

In particular, the program instructions are configured to cause the at least one infusion device processor to receive a concentration input (Cₚ) that is indicative of a concentration of the pharmaceutical preparation in the active agent chamber. The concentration may be a concentration of active agent in the pharmaceutical preparation. The concentration input (Cₚ) may be received via an input provided by a user. For example, the concentration input (Cₚ) may be input using the user interface 22. Alternatively, the concentration input (Cₚ) may be retrieved from the infusion device memory. Throughout this description, the concentration input (Cₚ) may be a concentration of a drug in, or delivered from the active agent chamber.

The program instructions are further configured to cause the at least one infusion device processor to receive a volume input (Vₚ) that is indicative of a volume of the pharmaceutical preparation. This may be a volume of the pharmaceutical preparation in the active agent chamber 98. The volume input (Vₚ) may be received via an input provided by a user. For example, the volume input (Vₚ) may be input using the user interface 22. Alternatively, the volume input (Vₚ) may be retrieved from the infusion device memory.

The program instructions are further configured to cause the at least one infusion device processor to receive a dilution chamber volume input (V_{d}). The dilution chamber volume input (V_{d}) is indicative of a volume of the dilution chamber 100. The dilution chamber volume input (V_{d}) may be received via an input provided by a user. For example, the dilution chamber volume input (V_{d}) may be input using the user interface 22. Alternatively, the dilution chamber volume input (V_{d}) may be retrieved from the infusion device memory.

The program instructions are further configured to cause the at least one infusion device processor to receive a time input (i). The time input (i) is indicative of a time window over which the pharmaceutical preparation is to be administered. The time input (i) may be received via an input provided by a user. For example, the time input (i) may be input using the user interface 22. Alternatively, the time input (i) may be retrieved from the infusion device memory. The time window comprises a first time window and a second time window.

The program instructions are further configured to cause the at least one infusion device processor to receive an infusion number input (τ). The infusion number input (τ) is indicative of a number of infusion intervals per minute over which a first infusion modelling function and/or a second infusion modelling function are to be numerically approximated over the time window. The infusion modelling function may be the Wood function if FIG. 47C to 47F. The infusion number input (τ) may be received via an input provided by a user. For example, the infusion number input (τ) may be input using the user interface 22. Alternatively, the infusion number input (τ) may be retrieved from the infusion device memory.

The program instructions are further configured to cause the at least one infusion device processor to receive a number of infusion steps (h) that are to be executed during the time window. A first number of infusion steps (h₁) are to be executed during the first time window. A second number of infusion steps (h₂) are to be executed during the second time window. Receiving the number of infusion steps (h) that are to be executed during the time window may comprise receiving an infusion step input that is indicative of the number of infusion steps (h). Alternatively, determining the number of infusion steps (h) that are to be executed during the time window may comprise retrieving the number of infusion steps (h) from the infusion device memory. Receiving the number of infusion steps (h) that are to be executed during the time window may comprise multiplying the time input (i) and the infusion number input (τ).

The at least one infusion device processor numerically approximates the infusion modelling function. This may be a first numerical approximation. In particular, the at least one infusion device processor numerically approximates the infusion modelling function over the first time window. To numerically approximate the infusion modelling function over the first time window, the at least one infusion device processor may perform the functionality described below. That is, numerically approximating the infusion modelling function may comprise the functionality described below. The first infusion modelling function may be the Kelly function. Numerically approximating the first infusion modelling function over the first time window may comprises numerically approximating the Kelly function. This may be performed as previously described.

The at least one processor determines a number of infusion intervals of the first time window. Determining the number of infusion intervals within the first time window of the first numerical approximation comprises multiplying the time input (i) and the infusion number input (τ).

The at least one processor determines an initiating target flow rate parameter (K(0)_{initiating}) The initiating target flow rate parameter is indicative of a target flow rate of the pharmaceutical preparation to be output into the dilution chamber during an initiating infusion interval of the first numerical approximation. The at least one processor may determine the initiating target flow rate parameter (K(0)_{initiating}) as previously described.

The at least one processor determines an initiating pharmaceutical preparation concentration. The initiating pharmaceutical preparation concentration is indicative of an approximated concentration of the pharmaceutical preparation in the dilution chamber after the initiating infusion interval of the first numerical approximation. The at least one processor may determine the initiating pharmaceutical preparation concentration as previously described.

The at least one processor iteratively determines a subsequent target flow rate and a subsequent pharmaceutical preparation concentration for each of a plurality of subsequent infusion intervals of the first numerical approximation. The subsequent target flow rates of the first numerical approximation are each indicative of a target flow rate of the pharmaceutical preparation to be output by the medication delivery apparatus during a respective subsequent infusion interval of the first numerical approximation. The subsequent pharmaceutical preparation concentrations of the first numerical approximation are each indicative of a subsequent approximated concentration of the pharmaceutical preparation in the dilution chamber 100 after the respective subsequent infusion interval. Each of the subsequent target flow rates of the first numerical approximation is determined based at least in part on the subsequent pharmaceutical preparation concentration of a previous infusion interval of the respective infusion interval. Each of the subsequent pharmaceutical preparation concentrations of the first numerical approximation is determined based at least in part on the subsequent target flow rate of the respective subsequent infusion interval.

The at least one infusion device processor numerically approximates the second infusion modelling function. This may be a second numerical approximation. In particular, the at least one infusion device processor numerically approximates the second infusion modelling function over the second time window. To numerically approximate the second infusion modelling function over the second time window, the at least one infusion device processor may perform the functionality described below. That is, numerically approximating the infusion modelling function may comprise the functionality described below.

The at least one processor iteratively determines a subsequent target flow rate, a subsequent dilution chamber volume and a subsequent pharmaceutical preparation concentration for each of a plurality of subsequent infusion intervals of the second numerical approximation. This may be done as previously described herein. For example, this may be done as previously described with reference to the numerical approximation of the Kelly function.

In some embodiments, determining the subsequent target flow rates of the second numerical approximation comprises determining a flow rate parameter Wₙ for each of the subsequent target flow rates of the second numerical approximation. The at least one infusion device processor may do this by calculating:

$${W}_{n} = \frac{Dose {( t )}_{n} * τ}{{C}_{d ( n - 1 )}}$$

where n is the number of the relevant infusion interval, C_{d(n-1)} is a subsequent pharmaceutical preparation concentration of a previous infusion interval of the nth infusion interval and Dose(t)ₙ is a target dose.

Determining the target dose Dose(t)ₙ may comprise determining a dose of a Tansy function T(t), by calculating:

$${\int }_{\frac{n - 1}{τ}}^{\frac{n}{τ}} T ( t ) dt \times {C}_{p}$$

where T(t) is the Tansy function.

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} ) .$$

The subsequent dilution chamber volumes are each indicative of a volume of the dilution chamber after a preceding infusion interval of the respective infusion interval. The subsequent pharmaceutical preparation concentrations of the second numerical approximation are each indicative of a subsequent approximated concentration of the pharmaceutical preparation in the dilution chamber after the respective subsequent infusion interval.

In some embodiments, determining the subsequent dilution chamber volumes of the second numerical approximation comprises calculating, for each subsequent dilution chamber volume:

$${V ( d )}_{n} = {V ( d )}_{n - 1} - ( γ \times \frac{{W}_{n}}{τ} )$$

where V(d)ₙ is the volume of the dilution chamber for the nth infusion interval of the second numerical approximation, V(d)ₙ₋₁ is the volume of the dilution chamber for the n−1 th infusion interval of the second numerical approximation, and y is a proportion of reduction in volume of the dilution chamber relative to a volume of fluid exiting the dilution chamber.

In some embodiments, determining the subsequent pharmaceutical preparation concentrations of the second numerical approximation comprises calculating, for each pharmaceutical preparation concentration:

$${C}_{d ( n )} = \frac{( ( 1 - γ ) \times {W}_{n} \times {C}_{p} ) - ( γ \times {W}_{n} \times {C}_{d ( n - 1 )} ) + ( {C}_{d ( n - 1 )} \times {V ( d )}_{n} \times τ )}{{V ( d )}_{n} \times τ}$$

where C_{d(n)} is the subsequent pharmaceutical preparation concentration for the nth infusion interval of the second numerical approximation and C_{d(n-1)} is the subsequent pharmaceutical preparation concentration for the n−1 th infusion interval of the second numerical approximation.

Each of the subsequent target flow rates of the second numerical approximation is determined based at least in part on the subsequent pharmaceutical preparation concentration of a previous infusion interval of the respective infusion interval. The subsequent pharmaceutical preparation concentrations of the second numerical approximation are determined based at least in part on the subsequent target flow rate of the respective subsequent infusion intervals and the a corresponding subsequent dilution chamber volume.

The at least one infusion device processor determines a first infusion volume for each of the first number of the infusion steps (h₁), based at least in part on the first numerical approximation. This may be executed, for example, as shown in FIGS. 47C to 47F. This may be done, for example, as previously described with reference to the Kelly function.

The at least one infusion device processor determines a second infusion volume for each of the second number of infusion steps (h₂). The at least one infusion device processor may determine a second infusion volume for each of the second number of infusion steps (h₂) based at least in part on the second numerical approximation. This may be done as is shown in FIGS. 47C to 47F.

In some embodiments, determining the second infusion volume for one of the second number of infusion steps (h₂) comprises calculating:

$${V}_{step ( x )} = {\sum }_{n = \frac{( x - 1 ) ( i \times τ )}{h}}^{n = \frac{( x ) ( i \times τ )}{h}} ( {W}_{n} \times \frac{1}{τ} )$$

where Vₛₜₑₚ₍ₓ₎ is the infusion volume of the xth infusion step of the second number of the infusion steps (h₂).

The first and second infusion volumes are indicative of a volume of the pharmaceutical preparation that is to be output by the medication delivery apparatus during the respective infusion steps. For example, one of the first infusion volumes is indicative of a volume of the pharmaceutical preparation that is to be output by the medication delivery apparatus 136 during an infusion step of the first number of infusion steps. Similarly, one of the second infusion volumes is indicative of a volume of the pharmaceutical preparation that is to be output by the medication delivery apparatus 136 during an infusion step of the second number of infusion steps.

In some embodiments, the program instructions are further configured to cause the at least one infusion device processor to determine an infusion rate for each of the infusion steps (h), and wherein determining the infusion rate for one of the infusion steps comprises calculating

$$\frac{{V}_{step ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator such that the determined first infusion volume or second infusion volume for each infusion step is output by the medication delivery apparatus during the respective infusion step at the determined infusion rate.

In some embodiments, the program instructions are further configured to cause the at least one infusion device processor to actuate the infusion device actuator such that the determined first infusion volume or second infusion volume for each infusion step is delivered according to a constant-rate profile or a linearly-changing rate profile.

In some embodiments, the program instructions are further configured to cause the at least one infusion device processor to actuate the infusion device actuator such that the determined first infusion volume or second infusion volume for each infusion step is output by the medication delivery apparatus during the respective subsequent infusion step in bursts.

In some embodiments, the first infusion modelling function is the Kelly function and the second infusion modelling function is the Wood function.

One or more of the above steps may be executed as described and/or shown in FIGS. 47C to 47F.

In accordance with a particular arrangement, the infusion processes based on the functions such as the Sadleir, Diocles and Staggered plunger functions may be complemented using a Pulse-Width Modulation (PWM) digital dilution. FIG. 48 depicts particular arrangements of such PWM digital dilutions.

The PWM digital dilution enables use of multiple short injection pulses to, for example, enhance low volume mixing. The PWM digital dilution comprises delivering during a particular interval of time to the dilution chamber 32 or 100, all of the volume of active agent or fractions thereof as dictated by, for example, the Sadleir, Diocles and Staggered Plunger functions for the particular interval of time of the infusion process. All of the volume of active agent or fractions thereof are delivered to the dilution chamber 32 or 100 over one or more briefer periods of time within the particular internal of time, but at a higher flow rate when compared against the flow rate dictated by, for example, the Sadleir, Diocles and Staggered Plunger functions; thus, delivery of the active agent over one or more briefer periods of time within the particular internal of time act as “bursts”).

In particular, as mentioned before, the methods in accordance with the present embodiments of the disclosure comprise numerical methods that approximate the Sadleir, Diocles and Staggered plunger functions using a number of intervals of time in which the syringe driver 17 (to deliver a particular volume for each interval of time) runs at, for example, (1) a certain constant rate, or (2) a ramp rate from a starting rate to a finishing rate.

The PWM process is a modification of any of the functions (for example, Sadleir, Diocles and Staggered plunger functions) that are used with the dilution chambers 32 or 100. It allows for the total volume of active agent to be given during a particular interval of time (as dictated by any of the functions) to be given over one or more bursts. Each of the bursts delivers to the dilution chambers 32 or 100, the active agent at a greater rate but during a period of time that is shorter than the particular interval of time. This provides greater velocity for mixing, and a period of pause for mixing to occur before the next interval of time.

In a particular arrangement, the volume of active agent that is delivered to the dilution chambers 32 or 100 during the particular interval may be delivered at a slower “baseline” rate than the actual rate dictated by the functions (for example, Sadleir, Diocles and Staggered plunger functions), with one or more faster bursts occurring during the particular interval in order that the total volume of active agent delivered to the dilution chamber 32 or 100 (during the particular interval) is equivalent to the total volume of active agent that should be delivered as dictated by the functions (for example, Sadleir, Diocles and Staggered plunger functions) during the particular interval.

The PWM digital dilution may occur during one or more particular periods of time during the infusion process. The PWM digital dilution is particular useful for use during time intervals starting the infusion process where the flow rate is relatively low.

Further, The PMW digital dilution is particularly advantageous because it allows use of multiple short injections to enhance low-volume mixing.

Another advantage of the PMW digital dilution is that it permits the use of simpler infusion pumps which are capable of only one infusion rate to approximate the functions controlling the infusion process by varying the duration of active infusion rather than rate of active infusion to deliver a target volume over an interval.

### FURTHER EXAMPLES

According to a first aspect of the disclosure there is provided a method for delivering an active ingredient into a patient, the method comprising the steps of preparing a pharmaceutical preparation having a particular volume, the pharmaceutical preparation comprising a solvent and therapeutic dose of the active ingredient and administering to a patient the pharmaceutical preparation, wherein the pharmaceutical preparation is administered to the patient in such a manner that at a first stage of administration of the pharmaceutical preparation at least one portion of the therapeutic dose is administered to the patient for detection of a negative reaction in the patient.

In some embodiments, the therapeutic dose is administered to the patient for detection of an adverse reaction in the patient.

In some embodiments, the pharmaceutical preparation is administered to the patient at varying flow rates.

In some embodiments, the method further comprises accessing a drug library including a database which contains the maximum allowable drug administration rates for each particular drug that may be infused to patients to confirm whether the drug delivery rate exceeds the maximum allowable drug administration rate; and if it does then, the infusion rate will be reduced according to the maximum allowed infusion rate to give the maximum allowable drug administration rate.

In some embodiments, the flow rates vary over time following a curve as dictated by a particular function that at the first stage results in low flow rates such that the at least one portion of the therapeutic dose is administered during the first stage of the administration process.

In some embodiments, the particular function is the Tansy function.

In some embodiments, the Tansy function is given by the equation:

$$T ( t ) = \frac{Vp * ln ( {2}^{( \frac{30}{i} )} )}{{2}^{16} - 2} * {e}^{\frac{t}{2} ln ( {2}^{( \frac{30}{i} )} )}$$

$$T ( t ) = Tansy rate function ( ml / min )$$

$$Vp = primary syringe ( infusion ) volume$$

$$t = time ( min )$$

$$i = duration of infusion ( min )$$

In some embodiments, the method further comprises providing, to an infusion driver having a processor for following instructions of an algorithm used for calculating the equation of the Tansy function, the following variables:

a) volume of pharmaceutical preparation (Vₚ) to be administered to patient in ml, comprising an amount of drug (active ingredient in units of mass) and volume of solvent for mixing with the drug (the active ingredient); and

b) time over which the pharmaceutical preparation is to be administered in minutes (also referred to as the duration of infusion).

In some embodiments, the volume of pharmaceutical preparation to be delivered to the patient in ml (Vₚ or primary syringe (infusion) volume) comprises an amount of drug (active ingredient) mixed in a volume of solvent.

In some embodiments, the amount of therapeutic dose to be delivered to the patient is equal to the concentration of the drug in the solvent multiplied by the total volume of pharmaceutical preparation to be delivered to the patient.

In an arrangement, there may be provided the identity of the particular active ingredient (active ingredient name), dose of active ingredient, and/or maximum active ingredient administration rate (dose/min) for the particular active ingredient to ensure that the maximum active ingredient administration rate is not exceeded during the infusion process.

In some embodiments, the method further comprises the step of providing the pharmaceutical preparation to the entry point of the patient.

In some embodiments, the method further comprises the steps of calculating the flow rate (ml/min) of the pharmaceutical preparation at each point in time during the duration of the infusion as dictated by the Tansy function.

In some embodiments, the method further comprises calculating the cumulative volume of the pharmaceutical preparation infused at each point in time during the infusion as dictated by the Tansy function.

In some embodiments, the method further comprises the steps of programming the infusion driver for approximating the flow rate variation of the pharmaceutical preparation exiting the infusion driver to the flow rate variations as dictated by the Tansy function, wherein the steps comprises:

a) dividing the period of administration into number of infusion steps;

b) calculating the volume of each infusion step;

c) calculate the flow rate for each infusion step;

d) provision of the pharmaceutical preparation to the patient; and

e) delivering in sequential order the pharmaceutical preparation at a flow rate as calculated for each infusion step until culmination of the administration process.

In some embodiments, the step of calculating the flow rate for each infusion step calculates constant or linearly increasing (ramp) flow rates for each infusion step.

In an alternative arrangement, the method further comprises diluting the pharmaceutical preparation prior administration to the patient.

In some embodiments, dilution occurs as the pharmaceutical preparation is delivered from the infusion driver prior to administration to the patient by means of a dilution chamber.

In some embodiments, the dilution chamber contains a particular volume of diluent to which the pharmaceutical preparation will mix during the course of the infusion.

In some embodiments, the pharmaceutical preparation after exiting the dilution chamber comprises a lower concentration of the active ingredient (with respect to the concentration prior to entry into the dilution chamber).

In some embodiments, there is administered to the patient a particular fraction of the dose of the active ingredient, the dose being less than the dose administered at any point in time during the Tansy function.

In some embodiments, the dose is reduced by multiplying the dose as dictated by the Tansy function by

$$\frac{{V}_{p} - {V}_{d} ( 1 - {e}^{- \frac{{V}_{p}}{{V}_{d}}} )}{{V}_{p}}$$

of the dose of the active ingredient administered at any point in time during the Tansy function, wherein V_{d} is the volume of the dilution chamber and Vₚ is the volume of the pharmaceutical preparation prior administration to the patient.

In some embodiments, the flow rate of the pharmaceutical preparation of lower concentration is increased for the majority of the infusion while delivering an amount of pharmaceutical preparation into the patient that is no more than that when delivering the pharmaceutical preparation to the patient without reducing the concentration of the pharmaceutical preparation.

In some embodiments, the minimum flow rate of the pharmaceutical preparation of lower concentration exiting the dilution chamber is increased compared to that delivered by the Tansy method without exceeding the amount of active ingredient delivered at any point in time by the Tansy function.

In some embodiments, the pharmaceutical preparation after exiting the dilution chamber comprises a higher flow rate of a lower concentration of active ingredient and results in an active ingredient dose administered that is equal to

$$\frac{{V}_{p} - {V}_{d} ( 1 - {e}^{- \frac{{V}_{p}}{{V}_{d}}} )}{{V}_{p}}$$

multiplied by the dose administered at each point in time during the equivalent Tansy function, wherein V_{d} is the volume of the dilution chamber and Vₚ is the volume of the pharmaceutical preparation prior to administration to the patient.

In some embodiments, the method further comprises the step, at culmination of the administration process, of delivering any remaining pharmaceutical preparation contained in the dilution chamber to the patient.

Alternatively, the method further comprises the step of discarding the remaining pharmaceutical preparation contained in the chamber.

In some embodiments, the method further comprises providing, to an infusion driver having a processor for following instructions of an algorithm used for calculating the equation of the Sadleir function, the following variables:

a) Volume of the pharmaceutical preparation (Vₚ) in mL to be delivered to the patient, comprising of the volume of solution to give the correct therapeutic dose of drug (active ingredient);

b) Volume of dilution chamber (V_{d});

c) Concentration of drug in primary syringe (e.g. percent of therapeutic dose/ml or units of mass/mL);

d) Time (i) over which the pharmaceutical preparation is to be administered in minutes (also referred to as the duration of infusion); and

e) Number of intervals per minute (t) over which the Sadleir function is calculated.

f) In an arrangement, the identity of the particular drug (drug name), dose of drug, and/or maximum drug administration rate (dose/min) for the particular drug to ensure that the maximum drug administration rate is not exceeded during the infusion process.

In some embodiments, the method further comprises the steps of:

a) calculating the number of intervals during the infusion process over which the values of the dilution chamber concentration are calculated (the number of intervals per minute (t) multiplied by the duration of the infusion in minutes (i));

b) calculating initiating flow rate S(0)_{initiating} of the pharmaceutical preparation at the initiating interval prior commencement of the administration process during a particular time period being as long as any of the intervals of the plurality of subsequent intervals occurring after the initiating interval;

c) calculating the concentration of the active ingredient inside the dilution chamber after the initiating interval at commencement of the administration process;

d) calculating the flow rate as dictated by the Sadleir function for the first subsequent interval of the plurality of subsequent intervals after the initiating interval;

e) calculating the concentration of the active ingredient inside the dilution chamber after occurrence of any of the intervals of the plurality of intervals; and

f) calculating the flow rate as dictated by the Sadleir function for each of the second and subsequent intervals of the plurality of intervals using the concentration of the active ingredient inside the dilution chamber prior to occurrence of each of the second and subsequent intervals.

In some embodiments, the method further comprises the step of calculating the volume administered in each of the plurality of subsequent intervals according to the flow rate as dictated by the Sadleir function for the plurality of subsequent intervals.

In some embodiments, the any of the intervals of the plurality of intervals comprise the first and subsequent intervals.

In some embodiments, the method further comprises the steps of programming the infusion driver for approximating the flow rate variation of the pharmaceutical preparation exiting the infusion driver to the rate flow variations as dictated by the Sadleir function, wherein the steps comprise:

a) dividing the period of administration into a number of infusion steps;

b) calculating the volume of each infusion step;

c) provision of the pharmaceutical preparation to the dilution chamber;

d) mixing the pharmaceutical preparation with the diluent contained in the dilution chamber

e) calculating the flow rate for the first infusion step and subsequent steps; and

f) delivering in sequential order the pharmaceutical preparation at a flow rate as calculated for each infusion step until culmination of the administration process.

In some embodiments, the method further comprises, at completion of the infusion process, the step of providing the diluted pharmaceutical preparation remaining in the dilution chamber to the patient.

In an alternative arrangement, the pharmaceutical preparation remaining in the dilution chamber at completion of the infusion is discarded.

In this alternative arrangement, prior commencement of the infusion process, either: (1) the concentration of the active ingredient in the pharmaceutical preparation in the infusion driver may be increased or (2) the volume of the pharmaceutical preparation in the infusion driver may be increased.

In some embodiments, the increased concentration is equal to the original concentration multiplied by the inverse of the correction factor, being 1/[(Vₚ−V_{d}(1−e^{-Vp/Vd}))/Vₚ].

In some embodiments, the increased volume of the pharmaceutical preparation is calculated by iterating the Kelly function algorithm to determine the final volume infused after completing the infusion.

According to a second aspect of the disclosure there is provided a system for delivering an active ingredient into a patient, the active ingredient being part of a pharmaceutical preparation having a particular volume, the pharmaceutical preparation comprising a solvent and therapeutic dose of the active ingredient, the system comprising an infusion driver having a processor for running instructions of an algorithm for approximating the flow rate variation of the pharmaceutical preparation such that the pharmaceutical preparation is administered to the patient in such a manner that at a first stage of administration of the pharmaceutical preparation at least one portion of the therapeutic dose is administered to the patient for detection of a negative reaction in the patient.

In some embodiments, the algorithm is configured in order that the pharmaceutical preparation exit the infusion driver the rate flow variations as dictated by the Tansy function.

In some embodiments, the system further comprises a dilution chamber fluidly connected between the infusion driver and the patient, the dilution chamber being adapted to reduce the concentration of the pharmaceutical preparation prior entry into the patient.

In some embodiments, the pharmaceutical preparation after exiting the dilution chamber comprises a lower concentration of the active ingredient (with respect to the concentration prior entry into the dilution chamber) such that during administration of the pharmaceutical preparation exiting the dilution chamber there is administered to the patient a particular dose of the active ingredient equal to the product of

$$\frac{{V}_{p} - {V}_{d} ( 1 - {e}^{- \frac{{V}_{p}}{{V}_{d}}} )}{{V}_{p}}$$

multiplied by the does administered at each point as dictated by the Tansy function, wherein V_{d} is the volume of the dilution chamber and Vₚ is the volume of the pharmaceutical preparation prior administration to the patient.

In some embodiments, the algorithm is configured for increasing the flow rate of the pharmaceutical preparation of lower concentration for delivering the same amount of pharmaceutical preparation into the patient

In some embodiments, the algorithm is configured for increasing the flow rate of the pharmaceutical preparation of lower concentration exiting the dilution chamber, for delivering an amount of pharmaceutical preparation into the patient equal to the product of:

$$\frac{{V}_{p} - {V}_{d} ( 1 - {e}^{- \frac{{V}_{p}}{{V}_{d}}} )}{{V}_{p}}$$

multiplied by the dose administered at any point in time as dictated by the Tansy function

In a further alternative arrangement, the method comprises delivering pharmaceutical preparation via a dilution chamber but results in an active ingredient dose administered that is equal to the dose administered at any point in time during the equivalent Tansy function, rather than a dose that is reduced by a fixed fraction as in the previous arrangement. This comprises using a pharmaceutical preparation that has an increased concentration of active ingredient or, alternatively, using a larger volume of pharmaceutical preparation infused over the same period. The alternative modifications are either to:

Increase the concentration of the prepared pharmaceutical preparation (‘Increased concentration Sadleir method’) so that the concentration is

$$\frac{{V}_{p}}{{V}_{p} - {V}_{d} ( 1 - {e}^{- \frac{{V}_{p}}{{V}_{d}}} )}$$

multiplied by the concentration of the equivalent pharmaceutical preparation for the previously described arrangements (Tansy method or Sadleir method). The infusion rates and volumes delivered over the course of the infusion process are unchanged compared to the previously described Sadleir method for the same Vp, Vd and i; or

Increase the volume of the pharmaceutical preparation (‘Increased volume Sadleir method’) delivered at increased rates over the same infusion duration and using the same pharmaceutical preparation concentration as for the previously described arrangements (Tansy method or Sadleir method). The total infusion volume is calculated by an iterative method, where the total volume (ξ) is estimated by iterating the Kelly function algorithm to determine that total volume that would be delivered over the course of the infusion, and the rate of infusion for each interval n is calculated using the Kelly function illustrated in FIGS. 29A and 29B.

In some embodiments, the infusion driver comprises memory means for storing a drug library, and database which contains the maximum allowable drug administration rate for each particular drug that may be infused to patients.

In some embodiments, the processor of the infusion driver runs instructions of an algorithm for accessing the drug library including the database which contains the maximum allowable drug administration rates for each particular drug that may be infused to patients to confirm whether the drug delivery rate exceeds the maximum allowable drug administration rate; and if it does then, the infusion rate will be reduced according to the maximum allowed infusion rate to give the maximum allowable drug administration rate.

In some embodiments, the dilution chamber comprises a container and a manifold connected to the container to permit fluid flow (1) from the infusion driver, via a first conduit and first inlet of the manifold, into the container and (2) from the container, via a first outlet of the manifold, to the patient via a conduit.

In some embodiments, the manifold comprises a second inlet to permit delivery of flushing fluid for flushing of the dilution chamber with the objective of delivering any drug remnant inside the dilution chamber into the patient.

In some embodiments, the manifold comprises a one way valve to allow fluid from the first inlet into the container but to impede fluid flow from the container back into the infusion driver through the first inlet.

In some embodiments, the dilution chamber comprises a catheter having a first end fluidly connected to the first inlet for receiving the pharmaceutical preparation from the infusion driver, and a second end extending into the container.

In one arrangement, the second end of the catheter comprises a blind end impeding fluid flow therethrough, and perforation traversing the sidewall of the catheter.

In another arrangement, the plurality of perforations are arranged in a spaced apart relationship along the length of the catheter and about the outer surface of the catheter permitting exit of the drug through the second end of the catheter in different directions.

In an alternative arrangement, the catheter comprises an end location of the second end of the catheter, the end location comprising the perforations and a sleeve surrounding the end location.

In some embodiments, the sleeve comprises a plurality of perforations arranged in a spaced apart relationship along the length of the end location and about the outer surface of the end location permitting exit of the pharmaceutical preparation through the sleeve in different directions.

In some embodiments, the sleeve is adapted to expand into a circular or elliptical shape during operation thereof.

In some embodiments, at least one of the perforations made in the sleeve traverse diagonally the sleeve in order for the fluid flow, exiting the sleeve through the perforations, is directed towards the first end of the catheter.

In an arrangement, the sleeve is perforated with three evenly spaced, 30 g (0.25 mm) perforations oriented at 60 degrees above the horizontal.

In a further alternative arrangement, the catheter comprises a conical-like truncated end with the enlarged area of the conical-like truncated end comprising the perforations.

In another alternative arrangement, the catheter has an open end permitting exit of fluid flow through the open end of the catheter and into the container.

In some embodiments, the catheter comprises a bubble trap.

In some embodiments, the bubble trap comprises a sleeve surrounding at least partially the first end of the catheter.

In some embodiments, the sleeve extends from a particular location within the manifold to a location outside the manifold such that the distal end of the sleeve is located within the dilution chamber.

In some embodiments, a fluid path is defined between the exterior wall of the catheter and the inner wall of the sleeve.

In some embodiments, the fluid path is adapted to deliver the diluted pharmaceutical preparation to be delivered through the outlet of the manifold to the patient.

In some embodiments, the sleeve extends from the location (within the manifold) where the catheter is attached to an outlet which is fluidly connected to the first inlet of the manifold for delivery of the pharmaceutical preparation flowing through the first inlet of the manifold for delivery into the catheter.

In some embodiments, the fluid path has an open end defined at the distal end of the sleeve for receiving the diluted pharmaceutical preparation, and a sealed end at the particular location within the manifold where the catheter is attached to the outlet for receiving the pharmaceutical preparation from the first inlet; the sealed end ensures that all the diluted pharmaceutical preparation coming from the dilution chamber is delivered to the outlet or delivery to the patient.

In some embodiments, the fluid path is fluidly connected to the outlet of the manifold for delivery of the pharmaceutical preparation into the patient.

In some embodiments, the fluid path comprises a first inlet defined between the distal end of the sleeve and the catheter, the first inlet being adapted to receive the pharmaceutical preparation for delivery into the patient.

In some embodiments, a second inlet is defined between the distal end of the sleeve and the distal end of the manifold onto which the container is connected, the second inlet being adapted to receive the bubbles that have been diverted by the distal end of the sleeve to avoid bubbles from being delivered to the patient.

In some embodiments, the manifold comprises venting means for relieving any excess pressure or removing air bubbles that may be contained in the manifold.

In some embodiments, the dilution chamber comprises a container that is adapted to be selectively displaced between an expanded condition and a contracted condition.

In some embodiments, the container comprises a syringe having a plunger adapted to be selectively displaced for displacing the container between the expanded condition and the contracted condition.

In an arrangement, the infusion processes based on the Sadleir function may be complemented using a Pulse-Width Modulation (PWM) digital dilution.

In some embodiments, the PWM digital dilution comprises delivering during a particular interval of time to the dilution chamber, all of the volume of pharmaceutical preparation or fractions thereof as dictated by the Sadleir function for the particular interval of time of the infusion process with all of the volume of pharmaceutical preparation or fractions thereof being delivered to the dilution chamber over one or more briefer periods of time within the particular internal of time, but at a higher flow rate when compared against the flow rate dictated by, the Sadleir function.

According to a third aspect of the disclosure there is provided a dilution chamber comprising a container and a manifold connected to the container to permit fluid flow (1) from the infusion driver, via a first conduit and first inlet of the manifold, into the container and (2) from the container via a first outlet of the manifold for delivery of the drug to the patient via a conduit.

In some embodiments, the manifold comprises a second inlet to permit delivery of flushing fluid for flushing of the dilution chamber with the objective of delivering any drug remnant inside the dilution chamber into the patient.

In some embodiments, the manifold comprises a one way valve to allow fluid from the first inlet into the container but to impede fluid flow from the container back into the infusion driver through the first inlet.

In some embodiments, the dilution chamber comprises a catheter having a first end fluidly connected to the first inlet for receiving the pharmaceutical preparation from the infusion driver, and a second end extending in the container.

In some embodiments, the dilution chamber comprises a container that is adapted to be selectively displaced between an expanded condition and a contracted condition.

In some embodiments, the container comprises a syringe having a plunger adapted to be selectively displaced for displacing the container between the expanded condition and the contracted condition.

According to a fourth aspect of the disclosure there is provided a catheter for insertion in the dilution chamber in accordance with the third aspect of the disclosure, the catheter having a first end fluidly connected to the first inlet of the dilution chamber for receiving the pharmaceutical preparation from the infusion driver, and a second end extending in the container.

In one arrangement, the second end of the catheter comprises a blind end impeding fluid flow therethrough, and perforations traversing the side wall of the catheter.

In another arrangement, the plurality of perforations arranged in a spaced apart relationship along the length of the catheter and about the outer surface of the catheter permitting exit of the drug through the second end of the catheter in different directions.

In an alternative arrangement, the catheter comprises an end location of the second end of the catheter, the end location comprising the perforations and a sleeve surrounding the end location.

In some embodiments, the sleeve comprises a plurality of perforations arranged in a spaced apart relationship along the length of the end location and about the outer surface of the end location permitting exit of the pharmaceutical preparation through the sleeve in different directions.

In some embodiments, the sleeve is adapted to expand into a circular or elliptical shape during operation thereof.

In some embodiments, at least one of the perforations made in the sleeve traverse diagonally the sleeve in order for the fluid flow, exiting the sleeve through the perforations, is directed towards the first end of the catheter.

In another alternative arrangement, the catheter comprises a blind end having plurality of perforations with the catheter being made out of a flexible material adapted to be expanded as the flow rate of the pharmaceutical preparation increases.

In a further alternative arrangement, the catheter comprises a conical-like truncated end with the enlarged area of the conical-like truncated end comprising the perforations.

In another alternative arrangement, the catheter has an open end permitting exit of fluid flow through the open end of the catheter and into the container.

In some embodiments, the catheter comprises a bubble trap.

In some embodiments, the bubble trap comprises a sleeve surrounding at least partially the first end of the catheter.

In some embodiments, the sleeve extends from a particular location within the manifold to a location outside the manifold such that the distal end of the sleeve is located within the dilution chamber.

In some embodiments, a fluid path is defined between the exterior wall of the catheter and the inner wall of the sleeve.

In some embodiments, the fluid path is adapted to deliver the diluted pharmaceutical preparation to be delivered through the outlet of the manifold to the patient.

In some embodiments, the sleeve extends from the location (within the manifold) where the catheter attached to an outlet which is fluidly connected to the first inlet of the manifold for delivery of the pharmaceutical preparation flowing through the first inlet of the manifold for delivery into the catheter.

In some embodiments, the fluid path is fluidly connected to the first outlet of the manifold for delivery of the pharmaceutical preparation into the patient.

In some embodiments, the fluid path comprises a first inlet defined between the distal end of the sleeve and the catheter, the first inlet being adapted to receive the pharmaceutical preparation for delivery into the patient.

In some embodiments, a second inlet is defined between the distal end of the sleeve and the distal end of the manifold onto which the container is connected, the second inlet being adapted to receive the bubbles that have been diverted by the distal end of the sleeve to avoid bubbles from being delivered to the patient.

In some embodiments, the manifold comprises venting means for relieving any excess pressure or removing air bubbles that may be contained in the manifold.

According to a fifth aspect of the disclosure there is provided a bubble trap for use in dilution chamber as defined in the third aspect of the disclosure, the bubble trap being adapted to deviate any bubble forming at the first end of the catheter located within the container of the dilution chamber and floating adjacent the catheter preventing any bubble from being delivered to the patient.

In some embodiments, the bubble trap comprises a sleeve surrounding at least partially the first end of the catheter.

In some embodiments, the sleeve extends from a particular location within the manifold to a location outside the manifold such that the distal end of the sleeve is located within the dilution chamber.

In some embodiments, a fluid path is defined between the exterior wall of the catheter and the inner wall of the sleeve.

In some embodiments, the fluid path is adapted to deliver the diluted pharmaceutical preparation to be delivered through the outlet of the manifold to the patient.

In some embodiments, the sleeve extends from the location (within the manifold) where the catheter attached to an outlet which is fluidly connected to the first inlet of the manifold for delivery of the pharmaceutical preparation flowing through the first inlet of the manifold for delivery into the catheter.

In some embodiments, the fluid path is fluidly connected to the first outlet of the manifold for delivery of the pharmaceutical preparation into the patient.

In some embodiments, the fluid path comprises a first inlet defined between the distal end of the sleeve and the catheter, the first inlet being adapted to receive the pharmaceutical preparation for delivery into the patient.

In some embodiments, a second inlet is defined between the distal end of the sleeve and the distal end of the manifold onto which the container is connected, the second inlet being adapted to receive the bubbles that have been diverted by the distal end of the sleeve to avoid bubbles from being delivered to the patient.

In some embodiments, the manifold comprises venting means for relieving any excess pressure or removing air bubbles that may be contained in the manifold.

In a particular arrangement of the first embodiment of the disclosure there is provided a method and system (referred to as the Tansy Method) for delivering to a patient via intravenous infusion, from a single pharmaceutical preparation container, the pharmaceutical drug. The dose is delivered at a rate that varies over the duration of the infusion such that orders of magnitude of different cumulative doses, and orders of magnitude of different rates of dose administration, are separated in time. For example, after 3% of the infusion time has elapsed, 0.001% of the cumulative dose has been administered. After 14% of the infusion time, 0.01% of the cumulative dose has been administered.

After 34%, 56%, 78% and 100% of the infusion times have elapsed, 0.1%, 1%, 10%, and 100% of the cumulative dose has been administered. Similarly, the rate of drug administration increases as the infusion progresses. The rate of drug administration after 11% of the infusion time has elapsed is 0.01% of maximal. After 34%, 56%, 78% and 100% of the infusion times, 0.1%, 1%, 10% and 100% of the maximum drug administration rate is achieved.

In a particular arrangement of the second embodiment of the disclosure (referred to as the Sadleir Method) describes a method and system of delivering to the patient the same profile of drug administration from a single pharmaceutical container (although the concentration of drug within the container will have to be increased by an amount dependent on characteristics of the delivery system if the same cumulative doses and dosing rates are to be achieved during the infusion), but in which a dilution chamber within the delivery apparatus reduces the concentration of drug in solution delivered to the patient as it is being delivered. This requires that the rate of fluid infusion during the early stages of the infusion be increased to compensate for the difference in delivered drug concentration. This increased rate of fluid infusion reduces errors or inaccuracies associated with low fluid infusion rates.

In some embodiments, some of the embodiments of the disclosure allow the delivery of cumulative doses or rates of dose administration in which orders of magnitude of change are separated in time. This allows a negative (or adverse) reaction to be detected during the course of a therapeutic infusion and the infusion to be stopped before a dose that would cause a more severe reaction has been administered. Alternatively, it may induce desensitization, preventing or reducing the severity of a reaction in a patient that would otherwise have suffered a negative (or adverse) reaction.

According to a sixth aspect of the disclosure there is provided a dilution chamber comprising a container defining an inner volume and having at least one inlet for receiving at least one first fluid and an outlet for discharging a second fluid, a first plunger for applying a pushing force to at least the first fluid and a second plunger for dividing the inner volume of the container into a first chamber and a second chamber, wherein the second plunger is adapted to allow fluid flow between the first chamber and the second chamber.

In some embodiments, the second comprises valve means for allowing fluid flow between the first chamber and the second chamber.

In some embodiments, the second chamber is fluidly connected to the outlet for allowing the fluid contained in the second chamber to be discharged from the container for infusion into a patient.

In some embodiments, the valve means comprise a check valve impeding fluid flow from the second chamber into the first chamber.

In some embodiments, the outlet is adapted to permit a third fluid to enter the second chamber.

In some embodiments, the inlet is adapted to permit the first fluid to enter the first chamber.

In some embodiments, the second plunger comprises stirring means for mixing the first and third fluid for generating the second fluid when the first fluid enters the second chamber due to being applied the pushing force generated by the first plunger.

In some embodiments, the stirring means are driven by fluid flow flowing through the valve means of the second plunger.

In some embodiments, the container comprises a barrel of a syringe, the first plunger being the plunger of the syringe.

In some embodiments, the syringe is adapted to be received by a syringe driver, the syringe driver being adapted for driving of the first plunger to apply a pushing force during a first period of time to the first fluid contained in the first chamber for delivering the first fluid into the second chamber for mixing of the first fluid with the third fluid contained in the second chamber for generating the second fluid.

In some embodiments, the syringe driver is adapted to apply a pushing force during a second period of time to the first plunger for moving the second plunger for discharging the second fluid via the outlet into a conduit for infusion into a patient.

In some embodiments, the syringe driver is controlled by algorithms replicating a Diocles function.

In some embodiments, the dose administered over time according to the Diocles function is equal to the dose administered over time for the equivalent Tansy function with the same volume of pharmaceutical preparation (Vₚ), concentration of drug from the primary pharmaceutical container (Cₚ) and duration of the infusion process (i).

In a particular arrangement, the dilution chamber comprises a plunger lock for keeping the first plunger in a particular position, the particular position being such that during insertion of the first fluid into the first chamber, the first fluid enters the second chamber for mixing with the third fluid for generating the second fluid.

In some embodiments, the dilution chamber comprising the plunger lock is adapted to be fluidly connected to a syringe driver having a syringe comprising the first fluid.

In some embodiments, the syringe driver is adapted for driving of the plunger of the syringe to apply a pushing force during a first period of time for delivering the first fluid to the dilution chamber comprising the third fluid for generating the second fluid

In some embodiments, the concentration of the first fluid contained in the dilution chamber increases during the process of infusion of the second fluid to the patient.

In some embodiments, the syringe driver is controlled by algorithms replicating the Sadleir infusion protocol for delivering the first fluid to the dilution chamber having the plunger lock.

In some embodiments, the first fluid is a pharmaceutical preparation comprising an active agent, the third fluid a diluent, and the second fluid comprises a pharmaceutical composition prepared by mixing the first and third fluid.

In some embodiments, the concentration of the active agent contained in the dilution chamber increases during the process of infusion of the pharmaceutical composition to the patient.

In an arrangement, the infusion processes based on the Diocles function may be complemented using a Pulse-Width Modulation (PWM) digital dilution.

In some embodiments, the PWM digital dilution comprises delivering during a particular interval of time to the dilution chamber, all of the volume of active agent or fractions thereof as dictated by the Diocles function for the particular interval of time of the infusion process with all of the volume of active agent or fractions thereof being delivered to the dilution chamber over one or more briefer periods of time within the particular internal of time, but at a higher flow rate when compared against the flow rate dictated by, the Diocles function.

According to a seventh aspect of the disclosure there is provided a dilution chamber comprising a first chamber and a second chamber fluidly connected with respect to each other, a first piston to be slideably received within the first chamber for applying a pushing force to a first fluid contained in the first chamber for delivering the first fluid to the second chamber, and a second piston to be slideably received within the second chamber for applying a pushing force to a second fluid contained in the second chamber, wherein the first piston is adapted to apply the pushing force during a first period of time and the second piston is adapted to apply the pushing force during a second period of time, the first period of time starting before the second period of time.

In some embodiments, the dilution chamber further comprises an outlet fluidly connected to the second chamber for delivering a third fluid being the mixture of the first and second fluid to a patient.

In some embodiments, the dilution chamber further comprises a piston assembly having the first and second pistons, wherein the first piston is longer than the second piston.

In some embodiments, the first chamber is adapted to receive a syringe containing the first fluid and being adapted to receive the first piston.

In some embodiments, the dilution chamber is adapted to be received by a syringe driver, the syringe driver being adapted for driving of the piston assembly.

In some embodiments, the syringe driver is controlled by algorithms replicating the Kelly infusion protocol during a first period of time and the syringe driver is controlled by algorithms replicating the Wood infusion protocol during a second period of time.

In some embodiments, the dose administered over time according to the Kelly function during the first period of time and the Wood function during the second period of time is equal to the dose administered over time for the equivalent Tansy function with the same volume of pharmaceutical preparation (Vₚ), concentration of drug from the primary pharmaceutical container (Cₚ) and duration of the infusion process (i).

In some embodiments, the first fluid comprises pharmaceutical preparation comprising an active agent, the second fluid a diluent and the third fluid comprises a pharmaceutical composition prepared by mixing the first and second fluid.

In some embodiments, the concentration of the first fluid contained in the dilution chamber increases during the process of infusion of the second fluid to the patient.

In an arrangement, the infusion processes based on the Staggered Plunger function may be complemented using a Pulse-Width Modulation (PWM) digital dilution.

In some embodiments, the PWM digital dilution comprises delivering during a particular interval of time to the dilution chamber, all of the volume of pharmaceutical preparation or fractions thereof as dictated by the Staggered Plunger function for the particular interval of time of the infusion process with all of the volume of pharmaceutical preparation or fractions thereof being delivered to the dilution chamber over one or more briefer periods of time within the particular internal of time, but at a higher flow rate when compared against the flow rate dictated by, the Staggered Plunger.

In some embodiments, there is provided a medication delivery apparatus. The medication delivery apparatus may comprise: a first plunger; a second plunger; and a container configured to receive the second plunger and at least a portion of the first plunger; wherein: the container and the second plunger together define a dilution chamber that is configured to receive a diluent, the dilution chamber comprising a dilution chamber opening, the dilution chamber opening being defined by the container; the first plunger, the container and the second plunger together define an active agent chamber that is configured to receive a pharmaceutical preparation, the active agent chamber comprising a first active agent chamber opening configured to receive the at least a portion of the first plunger; and the second plunger comprises a valve configured to control a flow of pharmaceutical preparation from the active agent chamber to the dilution chamber in response to applied pressure.

In some embodiments, the first plunger and the second plunger are each configured to be displaced with respect to a longitudinal axis of the container.

In some embodiments, the second plunger is disposed between the first plunger and the dilution chamber opening.

In some embodiments, the active agent chamber comprises a second active agent chamber opening in a wall of the container.

In some embodiments, the active agent chamber is configured to receive the pharmaceutical preparation through the second active agent chamber opening.

In some embodiments, the second plunger is disposed between the second active agent chamber opening and the dilution chamber opening.

In some embodiments, the container defines an inner container surface.

In some embodiments, the first plunger comprises a first plunger sealing surface that is configured to seal with the inner container surface to inhibit fluid flow between the inner container surface and the first plunger sealing surface.

In some embodiments, the second plunger comprises a second plunger sealing surface that is configured to seal with the inner container surface to inhibit fluid flow between the inner container surface and the second plunger sealing surface.

In some embodiments, the valve comprises an inlet side and an outlet side.

In some embodiments, the valve is configured to move from a closed position to an open position upon application of pressure to the inlet side.

In some embodiments, the valve is configured to move from the open position to the closed position upon removal of the pressure applied to the inlet side.

In some embodiments, the valve is biased toward the closed position.

In some embodiments, the valve comprises a plurality of flaps that are configured to separate upon application of pressure to the inlet side.

In some embodiments, the medication delivery apparatus further comprises a conduit. The conduit may be configured to be fluidly connected to the dilution chamber opening.

In some embodiments, the conduit is of a predetermined volume.

In some embodiments of the present disclosure, there is provided a medication delivery system. The medication delivery system comprises the medication delivery apparatus and an infusion device. The infusion device comprises at least one infusion device processor; and infusion device memory storing program instructions accessible by the at least one infusion device processor.

In some embodiments, the program instructions are configured to cause the at least one infusion device processor to: receive a volume input (Vₚ) that is indicative of a volume of the pharmaceutical preparation, receive a time input (i) that is indicative of a time over which the pharmaceutical preparation is to be administered; receive a number of infusion steps (h) that are to be executed during the time over which the pharmaceutical preparation is to be administered; determine a pharmaceutical preparation output volume for each of the infusion steps of the number of infusion steps, each pharmaceutical preparation output volume corresponding to a volume of the pharmaceutical preparation that is to be output by the medication delivery apparatus during the respective infusion step; determine a target flow rate of each infusion step, each target flow rate being indicative of a target flow rate of the pharmaceutical preparation to be output by the medication delivery apparatus during the respective infusion step, wherein each target flow rate is determined based at least in part on the pharmaceutical preparation output volume of the respective infusion step; and actuate an infusion device actuator to displace the first plunger such that the pharmaceutical preparation is output by the medication delivery apparatus at the respective target flow rate during each infusion step.

In some embodiments, the program instructions are further configured to cause the at least one infusion device processor to receive a pharmaceutical preparation input, the pharmaceutical preparation input being indicative of one or more of: an identity of the pharmaceutical preparation; a dose of the pharmaceutical preparation; and a maximum pharmaceutical preparation administration rate.

In some embodiments, the target flow rate is limited at the maximum pharmaceutical preparation administration rate, such that the target flow rate does not exceed the maximum pharmaceutical preparation administration rate during infusion.

In some embodiments, receiving the number of infusion steps comprises: receiving an infusion step input that is indicative of the number of infusion steps; or retrieving the number of infusion steps from the infusion device memory.

In some embodiments, determining the pharmaceutical preparation output volume for each of the number of infusion steps comprises integrating a Tansy function between a first time that corresponds to a start of the relevant infusion step, and a second time that corresponds to an end of the relevant infusion step.

In some embodiments, the Tansy function T(t) is defined by:

$$T ( t ) = \frac{{V}_{p} \times ln {2}^{( \frac{30}{i} )}}{{2}^{16} - 2} {e}^{\frac{t}{2} ln {2}^{( \frac{30}{i} )}}$$

where:
V_{P} is the volume input;
t is the time; and
i is the time input.

In some embodiments, determining the pharmaceutical preparation output volume for each of the number of infusion steps comprises calculating:

∫ₙ₋₁ⁿT(t)dt

In some embodiments, determining the target flow rate of each infusion step comprises dividing the pharmaceutical preparation output volume of a respective infusion step by a length of that infusion step.

In some embodiments, determining the target flow rate of each infusion step comprises determining an initial target flow rate and a final target flow rate for each infusion step, wherein the initial target flow rate of a respective infusion step is equal to the final target flow rate of a preceding infusion step, and the final target flow rate of the respective infusion step is equal to the initial target flow rate of the following infusion step.

In some embodiments, the program instructions are configured to cause the at least one infusion device processor to: receive: a concentration input (Cₚ) that is indicative of a concentration of the pharmaceutical preparation in the active agent chamber; a volume input (Vₚ) that is indicative of a volume of the pharmaceutical preparation that is to be infused, a dilution chamber volume input (V_{d}) that is indicative of a volume of the dilution chamber; a time input (i) that is indicative of a time window over which the pharmaceutical preparation is to be administered; an infusion number input (τ) that is indicative of a number of infusion intervals per minute over which an infusion modelling function is to be numerically approximated over the time window; a number of infusion steps (h) that are to be executed during the time window; numerically approximate the infusion modelling function over the time window, wherein numerically approximating the infusion modelling function comprises: determining a number of infusion intervals within the time window; determining an initiating target flow rate parameter (S(0)_{initiating}), the initiating target flow rate parameter being indicative of a target flow rate of the pharmaceutical preparation to be output by the medication delivery apparatus during an initiating infusion interval of the numerical approximation; determining an initiating pharmaceutical preparation concentration, the initiating pharmaceutical preparation concentration being indicative of an approximated concentration of the pharmaceutical preparation in the dilution chamber after the initiating infusion interval of the numerical approximation; iteratively determining a subsequent target flow rate and a subsequent pharmaceutical preparation concentration for each of a plurality of subsequent infusion intervals of the numerical approximation, wherein: the subsequent target flow rates are each indicative of a target flow rate of the pharmaceutical preparation to be output by the medication delivery apparatus during a respective subsequent infusion interval of the numerical approximation; the subsequent pharmaceutical preparation concentrations are each indicative of a subsequent approximated concentration of the pharmaceutical preparation in the dilution chamber after the respective subsequent infusion interval; each of the subsequent target flow rates is determined based at least in part on the subsequent pharmaceutical preparation concentration of a previous infusion interval of the respective infusion interval; and each of the subsequent pharmaceutical preparation concentrations is determined based at least in part on the subsequent target flow rate of the respective subsequent infusion interval; determine an infusion volume for each of the number of infusion steps (h), based at least in part on the numerical approximation, the infusion volumes being indicative of a volume of the pharmaceutical preparation that is to be output by the medication delivery apparatus during the respective infusion step; and actuate an infusion device actuator to displace the first plunger such that the first infusion volume or the second infusion volume for each infusion step is output by the medication delivery apparatus during the respective infusion step.

In some embodiments, receiving the number of infusion steps that are to be executed during the time over which the pharmaceutical preparation is to be administered comprises: receiving an infusion step input that is indicative of the number of infusion steps; or retrieving the number of infusion steps from the infusion device memory.

In some embodiments, the program instructions are further configured to cause the at least one infusion device processor to receive a pharmaceutical preparation input, the pharmaceutical preparation input being indicative of one or more of: an identity of the pharmaceutical preparation; a dose of the pharmaceutical preparation; and a maximum pharmaceutical preparation administration rate.

In some embodiments, the subsequent target flow rates are limited at the maximum pharmaceutical preparation administration rate, such that the subsequent target flow rates do not exceed the maximum pharmaceutical preparation administration rate.

In some embodiments, determining the number of infusion intervals within the time window of the numerical approximation comprises multiplying the time input (i) and the infusion number input (τ).

In some embodiments, determining the initiating target flow rate parameter (S(0)_{initiating}) comprises calculating:

$$\sqrt{( ( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) ) * ( \frac{{V}_{p} - ( {V}_{d} * ( 1 - {e}^{- \frac{{V}_{p}}{{V}_{d}}} ) )}{{V}_{p}} ) ) * {τ}^{2} * {V}_{d}} .$$

In some embodiments, determining the initiating pharmaceutical preparation concentration comprises calculating:

$${C}_{{d}_{( o )}} = \frac{( {S ( 0 )}_{initiating} \times {C}_{p} ) - ( {S ( 0 )}_{initiating} \times {C}_{{d}_{( - 1 )}} ) + ( {C}_{{d}_{( - 1 )}} \times {V}_{d} \times τ )}{{V}_{d} \times τ}$$

where C_{d}_{_{(−1)}}=0 and C_{d}_{₍ₒ₎} is the initiating pharmaceutical preparation concentration.

In some embodiments, determining a subsequent target flow rate for one of the plurality of subsequent infusion intervals of the numerical approximation comprises determining a flow rate parameter Sₙ where n is the number of the relevant infusion interval; and wherein determining the flow rate parameter Sₙ comprises determining a dose parameter D_{mtf}(t)ₙ.

In some embodiments, determining the dose parameter D_{mtf}(t)ₙ comprises calculating:

$${{D}_{mtf} ( t )}_{n} = {\int }_{\frac{n - 1}{τ}}^{\frac{n}{τ}} T ( t ) dt \times {C}_{p} \times ( \frac{{V}_{p} - ( {V}_{d} \times ( 1 - {e}^{- \frac{{V}_{p}}{{V}_{d}}} ) )}{{V}_{p}} )$$

where:
T(t) is a Tansy rate function;
Cₚ is the concentration input;
Vₚ is the volume input;
V_{ci} is the dilution chamber volume input;
n is the number of the relevant infusion interval; and
τ is the infusion number input.

In some embodiments, determining the flow rate parameter Sₙ comprises calculating:

$${S}_{n} = \frac{{{D}_{mtf} ( t )}_{n} \times τ}{{C}_{d ( n - 1 )}}$$

where n is the number of the relevant infusion interval, C_{d(n-1)} is a subsequent pharmaceutical preparation concentration of a previous infusion interval of the nth infusion interval and D_{mtf}(t)ₙ is the dose parameter.

In some embodiments, determining the subsequent pharmaceutical preparation concentrations of the numerical approximation comprises calculating:

$${C}_{d ( n )} = \frac{( {S}_{n} \times {C}_{p} ) - ( {S}_{n} \times {C}_{d ( n - 1 )} ) + ( {C}_{d ( n - 1 )} \times {V}_{d} \times τ )}{{V}_{d} \times τ}$$

where C_{d(n)} is the subsequent pharmaceutical preparation concentration for the n th infusion interval of the numerical approximation and C_{d(n-1)} is the subsequent pharmaceutical preparation concentration for the n−1 th infusion interval of the numerical approximation.

In some embodiments, determining the initiating target flow rate (S(0)_{initiating}) comprises calculating:

$$\sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) \times {V}_{d} \times {τ}^{2}} .$$

In some embodiments, determining the dose parameter comprises determining a dose of the Tansy function, by calculating:

$${\int }_{\frac{n - 1}{τ}}^{\frac{n}{τ}} T ( t ) dt \times {C}_{p} .$$

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) ln {2}^{( \frac{30}{i} )}} - \frac{2 {V}_{p}}{{2}^{16} - 2} ) - ( \frac{2 {V}_{p}}{{2}^{16} - 2} {e}^{( \frac{n - 1}{2 τ} ) ln {2}^{( \frac{30}{i} )}} - \frac{2 {V}_{p}}{{2}^{16} - 2} ) .$$

In some embodiments, determining the infusion volume for one of the infusion steps comprises calculating:

$${V}_{step ( x )} = {{\sum }^{n = \frac{( x ) ( i \times τ )}{h}}}_{n = \frac{( x - 1 ) ( i \times τ )}{h}} ( {S}_{n} \times \frac{1}{τ} )$$

where Vₛₜₑₚ₍ₓ₎ is the infusion volume of the xth infusion step.

In some embodiments, the program instructions are further configured to cause the at least one infusion device processor to determine an infusion rate for each of the infusion steps, and wherein determining the infusion rate for one of the infusion steps comprises calculating

$$\frac{{V}_{step ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator such that the determined infusion volume for each infusion step is output by the medication delivery apparatus during the respective infusion step at the determined infusion rate.

In some embodiments, the program instructions are further configured to cause the at least one infusion device processor to actuate the infusion device actuator such that the determined infusion volume for each infusion step is delivered according to a constant-rate profile or a linearly-changing rate profile.

In some embodiments, the program instructions are further configured to cause the at least one infusion device processor to actuate the infusion device actuator such that the determined infusion volume for each infusion step is output by the medication delivery apparatus during the respective subsequent infusion step in bursts.

In some embodiments, the concentration input Cₚ is increased by a factor of

$$( \frac{{V}_{p}}{{V}_{p} - ( {V}_{d} ( 1 - {e}^{- \frac{{V}_{p}}{{V}_{d}}} ) )} ) .$$

In some embodiments, the infusion modelling function is a Sadleir function.

In some embodiments, the program instructions are configured to cause the at least one infusion device processor to: receive: a concentration input (Cₚ) that is indicative of a concentration of the pharmaceutical preparation in the active agent chamber; a volume input (Vₚ) that is indicative of a volume of the pharmaceutical preparation, a dilution chamber volume input (V_{d}) that is indicative of a volume of the dilution chamber; a time input (i) that is indicative of a time window over which the pharmaceutical preparation is to be administered, the time window comprising a first time window and a second time window; an infusion number input (τ) that is indicative of a number of infusion intervals per minute over which an infusion modelling function is to be numerically approximated over the first time window; a number of infusion steps (h) that are to be executed during the tine window, wherein a first number of the infusion steps (h₁) are to be executed during the first time window and a second number of the infusion steps (h₂) are to be executed during the second time window; numerically approximate the infusion modelling function over the first time window, wherein numerically approximating the infusion modelling function comprises: determining a number of infusion intervals of the first time window; determining an initiating target flow rate parameter (K(0)_{initiating}), the initiating target flow rate parameter being indicative of a target flow rate of the pharmaceutical preparation to be output into the dilution chamber during an initiating infusion interval of the numerical approximation; determining an initiating pharmaceutical preparation concentration, the initiating pharmaceutical preparation concentration being indicative of an approximated concentration of the pharmaceutical preparation in the dilution chamber after the initiating infusion interval of the numerical approximation; iteratively determining a subsequent target flow rate and a subsequent pharmaceutical preparation concentration for each of a plurality of subsequent infusion intervals of the numerical approximation; wherein the subsequent target flow rates are each indicative of a target flow rate of the pharmaceutical preparation to be output by the medication delivery apparatus during a respective subsequent infusion interval of the numerical approximation; the subsequent pharmaceutical preparation concentrations are each indicative of a subsequent approximated concentration of the pharmaceutical preparation in the dilution chamber after the respective subsequent infusion interval; each of the subsequent target flow rates is determined based at least in part on the subsequent pharmaceutical preparation concentration of a previous infusion interval of the respective infusion interval; and each of the subsequent pharmaceutical preparation concentrations is determined based at least in part on the subsequent target flow rate of the respective subsequent infusion interval; determine a first infusion volume for each of the first number of the infusion steps (h₁), based at least in part on the numerical approximation, the infusion volume being indicative of a volume of the pharmaceutical preparation that is to be output by the medication delivery apparatus during the respective infusion step; determine a number of infusion intervals of the second time window; determine a target dose Dose(t)ₙ for each of the number of infusion intervals of the second time window; determine a target flow rate Dₙ for each of the number of infusion intervals of the second time window, based at least in part on the target dose for the respective infusion interval; determine a second infusion volume for each of the second number of infusion steps (h₂) based at least in part on the target flow rate; and actuate an infusion device actuator to displace the first plunger such that the first infusion volume or the second infusion volume for each infusion step (h) is output by the medication delivery apparatus during the respective infusion step.

In some embodiments, receiving the number of infusion steps that are to be executed during the time window comprises: receiving an infusion step input that is indicative of the number of infusion steps; or retrieving the number of infusion steps from the infusion device memory.

In some embodiments, the program instructions are further configured to cause the at least one infusion device processor to receive a pharmaceutical preparation input, the pharmaceutical preparation input being indicative of one or more of: an identity of the pharmaceutical preparation; a dose of the pharmaceutical preparation; and a maximum pharmaceutical preparation administration rate.

In some embodiments, the subsequent target flow rates are limited at the maximum pharmaceutical preparation administration rate, such that the subsequent target flow rates do not exceed the maximum pharmaceutical preparation administration rate.

In some embodiments, determining the number of infusion intervals within the time window of the numerical approximation comprises multiplying the time input (i) and the infusion number input (τ).

In some embodiments, determining the initiating target flow rate parameter (K(0)_{initiating}) comprises calculating:

$${K ( 0 )}_{initiating} = \sqrt{( \frac{2 {V}_{p}}{{2}^{16} - 2} {e}^{\frac{1}{2 τ} ln ( {2}^{\frac{30}{i}} )} - \frac{2 {V}_{p}}{{2}^{16} - 2} ) \times {V}_{d} \times {τ}^{2}} .$$

In some embodiments, determining the initiating pharmaceutical preparation concentration comprises calculating:

$${C}_{{d}_{( o )}} = \frac{( {K ( 0 )}_{initiating} \times {C}_{p} ) - ( {K ( 0 )}_{initiating} \times {C}_{{d}_{( - 1 )}} ) + ( {C}_{{d}_{( - 1 )}} \times {V}_{d} \times τ )}{{V}_{d} \times τ} \qquad ( 0 )$$

where C_{d}_{_{(−1)}}=0 and C_{d}_{₍ₒ₎} is the initiating pharmaceutical preparation concentration.

In some embodiments, determining the subsequent target flow rates comprises determining a flow rate parameter Kₙ for each of the subsequent target flow rates by calculating:

$${K}_{n} = \frac{{Dose ( t )}_{n} * τ}{{C}_{d ( n - 1 )}}$$

where n is the number of the relevant infusion interval, C_{d(n-1)} is a subsequent pharmaceutical preparation concentration of a previous infusion interval of the nth infusion interval and Dose(t)ₙ is the target dose of the respective infusion interval of the first time window.

In some embodiments, determining the target dose Dose(t)ₙ comprises determining a dose of a Tansy function T(t), by calculating:

$${\int }_{\frac{n - 1}{τ}}^{\frac{n}{τ}} T ( t ) d t \times {C}_{p}$$

where T(t) is the Tansy function.

In some embodiments,

$${\int }_{\frac{n}{n - τ}}^{\frac{n}{τ}} T ( t ) dt$$

is equal to:

$$( \frac{2 {V}_{p}}{{2}^{16} - 2} {e}^{( \frac{n}{2 τ} ) l n 2 ( \frac{30}{i} )} - \frac{2 {V}_{p}}{{2}^{16} - 2} ) - ( \frac{2 {V}_{p}}{{2}^{16} - 2} {e}^{( \frac{n - 1}{2 τ} ) l n 2 ( \frac{30}{i} )} - \frac{2 {V}_{p}}{{2}^{16} - 2} ) .$$

In some embodiments, determining the subsequent pharmaceutical preparation concentrations comprises calculating:

$${C}_{d ( n )} = \frac{( {K}_{n} \times {C}_{p} ) - ( {K}_{n} \times {C}_{d ( n - 1 )} ) + ( {C}_{d ( n - 1 )} \times {V}_{d} \times τ )}{{V}_{d} \times τ}$$

where C_{d(n)} is the subsequent pharmaceutical preparation concentration for the n th infusion interval and C_{d(n-1)} is the subsequent pharmaceutical preparation concentration for the n−1 th infusion interval.

In some embodiments, determining the first infusion volume for one of the first number of the infusion steps (h₁) comprises calculating:

$${V}_{s t e p ( x )} = {\sum }_{n = \frac{( x - 1 ) ( i \times τ )}{h}}^{n = \frac{( x ) ( i \times τ )}{h}} ( {K}_{n} \times \frac{1}{τ} )$$

where Vₛₜₑₚ₍ₓ₎ is the infusion volume of the xth infusion step of the first number of the infusion steps (h₁).

In some embodiments, determining a target flow rate Dₙ for each of the number of infusion intervals of the second time window comprises calculating:

$${\int }_{\frac{n - 1}{τ}}^{\frac{n}{τ}} T ( t ) d t \times \frac{{C}_{p}}{{C}_{d c}}$$

where C_{dc} is a concentration of the pharmaceutical preparation in the dilution chamber at a point when the active agent chamber is empty.

In some embodiments, determining the second infusion volume for one of the second number of the infusion steps (h₂) comprises calculating:

$${V}_{s t e p ( x )} = {\sum }_{n = \frac{( x - 1 ) ( i \times τ )}{h}}^{n = \frac{( x ) ( i \times τ )}{h}} ( {D}_{n} \times \frac{1}{τ} )$$

where Vₛₜₑₚ₍ₓ₎ is the infusion volume of the xth infusion step of the second number of the infusion steps (h₂) and Dₙ is the target flow rate for one of the number of infusion intervals of the second time window.

In some embodiments, the program instructions are further configured to cause the at least one infusion device processor to determine an infusion rate for each of the infusion steps (h), and wherein determining the infusion rate for one of the infusion steps comprises calculating

$$\frac{{V}_{s t e p ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator such that the determined infusion volume for each infusion step is output by the medication delivery apparatus during the respective infusion step at the determined infusion rate.

In some embodiments, the program instructions are further configured to cause the at least one infusion device processor to actuate the infusion device actuator such that the determined infusion volume for each infusion step is delivered according to a constant-rate profile or a linearly-changing rate profile.

In some embodiments, the program instructions are further configured to cause the at least one infusion device processor to actuate the infusion device actuator such that the determined infusion volume for each infusion step is output by the medication delivery apparatus during the respective subsequent infusion step in bursts.

In some embodiments, the infusion modelling function is a Kelly function.

In some embodiments, there is provided a medication delivery apparatus. The medication delivery apparatus may comprise: a plunger; a container configured to receive at least a portion of the plunger; and a dilution chamber fluidly connectable to the container, the dilution chamber being configured to receive a diluent; wherein: the plunger and the container together define an active agent chamber that is configured to receive a pharmaceutical preparation, the active agent chamber comprising an active agent chamber opening configured to receive the at least a portion of the plunger and an active agent chamber outlet; the dilution chamber is configured to receive the pharmaceutical preparation from the active agent chamber outlet, the dilution chamber comprising a dilution chamber outlet; and the plunger is configured to be displaced to: displace the pharmaceutical preparation in the active agent chamber through the active agent chamber outlet and into the dilution chamber, thereby producing a diluted pharmaceutical preparation; and displace the diluted pharmaceutical preparation in the dilution chamber through the dilution chamber outlet.

In some embodiments, the medication delivery apparatus further comprises: a second inlet configured to receive flushing fluid; a one-way valve configured to enable fluid from the active agent chamber to enter the dilution chamber and to inhibit fluid in the displacement chamber from entering the active agent chamber; and a multiway valve configured to be actuated between a first position and a second position; wherein the multiway valve is configured to: enable flushing fluid from the second inlet into the dilution chamber whilst inhibiting displacement of the pharmaceutical preparation into the dilution chamber when in the first position, and enable displacement of the pharmaceutical preparation into the dilution chamber and inhibit flushing fluid from entering the dilution chamber when in the second position.

In some embodiments, the medication delivery apparatus further comprises a first conduit configured to fluidly connect the active agent chamber outlet and a dilution chamber inlet.

In some embodiments, the medication delivery apparatus further comprises a catheter configured to be at least partially disposed within the dilution chamber.

In some embodiments, the catheter comprises: a catheter body comprising: a hollow core that defines a catheter body fluid flow path; and a plurality of catheter body perforations disposed at an end portion of the catheter, each of the plurality of catheter body perforations extending between the hollow core and an exterior of the catheter body; a blind end; and a flexible sleeve that is connected to the end portion, the flexible sleeve comprising a plurality of sleeve perforations extending between an inner surface of the sleeve and an outer surface of the sleeve such that a pharmaceutical preparation catheter flow path is defined between the hollow core and each of the plurality of sleeve perforations via the plurality of catheter body perforations.

In some embodiments, the catheter is configured to fluidly connect to a second end of the first conduit.

In some embodiments, the end portion is configured to be disposed within the dilution chamber.

In some embodiments, the catheter comprises a bubble trap.

In some embodiments, the medication delivery apparatus further comprises a manifold, the manifold being configured to connect to the dilution chamber.

In some embodiments, the manifold comprises a manifold inlet and a manifold outlet, the manifold inlet being configured to receive the pharmaceutical preparation from the dilution chamber, and the manifold outlet being configured to connect to a second conduit enabling the pharmaceutical preparation to be delivered to a patient.

In some embodiments, there is provided a medication delivery system. The medication delivery system comprises the medication delivery apparatus and an infusion device. The infusion device comprises at least one infusion device processor; and infusion device memory storing program instructions accessible by the at least one infusion device processor.

In some embodiments, the program instructions are configured to cause the at least one infusion device processor to: receive a volume input (Vₚ) that is indicative of a volume of a pharmaceutical preparation; receive a time input (i) that is indicative of a time over which the pharmaceutical preparation is to be administered; determine a number of infusion steps that are to be executed during the time over which the pharmaceutical preparation is to be administered; determine a pharmaceutical preparation output volume for each of the infusion steps of the number of infusion steps, each pharmaceutical preparation output volume corresponding to a volume of the pharmaceutical preparation that is to be output by the medication delivery apparatus during the respective infusion step; determine a target flow rate of each infusion step, each target flow rate being indicative of a target flow rate of the pharmaceutical preparation to be output by the medication delivery apparatus during the respective infusion step, wherein each target flow rate is determined based at least in part on the pharmaceutical preparation output volume of the respective infusion step; and actuate an infusion device actuator to displace the plunger such that the pharmaceutical preparation is output by the medication delivery apparatus at the respective target flow rate during each infusion step.

In some embodiments, the program instructions are configured to cause the at least one infusion device processor to: receive: a concentration input (Cₚ) that is indicative of a concentration of the pharmaceutical preparation in the active agent chamber; a volume input (Vₚ) that is indicative of a volume of the pharmaceutical preparation that is to be infused, a dilution chamber volume input (V_{d}) that is indicative of a volume of the dilution chamber; a time input (i) that is indicative of a time window over which the pharmaceutical preparation is to be administered; an infusion number input (τ) that is indicative of a number of infusion intervals per minute over which an infusion modelling function is to be numerically approximated over the time window; a number of infusion steps (h) that are to be executed during the time window; numerically approximate the infusion modelling function over the time window, wherein numerically approximating the infusion modelling function comprises: determining a number of infusion intervals within the time window; determining an initiating target flow rate parameter (S(0)_{initiating}), the initiating target flow rate parameter being indicative of a target flow rate of the pharmaceutical preparation to be output by the medication delivery apparatus during an initiating infusion interval of the numerical approximation; determining an initiating pharmaceutical preparation concentration, the initiating pharmaceutical preparation concentration being indicative of an approximated concentration of the pharmaceutical preparation in the dilution chamber after the initiating infusion interval of the numerical approximation; iteratively determining a subsequent target flow rate and a subsequent pharmaceutical preparation concentration for each of a plurality of subsequent infusion intervals of the numerical approximation, wherein: the subsequent target flow rates are each indicative of a target flow rate of the pharmaceutical preparation to be output by the medication delivery apparatus during a respective subsequent infusion interval of the numerical approximation; the subsequent pharmaceutical preparation concentrations are each indicative of a subsequent approximated concentration of the pharmaceutical preparation in the dilution chamber after the respective subsequent infusion interval; each of the subsequent target flow rates is determined based at least in part on the subsequent pharmaceutical preparation concentration of a previous infusion interval of the respective infusion interval; and each of the subsequent pharmaceutical preparation concentrations is determined based at least in part on the subsequent target flow rate of the respective subsequent infusion interval; determine an infusion volume for each of the number of infusion steps (h), based at least in part on the numerical approximation, the infusion volume being indicative of a volume of the pharmaceutical preparation that is to be output by the medication delivery apparatus during the respective infusion step; and actuate an infusion device actuator to displace the plunger such that the determined infusion volume for each infusion step is output by the medication delivery apparatus during the respective infusion step.

In some embodiments, the program instructions are configured to cause the at least one infusion device processor to: receive: a concentration input (Cₚ) that is indicative of a concentration of the pharmaceutical preparation in the active agent chamber; a volume input (Vₚ) that is indicative of a volume of the pharmaceutical preparation, a dilution chamber volume input (V_{d}) that is indicative of a volume of the dilution chamber; a time input (i) that is indicative of a time window over which the pharmaceutical preparation is to be administered, the time window comprising a first time window and a second time window; an infusion number input (τ) that is indicative of a number of infusion intervals per minute over which an infusion modelling function is to be numerically approximated over the first time window; a number of infusion steps (h) that are to be executed during the tine window, wherein a first number of the infusion steps (h₁) are to be executed during the first time window and a second number of the infusion steps (h₂) are to be executed during the second time window; numerically approximate the infusion modelling function over the first time window, wherein numerically approximating the infusion modelling function comprises: determining a number of infusion intervals of the first time window; determining an initiating target flow rate parameter (K(0)_{initiating}), the initiating target flow rate parameter being indicative of a target flow rate of the pharmaceutical preparation to be output into the dilution chamber during an initiating infusion interval of the numerical approximation; determining an initiating pharmaceutical preparation concentration, the initiating pharmaceutical preparation concentration being indicative of an approximated concentration of the pharmaceutical preparation in the dilution chamber after the initiating infusion interval of the numerical approximation; iteratively determining a subsequent target flow rate and a subsequent pharmaceutical preparation concentration for each of a plurality of subsequent infusion intervals of the numerical approximation; wherein the subsequent target flow rates are each indicative of a target flow rate of the pharmaceutical preparation to be output by the medication delivery apparatus during a respective subsequent infusion interval of the numerical approximation; the subsequent pharmaceutical preparation concentrations are each indicative of a subsequent approximated concentration of the pharmaceutical preparation in the dilution chamber after the respective subsequent infusion interval; each of the subsequent target flow rates is determined based at least in part on the subsequent pharmaceutical preparation concentration of a previous infusion interval of the respective infusion interval; and each of the subsequent pharmaceutical preparation concentrations is determined based at least in part on the subsequent target flow rate of the respective subsequent infusion interval; determine a first infusion volume for each of the first number of the infusion steps (h₁), based at least in part on the numerical approximation, the infusion volume being indicative of a volume of the pharmaceutical preparation that is to be output by the medication delivery apparatus during the respective infusion step; determine a number of infusion intervals of the second time window; determine a target dose Dose(t)ₙ for each of the number of infusion intervals of the second time window; determine a target flow rate Dₙ for each of the number of infusion intervals of the second time window, based at least in part on the target dose for the respective infusion interval; determine a second infusion volume for each of the second number of infusion steps (h₂) based at least in part on the target flow rate; and actuate an infusion device actuator to displace the plunger such that the first infusion volume or the second infusion volume for each infusion step (h) is output by the medication delivery apparatus during the respective infusion step.

In some embodiments, there is provided a method for delivering a pharmaceutical preparation into a patient. The method may comprise: receiving a volume input (Vₚ) that is indicative of a volume of a pharmaceutical preparation, receiving a time input (i) that is indicative of a time over which the pharmaceutical preparation is to be administered; determining a number of infusion steps that are to be executed during the time over which the pharmaceutical preparation is to be administered; determining a pharmaceutical preparation output volume for each of the infusion steps of the number of infusion steps, each pharmaceutical preparation output volume corresponding to a volume of the pharmaceutical preparation that is to be output by a medication delivery apparatus during the respective infusion step; determining a target flow rate of each infusion step, each target flow rate being indicative of a target flow rate of the pharmaceutical preparation to be output by the medication delivery apparatus during the respective infusion step, wherein each target flow rate is determined based at least in part on the pharmaceutical preparation output volume of the respective infusion step; and actuating an infusion device actuator to such that the pharmaceutical preparation is output by the medication delivery apparatus at the respective target flow rate during each infusion step.

In some embodiments, there is provided a method for delivering a pharmaceutical preparation into a patient; the method comprising: receiving: a concentration input (Cₚ) that is indicative of a concentration of a pharmaceutical preparation in an active agent chamber of a medication delivery apparatus; a volume input (Vₚ) that is indicative of a volume of the pharmaceutical preparation that is to be infused, a dilution chamber volume input (V_{d}) that is indicative of a volume of a dilution chamber of the medication delivery apparatus; a time input (i) that is indicative of a time window over which the pharmaceutical preparation is to be administered; an infusion number input (τ) that is indicative of a number of infusion intervals per minute over which an infusion modelling function is to be numerically approximated over the time window; and a number of infusion steps (h) that are to be executed during the time window; numerically approximate the infusion modelling function over the time window, wherein numerically approximating the infusion modelling function comprises: determining a number of infusion intervals within the time window; determining an initiating target flow rate parameter (S(0)_{initiating}), the initiating target flow rate parameter being indicative of a target flow rate of the pharmaceutical preparation to be output by the medication delivery apparatus during an initiating infusion interval of the numerical approximation; determining an initiating pharmaceutical preparation concentration, the initiating pharmaceutical preparation concentration being indicative of an approximated concentration of the pharmaceutical preparation in the dilution chamber after the initiating infusion interval of the numerical approximation; iteratively determining a subsequent target flow rate and a subsequent pharmaceutical preparation concentration for each of a plurality of subsequent infusion intervals of the numerical approximation, wherein: the subsequent target flow rates are each indicative of a target flow rate of the pharmaceutical preparation to be output by the medication delivery apparatus during a respective subsequent infusion interval of the numerical approximation; the subsequent pharmaceutical preparation concentrations are each indicative of a subsequent approximated concentration of the pharmaceutical preparation in the dilution chamber after the respective subsequent infusion interval; each of the subsequent target flow rates is determined based at least in part on the subsequent pharmaceutical preparation concentration of a previous infusion interval of the respective infusion interval; and each of the subsequent pharmaceutical preparation concentrations is determined based at least in part on the subsequent target flow rate of the respective subsequent infusion interval; determining an infusion volume for each of the number of infusion steps (h), based at least in part on the numerical approximation, the infusion volume being indicative of a volume of the pharmaceutical preparation that is to be output by the medication delivery apparatus during the respective infusion step; and actuating an infusion device actuator to displace a plunger within a chamber of the medication delivery apparatus such that the determined infusion volume for each infusion step is output by the medication delivery apparatus during the respective infusion step.

In some embodiments, there is provided a method for delivering a pharmaceutical preparation into a patient; the method comprising: receiving: a concentration input (Cₚ) that is indicative of a concentration of a pharmaceutical preparation in an active agent chamber of a medication delivery apparatus; a volume input (Vₚ) that is indicative of a volume of the pharmaceutical preparation, a dilution chamber volume input (V_{d}) that is indicative of a volume of a dilution chamber of the medication delivery apparatus; a time input (i) that is indicative of a time window over which the pharmaceutical preparation is to be administered, the time window comprising a first time window and a second time window; an infusion number input (τ) that is indicative of a number of infusion intervals per minute over which an infusion modelling function is to be numerically approximated over the first time window; a number of infusion steps (h) that are to be executed during the tine window, wherein a first number of the infusion steps (h₁) are to be executed during the first time window and a second number of the infusion steps (h₂) are to be executed during the second time window; numerically approximate the infusion modelling function over the first time window, wherein numerically approximating the infusion modelling function comprises: determining a number of infusion intervals of the first time window; determining an initiating target flow rate parameter (K(0)_{initiating}), the initiating target flow rate parameter being indicative of a target flow rate of the pharmaceutical preparation to be output into the dilution chamber during an initiating infusion interval of the numerical approximation; determining an initiating pharmaceutical preparation concentration, the initiating pharmaceutical preparation concentration being indicative of an approximated concentration of the pharmaceutical preparation in the dilution chamber after the initiating infusion interval of the numerical approximation; iteratively determining a subsequent target flow rate and a subsequent pharmaceutical preparation concentration for each of a plurality of subsequent infusion intervals of the numerical approximation; wherein: the subsequent target flow rates are each indicative of a target flow rate of the pharmaceutical preparation to be output by the medication delivery apparatus during a respective subsequent infusion interval of the numerical approximation; the subsequent pharmaceutical preparation concentrations are each indicative of a subsequent approximated concentration of the pharmaceutical preparation in the dilution chamber after the respective subsequent infusion interval; each of the subsequent target flow rates is determined based at least in part on the subsequent pharmaceutical preparation concentration of a previous infusion interval of the respective infusion interval; and each of the subsequent pharmaceutical preparation concentrations is determined based at least in part on the subsequent target flow rate of the respective subsequent infusion interval; determining a first infusion volume for each of the first number of the infusion steps (h₁), based at least in part on the numerical approximation, the infusion volume being indicative of a volume of the pharmaceutical preparation that is to be output by the medication delivery apparatus during the respective infusion step; determining a number of infusion intervals of the second time window; determining a target dose Dose(t)ₙ for each of the number of infusion intervals of the second time window; determining a target flow rate Dₙ for each of the number of infusion intervals of the second time window, based at least in part on the target dose for the respective infusion interval; determining a second infusion volume for each of the second number of infusion steps (h₂) based at least in part on the target flow rate; and actuating an infusion device actuator to displace a plunger within a chamber of the medication delivery apparatus such that the first infusion volume or the second infusion volume for each infusion step (h) is output by the medication delivery apparatus during the respective infusion step.

In some embodiments, there is provided a medication delivery apparatus. The medication delivery apparatus may comprise: a medication delivery apparatus body; a first plunger configured to be slidably received within the medication delivery apparatus body; a first chamber that is configured to receive a pharmaceutical preparation; and a second chamber that is configured to receive a diluent; wherein: the first plunger is configured to: force a portion of the pharmaceutical preparation into the second chamber to mix with the diluent to form a diluted pharmaceutical preparation; and force the diluted pharmaceutical preparation out of an outlet of the second chamber.

In some embodiments, there is provided a method for delivering an active ingredient into a patient, the method comprising the steps of preparing a pharmaceutical preparation having a particular volume, the pharmaceutical preparation comprising a solvent and therapeutic dose of the active ingredient and administering to a patient the pharmaceutical preparation, wherein the pharmaceutical preparation is administered to the patient in such a manner that at a first stage of administration of the pharmaceutical preparation at least one portion of the therapeutic dose is administered to the patient for detection of a negative reaction in the patient.

In some embodiments, there is provided a system for delivering an active ingredient into a patient, the active ingredient being part of a pharmaceutical preparation having a particular volume, the pharmaceutical preparation comprising a solvent and therapeutic dose of the active ingredient, the system comprising an infusion driver having a processor for running instructions of an algorithm for approximating the flow rate variation of the pharmaceutical preparation such that the pharmaceutical preparation is administered to the patient in such a manner that at a first stage of administration of the pharmaceutical preparation at least one portion of the therapeutic dose is administered to the patient for detection of a negative reaction in the patient.

In some embodiments, there is provided a dilution chamber comprising a container and a manifold connected to the container to permit fluid flow from the infusion driver, via a first conduit and first inlet of the manifold, into the container and from the container via a first outlet of the manifold for delivery of the drug to the patient via a conduit.

In some embodiments, there is provided a catheter for insertion in the dilution chamber, the catheter having a first end fluidly connected to the first inlet of the dilution chamber for receiving the pharmaceutical preparation from the infusion driver, and a second end extending in the container.

In some embodiments, there is provided a bubble trap for use in conjunction with the catheter, the bubble trap being adapted to deviate any bubble forming at the first end of the catheter located within the container of the dilution chamber and floating adjacent the catheter preventing any bubble from being delivered to the patient.

In some embodiments, there is provided a dilution chamber comprising a container defining an inner volume and having at least one inlet for receiving at least one first fluid and an outlet for discharging a second fluid, a first plunger for applying a pushing force to at least the first fluid and a second plunger for dividing the inner volume of the container into a first chamber and a second chamber, wherein the second plunger is adapted to allow fluid flow between the first chamber and the second chamber.

In some embodiments, there is provided a dilution chamber comprising a first chamber and a second chamber fluidly connected with respect to each other, a first piston to be slideably received within the first chamber for applying a pushing force to a first fluid contained in the first chamber for delivering the first fluid to the second chamber, and a second piston to be slideably received within the second chamber for applying a pushing force to a second fluid contained in the second chamber, wherein the first piston is adapted to apply the pushing force during a first period of time and the second piston is adapted to apply the pushing force during a second period of time, the first period of time starting before the second period of time.

In some embodiments, there is provided a medication delivery apparatus. The medication delivery apparatus may comprise: a first plunger; a second plunger; a first container configured to receive at least a portion of the first plunger; a second container configured to receive at least a portion of the second plunger; wherein: the first container and the first plunger together define an active agent chamber that is configured to receive a pharmaceutical preparation, the active agent chamber comprising an active agent chamber opening; the second container and the second plunger together define a dilution chamber that is configured to receive a diluent, the dilution chamber comprising a dilution chamber opening; the first plunger is configured to be actuated to apply a pushing force to the pharmaceutical preparation within the first container to deliver the pharmaceutical preparation to the second container; and the second plunger is configured to be actuated to apply a pushing force to the pharmaceutical preparation within the second container to push the pharmaceutical preparation through a medication delivery apparatus outlet.

In some embodiments, the medication delivery apparatus further comprises a valve configured to enable fluid from the active agent chamber to enter the dilution chamber and to inhibit fluid in the dilution chamber from entering the active agent chamber.

In some embodiments, the first container and the second container are connected by a conduit.

In some embodiments, there is provided a medication delivery system. The medication delivery system comprises the medication delivery apparatus and an infusion device. The infusion device comprises at least one infusion device processor; and infusion device memory storing program instructions accessible by the at least one infusion device processor.

In some embodiments, the program instructions are configured to cause the at least one infusion device processor to: receive: a concentration input (Cₚ) that is indicative of a concentration of the pharmaceutical preparation in the active agent chamber; a volume input (Vₚ) that is indicative of a volume of the pharmaceutical preparation, a dilution chamber volume input (V_{d}) that is indicative of a volume of the dilution chamber; a time input (i) that is indicative of a time window over which the pharmaceutical preparation is to be administered, the time window comprising a first time window and a second time window; an infusion number input (τ) that is indicative of a number of infusion intervals per minute over which a first infusion modelling function and a second infusion modelling function are to be numerically approximated over the time window; a number of infusion steps (h) that are to be executed during the time window, wherein a first number of the infusion steps (h₁) are to be executed during the first time window and a second number of the infusion steps (h₂) are to be executed during the second time window; numerically approximate the first infusion modelling function over the first time window, the numerical approximation of the first infusion modelling function over the first time window being a first numerical approximation, wherein numerically approximating the first infusion modelling function comprises: determining a first number of infusion intervals within the first time window; determining an initiating target flow rate parameter (K(0)_{initiating}), the initiating target flow rate parameter being indicative of a target flow rate of the pharmaceutical preparation to be output into the dilution chamber during an initiating infusion interval of the first numerical approximation; determining an initiating pharmaceutical preparation concentration, the initiating pharmaceutical preparation concentration being indicative of an approximated concentration of the pharmaceutical preparation in the dilution chamber after the initiating infusion interval of the first numerical approximation; iteratively determining a subsequent target flow rate and a subsequent pharmaceutical preparation concentration for each of a plurality of subsequent infusion intervals of the first numerical approximation; wherein the subsequent target flow rates of the first numerical approximation are each indicative of a target flow rate of the pharmaceutical preparation to be output by the medication delivery apparatus during a respective subsequent infusion interval of the first numerical approximation; the subsequent pharmaceutical preparation concentrations of the first numerical approximation are each indicative of a subsequent approximated concentration of the pharmaceutical preparation in the dilution chamber after the respective subsequent infusion interval; each of the subsequent target flow rates of the first numerical approximation is determined based at least in part on the subsequent pharmaceutical preparation concentration of a previous infusion interval of the respective infusion interval; and each of the subsequent pharmaceutical preparation concentrations of the first numerical approximation is determined based at least in part on the subsequent target flow rate of the respective subsequent infusion interval; numerically approximate the second infusion modelling function over the second time window, the numerical approximation of the second infusion modelling function over the second time window being a second numerical approximation, wherein numerically approximating the second infusion modelling function comprises: iteratively determining a subsequent target flow rate, a subsequent dilution chamber volume and a subsequent pharmaceutical preparation concentration for each of a plurality of subsequent infusion intervals of the second numerical approximation; wherein: the subsequent target flow rates of the second numerical approximation are each indicative of a target flow rate of the pharmaceutical preparation to be output by the medication delivery apparatus during a respective subsequent infusion interval of the second numerical approximation; the subsequent dilution chamber volumes are each indicative of a volume of the dilution chamber after a preceding infusion interval of the respective infusion interval; the subsequent pharmaceutical preparation concentrations of the second numerical approximation are each indicative of a subsequent approximated concentration of the pharmaceutical preparation in the dilution chamber after the respective subsequent infusion interval; each of the subsequent target flow rates of the second numerical approximation is determined based at least in part on the subsequent pharmaceutical preparation concentration of a previous infusion interval of the respective infusion interval; and the subsequent pharmaceutical preparation concentrations of the second numerical approximation are determined based at least in part on the subsequent target flow rate of the respective subsequent infusion intervals and the a corresponding subsequent dilution chamber volume; determine a first infusion volume for each of the first number of the infusion steps (h₁), based at least in part on the first numerical approximation; determine a second infusion volume for each of the second number of infusion steps (h₂), based at least in part on the second numerical approximation, the first and second infusion volumes being indicative of a volume of the pharmaceutical preparation that is to be output by the medication delivery apparatus during the respective infusion steps; and actuate an infusion device actuator to displace the first plunger and/or the second plunger such that the first infusion volume or the second infusion volume for each infusion step (h) is output by the medication delivery apparatus during the respective infusion step.

In some embodiments, the first infusion modelling function is a Kelly function.

In some embodiments, numerically approximating the first infusion modelling function over the first time window comprises numerically approximating the Kelly function.

In some embodiments, determining the subsequent target flow rates of the second numerical approximation comprises determining a flow rate parameter Wₙ for each of the subsequent target flow rates of the second numerical approximation by calculating:

$${W}_{n} = \frac{D o s {e ( t )}_{n} * τ}{{C}_{d ( n - 1 )}}$$

where n is the number of the relevant infusion interval, C_{d(n-1)} is a subsequent pharmaceutical preparation concentration of a previous infusion interval of the nth infusion interval and Dose(t)ₙ is a target dose.

In some embodiments, determining the target dose Dose(t)ₙ comprises determining a dose of a Tansy function T(t), by calculating:

$${\int }_{\frac{n - 1}{τ}}^{\frac{n}{τ}} T ( t ) d t \times {C}_{p}$$

where T(t) is the Tansy function.

In some embodiments,

$${\int }_{\frac{n - 1}{τ}}^{\frac{n}{τ}} T ( t ) dt$$

is equal to:

$$( \frac{2 {V}_{p}}{{2}^{16} - 2} {e}^{( \frac{n}{2 τ} ) l n 2 ( \frac{30}{i} )} - \frac{2 {V}_{p}}{{2}^{16} - 2} ) - ( \frac{2 {V}_{p}}{{2}^{16} - 2} {e}^{( \frac{n - 1}{2 τ} ) l n 2 ( \frac{30}{i} )} - \frac{2 {V}_{p}}{{2}^{16} - 2} ) .$$

In some embodiments, determining the subsequent dilution chamber volumes of the second numerical approximation comprises calculating:

$${V ( d )}_{n} = {V ( d )}_{n - 1} - ( γ \times \frac{{W}_{n}}{τ} )$$

where V(d)ₙ is the volume of the dilution chamber for the nth infusion interval of the second numerical approximation, V(d)ₙ₋₁ is the volume of the dilution chamber for the n−1 th infusion interval of the second numerical approximation, and y is a proportion of reduction in volume of the dilution chamber relative to a volume of fluid exiting the dilution chamber.

In some embodiments, determining the subsequent pharmaceutical preparation concentrations of the second numerical approximation comprises calculating:

$${C}_{d ( n )} = \frac{( ( 1 - γ ) \times {W}_{n} \times {C}_{p} ) - ( γ \times {W}_{n} \times {C}_{d ( n - 1 )} ) + ( {C}_{d ( n - 1 )} \times {V ( d )}_{n} \times τ )}{{V ( d )}_{n} \times τ}$$

where C_{d(n)} is the subsequent pharmaceutical preparation concentration for the nth infusion interval of the second numerical approximation and C_{d(n-1)} is the subsequent pharmaceutical preparation concentration for the n−1 th infusion interval of the second numerical approximation.

In some embodiments, determining the second infusion volume for one of the second number of infusion steps (h₂) comprises calculating:

$${V}_{s t e p ( x )} = {\sum }_{n = \frac{( x - 1 ) ( i \times τ )}{h}}^{n = \frac{( x ) ( i \times τ )}{h}} ( {W}_{n} \times \frac{1}{τ} )$$

where Vₛₜₑₚ₍ₓ₎ is the infusion volume of the xth infusion step of the second number of the infusion steps (h₂).

In some embodiments, the program instructions are further configured to cause the at least one infusion device processor to determine an infusion rate for each of the infusion steps (h), and wherein determining the infusion rate for one of the infusion steps comprises calculating

$$\frac{{V}_{s t e p ( x )} \times h}{i} ,$$

where Vₛₜₑₚ₍ₓ₎ is the infusion volume of the xth infusion step.

In some embodiments, the program instructions are further configured to cause the at least one infusion device processor to actuate the infusion device actuator such that the determined first infusion volume or second infusion volume for each infusion step is output by the medication delivery apparatus during the respective infusion step at the determined infusion rate.

In some embodiments, the program instructions are further configured to cause the at least one infusion device processor to actuate the infusion device actuator such that the determined first infusion volume or second infusion volume for each infusion step is delivered according to a constant-rate profile or a linearly-changing rate profile.

In some embodiments, the program instructions are further configured to cause the at least one infusion device processor to actuate the infusion device actuator such that the determined first infusion volume or second infusion volume for each infusion step is output by the medication delivery apparatus during the respective subsequent infusion step in bursts.

In some embodiments, the first infusion modelling function is a Kelly function and the second infusion modelling function is a Wood function.

Modifications and variations as would be apparent to a skilled addressee are deemed to be within the scope of the present disclosure.

Further, it should be appreciated that the scope of the disclosure is not limited to the scope of the embodiments disclosed.

Throughout this specification, unless the context requires otherwise, the word “comprise” or variations such as “comprises” or “comprising”, will be understood to imply the inclusion of a stated integer or group of integers but not the exclusion of any other integer or group of integers.

1. An infusion device configured to control a medication delivery apparatus to intravenously deliver a pharmaceutical preparation to a patient, the infusion device comprising a processor and a memory storing instructions executable by the processor to cause the medication delivery apparatus to deliver the pharmaceutical preparation to the patient according to a predetermined dose profile, wherein the predetermined dose profile is designed to deliver a therapeutic dose of the pharmaceutical preparation to the patient over a predetermined infusion time in a manner which facilitates safe detection of an adverse reaction of the patient to the pharmaceutical preparation, or desensitization the patient to the pharmaceutical preparation, during a first stage of administering the therapeutic dose.

2. The infusion device of claim 1, wherein the predetermined dose profile is such that the dose rate varies over the predetermined infusion time.

3. The infusion device of claim 1, wherein the dose rate increases over time in the period between 14% of the infusion time and 78% of the infusion time.

4. The infusion device of claim 1, wherein the predetermined dose profile is such that the cumulative dose delivered to the patient increases exponentially, or increases at a rate that increases over time, for at least a portion of the predetermined infusion time.

5. The infusion device any one of claim 1, wherein the predetermined dose profile is such that cumulative dose delivered to the patient increases exponentially, or increases at a rate that increases over time, over a time period between a first time at which 0.1% of the cumulative dose has been delivered to the patient and a second time at which 10% of the cumulative dose has been delivered to the patient.

6. The infusion device of claim 1, wherein the predetermined dose profile is such that, after 56% or alternatively 34% of the infusion time, the cumulative dose delivered to the patient is no more than 1% of the therapeutic dose, and/or after 14% of infusion time the cumulative dose delivered to the patient is no more than 0.6% of the therapeutic dose.

7. The infusion device of claim 1, wherein the predetermined dose profile is such that the dose rate increases exponentially, or increases at a rate that increases over time, for at least a portion of the predetermined infusion time.

8. The infusion device of claim 1, wherein the predetermined dose profile is such that dose rate increases exponentially, or increases at a rate which increases over time, in the time period between 14% and 78% of the predetermined infusion time.

9. The infusion device of claim 1, wherein the predetermined dose profile is such that there is a first time period between the cumulative dose reaching 0.01% and 0.1% and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes between 2 minutes and 10 minutes, and at least the latent period of adverse reaction.

10. The infusion device of claim 1, wherein the predetermined dose profile is such that successive orders of magnitude of cumulative dose, such as 0.01%, 0.1%, 1% and 10% of the therapeutic dose, are separated in time from each other by periods of time; wherein the periods of time are selected from the group comprising: at least 6 minutes, at least 5 minutes, at least 4 minutes, at least 3 minutes between 2 minutes and 10 minutes, and at least the latent period of adverse reaction.

11. The infusion device of claim 1, wherein the predetermined dose profile is such that delivering the first 0.01% of the therapeutic dose takes longer than 0.01% of the predetermined infusion time.

12. The infusion device of claim 4, wherein the dose profile is such that a time at which the cumulative dose reaches 10% of the therapeutic dose and a time at which the dose rate reaches 10% of the maximum dose rate are the same plus or minus 5% of the predetermined infusion time.

13. The infusion device of claim 4, wherein the dose profile is such that a time at which the cumulative dose reaches 50% of the therapeutic dose and a time at which the dose rate reaches 50% of the maximum dose rate are the same plus or minus 5% of the predetermined infusion time.

14. The infusion device of claim 4, wherein the dose profile is such that a time at which the cumulative dose reaches 1% of the therapeutic dose and a time at which the dose rate reaches 1% of the maximum dose rate are the same plus or minus 5% of the predetermined infusion time.

15. The infusion device of claim 3, wherein the dose profile has a maximum dose rate and the dose profile is such that the time which it takes for the cumulative dose to reach 0.1%, 1% and/or 10% of the therapeutic dose respectively is substantially the same as the time which it takes for the dose rate to reach 0.1%, 1% and/or 10% of the maximum dose rate.

16. The infusion device of claim 1, wherein the predetermined dose profile delivers the therapeutic dose over a predetermined infusion time which is between 20 minutes and 180 minutes.

17. The infusion device of claim 1, together with a medication delivery apparatus, wherein the medication delivery apparatus comprises an active agent chamber for receiving the pharmaceutical preparation and a dilution chamber for receiving pharmaceutical preparation ejected from the active agent chamber and diluting the pharmaceutical preparation with a diluent, the dilution chamber comprising an dilution chamber outlet for delivering the diluted pharmaceutical preparation to the patient.

18. The infusion device of claim 17, wherein the infusion device configured such that the dilution chamber has a fixed volume in a first portion of the predetermined infusion time and a variable volume in a second portion of the predetermined infusion time.

19. The infusion device of claim 17, wherein the predetermined dose profile is such that the concentration of the pharmaceutical preparation in the dilution chamber increases during the process of infusion to the patient.

20. The infusion device of claim 17, wherein the predetermined dose profile delivers the therapeutic dose over a predetermined infusion time in a manner such that a flow rate of pharmaceutical preparation into the dilution chamber starts at a higher level and decreases during an initial stage to a minimum flow rate and then increases.

21. The infusion device of claim 20, wherein the minimum flow rate is reached within the first 10% or first 15% of the predetermined infusion time.

22. The infusion device of claim 1, wherein the infusion device comprises:
a first plunger;
a second plunger; and
a container configured to receive the second plunger and at least a portion of the first plunger;
wherein the dilution chamber is defined by the container and the second plunger and the dilution chamber outlet comprises a dilution chamber opening defined by the container; wherein the active agent chamber is defined by the first plunger, the container and the second plunger, and the active agent chamber comprises a first active agent chamber opening configured to receive the at least a portion of the first plunger; and
the second plunger comprises a valve configured to control a flow of pharmaceutical preparation from the active agent chamber to the dilution chamber in response to applied pressure.

23. The infusion device of claim 22, wherein:
the active agent chamber comprises a second active agent chamber opening in a wall of the container; and
the active agent chamber is configured to receive the pharmaceutical preparation through the second active agent chamber opening.

24. The infusion device of claim 1, wherein the instructions include instructions to:
receive a volume input (Vp) that is indicative of a volume of the pharmaceutical preparation,
receive a time input (i) that is indicative of a time over which the pharmaceutical preparation is to be administered;
determine a number of infusion steps (h) that are to be executed during the time over which the pharmaceutical preparation is to be administered;
determine a pharmaceutical preparation output volume for each of the infusion steps of the number of infusion steps, each pharmaceutical preparation output volume corresponding to a volume of the pharmaceutical preparation that is to be output by the medication delivery apparatus during the respective infusion step;
determine a target flow rate of each infusion step, each target flow rate being indicative of a target flow rate of the pharmaceutical preparation to be output by the medication delivery apparatus during the respective infusion step, wherein each target flow rate is determined based at least in part on the pharmaceutical preparation output volume of the respective infusion step; and
actuate an infusion device actuator to displace the first plunger such that the pharmaceutical preparation is output by the medication delivery apparatus at the respective target flow rate during each infusion step.

25. The infusion device of any claim 1, wherein the instructions include instructions to:
receive:
a concentration input (Cₚ) that is indicative of a concentration of the pharmaceutical preparation in the active agent chamber;
a volume input (Vₚ) that is indicative of a volume of the pharmaceutical preparation that is to be infused,
a dilution chamber volume input (V_{d}) that is indicative of a volume of the dilution chamber;
a time input (i) that is indicative of a time window over which the pharmaceutical preparation is to be administered;
determine a number of infusion steps (h) that are to be executed during the time window;
model the infusion over the time window based on an infusion modelling function wherein modelling the infusion comprises determining a target flow rate of the pharmaceutical preparation to the patient and a concentration of the pharmaceutical preparation in the dilution chamber for each of the number of infusion steps;
determine an infusion volume for each of the number of infusion steps (h), based at least in part on said infusion modelling, the infusion volumes being indicative of a volume of the pharmaceutical preparation that is to be output by the medication delivery apparatus during the respective infusion step; and
actuate an infusion device actuator to displace the first plunger such that the determined infusion volume for each infusion step is output by the medication delivery apparatus during the respective infusion step.

26. The infusion device of claim 1, wherein the predetermined dose profile is such that the dose rate doubles over a period of time during the infusion, the period of time over which the dose rate doubles being 1.33 minutes to 12 minutes.

27. The infusion device of claim 1, wherein the predetermined dose profile is such that, for at least part of the infusion, the dose rate doubles every X minutes, where X is in the range 1.33 minutes to 12 minutes.

28. A method for delivering an active ingredient into a patient, the method comprising the steps of preparing a pharmaceutical preparation having a particular volume, the pharmaceutical preparation comprising a solvent and therapeutic dose of the active ingredient and intravenously administering the pharmaceutical preparation to the patient, wherein the pharmaceutical preparation is intravenously administered to the patient in in accordance with a predetermined dose profile over a predetermined infusion time in a manner such that at a first stage of administration of the pharmaceutical preparation at least one portion of the therapeutic dose is administered to the patient for detection of a negative reaction in the patient; wherein the predetermined dose profile has at least one of the following properties:
the predetermined dose profile is such that after 14% of the predetermined infusion time, the cumulative dose delivered to the patient is no more than 0.01% of the therapeutic dose;
the predetermined dose profile is such that the cumulative dose delivered to the patient increases exponentially, or at an increasing rate over time, for at least a portion of the predetermined infusion time, such as between 14% and 78% of the predetermined infusion time;
the predetermined dose profile is such that the dose rate increases exponentially, or at a rate which increases over time, for at least a portion of the predetermined infusion time, such as between 14% and 78% of the predetermined infusion time;
the predetermined dose profile is such that there is a first time period between the cumulative dose reaching 0.01% and 0.1% and a second time period between the cumulative dose reaching 0.1% and 1% of the therapeutic dose; wherein the first period of time and the second period of time are selected from the group comprising: at least 6 minutes, at least 5 minutes, at least 4 minutes, at least 3 minutes, between 2 minutes and 10 minutes, and at least the period of latent anaphylactic reaction; and/or
the predetermined dose profile is such that for at least a portion of the infusion, such as between 14% and 78% of the predetermined infusion time, successive orders of magnitude of cumulative dose, such as 0.01%, 0.1% and 1% etc. of the therapeutic dose, are separated in time from each other by a period of time; wherein the period of time is selected from the group comprising: at least 6 minutes, at least 5 minutes, at least 4 minutes, at least 3 minutes, between 2 minutes and 10 minutes, and at least the period of latent anaphylactic reaction.

29. The method of claim 28, wherein the method comprises controlling and/or programming an infusion device to deliver the pharmaceutical preparation according to the predetermined dose profile.

30. The method of claim 28, comprising checking the patient for an adverse reaction after 0.01%, 0.1% and 1% of the therapeutic dose has been delivered.

31. The method of claim 28, wherein the method uses a medication delivery apparatus comprising an active agent chamber for receiving the pharmaceutical preparation and a dilution chamber for receiving pharmaceutical preparation ejected from the active agent chamber and diluting the pharmaceutical preparation with a diluent, the dilution chamber comprising an dilution chamber outlet for delivering the diluted pharmaceutical preparation to the patient.

32. The method of claim 28, wherein the pharmaceutical preparation is intravenously administered to the patient using an infusion device according to claim 17 and wherein the method comprises attaching a tubing defining a conduit of a predetermined volume to the dilution chamber outlet and wherein the method further comprises controlling the infusion device to perform a priming process to prepare a first volume of diluted pharmaceutical preparation in the tubing prior to connecting the tubing to the patient for intravenous delivery to the patient, wherein the first volume of diluted pharmaceutical preparation forms a first part of the predetermined dose profile.

33. The method of claim 32 wherein the priming process is at a faster rate than an initial infusion rate of the diluted pharmaceutical preparation to the patient and/or wherein the priming process is controlled to create a target concentration profile of the pharmaceutical preparation in the tubing, wherein the concentration of the pharmaceutical preparation decreases along the length of the tubing from the dilution chamber outlet to an end of the tubing proximate the patient, and/or wherein a flow rate of the priming process is 10-20 ml/minute.
